SEQUENCE LISTING (367 sequences)

SEQ ID NO: 1 - protein, 12 aa, synthetic construct
  source 1..12: note = AvGn-CD16a-A2 VH CDR1 (Exemplary)
GGSISRGSYYWG

SEQ ID NO: 2 - protein, 16 aa, synthetic construct
  source 1..16: note = AvGn-CD16a-A2 VH CDR2 (Exemplary)
TIDYSGRTYYNPSLKS

SEQ ID NO: 3 - protein, 12 aa, synthetic construct
  source 1..12: note = AvGn-CD16a-A2 VH CDR3 (Exemplary)
AFPGEYFGVFDH

SEQ ID NO: 4 - protein, 12 aa, synthetic construct
  source 1..12: note = AvGn-CD16a-A2 VL CDR1 (Exemplary)
RASQSVSSSYLA

SEQ ID NO: 5 - protein, 7 aa, synthetic construct
  source 1..7: note = AvGn-CD16a-A2 VL CDR2 (Exemplary)
DASSRAT

SEQ ID NO: 6 - protein, 10 aa, synthetic construct
  source 1..10: note = AvGn-CD16a-A2 VL CDR3 (Exemplary)
QQYGSSSLIT

SEQ ID NO: 7 - protein, 10 aa, synthetic construct
  source 1..10: note = AvGn-CD16a-A2 VH CDR1 (IMGT)
GGSISRGSYY

SEQ ID NO: 8 - protein, 8 aa, synthetic construct
  source 1..8: note = AvGn-CD16a-A2 VH CDR2 (IMGT)
TIDYSGRT

SEQ ID NO: 9 - protein, 14 aa, synthetic construct
  source 1..14: note = AvGn-CD16a-A2 VH CDR3 (IMGT)
ARAFPGEYFGVFDH

SEQ ID NO: 10 - protein, 7 aa, synthetic construct
  source 1..7: note = AvGn-CD16a-A2 VL CDR1 (IMGT)
QSVSSSY

SEQ ID NO: 11 - skipped

SEQ ID NO: 12 - protein, 10 aa, synthetic construct
  source 1..10: note = AvGn-CD16a-A2 VL CDR3 (IMGT)
QQYGSSSLIT

SEQ ID NO: 13 - protein, 7 aa, synthetic construct
  source 1..7: note = AvGn-CD16a-A2 VH CDR1 (Kabat)
RGSYYWG

SEQ ID NO: 14 - protein, 16 aa, synthetic construct
  source 1..16: note = AvGn-CD16a-A2 VH CDR2 (Kabat)
TIDYSGRTYYNPSLKS

SEQ ID NO: 15 - protein, 12 aa, synthetic construct
  source 1..12: note = AvGn-CD16a-A2 VH CDR3 (Kabat)
AFPGEYFGVFDH

SEQ ID NO: 16 - protein, 12 aa, synthetic construct
  source 1..12: note = AvGn-CD16a-A2 VL CDR1 (Kabat)
RASQSVSSSYLA

SEQ ID NO: 17 - protein, 7 aa, synthetic construct
  source 1..7: note = AvGn-CD16a-A2 VL CDR2 (Kabat)
DASSRAT

SEQ ID NO: 18 - protein, 10 aa, synthetic construct
  source 1..10: note = AvGn-CD16a-A2 VL CDR3 (Kabat)
QQYGSSSLIT

SEQ ID NO: 19 - protein, 9 aa, synthetic construct
  source 1..9: note = AvGn-CD16a-A2 VH CDR1 (Chothia)
GGSISRGSY

SEQ ID NO: 20 - protein, 5 aa, synthetic construct
  source 1..5: note = AvGn-CD16a-A2 VH CDR2 (Chothia)
DYSGR

SEQ ID NO: 21 - protein, 12 aa, synthetic construct
  source 1..12: note = AvGn-CD16a-A2 VH CDR3 (Chothia)
AFPGEYFGVFDH

SEQ ID NO: 22 - protein, 12 aa, synthetic construct
  source 1..12: note = AvGn-CD16a-A2 VL CDR1 (Chothia)
RASQSVSSSYLA

SEQ ID NO: 23 - protein, 7 aa, synthetic construct
  source 1..7: note = AvGn-CD16a-A2 VL CDR2 (Chothia)
DASSRAT

SEQ ID NO: 24 - protein, 10 aa, synthetic construct
  source 1..10: note = AvGn-CD16a-A2 VL CDR3 (Chothia)
QQYGSSSLIT

SEQ ID NO: 25 - protein, 8 aa, synthetic construct
  source 1..8: note = AvGn-CD16a-A2 VH CDR1 (Contact)
SRGSYYWG

SEQ ID NO: 26 - protein, 12 aa, synthetic construct
  source 1..12: note = AvGn-CD16a-A2 VH CDR2 (Contact)
WIGTIDYSGRTY

SEQ ID NO: 27 - protein, 13 aa, synthetic construct
  source 1..13: note = AvGn-CD16a-A2 VH CDR3 (Contact)
ARAFPGEYFGVFD

SEQ ID NO: 28 - protein, 8 aa, synthetic construct
  source 1..8: note = AvGn-CD16a-A2 VL CDR1 (Contact)
SSSYLAWY

SEQ ID NO: 29 - protein, 10 aa, synthetic construct
  source 1..10: note = AvGn-CD16a-A2 VL CDR2 (Contact)
LLIYDASSRA

SEQ ID NO: 30 - protein, 9 aa, synthetic construct
  source 1..9: note = AvGn-CD16a-A2 VL CDR3 (Contact)
QQYGSSSLI

SEQ ID NO: 31 - protein, 12 aa, synthetic construct
  source 1..12: note = AvGn-CD16a-A2 VH CDR1 (AbM)
GGSISRGSYYWG

SEQ ID NO: 32 - protein, 9 aa, synthetic construct
  source 1..9: note = AvGn-CD16a-A2 VH CDR2 (AbM)
TIDYSGRTY

SEQ ID NO: 33 - protein, 12 aa, synthetic construct
  source 1..12: note = AvGn-CD16a-A2 VH CDR3 (AbM)
AFPGEYFGVFDH

SEQ ID NO: 34 - protein, 12 aa, synthetic construct
  source 1..12: note = AvGn-CD16a-A2 VL CDR1 (AbM)
RASQSVSSSYLA

SEQ ID NO: 35 - protein, 7 aa, synthetic construct
  source 1..7: note = AvGn-CD16a-A2 VL CDR2 (AbM)
DASSRAT

SEQ ID NO: 36 - protein, 10 aa, synthetic construct
  source 1..10: note = AvGn-CD16a-A2 VL CDR3 (AbM)
QQYGSSSLIT

SEQ ID NO: 37 - protein, 122 aa, synthetic construct
  source 1..122: note = AvGn-CD16a-A2 VH Sequence
QLQLQESGPGLVKPSETLSLTCTVSGGSISRGSYYWGWIRQPPGKGLEWIGTIDYSGRTYYNPSLKSRVTISVDTSKNQFSLKLSSVTAADTAVYYCARAFPGEYFGVFDHWGQGTLVTVSS

SEQ ID NO: 38 - protein, 109 aa, synthetic construct
  source 1..109: note = AvGn-CD16a-A2 VL Sequence
EIVLTQSPGTLSLSPGERATLSCRASQSVSSSYLAWYQQKPGQAPRLLIYDASSRATGIPDRFSGSGSGTDFTLTISRLEPEDFAVYYCQQYGSSSLITFGQGTKVEIK

SEQ ID NO: 39 - protein, 12 aa, synthetic construct
  source 1..12: note = AvGn-CD16a-#1A2 VH CDR1 (Exemplary)
GGSIGRGSYYWG

SEQ ID NO: 40 - protein, 16 aa, synthetic construct
  source 1..16: note = AvGn-CD16a-#1A2 VH CDR2 (Exemplary)
TIDYSGRTYYNPSLKS

SEQ ID NO: 41 - protein, 12 aa, synthetic construct
  source 1..12: note = AvGn-CD16a-#1A2 VH CDR3 (Exemplary)
AFPGEYFAVFDH

SEQ ID NO: 42 - protein, 12 aa, synthetic construct
  source 1..12: note = AvGn-CD16a-#1A2 VL CDR1 (Exemplary)
RASQSVSSIYLA

SEQ ID NO: 43 - protein, 7 aa, synthetic construct
  source 1..7: note = AvGn-CD16a-#1A2 VL CDR2 (Exemplary)
DASSRAT

SEQ ID NO: 44 - protein, 10 aa, synthetic construct
  source 1..10: note = AvGn-CD16a-#1A2 VL CDR3 (Exemplary)
QQYGGSSLIT

SEQ ID NO: 45 - protein, 10 aa, synthetic construct
  source 1..10: note = AvGn-CD16a-#1A2 VH CDR1 (IMGT)
GGSIGRGSYY

SEQ ID NO: 46 - protein, 8 aa, synthetic construct
  source 1..8: note = AvGn-CD16a-#1A2 VH CDR2 (IMGT)
TIDYSGRT

SEQ ID NO: 47 - protein, 14 aa, synthetic construct
  source 1..14: note = AvGn-CD16a-#1A2 VH CDR3 (IMGT)
ARAFPGEYFAVFDH

SEQ ID NO: 48 - protein, 7 aa, synthetic construct
  source 1..7: note = AvGn-CD16a-#1A2 VL CDR1 (IMGT)
QSVSSIY

SEQ ID NO: 49 - skipped

SEQ ID NO: 50 - protein, 10 aa, synthetic construct
  source 1..10: note = AvGn-CD16a-#1A2 VL CDR3 (IMGT)
QQYGGSSLIT

SEQ ID NO: 51 - protein, 7 aa, synthetic construct
  source 1..7: note = AvGn-CD16a-#1A2 VH CDR1 (Kabat)
RGSYYWG

SEQ ID NO: 52 - protein, 16 aa, synthetic construct
  source 1..16: note = AvGn-CD16a-#1A2 VH CDR2 (Kabat)
TIDYSGRTYYNPSLKS

SEQ ID NO: 53 - protein, 12 aa, synthetic construct
  source 1..12: note = AvGn-CD16a-#1A2 VH CDR3 (Kabat)
AFPGEYFAVFDH

SEQ ID NO: 54 - protein, 12 aa, synthetic construct
  source 1..12: note = AvGn-CD16a-#1A2 VL CDR1 (Kabat)
RASQSVSSIYLA

SEQ ID NO: 55 - protein, 7 aa, synthetic construct
  source 1..7: note = AvGn-CD16a-#1A2 VL CDR2 (Kabat)
DASSRAT

SEQ ID NO: 56 - protein, 10 aa, synthetic construct
  source 1..10: note = AvGn-CD16a-#1A2 VL CDR3 (Kabat)
QQYGGSSLIT

SEQ ID NO: 57 - protein, 9 aa, synthetic construct
  source 1..9: note = AvGn-CD16a-#1A2 VH CDR1 (Chothia)
GGSIGRGSY

SEQ ID NO: 58 - protein, 5 aa, synthetic construct
  source 1..5: note = AvGn-CD16a-#1A2 VH CDR2 (Chothia)
DYSGR

SEQ ID NO: 59 - protein, 12 aa, synthetic construct
  source 1..12: note = AvGn-CD16a-#1A2 VH CDR3 (Chothia)
AFPGEYFAVFDH

SEQ ID NO: 60 - protein, 12 aa, synthetic construct
  source 1..12: note = AvGn-CD16a-#1A2 VL CDR1 (Chothia)
RASQSVSSIYLA

SEQ ID NO: 61 - protein, 7 aa, synthetic construct
  source 1..7: note = AvGn-CD16a-#1A2 VL CDR2 (Chothia)
DASSRAT

SEQ ID NO: 62 - protein, 10 aa, synthetic construct
  source 1..10: note = AvGn-CD16a-#1A2 VL CDR3 (Chothia)
QQYGGSSLIT

SEQ ID NO: 63 - protein, 8 aa, synthetic construct
  source 1..8: note = AvGn-CD16a-#1A2 VH CDR1 (Contact)
GRGSYYWG

SEQ ID NO: 64 - protein, 12 aa, synthetic construct
  source 1..12: note = AvGn-CD16a-#1A2 VH CDR2 (Contact)
WIGTIDYSGRTY

SEQ ID NO: 65 - protein, 13 aa, synthetic construct
  source 1..13: note = AvGn-CD16a-#1A2 VH CDR3 (Contact)
ARAFPGEYFAVFD

SEQ ID NO: 66 - protein, 8 aa, synthetic construct
  source 1..8: note = AvGn-CD16a-#1A2 VL CDR1 (Contact)
SSIYLAWY

SEQ ID NO: 67 - protein, 10 aa, synthetic construct
  source 1..10: note = AvGn-CD16a-#1A2 VL CDR2 (Contact)
LLIYDASSRA

SEQ ID NO: 68 - protein, 9 aa, synthetic construct
  source 1..9: note = AvGn-CD16a-#1A2 VL CDR3 (Contact)
QQYGGSSLI

SEQ ID NO: 69 - protein, 12 aa, synthetic construct
  source 1..12: note = AvGn-CD16a-#1A2 VH CDR1 (AbM)
GGSIGRGSYYWG

SEQ ID NO: 70 - protein, 9 aa, synthetic construct
  source 1..9: note = AvGn-CD16a-#1A2 VH CDR2 (AbM)
TIDYSGRTY

SEQ ID NO: 71 - protein, 12 aa, synthetic construct
  source 1..12: note = AvGn-CD16a-#1A2 VH CDR3 (AbM)
AFPGEYFAVFDH

SEQ ID NO: 72 - protein, 12 aa, synthetic construct
  source 1..12: note = AvGn-CD16a-#1A2 VL CDR1 (AbM)
RASQSVSSIYLA

SEQ ID NO: 73 - protein, 7 aa, synthetic construct
  source 1..7: note = AvGn-CD16a-#1A2 VL CDR2 (AbM)
DASSRAT

SEQ ID NO: 74 - protein, 10 aa, synthetic construct
  source 1..10: note = AvGn-CD16a-#1A2 VL CDR3 (AbM)
QQYGGSSLIT

SEQ ID NO: 75 - protein, 122 aa, synthetic construct
  source 1..122: note = AvGn-CD16a-#1A2 VH Sequence
QLQLQESGPGLVKPSETLSLTCTVSGGSIGRGSYYWGWIRQPPGKGLEWIGTIDYSGRTYYNPSLKSRVTISVDTSKNQFSLKLSSVTAADTAVYYCARAFPGEYFAVFDHWGQGTLVTVSS

SEQ ID NO: 76 - protein, 109 aa, synthetic construct
  source 1..109: note = AvGn-CD16a-#1A2 VL Sequence
EIVLTQSPGTLSLSPGERAILSCRASQSVSSIYLAWYQQKPGQAPRLLIYDASSRATGIPDRFSGSGSGTDFTLTISRLEPEDFAVYYCQQYGGSSLITFGQGTKVEIK

SEQ ID NO: 77 - protein, 12 aa, synthetic construct
  source 1..12: note = AvGn-CD16a-#2-1A2 VH CDR1 (Exemplary)
GGSISRGDYYWG

SEQ ID NO: 78 - protein, 16 aa, synthetic construct
  source 1..16: note = AvGn-CD16a-#2-1A2 VH CDR2 (Exemplary)
TVDYSGRTYYNPSLKS

SEQ ID NO: 79 - protein, 12 aa, synthetic construct
  source 1..12: note = AvGn-CD16a-#2-1A2 VH CDR3 (Exemplary)
AFPGEYFAVFDH

SEQ ID NO: 80 - protein, 12 aa, synthetic construct
  source 1..12: note = AvGn-CD16a-#2-1A2 VL CDR1 (Exemplary)
RASQSLSSSYLA

SEQ ID NO: 81 - protein, 7 aa, synthetic construct
  source 1..7: note = AvGn-CD16a-#2-1A2 VL CDR2 (Exemplary)
DAISRAT

SEQ ID NO: 82 - protein, 10 aa, synthetic construct
  source 1..10: note = AvGn-CD16a-#2-1A2 VL CDR3 (Exemplary)
QQYGSSRLIT

SEQ ID NO: 83 - protein, 10 aa, synthetic construct
  source 1..10: note = AvGn-CD16a-#2-1A2 VH CDR1 (IMGT)
GGSISRGDYY

SEQ ID NO: 84 - protein, 8 aa, synthetic construct
  source 1..8: note = AvGn-CD16a-#2-1A2 VH CDR2 (IMGT)
TVDYSGRT

SEQ ID NO: 85 - protein, 14 aa, synthetic construct
  source 1..14: note = AvGn-CD16a-#2-1A2 VH CDR3 (IMGT)
ARAFPGEYFAVFDH

SEQ ID NO: 86 - protein, 7 aa, synthetic construct
  source 1..7: note = AvGn-CD16a-#2-1A2 VL CDR1 (IMGT)
QSLSSSY

SEQ ID NO: 87 - skipped

SEQ ID NO: 88 - protein, 10 aa, synthetic construct
  source 1..10: note = AvGn-CD16a-#2-1A2 VL CDR3 (IMGT)
QQYGSSRLIT

SEQ ID NO: 89 - protein, 7 aa, synthetic construct
  source 1..7: note = AvGn-CD16a-#2-1A2 VH CDR1 (Kabat)
RGDYYWG

SEQ ID NO: 90 - protein, 16 aa, synthetic construct
  source 1..16: note = AvGn-CD16a-#2-1A2 VH CDR2 (Kabat)
TVDYSGRTYYNPSLKS

SEQ ID NO: 91 - protein, 12 aa, synthetic construct
  source 1..12: note = AvGn-CD16a-#2-1A2 VH CDR3 (Kabat)
AFPGEYFAVFDH

SEQ ID NO: 92 - protein, 12 aa, synthetic construct
  source 1..12: note = AvGn-CD16a-#2-1A2 VL CDR1 (Kabat)
RASQSLSSSYLA

SEQ ID NO: 93 - protein, 7 aa, synthetic construct
  source 1..7: note = AvGn-CD16a-#2-1A2 VL CDR2 (Kabat)
DAISRAT

SEQ ID NO: 94 - protein, 10 aa, synthetic construct
  source 1..10: note = AvGn-CD16a-#2-1A2 VL CDR3 (Kabat)
QQYGSSRLIT

SEQ ID NO: 95 - protein, 9 aa, synthetic construct
  source 1..9: note = AvGn-CD16a-#2-1A2 VH CDR1 (Chothia)
GGSISRGDY

SEQ ID NO: 96 - protein, 5 aa, synthetic construct
  source 1..5: note = AvGn-CD16a-#2-1A2 VH CDR2 (Chothia)
DYSGR

SEQ ID NO: 97 - protein, 12 aa, synthetic construct
  source 1..12: note = AvGn-CD16a-#2-1A2 VH CDR3 (Chothia)
AFPGEYFAVFDH

SEQ ID NO: 98 - protein, 12 aa, synthetic construct
  source 1..12: note = AvGn-CD16a-#2-1A2 VL CDR1 (Chothia)
RASQSLSSSYLA

SEQ ID NO: 99 - protein, 7 aa, synthetic construct
  source 1..7: note = AvGn-CD16a-#2-1A2 VL CDR2 (Chothia)
DAISRAT

SEQ ID NO: 100 - protein, 10 aa, synthetic construct
  source 1..10: note = AvGn-CD16a-#2-1A2 VL CDR3 (Chothia)
QQYGSSRLIT

SEQ ID NO: 101 - protein, 8 aa, synthetic construct
  source 1..8: note = AvGn-CD16a-#2-1A2 VH CDR1 (Contact)
SRGDYYWG

SEQ ID NO: 102 - protein, 12 aa, synthetic construct
  source 1..12: note = AvGn-CD16a-#2-1A2 VH CDR2 (Contact)
WIGTVDYSGRTY

SEQ ID NO: 103 - protein, 13 aa, synthetic construct
  source 1..13: note = AvGn-CD16a-#2-1A2 VH CDR3 (Contact)
ARAFPGEYFAVFD

SEQ ID NO: 104 - protein, 8 aa, synthetic construct
  source 1..8: note = AvGn-CD16a-#2-1A2 VL CDR1 (Contact)
SSSYLAWY

SEQ ID NO: 105 - protein, 10 aa, synthetic construct
  source 1..10: note = AvGn-CD16a-#2-1A2 VL CDR2 (Contact)
LLIYDAISRA

SEQ ID NO: 106 - protein, 9 aa, synthetic construct
  source 1..9: note = AvGn-CD16a-#2-1A2 VL CDR3 (Contact)
QQYGSSRLI

SEQ ID NO: 107 - protein, 12 aa, synthetic construct
  source 1..12: note = AvGn-CD16a-#2-1A2 VH CDR1 (AbM)
GGSISRGDYYWG

SEQ ID NO: 108 - protein, 9 aa, synthetic construct
  source 1..9: note = AvGn-CD16a-#2-1A2 VH CDR2 (AbM)
TVDYSGRTY

SEQ ID NO: 109 - protein, 12 aa, synthetic construct
  source 1..12: note = AvGn-CD16a-#2-1A2 VH CDR3 (AbM)
AFPGEYFAVFDH

SEQ ID NO: 110 - protein, 12 aa, synthetic construct
  source 1..12: note = AvGn-CD16a-#2-1A2 VL CDR1 (AbM)
RASQSLSSSYLA

SEQ ID NO: 111 - protein, 7 aa, synthetic construct
  source 1..7: note = AvGn-CD16a-#2-1A2 VL CDR2 (AbM)
DAISRAT

SEQ ID NO: 112 - protein, 10 aa, synthetic construct
  source 1..10: note = AvGn-CD16a-#2-1A2 VL CDR3 (AbM)
QQYGSSRLIT

SEQ ID NO: 113 - protein, 122 aa, synthetic construct
  source 1..122: note = AvGn-CD16a-#2-1A2 VH Sequence
QLQLQESGPGLVKPSETLSLTCTVSGGSISRGDYYWGWIRQPPGKGLEWIGTVDYSGRTYYNPSLKSRVTISVDTSKNQFSLKLSSVTAADTAVYYCARAFPGEYFAVFDHWGQGTLVTVSS

SEQ ID NO: 114 - protein, 109 aa, synthetic construct
  source 1..109: note = AvGn-CD16a-#2-1A2 VL Sequence
EIVLTQSPGTLSLSPGERATLSCRASQSLSSSYLAWYQQKPGQAPRLLIYDAISRATGIPDRFSGSGSGTDFTLTISRLEPEDFAVYYCQQYGSSRLITFGQGTKVEIK

SEQ ID NO: 115 - protein, 12 aa, synthetic construct
  source 1..12: note = AvGn-CD16a-#2-2A2 VH CDR1 (Exemplary)
GGSISRGDYYWG

SEQ ID NO: 116 - protein, 16 aa, synthetic construct
  source 1..16: note = AvGn-CD16a-#2-2A2 VH CDR2 (Exemplary)
TVDYSGRTYYNPSLKS

SEQ ID NO: 117 - protein, 12 aa, synthetic construct
  source 1..12: note = AvGn-CD16a-#2-2A2 VH CDR3 (Exemplary)
AFPGEYFAVFDH

SEQ ID NO: 118 - protein, 12 aa, synthetic construct
  source 1..12: note = AvGn-CD16a-#2-2A2 VL CDR1 (Exemplary)
RASQSVSSSYLA

SEQ ID NO: 119 - protein, 7 aa, synthetic construct
  source 1..7: note = AvGn-CD16a-#2-2A2 VL CDR2 (Exemplary)
GASSRAT

SEQ ID NO: 120 - protein, 10 aa, synthetic construct
  source 1..10: note = AvGn-CD16a-#2-2A2 VL CDR3 (Exemplary)
QQYGASSLIT

SEQ ID NO: 121 - protein, 10 aa, synthetic construct
  source 1..10: note = AvGn-CD16a-#2-2A2 VH CDR1 (IMGT)
GGSISRGDYY

SEQ ID NO: 122 - protein, 8 aa, synthetic construct
  source 1..8: note = AvGn-CD16a-#2-2A2 VH CDR2 (IMGT)
TVDYSGRT

SEQ ID NO: 123 - protein, 14 aa, synthetic construct
  source 1..14: note = AvGn-CD16a-#2-2A2 VH CDR3 (IMGT)
ARAFPGEYFAVFDH

SEQ ID NO: 124 - protein, 7 aa, synthetic construct
  source 1..7: note = AvGn-CD16a-#2-2A2 VL CDR1 (IMGT)
QSVSSSY

SEQ ID NO: 125 - skipped

SEQ ID NO: 126 - protein, 10 aa, synthetic construct
  source 1..10: note = AvGn-CD16a-#2-2A2 VL CDR3 (IMGT)
QQYGASSLIT

SEQ ID NO: 127 - protein, 7 aa, synthetic construct
  source 1..7: note = AvGn-CD16a-#2-2A2 VH CDR1 (Kabat)
RGDYYWG

SEQ ID NO: 128 - protein, 16 aa, synthetic construct
  source 1..16: note = AvGn-CD16a-#2-2A2 VH CDR2 (Kabat)
TVDYSGRTYYNPSLKS

SEQ ID NO: 129 - protein, 12 aa, synthetic construct
  source 1..12: note = AvGn-CD16a-#2-2A2 VH CDR3 (Kabat)
AFPGEYFAVFDH

SEQ ID NO: 130 - protein, 12 aa, synthetic construct
  source 1..12: note = AvGn-CD16a-#2-2A2 VL CDR1 (Kabat)
RASQSVSSSYLA

SEQ ID NO: 131 - protein, 7 aa, synthetic construct
  source 1..7: note = AvGn-CD16a-#2-2A2 VL CDR2 (Kabat)
GASSRAT

SEQ ID NO: 132 - protein, 10 aa, synthetic construct
  source 1..10: note = AvGn-CD16a-#2-2A2 VL CDR3 (Kabat)
QQYGASSLIT

SEQ ID NO: 133 - protein, 9 aa, synthetic construct
  source 1..9: note = AvGn-CD16a-#2-2A2 VH CDR1 (Chothia)
GGSISRGDY

SEQ ID NO: 134 - protein, 5 aa, synthetic construct
  source 1..5: note = AvGn-CD16a-#2-2A2 VH CDR2 (Chothia)
DYSGR

SEQ ID NO: 135 - protein, 12 aa, synthetic construct
  source 1..12: note = AvGn-CD16a-#2-2A2 VH CDR3 (Chothia)
AFPGEYFAVFDH

SEQ ID NO: 136 - protein, 12 aa, synthetic construct
  source 1..12: note = AvGn-CD16a-#2-2A2 VL CDR1 (Chothia)
RASQSVSSSYLA

SEQ ID NO: 137 - protein, 7 aa, synthetic construct
  source 1..7: note = AvGn-CD16a-#2-2A2 VL CDR2 (Chothia)
GASSRAT

SEQ ID NO: 138 - protein, 10 aa, synthetic construct
  source 1..10: note = AvGn-CD16a-#2-2A2 VL CDR3 (Chothia)
QQYGASSLIT

SEQ ID NO: 139 - protein, 8 aa, synthetic construct
  source 1..8: note = AvGn-CD16a-#2-2A2 VH CDR1 (Contact)
SRGDYYWG

SEQ ID NO: 140 - protein, 12 aa, synthetic construct
  source 1..12: note = AvGn-CD16a-#2-2A2 VH CDR2 (Contact)
WIGTVDYSGRTY

SEQ ID NO: 141 - protein, 13 aa, synthetic construct
  source 1..13: note = AvGn-CD16a-#2-2A2 VH CDR3 (Contact)
ARAFPGEYFAVFD

SEQ ID NO: 142 - protein, 8 aa, synthetic construct
  source 1..8: note = AvGn-CD16a-#2-2A2 VL CDR1 (Contact)
SSSYLAWY

SEQ ID NO: 143 - protein, 10 aa, synthetic construct
  source 1..10: note = AvGn-CD16a-#2-2A2 VL CDR2 (Contact)
LLIYGASSRA

SEQ ID NO: 144 - protein, 9 aa, synthetic construct
  source 1..9: note = AvGn-CD16a-#2-2A2 VL CDR3 (Contact)
QQYGASSLI

SEQ ID NO: 145 - protein, 12 aa, synthetic construct
  source 1..12: note = AvGn-CD16a-#2-2A2 VH CDR1 (AbM)
GGSISRGDYYWG

SEQ ID NO: 146 - protein, 9 aa, synthetic construct
  source 1..9: note = AvGn-CD16a-#2-2A2 VH CDR2 (AbM)
TVDYSGRTY

SEQ ID NO: 147 - protein, 12 aa, synthetic construct
  source 1..12: note = AvGn-CD16a-#2-2A2 VH CDR3 (AbM)
AFPGEYFAVFDH

SEQ ID NO: 148 - protein, 12 aa, synthetic construct
  source 1..12: note = AvGn-CD16a-#2-2A2 VL CDR1 (AbM)
RASQSVSSSYLA

SEQ ID NO: 149 - protein, 7 aa, synthetic construct
  source 1..7: note = AvGn-CD16a-#2-2A2 VL CDR2 (AbM)
GASSRAT

SEQ ID NO: 150 - protein, 10 aa, synthetic construct
  source 1..10: note = AvGn-CD16a-#2-2A2 VL CDR3 (AbM)
QQYGASSLIT

SEQ ID NO: 151 - protein, 122 aa, synthetic construct; identical to SEQ ID NO: 113
  source 1..122: note = AvGn-CD16a-#2-2A2 VH Sequence

SEQ ID NO: 152 - protein, 109 aa, synthetic construct
  source 1..109: note = AvGn-CD16a-#2-2A2 VL Sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGASSLITFGQGTKVEIK

SEQ ID NO: 153 - protein, 12 aa, synthetic construct
  source 1..12: note = AvGn-CD16a-#2-3A2 VH CDR1 (Exemplary)
GGSISRGDYYWG

SEQ ID NO: 154 - protein, 16 aa, synthetic construct
  source 1..16: note = AvGn-CD16a-#2-3A2 VH CDR2 (Exemplary)
TVDYSGRTYYNPSLKS

SEQ ID NO: 155 - protein, 12 aa, synthetic construct
  source 1..12: note = AvGn-CD16a-#2-3A2 VH CDR3 (Exemplary)
AFPGEYFAVFDH

SEQ ID NO: 156 - protein, 12 aa, synthetic construct
  source 1..12: note = AvGn-CD16a-#2-3A2 VL CDR1 (Exemplary)
RASQSVRSSYLA

SEQ ID NO: 157 - protein, 7 aa, synthetic construct
  source 1..7: note = AvGn-CD16a-#2-3A2 VL CDR2 (Exemplary)
DAFSRAT

SEQ ID NO: 158 - protein, 10 aa, synthetic construct
  source 1..10: note = AvGn-CD16a-#2-3A2 VL CDR3 (Exemplary)
QQYGSASLIT

SEQ ID NO: 159 - protein, 10 aa, synthetic construct
  source 1..10: note = AvGn-CD16a-#2-3A2 VH CDR1 (IMGT)
GGSISRGDYY

SEQ ID NO: 160 - protein, 8 aa, synthetic construct
  source 1..8: note = AvGn-CD16a-#2-3A2 VH CDR2 (IMGT)
TVDYSGRT

SEQ ID NO: 161 - protein, 14 aa, synthetic construct
  source 1..14: note = AvGn-CD16a-#2-3A2 VH CDR3 (IMGT)
ARAFPGEYFAVFDH

SEQ ID NO: 162 - protein, 7 aa, synthetic construct
  source 1..7: note = AvGn-CD16a-#2-3A2 VL CDR1 (IMGT)
QSVRSSY

SEQ ID NO: 163 - skipped

SEQ ID NO: 164 - protein, 10 aa, synthetic construct
  source 1..10: note = AvGn-CD16a-#2-3A2 VL CDR3 (IMGT)
QQYGSASLIT

SEQ ID NO: 165 - protein, 7 aa, synthetic construct
  source 1..7: note = AvGn-CD16a-#2-3A2 VH CDR1 (Kabat)
RGDYYWG

SEQ ID NO: 166 - protein, 16 aa, synthetic construct
  source 1..16: note = AvGn-CD16a-#2-3A2 VH CDR2 (Kabat)
TVDYSGRTYYNPSLKS

SEQ ID NO: 167 - protein, 12 aa, synthetic construct
  source 1..12: note = AvGn-CD16a-#2-3A2 VH CDR3 (Kabat)
AFPGEYFAVFDH

SEQ ID NO: 168 - protein, 12 aa, synthetic construct
  source 1..12: note = AvGn-CD16a-#2-3A2 VL CDR1 (Kabat)
RASQSVRSSYLA

SEQ ID NO: 169 - protein, 7 aa, synthetic construct
  source 1..7: note = AvGn-CD16a-#2-3A2 VL CDR2 (Kabat)
DAFSRAT

SEQ ID NO: 170 - protein, 10 aa, synthetic construct
  source 1..10: note = AvGn-CD16a-#2-3A2 VL CDR3 (Kabat)
QQYGSASLIT

SEQ ID NO: 171 - protein, 9 aa, synthetic construct
  source 1..9: note = AvGn-CD16a-#2-3A2 VH CDR1 (Chothia)
GGSISRGDY

SEQ ID NO: 172 - protein, 5 aa, synthetic construct
  source 1..5: note = AvGn-CD16a-#2-3A2 VH CDR2 (Chothia)
DYSGR

SEQ ID NO: 173 - protein, 12 aa, synthetic construct
  source 1..12: note = AvGn-CD16a-#2-3A2 VH CDR3 (Chothia)
AFPGEYFAVFDH

SEQ ID NO: 174 - protein, 12 aa, synthetic construct
  source 1..12: note = AvGn-CD16a-#2-3A2 VL CDR1 (Chothia)
RASQSVRSSYLA

SEQ ID NO: 175 - protein, 7 aa, synthetic construct
  source 1..7: note = AvGn-CD16a-#2-3A2 VL CDR2 (Chothia)
DAFSRAT

SEQ ID NO: 176 - protein, 10 aa, synthetic construct
  source 1..10: note = AvGn-CD16a-#2-3A2 VL CDR3 (Chothia)
QQYGSASLIT

SEQ ID NO: 177 - protein, 8 aa, synthetic construct
  source 1..8: note = AvGn-CD16a-#2-3A2 VH CDR1 (Contact)
SRGDYYWG

SEQ ID NO: 178 - protein, 12 aa, synthetic construct
  source 1..12: note = AvGn-CD16a-#2-3A2 VH CDR2 (Contact)
WIGTVDYSGRTY

SEQ ID NO: 179 - protein, 13 aa, synthetic construct
  source 1..13: note = AvGn-CD16a-#2-3A2 VH CDR3 (Contact)
ARAFPGEYFAVFD

SEQ ID NO: 180 - protein, 8 aa, synthetic construct
  source 1..8: note = AvGn-CD16a-#2-3A2 VL CDR1 (Contact)
RSSYLAWY

SEQ ID NO: 181 - protein, 10 aa, synthetic construct
  source 1..10: note = AvGn-CD16a-#2-3A2 VL CDR2 (Contact)
LLIYDAFSRA

SEQ ID NO: 182 - protein, 9 aa, synthetic construct
  source 1..9: note = AvGn-CD16a-#2-3A2 VL CDR3 (Contact)
QQYGSASLI

SEQ ID NO: 183 - protein, 12 aa, synthetic construct
  source 1..12: note = AvGn-CD16a-#2-3A2 VH CDR1 (AbM)
GGSISRGDYYWG

SEQ ID NO: 184 - protein, 9 aa, synthetic construct
  source 1..9: note = AvGn-CD16a-#2-3A2 VH CDR2 (AbM)
TVDYSGRTY

SEQ ID NO: 185 - protein, 12 aa, synthetic construct
  source 1..12: note = AvGn-CD16a-#2-3A2 VH CDR3 (AbM)
AFPGEYFAVFDH

SEQ ID NO: 186 - protein, 12 aa, synthetic construct
  source 1..12: note = AvGn-CD16a-#2-3A2 VL CDR1 (AbM)
RASQSVRSSYLA

SEQ ID NO: 187 - protein, 7 aa, synthetic construct
  source 1..7: note = AvGn-CD16a-#2-3A2 VL CDR2 (AbM)
DAFSRAT

SEQ ID NO: 188 - protein, 10 aa, synthetic construct
  source 1..10: note = AvGn-CD16a-#2-3A2 VL CDR3 (AbM)
QQYGSASLIT

SEQ ID NO: 189 - protein, 122 aa, synthetic construct; identical to SEQ ID NO: 113
  source 1..122: note = AvGn-CD16a-#2-3A2 VH Sequence

SEQ ID NO: 190 - protein, 109 aa, synthetic construct
  source 1..109: note = AvGn-CD16a-#2-3A2 VL Sequence
EIVLTQSPGTLSLSPGERATLSCRASQSVRSSYLAWYQQKPGQAPRLLIYDAFSRATGIPDRFSGSGSGTDFTLTISRLEPEDFAVYYCQQYGSASLITFGQGTKVEIK

SEQ ID NO: 191 - protein, 12 aa, synthetic construct
  source 1..12: note = AvGn-CD16a-#3A2 VH CDR1 (Exemplary)
GGSIGRGDYYWG

SEQ ID NO: 192 - protein, 16 aa, synthetic construct
  source 1..16: note = AvGn-CD16a-#3A2 VH CDR2 (Exemplary)
TIDYSGRTYYNPSLKS

SEQ ID NO: 193 - protein, 12 aa, synthetic construct
  source 1..12: note = AvGn-CD16a-#3A2 VH CDR3 (Exemplary)
AFPGEYFGVFDH

SEQ ID NO: 194 - protein, 12 aa, synthetic construct
  source 1..12: note = AvGn-CD16a-#3A2 VL CDR1 (Exemplary)
RASQSVSSSYLA

SEQ ID NO: 195 - protein, 7 aa, synthetic construct
  source 1..7: note = AvGn-CD16a-#3A2 VL CDR2 (Exemplary)
DATSRAT

SEQ ID NO: 196 - protein, 10 aa, synthetic construct
  source 1..10: note = AvGn-CD16a-#3A2 VL CDR3 (Exemplary)
QQYDSSSLIT

SEQ ID NO: 197 - protein, 10 aa, synthetic construct
  source 1..10: note = AvGn-CD16a-#3A2 VH CDR1 (IMGT)
GGSIGRGDYY

SEQ ID NO: 198 - protein, 8 aa, synthetic construct
  source 1..8: note = AvGn-CD16a-#3A2 VH CDR2 (IMGT)
TIDYSGRT

SEQ ID NO: 199 - protein, 14 aa, synthetic construct
  source 1..14: note = AvGn-CD16a-#3A2 VH CDR3 (IMGT)
ARAFPGEYFGVFDH

SEQ ID NO: 200 - protein, 7 aa, synthetic construct
  source 1..7: note = AvGn-CD16a-#3A2 VL CDR1 (IMGT)
QSVSSSY

SEQ ID NO: 201 - skipped

SEQ ID NO: 202 - protein, 10 aa, synthetic construct
  source 1..10: note = AvGn-CD16a-#3A2 VL CDR3 (IMGT)
QQYDSSSLIT

SEQ ID NO: 203 - protein, 7 aa, synthetic construct
  source 1..7: note = AvGn-CD16a-#3A2 VH CDR1 (Kabat)
RGDYYWG

SEQ ID NO: 204 - protein, 16 aa, synthetic construct
  source 1..16: note = AvGn-CD16a-#3A2 VH CDR2 (Kabat)
TIDYSGRTYYNPSLKS

SEQ ID NO: 205 - protein, 12 aa, synthetic construct
  source 1..12: note = AvGn-CD16a-#3A2 VH CDR3 (Kabat)
AFPGEYFGVFDH

SEQ ID NO: 206 - protein, 12 aa, synthetic construct
  source 1..12: note = AvGn-CD16a-#3A2 VL CDR1 (Kabat)
RASQSVSSSYLA

SEQ ID NO: 207 - protein, 7 aa, synthetic construct
  source 1..7: note = AvGn-CD16a-#3A2 VL CDR2 (Kabat)
DATSRAT

SEQ ID NO: 208 - protein, 10 aa, synthetic construct
  source 1..10: note = AvGn-CD16a-#3A2 VL CDR3 (Kabat)
QQYDSSSLIT

SEQ ID NO: 209 - protein, 9 aa, synthetic construct
  source 1..9: note = AvGn-CD16a-#3A2 VH CDR1 (Chothia)
GGSIGRGDY

SEQ ID NO: 210 - protein, 5 aa, synthetic construct
  source 1..5: note = AvGn-CD16a-#3A2 VH CDR2 (Chothia)
DYSGR

SEQ ID NO: 211 - protein, 12 aa, synthetic construct
  source 1..12: note = AvGn-CD16a-#3A2 VH CDR3 (Chothia)
AFPGEYFGVFDH

SEQ ID NO: 212 - protein, 12 aa, synthetic construct
  source 1..12: note = AvGn-CD16a-#3A2 VL CDR1 (Chothia)
RASQSVSSSYLA

SEQ ID NO: 213 - protein, 7 aa, synthetic construct
  source 1..7: note = AvGn-CD16a-#3A2 VL CDR2 (Chothia)
DATSRAT

SEQ ID NO: 214 - protein, 10 aa, synthetic construct
  source 1..10: note = AvGn-CD16a-#3A2 VL CDR3 (Chothia)
QQYDSSSLIT

SEQ ID NO: 215 - protein, 8 aa, synthetic construct
  source 1..8: note = AvGn-CD16a-#3A2 VH CDR1 (Contact)
GRGDYYWG

SEQ ID NO: 216 - protein, 12 aa, synthetic construct
  source 1..12: note = AvGn-CD16a-#3A2 VH CDR2 (Contact)
WIGTIDYSGRTY

SEQ ID NO: 217 - protein, 13 aa, synthetic construct
  source 1..13: note = AvGn-CD16a-#3A2 VH CDR3 (Contact)
ARAFPGEYFGVFD

SEQ ID NO: 218 - protein, 8 aa, synthetic construct
  source 1..8: note = AvGn-CD16a-#3A2 VL CDR1 (Contact)
SSSYLAWY

SEQ ID NO: 219 - protein, 10 aa, synthetic construct
  source 1..10: note = AvGn-CD16a-#3A2 VL CDR2 (Contact)
LLIYDATSRA

SEQ ID NO: 220 - protein, 9 aa, synthetic construct
  source 1..9: note = AvGn-CD16a-#3A2 VL CDR3 (Contact)
QQYDSSSLI

SEQ ID NO: 221 - protein, 12 aa, synthetic construct
  source 1..12: note = AvGn-CD16a-#3A2 VH CDR1 (AbM)
GGSIGRGDYYWG

SEQ ID NO: 222 - protein, 9 aa, synthetic construct
  source 1..9: note = AvGn-CD16a-#3A2 VH CDR2 (AbM)
TIDYSGRTY

SEQ ID NO: 223 - protein, 12 aa, synthetic construct
  source 1..12: note = AvGn-CD16a-#3A2 VH CDR3 (AbM)
AFPGEYFGVFDH

SEQ ID NO: 224 - protein, 12 aa, synthetic construct
  source 1..12: note = AvGn-CD16a-#3A2 VL CDR1 (AbM)
RASQSVSSSYLA

SEQ ID NO: 225 - protein, 7 aa, synthetic construct
  source 1..7: note = AvGn-CD16a-#3A2 VL CDR2 (AbM)
DATSRAT

SEQ ID NO: 226 - protein, 10 aa, synthetic construct
  source 1..10: note = AvGn-CD16a-#3A2 VL CDR3 (AbM)
QQYDSSSLIT

SEQ ID NO: 227 - protein, 122 aa, synthetic construct
  source 1..122: note = AvGn-CD16a-#3A2 VH Sequence
QLQLQESGPGLVKPSETLSLTCTVSGGSIGRGDYYWGWIRQPPGKGLEWIGTIDYSGRTYYNPSLKSRVTISVDTSKNQFSLKLSSVTAADTAVYYCARAFPGEYFGVFDHWGQGTLVTVSS

SEQ ID NO: 228 - protein, 109 aa, synthetic construct
  source 1..109: note = AvGn-CD16a-#3A2 VL Sequence
EIVLTQSPGTLSLSPGERATLSCRASQSVSSSYLAWYQQKPGQAPRLLIYDATSRATGIPDRFSGSGSGTDFTLTISRLEPEDFAVYYCQQYDSSSLITFGQGTKVEIK

SEQ ID NO: 229 - protein, 12 aa, synthetic construct
  source 1..12: note = AvGn-CD16a-#6A2 VH CDR1 (Exemplary)
GGSIGRGDYYWG

SEQ ID NO: 230 - protein, 16 aa, synthetic construct
  source 1..16: note = AvGn-CD16a-#6A2 VH CDR2 (Exemplary)
TIDYSGRTYYNPSLKS

SEQ ID NO: 231 - protein, 12 aa, synthetic construct
  source 1..12: note = AvGn-CD16a-#6A2 VH CDR3 (Exemplary)
AFPGEYFGVFDH

SEQ ID NO: 232 - protein, 12 aa, synthetic construct
  source 1..12: note = AvGn-CD16a-#6A2 VL CDR1 (Exemplary)
RASQSVSSSYLA

SEQ ID NO: 233 - protein, 7 aa, synthetic construct
  source 1..7: note = AvGn-CD16a-#6A2 VL CDR2 (Exemplary)
GASSRAT

SEQ ID NO: 234 - protein, 10 aa, synthetic construct
  source 1..10: note = AvGn-CD16a-#6A2 VL CDR3 (Exemplary)
QQYGSSPLIT

SEQ ID NO: 235 - protein, 10 aa, synthetic construct
  source 1..10: note = AvGn-CD16a-#6A2 VH CDR1 (IMGT)
GGSIGRGDYY

SEQ ID NO: 236 - protein, 8 aa, synthetic construct
  source 1..8: note = AvGn-CD16a-#6A2 VH CDR2 (IMGT)
TIDYSGRT

SEQ ID NO: 237 - protein, 14 aa, synthetic construct
  source 1..14: note = AvGn-CD16a-#6A2 VH CDR3 (IMGT)
ARAFPGEYFGVFDH

SEQ ID NO: 238 - protein, 7 aa, synthetic construct
  source 1..7: note = AvGn-CD16a-#6A2 VL CDR1 (IMGT)
QSVSSSY

SEQ ID NO: 239 - skipped

SEQ ID NO: 240 - protein, 10 aa, synthetic construct
  source 1..10: note = AvGn-CD16a-#6A2 VL CDR3 (IMGT)
QQYGSSPLIT

SEQ ID NO: 241 - protein, 7 aa, synthetic construct
  source 1..7: note = AvGn-CD16a-#6A2 VH CDR1 (Kabat)
RGDYYWG

SEQ ID NO: 242 - protein, 16 aa, synthetic construct
  source 1..16: note = AvGn-CD16a-#6A2 VH CDR2 (Kabat)
TIDYSGRTYYNPSLKS

SEQ ID NO: 243 - protein, 12 aa, synthetic construct
  source 1..12: note = AvGn-CD16a-#6A2 VH CDR3 (Kabat)
AFPGEYFGVFDH

SEQ ID NO: 244 - protein, 12 aa, synthetic construct
  source 1..12: note = AvGn-CD16a-#6A2 VL CDR1 (Kabat)
RASQSVSSSYLA

SEQ ID NO: 245 - protein, 7 aa, synthetic construct
  source 1..7: note = AvGn-CD16a-#6A2 VL CDR2 (Kabat)
GASSRAT

SEQ ID NO: 246 - protein, 10 aa, synthetic construct
  source 1..10: note = AvGn-CD16a-#6A2 VL CDR3 (Kabat)
QQYGSSPLIT

SEQ ID NO: 247 - protein, 9 aa, synthetic construct
  source 1..9: note = AvGn-CD16a-#6A2 VH CDR1 (Chothia)
GGSIGRGDY

SEQ ID NO: 248 - protein, 5 aa, synthetic construct
  source 1..5: note = AvGn-CD16a-#6A2 VH CDR2 (Chothia)
DYSGR

SEQ ID NO: 249 - protein, 12 aa, synthetic construct
  source 1..12: note = AvGn-CD16a-#6A2 VH CDR3 (Chothia)
AFPGEYFGVFDH

SEQ ID NO: 250 - protein, 12 aa, synthetic construct
  source 1..12: note = AvGn-CD16a-#6A2 VL CDR1 (Chothia)
RASQSVSSSYLA

SEQ ID NO: 251 - protein, 7 aa, synthetic construct
  source 1..7: note = AvGn-CD16a-#6A2 VL CDR2 (Chothia)
GASSRAT

SEQ ID NO: 252 - protein, 10 aa, synthetic construct
  source 1..10: note = AvGn-CD16a-#6A2 VL CDR3 (Chothia)
QQYGSSPLIT

SEQ ID NO: 253 - protein, 8 aa, synthetic construct
  source 1..8: note = AvGn-CD16a-#6A2 VH CDR1 (Contact)
GRGDYYWG

SEQ ID NO: 254 - protein, 12 aa, synthetic construct
  source 1..12: note = AvGn-CD16a-#6A2 VH CDR2 (Contact)
WIGTIDYSGRTY

SEQ ID NO: 255 - protein, 13 aa, synthetic construct
  source 1..13: note = AvGn-CD16a-#6A2 VH CDR3 (Contact)
ARAFPGEYFGVFD

SEQ ID NO: 256 - protein, 8 aa, synthetic construct
  source 1..8: note = AvGn-CD16a-#6A2 VL CDR1 (Contact)
SSSYLAWY

SEQ ID NO: 257 - protein, 10 aa, synthetic construct
  source 1..10: note = AvGn-CD16a-#6A2 VL CDR2 (Contact)
LLIYGASSRA

SEQ ID NO: 258 - protein, 9 aa, synthetic construct
  source 1..9: note = AvGn-CD16a-#6A2 VL CDR3 (Contact)
QQYGSSPLI

SEQ ID NO: 259 - protein, 12 aa, synthetic construct
  source 1..12: note = AvGn-CD16a-#6A2 VH CDR1 (AbM)
GGSIGRGDYYWG

SEQ ID NO: 260 - protein, 9 aa, synthetic construct
  source 1..9: note = AvGn-CD16a-#6A2 VH CDR2 (AbM)
TIDYSGRTY

SEQ ID NO: 261 - protein, 12 aa, synthetic construct
  source 1..12: note = AvGn-CD16a-#6A2 VH CDR3 (AbM)
AFPGEYFGVFDH

SEQ ID NO: 262 - protein, 12 aa, synthetic construct
  source 1..12: note = AvGn-CD16a-#6A2 VL CDR1 (AbM)
RASQSVSSSYLA

SEQ ID NO: 263 - protein, 7 aa, synthetic construct
  source 1..7: note = AvGn-CD16a-#6A2 VL CDR2 (AbM)
GASSRAT

SEQ ID NO: 264 - protein, 10 aa, synthetic construct
  source 1..10: note = AvGn-CD16a-#6A2 VL CDR3 (AbM)
QQYGSSPLIT

SEQ ID NO: 265 - protein, 122 aa, synthetic construct; identical to SEQ ID NO: 227
  source 1..122: note = AvGn-CD16a-#6A2 VH Sequence

SEQ ID NO: 266 - protein, 109 aa, synthetic construct
  source 1..109: note = AvGn-CD16a-#6A2 VL Sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLITFGQGTKVEIK

SEQ ID NO: 267 - protein, 12 aa, synthetic construct
  source 1..12: note = AvGn-CD16a-#10A2 VH CDR1 (Exemplary)
GGSISRGDYYWG

SEQ ID NO: 268 - protein, 16 aa, synthetic construct
  source 1..16: note = AvGn-CD16a-#10A2 VH CDR2 (Exemplary)
TIDYSGRSYYNPSLKS

SEQ ID NO: 269 - protein, 12 aa, synthetic construct
  source 1..12: note = AvGn-CD16a-#10A2 VH CDR3 (Exemplary)
AFPGEYFGVFDH

SEQ ID NO: 270 - protein, 12 aa, synthetic construct
  source 1..12: note = AvGn-CD16a-#10A2 VL CDR1 (Exemplary)
RASQRVSSSYLA

SEQ ID NO: 271 - protein, 7 aa, synthetic construct
  source 1..7: note = AvGn-CD16a-#10A2 VL CDR2 (Exemplary)
DAISRAT

SEQ ID NO: 272 - protein, 10 aa, synthetic construct
  source 1..10: note = AvGn-CD16a-#10A2 VL CDR3 (Exemplary)
QQYGSSSLIT

SEQ ID NO: 273 - protein, 10 aa, synthetic construct
  source 1..10: note = AvGn-CD16a-#10A2 VH CDR1 (IMGT)
GGSISRGDYY

SEQ ID NO: 274 - protein, 8 aa, synthetic construct
  source 1..8: note = AvGn-CD16a-#10A2 VH CDR2 (IMGT)
TIDYSGRS

SEQ ID NO: 275 - protein, 14 aa, synthetic construct
  source 1..14: note = AvGn-CD16a-#10A2 VH CDR3 (IMGT)
ARAFPGEYFGVFDH

SEQ ID NO: 276 - protein, 7 aa, synthetic construct
  source 1..7: note = AvGn-CD16a-#10A2 VL CDR1 (IMGT)
QRVSSSY

SEQ ID NO: 277 - skipped

SEQ ID NO: 278 - protein, 10 aa, synthetic construct
  source 1..10: note = AvGn-CD16a-#10A2 VL CDR3 (IMGT)
QQYGSSSLIT

SEQ ID NO: 279 - protein, 7 aa, synthetic construct
  source 1..7: note = AvGn-CD16a-#10A2 VH CDR1 (Kabat)
RGDYYWG

SEQ ID NO: 280 - protein, 16 aa, synthetic construct
  source 1..16: note = AvGn-CD16a-#10A2 VH CDR2 (Kabat)
TIDYSGRSYYNPSLKS

SEQ ID NO: 281 - protein, 12 aa, synthetic construct
  source 1..12: note = AvGn-CD16a-#10A2 VH CDR3 (Kabat)
AFPGEYFGVFDH

SEQ ID NO: 282 - protein, 12 aa, synthetic construct
  source 1..12: note = AvGn-CD16a-#10A2 VL CDR1 (Kabat)
RASQRVSSSYLA

SEQ ID NO: 283 - protein, 7 aa, synthetic construct
  source 1..7: note = AvGn-CD16a-#10A2 VL CDR2 (Kabat)
DAISRAT

SEQ ID NO: 284 - protein, 10 aa, synthetic construct
  source 1..10: note = AvGn-CD16a-#10A2 VL CDR3 (Kabat)
QQYGSSSLIT

SEQ ID NO: 285 - protein, 9 aa, synthetic construct
  source 1..9: note = AvGn-CD16a-#10A2 VH CDR1 (Chothia)
GGSISRGDY

SEQ ID NO: 286 - protein, 5 aa, synthetic construct
  source 1..5: note = AvGn-CD16a-#10A2 VH CDR2 (Chothia)
DYSGR

SEQ ID NO: 287 - protein, 12 aa, synthetic construct
  source 1..12: note = AvGn-CD16a-#10A2 VH CDR3 (Chothia)
AFPGEYFGVFDH

SEQ ID NO: 288 - protein, 12 aa, synthetic construct
  source 1..12: note = AvGn-CD16a-#10A2 VL CDR1 (Chothia)
RASQRVSSSYLA

SEQ ID NO: 289 - protein, 7 aa, synthetic construct
  source 1..7: note = AvGn-CD16a-#10A2 VL CDR2 (Chothia)
DAISRAT

SEQ ID NO: 290 - protein, 10 aa, synthetic construct
  source 1..10: note = AvGn-CD16a-#10A2 VL CDR3 (Chothia)
QQYGSSSLIT

SEQ ID NO: 291 - protein, 8 aa, synthetic construct
  source 1..8: note = AvGn-CD16a-#10A2 VH CDR1 (Contact)
SRGDYYWG

SEQ ID NO: 292 - protein, 12 aa, synthetic construct
  source 1..12: note = AvGn-CD16a-#10A2 VH CDR2 (Contact)
WIGTIDYSGRSY

SEQ ID NO: 293 - protein, 13 aa, synthetic construct
  source 1..13: note = AvGn-CD16a-#10A2 VH CDR3 (Contact)
ARAFPGEYFGVFD

SEQ ID NO: 294 - protein, 8 aa, synthetic construct
  source 1..8: note = AvGn-CD16a-#10A2 VL CDR1 (Contact)
SSSYLAWY

SEQ ID NO: 295 - protein, 10 aa, synthetic construct
  source 1..10: note = AvGn-CD16a-#10A2 VL CDR2 (Contact)
LLIYDAISRA

SEQ ID NO: 296 - protein, 9 aa, synthetic construct
  source 1..9: note = AvGn-CD16a-#10A2 VL CDR3 (Contact)
QQYGSSSLI

SEQ ID NO: 297 - protein, 12 aa, synthetic construct
  source 1..12: note = AvGn-CD16a-#10A2 VH CDR1 (AbM)
GGSISRGDYYWG

SEQ ID NO: 298 - protein, 9 aa, synthetic construct
  source 1..9: note = AvGn-CD16a-#10A2 VH CDR2 (AbM)
TIDYSGRSY

SEQ ID NO: 299 - protein, 12 aa, synthetic construct
  source 1..12: note = AvGn-CD16a-#10A2 VH CDR3 (AbM)
AFPGEYFGVFDH

SEQ ID NO: 300 - protein, 12 aa, synthetic construct
  source 1..12: note = AvGn-CD16a-#10A2 VL CDR1 (AbM)
RASQRVSSSYLA

SEQ ID NO: 301 - protein, 7 aa, synthetic construct
  source 1..7: note = AvGn-CD16a-#10A2 VL CDR2 (AbM)
DAISRAT

SEQ ID NO: 302 - protein, 10 aa, synthetic construct
  source 1..10: note = AvGn-CD16a-#10A2 VL CDR3 (AbM)
QQYGSSSLIT

SEQ ID NO: 303 - protein, 122 aa, synthetic construct
  source 1..122: note = AvGn-CD16a-#10A2 VH Sequence
QLQLQESGPGLVKPSETLSLTCTVSGGSISRGDYYWGWIRQPPGKGLEWIGTIDYSGRSYYNPSLKSRVTISVDTSKNQFSLKLSSVTAADTAVYYCARAFPGEYFGVFDHWGQGTLVTVSS

SEQ ID NO: 304 - protein, 109 aa, synthetic construct
  source 1..109: note = AvGn-CD16a-#10A2 VL Sequence
EIVLTQSPGTLSLSPGERATLSCRASQRVSSSYLAWYQQKPGQAPRLLIYDAISRATGIPDRFSGSGSGTDFTLTISRLEPEDFAVYYCQQYGSSSLITFGQGTKVEIK

SEQ ID NO: 305 - protein, 10 aa, synthetic construct
  source 1..10: note = AvGn-CD16a-#55 VH CDR1 (Exemplary)
GFTFSNYAMS

SEQ ID NO: 306 - protein, 17 aa, synthetic construct
  source 1..17: note = AvGn-CD16a-#55 VH CDR2 (Exemplary)
AISGGGSYTYYADSVKG

SEQ ID NO: 307 - protein, 11 aa, synthetic construct
  source 1..11: note = AvGn-CD16a-#55 VH CDR3 (Exemplary)
TYLRRWYYFDY

SEQ ID NO: 308 - protein, 11 aa, synthetic construct
  source 1..11: note = AvGn-CD16a-#55 VL CDR1 (Exemplary)
RASQTITSYLN

SEQ ID NO: 309 - protein, 7 aa, synthetic construct
  source 1..7: note = AvGn-CD16a-#55 VL CDR2 (Exemplary)
AASSLQS

SEQ ID NO: 310 - protein, 9 aa, synthetic construct
  source 1..9: note = AvGn-CD16a-#55 VL CDR3 (Exemplary)
QQSYTSPVT

SEQ ID NO: 311 - protein, 8 aa, synthetic construct
  source 1..8: note = AvGn-CD16a-#55 VH CDR1 (IMGT)
GFTFSNYA

SEQ ID NO: 312 - protein, 8 aa, synthetic construct
  source 1..8: note = AvGn-CD16a-#55 VH CDR2 (IMGT)
ISGGGSYT

SEQ ID NO: 313 - protein, 13 aa, synthetic construct
  source 1..13: note = AvGn-CD16a-#55 VH CDR3 (IMGT)
ARTYLRRWYYFDY

SEQ ID NO: 314 - protein, 6 aa, synthetic construct
  source 1..6: note = AvGn-CD16a-#55 VL CDR1 (IMGT)
QTITSY

SEQ ID NO: 315 - skipped

SEQ ID NO: 316 - protein, 9 aa, synthetic construct
  source 1..9: note = AvGn-CD16a-#55 VL CDR3 (IMGT)
QQSYTSPVT

SEQ ID NO: 317 - protein, 5 aa, synthetic construct
  source 1..5: note = AvGn-CD16a-#55 VH CDR1 (Kabat)
NYAMS

SEQ ID NO: 318 - protein, 17 aa, synthetic construct
  source 1..17: note = AvGn-CD16a-#55 VH CDR2 (Kabat)
AISGGGSYTYYADSVKG

SEQ ID NO: 319 - protein, 11 aa, synthetic construct
  source 1..11: note = AvGn-CD16a-#55 VH CDR3 (Kabat)
TYLRRWYYFDY

SEQ ID NO: 320 - protein, 11 aa, synthetic construct
  source 1..11: note = AvGn-CD16a-#55 VL CDR1 (Kabat)
RASQTITSYLN

SEQ ID NO: 321 - protein, 7 aa, synthetic construct
  source 1..7: note = AvGn-CD16a-#55 VL CDR2 (Kabat)
AASSLQS

SEQ ID NO: 322 - protein, 9 aa, synthetic construct
  source 1..9: note = AvGn-CD16a-#55 VL CDR3 (Kabat)
QQSYTSPVT

SEQ ID NO: 323 - protein, 7 aa, synthetic construct
  source 1..7: note = AvGn-CD16a-#55 VH CDR1 (Chothia)
GFTFSNY

SEQ ID NO: 324 - protein, 6 aa, synthetic construct
  source 1..6: note = AvGn-CD16a-#55 VH CDR2 (Chothia)
SGGGSY

SEQ ID NO: 325 - protein, 11 aa, synthetic construct
  source 1..11: note = AvGn-CD16a-#55 VH CDR3 (Chothia)
TYLRRWYYFDY

SEQ ID NO: 326 - protein, 11 aa, synthetic construct
  source 1..11: note = AvGn-CD16a-#55 VL CDR1 (Chothia)
RASQTITSYLN

SEQ ID NO: 327 - protein, 7 aa, synthetic construct
  source 1..7: note = AvGn-CD16a-#55 VL CDR2 (Chothia)
AASSLQS

SEQ ID NO: 328 - protein, 9 aa, synthetic construct
  source 1..9: note = AvGn-CD16a-#55 VL CDR3 (Chothia)
QQSYTSPVT

SEQ ID NO: 329 - protein, 6 aa, synthetic construct
  source 1..6: note = AvGn-CD16a-#55 VH CDR1 (Contact)
SNYAMS

SEQ ID NO: 330 - protein, 13 aa, synthetic construct
  source 1..13: note = AvGn-CD16a-#55 VH CDR2 (Contact)
WVSAISGGGSYTY

SEQ ID NO: 331 - protein, 12 aa, synthetic construct
  source 1..12: note = AvGn-CD16a-#55 VH CDR3 (Contact)
ARTYLRRWYYFD

SEQ ID NO: 332 - protein, 7 aa, synthetic construct
  source 1..7: note = AvGn-CD16a-#55 VL CDR1 (Contact)
TSYLNWY

SEQ ID NO: 333 - protein, 10 aa, synthetic construct
  source 1..10: note = AvGn-CD16a-#55 VL CDR2 (Contact)
LLIYAASSLQ

SEQ ID NO: 334 - protein, 8 aa, synthetic construct
  source 1..8: note = AvGn-CD16a-#55 VL CDR3 (Contact)
QQSYTSPV

SEQ ID NO: 335 - protein, 10 aa, synthetic construct
  source 1..10: note = AvGn-CD16a-#55 VH CDR1 (AbM)
GFTFSNYAMS

SEQ ID NO: 336 - protein, 10 aa, synthetic construct
  source 1..10: note = AvGn-CD16a-#55 VH CDR2 (AbM)
AISGGGSYTY

SEQ ID NO: 337 - protein, 11 aa, synthetic construct
  source 1..11: note = AvGn-CD16a-#55 VH CDR3 (AbM)
TYLRRWYYFDY

SEQ ID NO: 338 - protein, 11 aa, synthetic construct
  source 1..11: note = AvGn-CD16a-#55 VL CDR1 (AbM)
RASQTITSYLN

SEQ ID NO: 339 - protein, 7 aa, synthetic construct
  source 1..7: note = AvGn-CD16a-#55 VL CDR2 (AbM)
AASSLQS

SEQ ID NO: 340 - protein, 9 aa, synthetic construct
  source 1..9: note = AvGn-CD16a-#55 VL CDR3 (AbM)
QQSYTSPVT

SEQ ID NO: 341 - protein, 120 aa, synthetic construct
  source 1..120: note = AvGn-CD16a-#55 VH Sequence
EVQLLESGGGLVQPGGSLRLSCAASGFTFSNYAMSWVRQAPGKGLEWVSAISGGGSYTYYADSVKGRFTISRDNSKNTLYLQMNSLRAEDTAVYYCARTYLRRWYYFDYWGQGTLVTVSS

SEQ ID NO: 342 - protein, 107 aa, synthetic construct
  source 1..107: note = AvGn-CD16a-#55 VL Sequence
DIQMTQSPSSLSASVGDRVTITCRASQTITSYLNWYQQKPGKAPKLLIYAASSLQSGVPSRFSGSGSGTDFTLTISSLQPEDFATYYCQQSYTSPVTFGQGTKVEIK

SEQ ID NO: 343 - protein, 124 aa, synthetic construct
  source 1..124: note = Anti-CD19 Antibody VH Sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 344 - protein, 111 aa, synthetic construct
  source 1..111: note = Anti-CD19 Antibody VL Sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 345 - protein, 250 aa, synthetic construct
  source 1..250: note = Anti-CD19 Antibody scFv Sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 346 - protein, 246 aa, synthetic construct
  source 1..246: note = AvGn-CD16a-A2 scFv Sequence
EIVLTQSPGTLSLSPGERATLSCRASQSVSSSYLAWYQQKPGQAPRLLIYDASSRATGIPDRFSGSGSGTDFTLTISRLEPEDFAVYYCQQYGSSSLITFGQGTKVEIKGGGGSGGGGSGGGGSQLQLQESGPGLVKPSETLSLTCTVSGGSISRGSYYWGWIRQPPGKGLEWIGTIDYSGRTYYNPSLKSRVTISVDTSKNQFSLKLSSVTAADTAVYYCARAFPGEYFGVFDHWGQGTLVTVSS

SEQ ID NO: 347 - protein, 192 aa, synthetic construct
  source 1..192: note = CD16a-F
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 348 - protein, 192 aa, synthetic construct
  source 1..192: note = CD16a-V
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 349 - protein, 184 aa, synthetic construct
  source 1..184: note = CD16b-NA1
GMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTIS

SEQ ID NO: 350 - protein, 184 aa, synthetic construct
  source 1..184: note = CD16b-NA2
GMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTIS

SEQ ID NO: 351 - protein, 192 aa, synthetic construct
  source 1..192: note = Cynomolgus CD16a
GMRAEDLPKAVVFLEPQWYRVLEKDSVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQ

SEQ ID NO: 352 - protein, 254 aa, synthetic construct
  source 1..254: note = Full-length CD16a-F
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 353 - protein, 254 aa, synthetic construct
  source 1..254: note = Full-length CD16a-V
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 354 - protein, 233 aa, synthetic construct
  source 1..233: note = Full-length CD16b-NA1
MWQLLLPTALLLLVSAGMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 355 - protein, 233 aa, synthetic construct
  source 1..233: note = Full-length CD16b-NA2
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 356 - protein, 254 aa, synthetic construct
  source 1..254: note = Full-length Cynomolgus CD16a
MWQLLLPTALLLLVSAGMRAEDLPKAVVFLEPQWYRVLEKDSVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQVSFCLVMVLLFAVDTGLYFSVKKSVPSSTRDWEDHKFKWSKDPQDK

SEQ ID NO: 357 - protein, 505 aa, synthetic construct
  source 1..505: note = CD19-CD16a (A2) scFv-scFv construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGSGGSGGSEIVLTQSPGTLSLSPGERATLSCRASQSVSSSYLAWYQQKPGQAPRLLIYDASSRATGIPDRFSGSGSGTDFTLTISRLEPEDFAVYYCQQYGSSSLITFGQGTKVEIKGGGGSGGGGSGGGGSQLQLQESGPGLVKPSETLSLTCTVSGGSISRGSYYWGWIRQPPGKGLEWIGTIDYSGRTYYNPSLKSRVTISVDTSKNQFSLKLSSVTAADTAVYYCARAFPGEYFGVFDHWGQGTLVTVSS

SEQ ID NO: 358 - protein, 15 aa, synthetic construct
  source 1..15: note = Linker
GGGGSGGGGSGGGGS

SEQ ID NO: 359 - protein, 9 aa, synthetic construct
  source 1..9: note = Linker
GGSGGSGGS

SEQ ID NO: 360 - protein, 208 aa, synthetic construct
  source 1..208: note = CD16a-176F
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 361 - protein, 208 aa, synthetic construct
  source 1..208: note = CD16a-176V
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 362 - protein, 208 aa, synthetic construct
  source 1..208: note = CD16b-NA1
MWQLLLPTALLLLVSAGMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQ

SEQ ID NO: 363 - protein, 208 aa, synthetic construct
  source 1..208: note = CD16b-NA2
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQ

SEQ ID NO: 364 - protein, 100 aa, synthetic construct
  source 1..100: note = Q8SPW2 - FCG3A_MACFA
MWQLLLPTALLLLVSAGMRAEDLPKAVVFLEPQWYRVLEKDRVTLKCQGAYSPEDNSTRWFHNESLISSQTSSYFIAAARVNNSGEYRCQTSLSTLSDPV

SEQ ID NO: 365 - protein, 100 aa, synthetic construct
  source 1..100: note = O75015 - FCG3B_NA2_HUMAN
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPV

SEQ ID NO: 366 - protein, 100 aa, synthetic construct
  source 1..100: note = P08637 - FCG3A_HUMAN
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPV

SEQ ID NO: 367 - protein, 100 aa, synthetic construct
  source 1..100: note = O75015-1 - FCG3B_NA1_HUMAN
MWQLLLPTALLLLVSAGMRTEDLPKAVVFLEPQWYRVLEKDSVTLKCQGAYSPEDNSTQWFHNENLISSQASSYFIDAATVDDSGEYRCQTNLSTLSDPV

(19) United States
(12) Patent Application Publication
(10) Pub. No.: US 2026/0308258 A1
(43) Pub. Date: Oct. 8, 2026

(54) AGENTS THAT BIND TO CD16A AND USES THEREOF

(71) Applicant: AvantGen, Inc., San Diego, CA (US)

(72) Inventors: Xiaomin FAN, San Diego, CA (US); Wuxiang LIAO, San Diego, CA (US); Christine TUMANUT, Spring Valley, CA (US)

(21) Appl. No.: 19/483,168

(22) PCT Filed: May 10, 2024
(86) PCT No.: PCT/US2024/028920

§ 371 Date: Nov. 10, 2025

Related U.S. Application Data
(60) Provisional application No. 63/465,785, filed on May 11, 2023.
(60) Provisional application No. 63/508,869, filed on Jun. 16, 2023.

(51) Int. Cl. C07K 16/28; A61P 35/00; A61P 37/04

(52) U.S. Cl. CPC C07K 16/283; A61P 35/00; A61P 37/04; C07K 16/2803; C07K 2317/31; C07K 2317/34; C07K 2317/524; C07K 2317/526; C07K 2317/565; C07K 2317/622; C07K 2317/92

(57) ABSTRACT

The present disclosure provides binding agents that bind to CD 16a (e.g., antibodies, multispecific antibodies, antigen binding fragments) and uses thereof.

### CROSS-REFERENCE TO RELATED APPLICATIONS

This application claims the benefit of U.S. Provisional Patent Application No. 63/508,869, filed Jun. 16, 2023 and U.S. Provisional Patent Application No. 63/465,785, filed May 11, 2023, the disclosure of each of which is incorporated by reference herein it its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “13366-014-228_SEQ_LISTING.xml”, was created on May 8, 2024, and is 389,040 bytes in size.

### 1. FIELD

The present disclosure relates generally to binding agents (such as antibodies, multispecific antibodies, and antigen binding fragments thereof) that bind to CD16a, and methods of use thereof.

### 2. BACKGROUND

Natural killer (NK) cells are part of the innate immune system and play a key role in the first line of self-defense against viral infections and transformed, malignant cells both through direct cytotoxic effects and through production of pro-inflammatory cytokines. NK cells express a variety of activating and inhibitory receptors and the overall balance of the two results in either a response to or tolerance of the target cells (Bryceson et al., Immunol Rev. 214:73-91(2006); Myers and Miller: Nat. Rev. Clin. Oncol. 18:85-100(2021)).

CD16 is the most potent activating receptor expressed by NK cells (Bryceson et al., Immunol Rev. 214:73-91(2006)). Two forms of CD16 exist: CD16a (UniProt #P08637), a transmembrane protein that is expressed largely on NK, monocytic cells, macrophages and dendritic cells and CD16b (UniProt #075015), a GPI-anchored membrane protein that is expressed on granulocytes (Ueda et al., J Immunol. 143(4): 1274-7(1989); Ravetch and Perussia, J Expt. Med. 170:481-497(1989); Selvaraj et al., J Immunol. 143:3283-3288 (1989)).

One hurdle to generating effective NK cell engager bispecific antibodies is that the extracellular domains (ECDs) of CD16a and CD16b are highly homologous and share approximately 97% identity and 98% similarity. Therefore, a highly specific antibody against CD16a that can distinguish between the ECDs of these two forms is needed to avoid untoward activation of neutrophils and other granulocytes (Wei et al., Exp, Mol. Pathol. 101:281-289(2016); Koch and Tesar, Trans Med. Hemotherapy 44:337-350(2017); U.S. Pat. Nos. 9,035,026; 9,701,750).

One additional hurdle to generating effective anti-CD16 antibody clones suitable for an NK cell engager platform is that two different alleles occur in humans for CD16a, a 176F and a 176V (numbering based on the full-length UniProt entry) variant that differ in affinity for IgG (Wu et al., J Clin. Invest. 100:1059-1070(1997); Koene et al., Blood 90:1109-1114(1997)), and two different alleles exist for CD16b, NA1 and NA2 (Nagarajan et al., J Biol. Chem. 270:25762-70(1995)).

### 3. SUMMARY

The present disclosure provides an antibody or antigen binding fragment thereof that binds to CD16a. In some embodiments, the antibody binds to both CD16a-F and CD16a-V.

The present disclosure provides an antibody or antigen binding fragment thereof that binds to CD16a but does not bind to CD16b. In some embodiments, the antibody or antigen binding fragment thereof does not bind to CD16b-NA1 and CD16b-NA2.

The present disclosure provides an antibody or antigen binding fragment thereof that binds CD16a, e.g., an antibody or antigen binding fragment provided in Tables 1-9.

The present disclosure further provides a multispecific antibody or antigen binding fragment thereof comprising a first binding arm that binds to CD16a and a second binding arm that binds to a second antigen. In some embodiments, the second antigen is expressed on a tumor cell. In some embodiments, the second antigen is CD19. In some embodiments, the second antigen is BCMA. The first binding domain contains a VH and a VL comprising CDRs identical to any antibody or fragment thereof provided herein, for example, in Tables 1-9. The second binding domain contains a VH and a VL comprising CDRs identical to any antibody or fragment thereof provided herein, for example, in Table 10.

In some embodiments of the multispecific antibody or fragment thereof provided herein, the first binding domain and the second binding domain can be linked through a peptide bond or a peptide linker. In some embodiments, the multispecific antibody or fragment thereof comprises one polypeptide chain comprising the first binding domain and the second binding domain, wherein the first binding domain comprises a first scFv that binds to CD16, and the second binding domain comprises a second scFv that binds to CD19. In some embodiments, the first scFv is linked to the N terminal of the second scFv. In some embodiments, the first scFv is linked to the C terminal of the second scFv. In some embodiments, the polypeptide chain further comprise a first CH2 and a first CH3 of an IgG Fc region. In some embodiments, the multispecific antibody or fragment comprises two identical polypeptide chains as provided herein. In some embodiments, the first polypeptide and the second polypeptide form a bivalent format through dimerization of the CH2 and/or CH3 regions. In some embodiments, the multispecific antibody or fragment thereof comprises a mutated Fcγ1 region to eliminate Fc binding activity to Fcγ receptors.

In addition, the present disclosure provides nucleic acids encoding such a binding agent provided herein (e.g., an antibody or fragment thereof), vectors comprising one or more of such nucleic acids, and cells expressing the same, and compositions comprising any one or more of the above. Methods or uses of such binding agents, nucleic acids, vectors, cells, and compositions are also provided herein.

### 4. BRIEF DESCRIPTION OF THE DRAWINGS

FIG. 1 shows the sequence homology for the ECD of the two alleles for CD16a and the two alleles of CD16b (CD16a-176F: SEQ ID NO: 360, CD16a-176V: SEQ ID NO: 361, CD16b-NA1: SEQ ID NO: 362, CD16b-NA2: SEQ ID NO: 363).

FIGS. 2A and 2B illustrate the removal of CD16b-NA1 reactive clones from a population of CD16a-F and CD16a-V binding clones followed by positive selection for yeast clones binding biotinylated CD16a detected with APC-labeled streptavidin. FIG. 2A. FACS3 where the P1 non-CD16b binders (P1 gate of the right most panel in the upper row) were collected and used for a positive sort performed with 100 nM CD16a-F. FIG. 2B. FACS4 performed with the P3 gate from the final step of FACS3 (P3 gate of the bottom right panel of FIG. 2A), where the positive sort of the CD16b subtracted population (P1 gate of the right most panel in the upper row) was performed with 100 nM CD16a-V.

FIGS. 3A and 3B illustrate specific binding of exemplary clones to recombinant CD16a-V, CD16a-F, CD16b, and a negative control antigen by ELISA (FIG. 3A) and to CD16a-, CD16b-expressing 293 cells or untransfected 293 cells (FIG. 3B).

FIGS. 4A-4L. FIG. 4A shows binding affinity of biotinylated CD16a-F (solid square), CD16a-V (-up-pointing triangle) and CD16-NA2 (-down-pointing triangle) recombinant protein to an exemplary clone in IgG format coated on the wells of an ELISA plate. FIG. 4B shows binding kinetics of exemplary clones #1A2, #2-2A2, and the reference clone in IgG1 format (with silenced Fc) to CD16a-F, CD16a-V, Cyno-CD16, CD16b-NA1, and CD16b-NA2 determined by BLI. FIG. 4C shows the binding curves of clone #2-2A2 in IgG format (with silenced Fc) to recombinant CD16 a(F/V) and CD16b(NA1/NA2) determined by ELISA. FIG. 4D shows binding kinetics of exemplary clones #1A2, #2-2A2, and reference clone in Fab format to CD16a-F, CD16a-V in presence and absence of human IgG at 10 mg/ml determined by BLI. FIGS. 4E-4J show binding affinity of exemplary clones #1A2, #2-2A2, the reference clone in IgG1 format (with silenced Fc), an irrelevant negative control clone (CTL), and the positive control clone (3G8), to CD16a-F expressing 293 cells (FIG. 4E), CD16a-V expressing 293 cells (FIG. 4F), cyno-CD16 expressing 293 cells (FIG. 4G), CD16b-NA1 expressing 293 cells (FIG. 4H), CD16b-NA2 expressing 293 cells (FIG. 4I), and primary NK cells (FIG. 4J). FIG. 4K shows binding affinity of exemplary clone #55, the reference clone, and the negative control clone (CTL) in IgG1 format (with silenced Fc), to primary NK cells. FIG. 4L shows Tm determination for IgG1 and Fab format for clones #1A2, #2-2A2, and the reference clone.

FIGS. 5A-5H. FIG. 5A shows competition assay for binding epitope determination of exemplary clones #1A2, #2-2A2, 3G8, and the reference clone. FIGS. 5B-5E show the binding curves of the exemplary clones #1A2 (FIG. 5B), #2-2A (FIG. 5C), #55 (FIG. 5E), and the reference clone (FIG. 5D), binding to CD16(F) mutants. FIG. 5F shows competition assay for binding epitope determination of exemplary clone #55, 3G8, and the reference clone. FIG. 5G shows the alignment of CD16a(F) with CD16b(NA1), CD16b(NA2) and cynomolgus CD16a. FIG. 5H shows the impact of deglycosylation by PNGase-F on binding curves of two exemplary clones, A2 and #55, to CD16a(F), CD16a(V), CD16b(NA1) and CD16b(NA2).

FIGS. 6A-6H. FIGS. 6A-6C illustrate the structure (FIG. 6A), purity after preparative size exclusion chromatography (SEC) (FIG. 6B), and activity (FIG. 6C) of an exemplary anti-CD16a antibody clone A2 coupled to an exemplary CD19 clone in an scFv-scFv antibody format in a CD16a activation assay using CD16a-V-expressing Jurkat NFAT luciferase reporter cells in the presence of CD19-expressing Raji cells or non-target expressing MDA-MD-231 breast carcinoma cells used as controls (Symbols: squares represent Raji cells, triangles represent MDA-MD-231 cells). FIGS. 6D and 6E show the reporter cell assay results (EC50 values) of exemplary anti-CD16a antibody clones #1A2, #2-2A2, and the reference clone in scFv-scFv antibody format in presence of human IgG (FIG. 6E) or in absence of human IgG (FIG. 6D). FIG. 6F shows the results (EC50 values) of activation of reporter cell assay without Raji cells. FIGS. 6G and 6H show the reporter cell assay results of exemplary anti-CD16a antibody clone #55, and the reference clone in scFv-scFv antibody format in presence of human IgG (FIG. 6H) or in absence of human IgG (FIG. 6G).

FIGS. 7A-7E FIGS. 7A-7C illustrate the structure (FIG. 7A), purity after preparative SEC (FIG. 7B), and activity (FIG. 7C) of an exemplary anti-CD16a antibody clone coupled to an exemplary CD19 clone in an IgG4 bivalent format in a CD16a activation assay using CD16a-V expressing Jurkat NFAT luciferase reporter cells in the presence of CD19-expressing Raji cells or non-target expressing MDA-MD-231 breast carcinoma cells (Symbols: triangles represent Raji cells, squares represent MDA-MD-231 cells). FIGS. 7D and 7E show the reporter cell assay results (EC50 values) of exemplary anti-CD16a antibody clones #1A2, #2-2A2, and the reference clone in scFv-scFv-IgG4 bivalent antibody format, CD19-IgG1 with Fc enhanced mutations (S239D and 1332E) in presence of human IgG (FIG. 7E) or in absence of human IgG (FIG. 7D).

FIG. 8 illustrates the binding of an exemplary anti-CD16a clone coupled to an exemplary anti-CD19 clone in a scFv-scFv-IgG4 bivalent antibody format to human NK cells shown by flow cytometry.

FIGS. 9A-9G FIGS. 9A-9C illustrate the cytotoxicity of an exemplary anti-CD16a clone A2 coupled to an exemplary anti-CD19 clone in a scFv-scFv-IgG4 bivalent antibody format to human NK cells isolated from patients with different CD16a allelotypes. FIG. 9A: cell killing induced by an exemplary clone using NK cells from patient #1 with a CD16a-176 F/V genotype. The * indicates baseline level of cell death in the absence of the scFv-scFv-IgG4 construct. FIG. 9B: cell killing induced by an exemplary clone using NK cells from patient #2 with a CD16a-176 F/F genotype. FIG. 9C: cell killing induced by an exemplary clone using NK cells from patient #3 with a CD16a-176 F/V genotype. FIG. 9D: cell killing induced by a variant of the exemplary clone variant using NK cells from patient #3 with a CD16a-176 F/V genotype. FIGS. 9E-9G illustrate the cytotoxicity of other exemplary anti-CD16a clones coupled to an exemplary anti-CD19 clone in a scFv-scFv-IgG4 bivalent antibody format to human NK cells.

FIG. 10 illustrates the binding activity or lack thereof of an exemplary anti-CD16a clone, and two variant derivatives as full-length IgG1 to neutrophils at a concentration of 200 nM compared to the binding activity of the APC-labeled secondary detection reagent alone (middle panel of the upper row) shown by flow cytometry. The presence of CD16b was confirmed by staining for CD16b with a pan CD16 clone, 3G8, labeled with phycoerythrin (PE; X-axis, top right-most panel) and an APC labeled anti-CD15, clone HI98, (Y axis top right panel)

FIGS. 11A-11C show lack of binding activity of exemplary anti-CD16a clones A2 (FIG. 11A), #2-2A2 (FIG. 11B), #55 (FIG. 11C) in IgG format (with silenced Fc) to human neutrophils with either an NA1/NA1, NA1/NA2 or NA2/NA2 genotype compared to the binding activity of the anti-CD16 clone (3G8), the reference clone in IgG format (with silenced Fc), and/or the negative control clone (CTL) in IgG format (with silenced Fc).

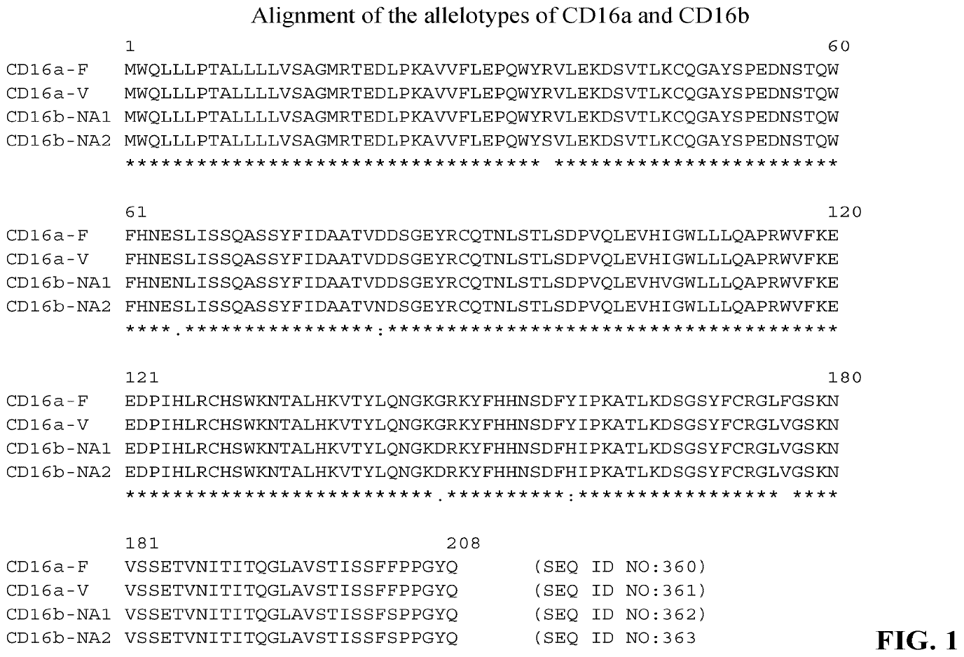

### 5. DETAILED DESCRIPTION

The present disclosure is based, at least in part, on novel binding agents that bind to CD16a. In certain aspects, such binding agents can bind both CD16a-F and CD16a-V but do not bind the two CD16b allelic forms, CD16b-NA1 and CD16b-NA2. Such agents include antibodies (e.g., monospecific or multispecific, including bispecific) that bind to CD16a, including antibodies that bind to human CD16a. In certain aspects, such binding agents are multispecific antibodies that bind to both CD16a and a tumor antigen (such as CD19) activating an anti-tumor response mediated by an immune cell. In addition, the binding agents provided herein are useful for the killing and/or removal of tumor cells. The binding agents provided herein are useful in compositions and in methods for treating a disease or disorder such as cancer.

As it would be understood, the section or subsection headings as used herein is for organizational purposes only and are not to be construed as limiting and/or separating the subject matter described.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “CD16a” refers to low affinity immunoglobulin gamma Fc region receptor III-A, a protein that in humans is encoded by the FCGR3A gene (UniProt #P08637). It is part of the cluster of differentiation cell surface molecules. Two functional polymorphisms of the CD16a have been described, 176 V and 176 F, sometimes called 158 V and 158 F, respectively, depending on when counted from the N-terminal of the mature protein or not. These two polymorphisms are also referred herein as “CD16a-V” and “CD16a-F.”

The term “CD16b” refers to low affinity immunoglobulin gamma Fc region receptor III-B, a protein that in humans is encoded by the FCGR3B gene (UniProt #075015). It is part of the cluster of differentiation cell surface molecules. Two different alleles exist for CD16b, NA1 and NA2, which are also referred herein as “CD16b-NA1” and “CD16b-NA2.”

The term “CD19” refers to Cluster of Differentiation 19 molecule, also known as B-lymphocyte antigen CD19, B-Lymphocyte Surface Antigen B4, T-Cell Surface Antigen Leu-12 and CVID3. It is a transmembrane protein that in humans is encoded by the gene CD19 (UniProt #P15391).

As used herein, the term “binding agent” or a grammatical equivalent thereof refers to a molecule (e.g., antibody) with one or more antigen-binding sites that binds an antigen. In some embodiments, a binding agent as described herein is an antibody (including a multispecific antibody and an antibody fragment, such as an antigen-binding fragment or an epitope-binding fragment) or other peptide-based molecule as well as a conjugate of an antibody, antibody fragment, or peptide-based molecule (e.g., an antibody-drug conjugate) that binds to CD16a(such as human CD16a) and/or tumor antigens (such as CD19).

The terms “antibody,” “immunoglobulin,” and “Ig” are used interchangeably herein, and are used in the broadest sense and specifically cover, for example polyclonal antibodies, monoclonal antibodies (including agonist, antagonist, neutralizing antibodies, full-length monoclonal antibodies), antibody compositions with polyepitopic or monoepitopic specificity, recombinantly produced antibodies, single domain (e.g., VHH) antibodies, monospecific antibodies, multispecific antibodies (including bispecific antibodies), synthetic antibodies, chimeric antibodies, humanized antibodies, or human versions of antibodies having full-length heavy and/or light chains. VHH as used herein refers to a domain antibody derived from a variable region of a heavy chain only antibody. Exemplary single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. VHH can also be derived from other species besides Camelidae that may produce heavy chain antibodies naturally devoid of light chain. Antibodies also include antibody fragments (and/or polypeptides that comprise antibody fragments) that retain CD16a binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable domain antibody, single variable domain, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) variable domains. For example, antibodies also include tetrameric antibodies comprising two heavy chain and two light chain molecules, an antibody light chain monomer, and an antibody heavy chain monomer. Thus, for example, the V region domain may be dimeric and contain VHH-VHH, VH-VH, VH-VL, or VL-VL dimers that bind CD16a and/or CD19. If desired, the VH and VL may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated, for example, into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9): 1126-1136, 2005). In some embodiments, antibodies comprising a VH and/or VL further contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monospecific” when used in reference to a binding agent (e.g., an antibody) as used herein denotes a binding agent that has one or more binding sites each of which binds to the same epitope of the same antigen.

The term “multispecific” when used in reference to a binding agent (e.g., an antibody) means that the binding agent is able to specifically bind to at least two distinct epitopes, for example two binding sites each formed by a pair of an antibody heavy chain variable domain (VH) and an antibody light chain variable domain (VL) or each formed by a pair of VHH domains binding to different antigens or to different epitopes on the same antigen. Such a bispecific binding agent (e.g., an antibody) may have a 1+1 format (comprising one binding site for a first antigen or epitope and one binding site for a second antigen or epitope). Other bispecific binding agent (e.g., an antibody) formats may be 2+1 or 1+2 formats (comprising two binding sites for a first antigen or epitope and one binding site for a second antigen or epitope) or 2+2 format (comprising two binding sites for a first antigen or epitope and two binding sites for a second antigen or epitope). When a bispecific binding agent (e.g., an antibody) comprises two antigen-binding sites, each may bind to a different epitope. Such a bispecific binding agent (e.g., an antibody) may bind to two different epitopes on the same antigen (e.g., epitopes on CD16a).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, or at least 90%, and in some embodiments at least 95%, 96%, 97%, 98%, or 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full-length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The term “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component or a drug (e.g., toxin). Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains or dimers of single chains.

As used herein, an “antigen” is a moiety or molecule that contains an epitope to which a binding agent (e.g., an antibody) can bind. As such, an antigen can be bound by an antibody. In some embodiments, the antigen, to which a binding agent (e.g., an antibody) described herein binds, is CD16a(e.g., human CD16a), or a fragment thereof, including a fragment that comprises one or more domains of CD16a. In some embodiments, the antigen, to which a binding agent (e.g., an antibody) described herein binds, is a tumor antigen, such as CD19 (e.g., human CD19), or a fragment thereof, including a fragment that comprises one or more domains of the tumor antigen, such as CD19.

As used herein, an “epitope” is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., CD16a or CD19. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three-dimensional protein structure. In other embodiments, an antibody requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, BIACORE™, KinExA 3000 instrument (Sapidyne Instruments, Boise, ID), the OctetQK384 system (ForteBio, Menlo Park, CA), or other assays known in the art. In some embodiments, an antibody or antigen-binding domain binds to or specifically binds to an antigen when it binds to the antigen with higher affinity than to any cross-reactive antigen as determined using experimental techniques, such as radioimmunoassays (RIAs) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen-binding domain to a “non-target” protein is less than about 10% of the binding of the antibody or antigen-binding domain to its particular target antigen, for example, as determined by fluorescence activated cell sorting (FACS) analysis or RIAs. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In another specific embodiment, molecules that specifically bind to CD16a do not bind to CD16b. In some embodiments “specifically binds” means, for instance, that a polypeptide or molecule binds a protein or target with a K_{D} of about 0.1 mM or less, but more usually less than about 1 μM. In some embodiments, “specifically binds” means that a polypeptide or molecule binds a target with a K_{D} of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding agent such as an antibody) and its binding partner (e.g., an antigen such as CD16a). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by biolayer interferometry (BLI) using, for example, the OctetQK384 system (ForteBio, Menlo Park, CA). Alternatively, the K_{D} may also be measured in a radiolabeled antigen-binding assay (RIA), for example, performed with the Fab version of an antibody of interest and its antigen (Chen, et al., (1999) J. Mol Biol 293:865-881) or using surface plasmon resonance (SPR) assays by BIACORE™, using, for example, a BIACORE™-2000 or a BIACORE™-3000 (BIACORE™, Inc., Piscataway, NJ). An “on-rate” or “rate of association” or “association rate” or “kₒₙ,” as well as an “off-rate” or “rate of dissociation” or “dissociation rate” or “k_{off},” can also be determined with the same SPR or BLI techniques described above using, for example, the OctetQK384 system (ForteBio, Menlo Park, CA) or a BIACORE™-2000 or a BIACORE™-3000 (BIACORE™, Inc., Piscataway, NJ), respectively.

The term “compete” or any grammatical variation thereof when used in the context of binding agents (e.g., antibodies) means binding agents that compete for the same epitope or binding site on a target, which includes competition between such binding agents as determined by an assay in which the binding agent under study prevents or inhibits the specific binding of a reference molecule (e.g., a reference ligand, or reference antigen-binding protein, such as a reference antibody) to a common antigen (e.g., CD16a or CD19). Numerous types of competitive binding assays can be used to determine if a test binding agent competes with a reference molecule for binding to CD16a(e.g., human CD16a) or CD19 (e.g., human CD19). Examples of assays that can be employed include solid phase direct or indirect radioimmunoassay (RIA); solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619 or Cheung, et al., (1990) Virology 176:546-552); solid phase direct labeled assay; solid phase direct labeled sandwich assay (see, e.g., Harlow and Lane, (1988) Antibodies, A Laboratory Manual, Cold Spring Harbor Press); solid phase direct label RIA using I-125 label (see, e.g., Morel et al., (1988) Molec. Immunol. 25:7-15); and direct labeled RIA (Moldenhauer et al., (1990) Scand. J. Immunol. 32:77-82). Typically, such an assay involves the use of a purified antigen (e.g., CD16a, such as human CD16a, or CD19, such as human CD19) bound to a solid surface or cells bearing either of an unlabeled test antigen-binding protein (e.g., test CD16a antibody or test CD19 antibody) or a labeled reference antigen-binding protein (e.g., reference CD16a antibody or reference CD19 antibody). Competitive inhibition may be measured by determining the amount of label bound to the solid surface or cells in the presence of the test antigen-binding protein. Usually, the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70% or 75%. In some instance, binding is inhibited by at least 80%, 85%, 90%, 95%, 96%, 97%, 98%, 99% or more.

As used herein, the term “constant region” or “constant domain” is a well-known antibody term of art and refers to an antibody portion, for example, a carboxyl terminal portion of a light and/or heavy chain which is not directly involved in binding of an antibody to an antigen but which can exhibit various effector functions, such as interaction with an Fc receptor. The term includes the portion of an immunoglobulin molecule having a generally more conserved amino acid sequence relative to an immunoglobulin variable domain.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis (such as antibody-dependent cellular phagocytosis, i.e., ADCP); down regulation of cell surface receptors (e.g., B cell receptor; BCR), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification (e.g., substituting, addition, or deletion), preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region described herein can possess at least about 80% homology with a native sequence Fc region and/or with an Fc region of a parent polypeptide, or at least about 90% homology therewith, for example, at least about 95% homology therewith. The variant Fc region herein described herein may have a loss of an effector function (e.g., silent Fc). An exemplary variant Fc region comprises LALA (Leu234Ala plus Leu235Ala) and Pro329Gly mutations to eliminate the Fcgr1 binding site for CD16.

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) or lambda (λ) based on the amino acid sequence of the constant domains. Light chain amino acid sequences are well known in the art.

The terms “antigen-binding fragment,” “antigen-binding domain,” “antigen-binding region,” and similar terms refer to that portion of an antibody, which comprises the amino acid residues that interact with an antigen and confer on the binding fragment, domain, or region its specificity and affinity for the antigen (e.g., the CDRs). “Antigen-binding fragment” as used herein includes “antibody fragment,” which comprises a portion of an antibody including one or more CDRs, such as the antigen-binding or variable region of the antibody.

Antibodies described herein include, but are not limited to, synthetic antibodies, monoclonal antibodies, recombinantly produced antibodies, multispecific antibodies (e.g., bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen-binding sites that bind to a CD16a antigen and/or a CD19 antigen.

Antibodies can be of any type (e.g., IgG, IgE, IgM, IgD, IgA or IgY), any class (e.g., IgG1, IgG2, IgG3, IgG4, IgAQ1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or a subclass thereof.

In some embodiments, an antibody is a 4-chain antibody unit comprising two heavy (H) chain/light (L) chain pairs. In further embodiments, the amino acid sequences of the H chains are identical and the amino acid sequences of the L chains are identical. In other embodiments, the amino acid sequences of the H chains are different from each other. Additionally or alternatively, the amino acid sequences of the L chains are different from each other. For example, an antibody comprises a first H/L chain pair and a second H/L chain pair, wherein the first H/L chain pair binds to a CD16a antigen and the second H/L chain pair binds to a non-CD16a tumor-target antigen (such as CD19). In some embodiments, an antibody is a 2-chain antibody unit comprising a scFv-scFv pair. In further embodiments, the amino acid sequences of the scFv are identical. In other embodiments, the amino acid sequence of the scFv are different from each other. For example, an antibody comprises a first scFv and a second scFv, wherein the first scFv binds to a CD16a antigen and the second scFv binds to a non-CD16a tumor target antigen (such as CD19). In some embodiments, the scFv-scFv chains comprise constant regions, for example, human constant regions. In some embodiments, an antibody is a 2-chain antibody unit comprising a VHH-VHH pair. In further embodiments, the amino acid sequences of the VHH are identical. In other embodiments, the amino acid sequence of the VHH are different from each other. For example, an antibody comprises a first VHH and a second VHH, wherein the first VHH binds to a CD16a antigen and the second VHH binds to a non-CD16a tumor target antigen (such as CD19). In some embodiments, the H and/or L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprises a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

An antibody or fragment thereof may preferentially bind to CD16a(such as human CD16a) and/or tumor target (such as human CD19), meaning that the antibody or fragment thereof binds CD16a and/or a tumor target (such as CD19) with greater affinity than it binds to a control protein (e.g., unrelated control proteins such as hen egg white lysozyme, or CD16b) and/or binds human CD16a and/or a tumor target (such as CD19) with greater affinity than it binds to an unrelated control protein. For example, the antibody or fragment thereof may specifically recognize and bind CD16a and/or a tumor target (such as CD19), or a portion of each thereof. “Specific binding” means that the antibody or fragment thereof binds to CD16a and/or a tumor target (such as CD19) with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind CD16a and/or a tumor target (such as CD19) substantially exclusively (e.g., is able to distinguish CD16a and/or the tumor target (such as CD19) from other known polypeptides, for example, by virtue of measurable differences in binding affinity). In some embodiments, a CD16a binding agent (e.g., an antibody) may react with CD16a sequences other than human CD16a sequences (e.g., cynomolgous monkey CD16a sequences). In other embodiments, a CD16a binding agent (e.g., an antibody) does not react with non-human (such as cynomolgous monkey) CD16a sequences. In some embodiments, a CD19 binding agent (e.g., an antibody) may react with CD19 sequences other than human CD19 sequences (e.g., cynomolgous monkey CD19 sequences). In other embodiments, a CD19 binding agent (e.g., an antibody) does not react with non-human (such as cynomolgous monkey) CD19 sequences.

The term “variable region” or “variable domain” refers to a portion of the light or heavy chains of an antibody that is generally located at the amino-terminal of the light or heavy chain, has a length of about 120 to 130 amino acids in the heavy chain and about 100 to 110 amino acids in the light chain, and is used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions (CDRs).” The variable regions of heavy and light chains each comprise four frameworks (FR1, FR2, FR3 and FR4), largely adopting a β sheet configuration, connected by three hypervariable regions, which form loops connecting, and in some cases forming part of, the β sheet structure. The hypervariable regions in each chain are held together in close proximity by the frameworks and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, (1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with its antigen. In specific embodiments, the variable region is a human variable region.

The term “hypervariable region,” “HVR,” “HV,” “complementarity determining region,” or “CDR” when used herein refers to the regions of an antibody variable region that are hypervariable in sequence and/or form structurally defined loops. Generally, antibodies comprise six hypervariable regions: three in the VH (H1 or VH CDR1, H2 or VH CDR2, and H3 or VH CDR3), and three in the VL (L1 or VL CDR1, L2 or VL CDR2, and L3 or VL CDR3). A number of hypervariable region delineations are in use and are encompassed herein. The Kabat CDRs are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT®) Information System (Lefranc et al., Dev. Comp. Immunol. 27(1):55-77(2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670(2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra) and is also illustrated below. Various systems known in the art or described herein represent different ways of delineating CDRs, and when they are used to define the same antibody, they are often considered equivalent. An Exemplary system, shown herein, combines Kabat and Chothia. The residues from each of these hypervariable regions or CDRs are exemplified in the table below.

Exemplary CDRs According to Various Numbering Systems

| | Exemplary | IMGT | Kabat | AbM | Chothia | Contact |
|---|---|---|---|---|---|---|
| V_{H} | 26-35 | 27-38 | 31-35 | 26-35 | 26-32 . . . 34 | 30-35 |
| CDR1 | | | | | | |
| V_{H} | 50-65 | 56-65 | 50-65 | 50-58 | 53-55 or | 47-58 |
| CDR2 | | | | | 52-56 | |
| V_{H} | 95-102 | 105-117 | 95-102 | 95-102 | 96-101 or | 93-101 |
| CDR3 | | | | | 95-102 | |
| V_{L} | 24-34 | 27-38 | 24-34 | 24-34 | 26-32 or | 30-36 |
| CDR1 | | | | | 24-34 | |
| V_{L} | 50-56 | 56-65 | 50-56 | 50-56 | 50-52 or | 46-55 |
| CDR2 | | | | | 50-56 | |
| V_{L} | 89-97 | 105-117 | 89-97 | 89-97 | 91-96 or | 89-96 |
| CDR3 | | | | | 89-97 | |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “vector” refers to a substance that is used to carry or include a nucleic acid sequence, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and/or inducible promoters, transcription enhancers, transcription terminators, and the like which are well known in the art. When two or more nucleic acid molecules are to be co-expressed (e.g., both an antibody heavy and light chain or an antibody VH and VL) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a binding agent as described herein), and it is further understood that expression levels can be optimized to obtain sufficient expression using methods well known in the art.

The term “pharmaceutically acceptable” as used herein means being approved by a regulatory agency of the federal or a state government, or listed in the U.S. Pharmacopeia, European Pharmacopeia or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete)) or vehicle. In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™ Other examples of pharmaceutically acceptable excipients are described in Remington and Gennaro, Remington's Pharmaceutical Sciences (18th ed. 1990). In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution. In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc. Compositions, including pharmaceutical compounds, may contain a prophylactically or therapeutically effective amount of an CD16a and/or CD19 binding agent (e.g., an antibody), for example, in isolated or purified form, together with a suitable amount of excipient so as to provide the form for proper administration to the subject (e.g., patient). The formulation should suit the mode of administration.

An “effective amount” is generally an amount sufficient to reduce the severity and/or frequency of symptoms, eliminate the symptoms and/or underlying cause, prevent or delay the occurrence of symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., an antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction, delay or amelioration of the advancement or progression of a given disease, disorder, or condition, (ii) reduction, delay or amelioration of the recurrence, development or onset of a given disease, disorder or conditions, and/or (iii) to improve or enhance the prophylactic or therapeutic effect of another therapy (e.g., a therapy other than the administration of an agent described herein). A “therapeutically effective amount” of a substance/molecule/agent of the present disclosure (e.g., a bispecific antibody) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a multispecific binding agent effective to “treat” a disease, disorder, or condition, in a subject or mammal.

The term “treating” or any grammatical variation thereof refers to reducing and/or ameliorating the severity and/or duration of a given disease, disorder or condition, and/or a symptom related thereto, such as (i) reduction, delay or amelioration of the advancement or progression of a given disease, disorder, or condition, (ii) reduction, delay or amelioration of the recurrence, development or onset of a given disease, disorder or conditions, and/or (iii) to improve or enhance the prophylactic or therapeutic effect of another therapy (e.g., a therapy other than the administration of a multispecific binding agent described herein).

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s).

The full therapeutic or prophylactic effect does not necessarily occur by administration of one dose, and may occur only after administration of a series of doses. Thus, a therapeutically or prophylactically effective amount may be administered in one or more administrations.

The terms “about” and “approximately” mean within 20%, within 15%, within 10%, within 9%, within 8%, within 7%, within 6%, within 5%, within 4%, within 3%, within 2%, within 1%, or less variation of a given value or range.

As used herein, comparative terms as used herein, such as reduce, decrease, increase, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 fold, or about 3 fold, or about 4 fold, or about 5 fold, or about 10 fold, or about 20 fold, or about 30 fold, or about 40 fold, or about 100 fold or higher than the reference. In some embodiments, such variation can refer to about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 95%, or about 96%, or about 97%, or about 98%, or about 99% of the reference.

As used in the present disclosure and claims, the singular forms “a”, “an” and “the” include plural forms unless the context clearly dictates otherwise.

In some embodiments, the terms “first,” “second,” “third,” “fourth” and similar in a component name are used to distinguish and identify more than one component sharing certain identity in their names. For example, “first antibody” and “second antibody” are used to distinguish two antibodies.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “optional” or “optionally” means that the subsequently described circumstance may or may not occur, so that the description includes instances wherein the circumstance occurs, and the instances wherein the circumstance does not occur.

### 5.2. Binding Agents

### 5.2.1. CD16a Binding Agents

In some embodiments, the present disclosure provides CD16a binding agents that can be used herein as an agent for enhancing immune responses and/or therapeutic agents. Such agents include antibodies (e.g., monospecific or multispecific, including bispecific) that bind to CD16a. Exemplary antibodies include polyclonal, monoclonal, humanized, human, bispecific, and heteroconjugate antibodies, as well as variants thereof having increased or decreased affinity or other properties.

In some embodiments, described herein are CD16a binding agents (e.g., antibodies) that bind to CD16a, including a CD16a polypeptide, a CD16a polypeptide fragment, a CD16a peptide or a CD16a epitope. In some embodiments, the CD16a binding agents are human or humanized antibodies (e.g., comprising human constant regions) that bind CD16a, including a CD16a polypeptide, a CD16a polypeptide fragment, a CD16a peptide or a CD16a epitope. In some embodiments, a CD16a binding agent (e.g., an antibody), such as a human CD16a binding agent, can bind to CD16a expressed on the surface of a mammalian (e.g., human) cell, including a CD16a expressing immune cell (e.g., an NK cell). In some embodiments, a CD16a binding agent (e.g., an antibody) binds a CD16a extracellular epitope exposed on a cell such as an immune cell. In some embodiments, described herein is a CD16a binding agent (e.g., an antibody) that binds to CD16a, such as human CD16a or a portion thereof. In some embodiments, CD16a is a human CD16a. In some embodiments, a CD16a binding agent is a human CD16a binding agent (e.g., an antibody that binds to human CD16a). In some embodiments, CD16a binding agents (e.g., antibodies) bind to both human and cyno CD16a. In other embodiments, CD16a binding agents (e.g., antibodies) bind to human CD16a but not to cyno CD16a.

In some embodiments, described herein is a CD16a binding agent (e.g., an antibody) that binds to CD16a-F. In some embodiments, described herein is a CD16a binding agent (e.g., an antibody) that binds to CD16a-V. In some embodiments, described herein is a CD16a binding agent (e.g., an antibody) that binds to CD16a-F and CD16a-V.

In some embodiments, the CD16a binding agent (e.g., an antibody) provided herein binds to CD16a(e.g., human CD16a) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by OCTET®, using, for example, an OCTET®Red96 system, or by BIACORE®, using, for example, a BIACORE®TM-2000 or a BIACORE®TM-3000. An “on-rate” or “rate of association” or “association rate” or “kon” may also be determined with the same biolayer interferometry (BLI) or surface plasmon resonance (SPR) techniques described above using, for example, the OCTET®Red96, the BIACORE®TM-2000, the BIACORE®TM-3000 system, the BIACORE®TM-8K, or the BIACORE®TM-8K+ system.

In some embodiments, the CD16a binding agent (e.g., an antibody) provided herein does not bind to CD16b(e.g., human CD16b and/or cyno CD16b). In some embodiments, the CD16a binding agent (e.g., an antibody) provided herein does not bind to CD16b-NA1. In some embodiments, the CD16a binding agent (e.g., an antibody) provided herein does not bind to CD16b-NA2. In some embodiments, the CD16a binding agent (e.g., an antibody) provided herein does not bind to CD16b-NA1 or CD16b-NA2. In some embodiments, the CD16a binding agent (e.g., an antibody) provided herein does not bind to human CD16b. In some embodiments, the CD16a binding agent (e.g., an antibody) provided herein does not bind to human CD16b or cyno CD16b. In other embodiments, the CD16a binding agent (e.g., an antibody) provided herein binds to CD16a(e.g., human CD16a) with higher affinity than to CD16b(e.g., human CD16b). In some embodiments, the binding affinity of the CD16a binding agent (e.g., an antibody) provided herein to CD16a(e.g., human CD16a) is at least 2 fold of that to CD16b(e.g., human CD16b). In some embodiments, the binding affinity of the CD16a binding agent (e.g., an antibody) provided herein to CD16a(e.g., human CD16a) is at least 5 fold of that to CD16b(e.g., human CD16b). In some embodiments, the binding affinity of the CD16a binding agent (e.g., an antibody) provided herein to CD16a(e.g., human CD16a) is at least 10 fold of that to CD16b (e.g., human CD16b). In some embodiments, the binding affinity of the CD16a binding agent (e.g., an antibody) provided herein to CD16a(e.g., human CD16a) is at least 100 fold of that to CD16b(e.g., human CD16b). In some embodiments, the binding affinity of the CD16a binding agent (e.g., an antibody) provided herein to CD16a(e.g., human CD16a) is at least 1000 fold of that to CD16b(e.g., human CD16b).

In some embodiments, the CD16a binding agents (e.g., antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in Tables 1-9. Accordingly, in some embodiments, a CD16a binding agent (e.g., an antibody) described herein comprises any one, any two, and/or all three heavy chain CDRs and/or any one, any two, and/or all three light chain CDRs from: (a) the antibody designated AvGn-CD16a-A2; (b) the antibody designated AvGn-CD16a- #1A2; (c) the antibody designated AvGn-CD16a- #2-1A2; (d) the antibody designated AvGn-CD16a- #2-2A2; (e) the antibody designated AvGn-CD16a- #2-3A2; (f) the antibody designated AvGn-CD16a- #3A2; (g) the antibody designated AvGn-CD16a- #6A2; (h) the antibody designated AvGn-CD16a- #10A2; and (i) the antibody designated AvGn-CD16a- #55, as shown in Tables 1-9. In some embodiments, a CD16a binding agent (e.g., an antibody) described herein comprises any one, any two, and/or all three heavy chain CDRs and any one, any two, and/or all three light chain CDRs from: (a) the antibody designated AvGn-CD16a-A2; (b) the antibody designated AvGn-CD16a- #1A2; (c) the antibody designated AvGn-CD16a- #2-1A2; (d) the antibody designated AvGn-CD16a- #2-2A2; (e) the antibody designated AvGn-CD16a- #2-3A2; (f) the antibody designated AvGn-CD16a- #3A2; (g) the antibody designated AvGn-CD16a- #6A2; (h) the antibody designated AvGn-CD16a- #10A2; and (i) the antibody designated AvGn-CD16a- #55, as shown in Tables 1-9.

In some embodiments, a CD16a binding agent (e.g., an antibody) comprises a VH region, which comprises a VH CDR1, a VH CDR2, and/or a VH CDR3, and/or a VL region, which comprises a VL CDR1, a VL CDR2, and/or a VL CDR3, of any one of the binding agents described herein (see, e.g., any one of Tables 1-9). Accordingly, in some embodiments, a CD16a binding agent (e.g., an antibody) described herein comprises any one, any two, and/or all three heavy chain CDRs and/or any one, any two, and/or all three light chain CDRs from Table 1. In some embodiments, a CD16a binding agent (e.g., an antibody) described herein comprises any one, any two, and/or all three heavy chain CDRs and/or any one, any two, and/or all three light chain CDRs from Table 2. In some embodiments, a CD16a binding agent (e.g., an antibody) described herein comprises any one, any two, and/or all three heavy chain CDRs and/or any one, any two, and/or all three light chain CDRs from Table 3. In some embodiments, a CD16a binding agent (e.g., an antibody) described herein comprises any one, any two, and/or all three heavy chain CDRs and/or any one, any two, and/or all three light chain CDRs from Table 4. In some embodiments, a CD16a binding agent (e.g., an antibody) described herein comprises any one, any two, and/or all three heavy chain CDRs and/or any one, any two, and/or all three light chain CDRs from Table 5. In some embodiments, a CD16a binding agent (e.g., an antibody) described herein comprises any one, any two, and/or all three heavy chain CDRs and/or any one, any two, and/or all three light chain CDRs from Table 6. In some embodiments, a CD16a binding agent (e.g., an antibody) described herein comprises any one, any two, and/or all three heavy chain CDRs and/or any one, any two, and/or all three light chain CDRs from Table 7. In some embodiments, a CD16a binding agent (e.g., an antibody) described herein comprises any one, any two, and/or all three heavy chain CDRs and/or any one, any two, and/or all three light chain CDRs from Table 8. In some embodiments, a CD16a binding agent (e.g., an antibody) described herein comprises any one, any two, and/or all three heavy chain CDRs and/or any one, any two, and/or all three light chain CDRs from Table 9.

In some embodiments, the CD16a binding agent provided herein (e.g., an antibody) comprises (i) a VH CDR1, a VH CDR2, and a VH CDR3 as set forth in a VH comprising the amino acid sequence of SEQ ID NO: 37, SEQ ID NO: 75, SEQ ID NO: 113, SEQ ID NO: 151, SEQ ID NO: 189, SEQ ID NO: 227, SEQ ID NO: 265, SEQ ID NO: 303, or SEQ ID NO: 341, and/or (ii) a VL CDR1, a VL CDR2, and a VL CDR3 as set forth in a VL comprising the amino acid sequence of SEQ ID NO: 38, SEQ ID NO: 76, SEQ ID NO: 114, SEQ ID NO: 152, SEQ ID NO: 190, SEQ ID NO: 228, SEQ ID NO: 266, SEQ ID NO: 304, or SEQ ID NO: 342.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 37 and/or a VL CDR1, a VL CDR2, and/or a VL CDR3 as set forth in a VL comprising the amino acid sequence of SEQ ID NO: 38. In some embodiments, the CD16a binding agent provided herein (e.g., an antibody) comprises a VH CDR1, a VH CDR2, and a VH CDR3 as set forth in a VH comprising the amino acid sequence of SEQ ID NO: 37, and a VL CDR1, a VL CDR2, and a VL CDR3 as set forth in a VL comprising the amino acid sequence of SEQ ID NO: 38. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia. Various exemplary CDR numbering systems are described and illustrated above in Section 5.1.

In some embodiments, the CD16a binding agent provided herein (e.g., an antibody) comprises (a) a VH region comprising a VH CDR1 having an amino acid sequence selected from the group consisting of SEQ ID NOs: 1, 7, 13, 19, 25, and 31; a VH CDR2 having an amino acid sequence selected from the group consisting of SEQ ID NOs: 2, 8, 14, 20, 26, and 32; and a VH CDR3 having an amino acid sequence selected from the group consisting of SEQ ID NOs: 3, 9, 15, 21, 27, and 33; and/or (b) a VL region comprising a VL CDR1 having an amino acid sequence selected from the group consisting of SEQ ID NOs: 4, 10, 16, 22, 28, and 34; a VL CDR2 having an amino acid sequence selected from the group consisting of SEQ ID NOs: 5, 11, 17, 23, 29, and 35; and a VL CDR3 having an amino acid sequence selected from the group consisting of SEQ ID NOs: 6, 12, 18, 24, 30, and 36.

In some embodiments, the CD16a binding agent provided herein (e.g., an antibody) comprises a VH region comprising a VH CDR1 comprising the amino acid sequence of SEQ ID NO: 1, a VH CDR2 comprising the amino acid sequence of SEQ ID NO: 2, and a VH CDR3 comprising the amino acid sequence of SEQ ID NO: 3; and a VL region comprising a VL CDR1 comprising the amino acid sequence of SEQ ID NO: 4, a VL CDR2 comprising the amino acid sequence of SEQ ID NO: 5, and a VL CDR3 comprising the amino acid sequence of SEQ ID NO: 6.

In some embodiments, the CD16a binding agent provided herein (e.g., an antibody) comprises a VH region comprising a VH CDR1 comprising the amino acid sequence of SEQ ID NO: 7, a VH CDR2 comprising the amino acid sequence of SEQ ID NO: 8, and a VH CDR3 comprising the amino acid sequence of SEQ ID NO: 9; and a VL region comprising a VL CDR1 comprising the amino acid sequence of SEQ ID NO: 10, a VL CDR2 comprising the amino acid sequence of SEQ ID NO: 11, and a VL CDR3 comprising the amino acid sequence of SEQ ID NO: 12.

In some embodiments, the CD16a binding agent provided herein (e.g., an antibody) comprises a VH region comprising a VH CDR1 comprising the amino acid sequence of SEQ ID NO: 13, a VH CDR2 comprising the amino acid sequence of SEQ ID NO: 14, and a VH CDR3 comprising the amino acid sequence of SEQ ID NO: 15; and a VL region comprising a VL CDR1 comprising the amino acid sequence of SEQ ID NO: 16, a VL CDR2 comprising the amino acid sequence of SEQ ID NO: 17, and a VL CDR3 comprising the amino acid sequence of SEQ ID NO: 18.

In some embodiments, the CD16a binding agent provided herein (e.g., an antibody) comprises a VH region comprising a VH CDR1 comprising the amino acid sequence of SEQ ID NO: 19, a VH CDR2 comprising the amino acid sequence of SEQ ID NO: 20, and a VH CDR3 comprising the amino acid sequence of SEQ ID NO: 21; and a VL region comprising a VL CDR1 comprising the amino acid sequence of SEQ ID NO: 22, a VL CDR2 comprising the amino acid sequence of SEQ ID NO: 23, and a VL CDR3 comprising the amino acid sequence of SEQ ID NO: 24.

In some embodiments, the CD16a binding agent provided herein (e.g., an antibody) comprises a VH region comprising a VH CDR1 comprising the amino acid sequence of SEQ ID NO: 25, a VH CDR2 comprising the amino acid sequence of SEQ ID NO: 26, and a VH CDR3 comprising the amino acid sequence of SEQ ID NO: 27; and a VL region comprising a VL CDR1 comprising the amino acid sequence of SEQ ID NO: 28, a VL CDR2 comprising the amino acid sequence of SEQ ID NO: 29, and a VL CDR3 comprising the amino acid sequence of SEQ ID NO: 30.

In some embodiments, the CD16a binding agent provided herein (e.g., an antibody) comprises a VH region comprising a VH CDR1 comprising the amino acid sequence of SEQ ID NO: 31, a VH CDR2 comprising the amino acid sequence of SEQ ID NO: 32, and a VH CDR3 comprising the amino acid sequence of SEQ ID NO: 33; and a VL region comprising a VL CDR1 comprising the amino acid sequence of SEQ ID NO: 34, a VL CDR2 comprising the amino acid sequence of SEQ ID NO: 35, and a VL CDR3 comprising the amino acid sequence of SEQ ID NO: 36.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 75 and/or a VL CDR1, a VL CDR2, and/or a VL CDR3 as set forth in a VL comprising the amino acid sequence of SEQ ID NO: 76. In some embodiments, the CD16a binding agent provided herein (e.g., an antibody) comprises a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 39, 45, 51, 57, 63, and 69; a VH CDR2 having an amino acid sequence selected from the group consisting of SEQ ID NOs: 40, 46, 52, 58, 64, and 70; and a VH CDR3 having an amino acid sequence selected from the group consisting of SEQ ID NOs: 41, 47, 53, 59, 65, and 71; and/or (b) a VL region comprising a VL CDR1 having an amino acid sequence selected from the group consisting of SEQ ID NOs: 42, 48, 54, 60, 66, and 72; a VL CDR2 having an amino acid sequence selected from the group consisting of SEQ ID NOs: 43, 49, 55, 61, 67, and 73; and a VL CDR3 having an amino acid sequence selected from the group consisting of SEQ ID NOs: 44, 50, 56, 62, 68, and 74.

In some embodiments, the CD16a binding agent provided herein (e.g., an antibody) comprises a VH region comprising a VH CDR1 comprising the amino acid sequence of SEQ ID NO: 39, a VH CDR2 comprising the amino acid sequence of SEQ ID NO: 40, and a VH CDR3 comprising the amino acid sequence of SEQ ID NO: 41; and a VL region comprising a VL CDR1 comprising the amino acid sequence of SEQ ID NO: 42, a VL CDR2 comprising the amino acid sequence of SEQ ID NO: 43, and a VL CDR3 comprising the amino acid sequence of SEQ ID NO: 44.

In some embodiments, the CD16a binding agent provided herein (e.g., an antibody) comprises a VH region comprising a VH CDR1 comprising the amino acid sequence of SEQ ID NO: 45, a VH CDR2 comprising the amino acid sequence of SEQ ID NO: 46, and a VH CDR3 comprising the amino acid sequence of SEQ ID NO: 47; and a VL region comprising a VL CDR1 comprising the amino acid sequence of SEQ ID NO: 48, a VL CDR2 comprising the amino acid sequence of SEQ ID NO: 49, and a VL CDR3 comprising the amino acid sequence of SEQ ID NO: 50.

In some embodiments, the CD16a binding agent provided herein (e.g., an antibody) comprises a VH region comprising a VH CDR1 comprising the amino acid sequence of SEQ ID NO: 51, a VH CDR2 comprising the amino acid sequence of SEQ ID NO: 52, and a VH CDR3 comprising the amino acid sequence of SEQ ID NO: 53; and a VL region comprising a VL CDR1 comprising the amino acid sequence of SEQ ID NO: 54, a VL CDR2 comprising the amino acid sequence of SEQ ID NO: 55, and a VL CDR3 comprising the amino acid sequence of SEQ ID NO: 56.

In some embodiments, the CD16a binding agent provided herein (e.g., an antibody) comprises a VH region comprising a VH CDR1 comprising the amino acid sequence of SEQ ID NO: 57, a VH CDR2 comprising the amino acid sequence of SEQ ID NO: 58, and a VH CDR3 comprising the amino acid sequence of SEQ ID NO: 59; and a VL region comprising a VL CDR1 comprising the amino acid sequence of SEQ ID NO: 60, a VL CDR2 comprising the amino acid sequence of SEQ ID NO: 61, and a VL CDR3 comprising the amino acid sequence of SEQ ID NO: 62.

In some embodiments, the CD16a binding agent provided herein (e.g., an antibody) comprises a VH region comprising a VH CDR1 comprising the amino acid sequence of SEQ ID NO: 63, a VH CDR2 comprising the amino acid sequence of SEQ ID NO: 64, and a VH CDR3 comprising the amino acid sequence of SEQ ID NO: 65; and a VL region comprising a VL CDR1 comprising the amino acid sequence of SEQ ID NO: 66, a VL CDR2 comprising the amino acid sequence of SEQ ID NO: 67, and a VL CDR3 comprising the amino acid sequence of SEQ ID NO: 68.

In some embodiments, the CD16a binding agent provided herein (e.g., an antibody) comprises a VH region comprising a VH CDR1 comprising the amino acid sequence of SEQ ID NO: 69, a VH CDR2 comprising the amino acid sequence of SEQ ID NO: 70, and a VH CDR3 comprising the amino acid sequence of SEQ ID NO: 71; and a VL region comprising a VL CDR1 comprising the amino acid sequence of SEQ ID NO: 72, a VL CDR2 comprising the amino acid sequence of SEQ ID NO: 73, and a VL CDR3 comprising the amino acid sequence of SEQ ID NO: 74.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 113 and/or a VL CDR1, a VL CDR2, and/or a VL CDR3 as set forth in a VL comprising the amino acid sequence of SEQ ID NO: 114. In some embodiments, the CD16a binding agent provided herein (e.g., an antibody) comprises a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 77, 83, 89, 95, 101, and 107; a VH CDR2 having an amino acid sequence selected from the group consisting of SEQ ID NOs: 78, 84, 90, 96, 102, and 108; and a VH CDR3 having an amino acid sequence selected from the group consisting of SEQ ID NOs: 79, 85, 91, 97, 103, and 109; and (b) a VL region comprising a VL CDR1 having an amino acid sequence selected from the group consisting of SEQ ID NOs: 80, 86, 92, 98, 104, and 110; a VL CDR2 having an amino acid sequence selected from the group consisting of SEQ ID NOs: 81, 87, 93, 99, 105, and 111; and a VL CDR3 having an amino acid sequence selected from the group consisting of SEQ ID NOs: 82, 88, 94, 100, 106, and 112.

In some embodiments, the CD16a binding agent provided herein (e.g., an antibody) comprises a VH region comprising a VH CDR1 comprising the amino acid sequence of SEQ ID NO: 77, a VH CDR2 comprising the amino acid sequence of SEQ ID NO: 78, and a VH CDR3 comprising the amino acid sequence of SEQ ID NO: 79; and a VL region comprising a VL CDR1 comprising the amino acid sequence of SEQ ID NO: 80, a VL CDR2 comprising the amino acid sequence of SEQ ID NO: 81, and a VL CDR3 comprising the amino acid sequence of SEQ ID NO: 82.

In some embodiments, the CD16a binding agent provided herein (e.g., an antibody) comprises a VH region comprising a VH CDR1 comprising the amino acid sequence of SEQ ID NO: 83, a VH CDR2 comprising the amino acid sequence of SEQ ID NO: 84, and a VH CDR3 comprising the amino acid sequence of SEQ ID NO: 85; and a VL region comprising a VL CDR1 comprising the amino acid sequence of SEQ ID NO: 86, a VL CDR2 comprising the amino acid sequence of SEQ ID NO: 87, and a VL CDR3 comprising the amino acid sequence of SEQ ID NO: 88.

In some embodiments, the CD16a binding agent provided herein (e.g., an antibody) comprises a VH region comprising a VH CDR1 comprising the amino acid sequence of SEQ ID NO: 89, a VH CDR2 comprising the amino acid sequence of SEQ ID NO: 90, and a VH CDR3 comprising the amino acid sequence of SEQ ID NO: 91; and a VL region comprising a VL CDR1 comprising the amino acid sequence of SEQ ID NO: 92, a VL CDR2 comprising the amino acid sequence of SEQ ID NO: 93, and a VL CDR3 comprising the amino acid sequence of SEQ ID NO: 94.

In some embodiments, the CD16a binding agent provided herein (e.g., an antibody) comprises a VH region comprising a VH CDR1 comprising the amino acid sequence of SEQ ID NO: 95, a VH CDR2 comprising the amino acid sequence of SEQ ID NO: 96, and a VH CDR3 comprising the amino acid sequence of SEQ ID NO: 97; and a VL region comprising a VL CDR1 comprising the amino acid sequence of SEQ ID NO: 98, a VL CDR2 comprising the amino acid sequence of SEQ ID NO: 99, and a VL CDR3 comprising the amino acid sequence of SEQ ID NO: 100.

In some embodiments, the CD16a binding agent provided herein (e.g., an antibody) comprises a VH region comprising a VH CDR1 comprising the amino acid sequence of SEQ ID NO: 101, a VH CDR2 comprising the amino acid sequence of SEQ ID NO: 102, and a VH CDR3 comprising the amino acid sequence of SEQ ID NO: 103; and a VL region comprising a VL CDR1 comprising the amino acid sequence of SEQ ID NO: 104, a VL CDR2 comprising the amino acid sequence of SEQ ID NO: 105, and a VL CDR3 comprising the amino acid sequence of SEQ ID NO: 106.

In some embodiments, the CD16a binding agent provided herein (e.g., an antibody) comprises a VH region comprising a VH CDR1 comprising the amino acid sequence of SEQ ID NO: 107, a VH CDR2 comprising the amino acid sequence of SEQ ID NO: 108, and a VH CDR3 comprising the amino acid sequence of SEQ ID NO: 109; and a VL region comprising a VL CDR1 comprising the amino acid sequence of SEQ ID NO: 110, a VL CDR2 comprising the amino acid sequence of SEQ ID NO: 111, and a VL CDR3 comprising the amino acid sequence of SEQ ID NO: 112.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 151, and/or a VL CDR1, a VL CDR2, and/or a VL CDR3 as set forth in a VL comprising the amino acid sequence of SEQ ID NO: 152. In some embodiments, the CD16a binding agent provided herein (e.g., an antibody) comprises a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 115, 121, 127, 133, 139, and 145; a VH CDR2 having an amino acid sequence selected from the group consisting of SEQ ID NOs: 116, 122, 128, 134, 140, and 146; and a VH CDR3 having an amino acid sequence selected from the group consisting of SEQ ID NOs: 117, 123, 129, 135, 141, and 147; and (b) a VL region comprising a VL CDR1 having an amino acid sequence selected from the group consisting of SEQ ID NOs: 118, 124, 130, 136, 142, and 148; a VL CDR2 having an amino acid sequence selected from the group consisting of SEQ ID NOs: 119, 125, 131, 137, 143, and 149; and a VL CDR3 having an amino acid sequence selected from the group consisting of SEQ ID NOs: 120, 126, 132, 138, 144, and 150.

In some embodiments, the CD16a binding agent provided herein (e.g., an antibody) comprises a VH region comprising a VH CDR1 comprising the amino acid sequence of SEQ ID NO: 115, a VH CDR2 comprising the amino acid sequence of SEQ ID NO: 116, and a VH CDR3 comprising the amino acid sequence of SEQ ID NO: 117; and a VL region comprising a VL CDR1 comprising the amino acid sequence of SEQ ID NO: 118, a VL CDR2 comprising the amino acid sequence of SEQ ID NO: 119, and a VL CDR3 comprising the amino acid sequence of SEQ ID NO: 120.

In some embodiments, the CD16a binding agent provided herein (e.g., an antibody) comprises a VH region comprising a VH CDR1 comprising the amino acid sequence of SEQ ID NO: 121, a VH CDR2 comprising the amino acid sequence of SEQ ID NO: 122, and a VH CDR3 comprising the amino acid sequence of SEQ ID NO: 123; and a VL region comprising a VL CDR1 comprising the amino acid sequence of SEQ ID NO: 124, a VL CDR2 comprising the amino acid sequence of SEQ ID NO: 125, and a VL CDR3 comprising the amino acid sequence of SEQ ID NO: 126.

In some embodiments, the CD16a binding agent provided herein (e.g., an antibody) comprises a VH region comprising a VH CDR1 comprising the amino acid sequence of SEQ ID NO: 127, a VH CDR2 comprising the amino acid sequence of SEQ ID NO: 128, and a VH CDR3 comprising the amino acid sequence of SEQ ID NO: 129; and a VL region comprising a VL CDR1 comprising the amino acid sequence of SEQ ID NO: 130, a VL CDR2 comprising the amino acid sequence of SEQ ID NO: 131, and a VL CDR3 comprising the amino acid sequence of SEQ ID NO: 132.

In some embodiments, the CD16a binding agent provided herein (e.g., an antibody) comprises a VH region comprising a VH CDR1 comprising the amino acid sequence of SEQ ID NO: 133, a VH CDR2 comprising the amino acid sequence of SEQ ID NO: 134, and a VH CDR3 comprising the amino acid sequence of SEQ ID NO: 135; and a VL region comprising a VL CDR1 comprising the amino acid sequence of SEQ ID NO: 136, a VL CDR2 comprising the amino acid sequence of SEQ ID NO: 137, and a VL CDR3 comprising the amino acid sequence of SEQ ID NO: 138.

In some embodiments, the CD16a binding agent provided herein (e.g., an antibody) comprises a VH region comprising a VH CDR1 comprising the amino acid sequence of SEQ ID NO: 139, a VH CDR2 comprising the amino acid sequence of SEQ ID NO: 140, and a VH CDR3 comprising the amino acid sequence of SEQ ID NO: 141; and a VL region comprising a VL CDR1 comprising the amino acid sequence of SEQ ID NO: 142, a VL CDR2 comprising the amino acid sequence of SEQ ID NO: 143, and a VL CDR3 comprising the amino acid sequence of SEQ ID NO: 144.

In some embodiments, the CD16a binding agent provided herein (e.g., an antibody) comprises a VH region comprising a VH CDR1 comprising the amino acid sequence of SEQ ID NO: 145, a VH CDR2 comprising the amino acid sequence of SEQ ID NO: 146, and a VH CDR3 comprising the amino acid sequence of SEQ ID NO: 147; and a VL region comprising a VL CDR1 comprising the amino acid sequence of SEQ ID NO: 148, a VL CDR2 comprising the amino acid sequence of SEQ ID NO: 149, and a VL CDR3 comprising the amino acid sequence of SEQ ID NO: 150.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 189, and/or a VL CDR1, a VL CDR2, and/or a VL CDR3 as set forth in a VL comprising the amino acid sequence of SEQ ID NO: 190. In some embodiments, the CD16a binding agent provided herein (e.g., an antibody) comprises a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 153, 159, 165, 171, 177, and 183; a VH CDR2 having an amino acid sequence selected from the group consisting of SEQ ID NOs: 154, 160, 166, 172, 178, and 184; and a VH CDR3 having an amino acid sequence selected from the group consisting of SEQ ID NOs: 155, 161, 167, 173, 179, and 185; and (b) a VL region comprising a VL CDR1 having an amino acid sequence selected from the group consisting of SEQ ID NOs: 156, 162, 168, 174, 180, and 186; a VL CDR2 having an amino acid sequence selected from the group consisting of SEQ ID NOs: 157, 163, 169, 175, 181, and 187; and a VL CDR3 having an amino acid sequence selected from the group consisting of SEQ ID NOs: 158, 164, 170, 176, 182, and 188.

In some embodiments, the CD16a binding agent provided herein (e.g., an antibody) comprises a VH region comprising a VH CDR1 comprising the amino acid sequence of SEQ ID NO: 153, a VH CDR2 comprising the amino acid sequence of SEQ ID NO: 154, and a VH CDR3 comprising the amino acid sequence of SEQ ID NO: 155; and a VL region comprising a VL CDR1 comprising the amino acid sequence of SEQ ID NO: 156, a VL CDR2 comprising the amino acid sequence of SEQ ID NO: 157, and a VL CDR3 comprising the amino acid sequence of SEQ ID NO: 158.

In some embodiments, the CD16a binding agent provided herein (e.g., an antibody) comprises a VH region comprising a VH CDR1 comprising the amino acid sequence of SEQ ID NO: 159, a VH CDR2 comprising the amino acid sequence of SEQ ID NO: 160, and a VH CDR3 comprising the amino acid sequence of SEQ ID NO: 161; and a VL region comprising a VL CDR1 comprising the amino acid sequence of SEQ ID NO: 162, a VL CDR2 comprising the amino acid sequence of SEQ ID NO: 163, and a VL CDR3 comprising the amino acid sequence of SEQ ID NO: 164.

In some embodiments, the CD16a binding agent provided herein (e.g., an antibody) comprises a VH region comprising a VH CDR1 comprising the amino acid sequence of SEQ ID NO: 165, a VH CDR2 comprising the amino acid sequence of SEQ ID NO: 166, and a VH CDR3 comprising the amino acid sequence of SEQ ID NO: 167; and a VL region comprising a VL CDR1 comprising the amino acid sequence of SEQ ID NO: 168, a VL CDR2 comprising the amino acid sequence of SEQ ID NO: 169, and a VL CDR3 comprising the amino acid sequence of SEQ ID NO: 170.

In some embodiments, the CD16a binding agent provided herein (e.g., an antibody) comprises a VH region comprising a VH CDR1 comprising the amino acid sequence of SEQ ID NO: 171, a VH CDR2 comprising the amino acid sequence of SEQ ID NO: 172, and a VH CDR3 comprising the amino acid sequence of SEQ ID NO: 173; and a VL region comprising a VL CDR1 comprising the amino acid sequence of SEQ ID NO: 174, a VL CDR2 comprising the amino acid sequence of SEQ ID NO: 175, and a VL CDR3 comprising the amino acid sequence of SEQ ID NO: 176.

In some embodiments, the CD16a binding agent provided herein (e.g., an antibody) comprises a VH region comprising a VH CDR1 comprising the amino acid sequence of SEQ ID NO: 177, a VH CDR2 comprising the amino acid sequence of SEQ ID NO: 178, and a VH CDR3 comprising the amino acid sequence of SEQ ID NO: 179; and a VL region comprising a VL CDR1 comprising the amino acid sequence of SEQ ID NO: 180, a VL CDR2 comprising the amino acid sequence of SEQ ID NO: 181, and a VL CDR3 comprising the amino acid sequence of SEQ ID NO: 182.

In some embodiments, the CD16a binding agent provided herein (e.g., an antibody) comprises a VH region comprising a VH CDR1 comprising the amino acid sequence of SEQ ID NO: 183, a VH CDR2 comprising the amino acid sequence of SEQ ID NO: 184, and a VH CDR3 comprising the amino acid sequence of SEQ ID NO: 185; and a VL region comprising a VL CDR1 comprising the amino acid sequence of SEQ ID NO: 186, a VL CDR2 comprising the amino acid sequence of SEQ ID NO: 187, and a VL CDR3 comprising the amino acid sequence of SEQ ID NO: 188.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 227, and/or a VL CDR1, a VL CDR2, and/or a VL CDR3 as set forth in a VL comprising the amino acid sequence of SEQ ID NO: 228. In some embodiments, the CD16a binding agent provided herein (e.g., an antibody) comprises a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 191, 197, 203, 209, 215, and 221; a VH CDR2 having an amino acid sequence selected from the group consisting of SEQ ID NOs: 192, 198, 204, 210, 216, and 222; and a VH CDR3 having an amino acid sequence selected from the group consisting of SEQ ID NOs: 193, 199, 205, 211, 217, and 223; and (b) a VL region comprising a VL CDR1 having an amino acid sequence selected from the group consisting of SEQ ID NOs: 194, 200, 206, 212, 218, and 224; a VL CDR2 having an amino acid sequence selected from the group consisting of SEQ ID NOs: 195, 201, 207, 213, 219, and 225; and a VL CDR3 having an amino acid sequence selected from the group consisting of SEQ ID NOs: 196, 202, 208, 214, 220, and 226.

In some embodiments, the CD16a binding agent provided herein (e.g., an antibody) comprises a VH region comprising a VH CDR1 comprising the amino acid sequence of SEQ ID NO: 191, a VH CDR2 comprising the amino acid sequence of SEQ ID NO: 192, and a VH CDR3 comprising the amino acid sequence of SEQ ID NO: 193; and a VL region comprising a VL CDR1 comprising the amino acid sequence of SEQ ID NO: 194, a VL CDR2 comprising the amino acid sequence of SEQ ID NO: 195, and a VL CDR3 comprising the amino acid sequence of SEQ ID NO: 196.

In some embodiments, the CD16a binding agent provided herein (e.g., an antibody) comprises a VH region comprising a VH CDR1 comprising the amino acid sequence of SEQ ID NO: 197, a VH CDR2 comprising the amino acid sequence of SEQ ID NO: 198, and a VH CDR3 comprising the amino acid sequence of SEQ ID NO: 199; and a VL region comprising a VL CDR1 comprising the amino acid sequence of SEQ ID NO: 200, a VL CDR2 comprising the amino acid sequence of SEQ ID NO: 201, and a VL CDR3 comprising the amino acid sequence of SEQ ID NO: 202.

In some embodiments, the CD16a binding agent provided herein (e.g., an antibody) comprises a VH region comprising a VH CDR1 comprising the amino acid sequence of SEQ ID NO: 203, a VH CDR2 comprising the amino acid sequence of SEQ ID NO: 204, and a VH CDR3 comprising the amino acid sequence of SEQ ID NO: 205; and a VL region comprising a VL CDR1 comprising the amino acid sequence of SEQ ID NO: 206, a VL CDR2 comprising the amino acid sequence of SEQ ID NO: 207, and a VL CDR3 comprising the amino acid sequence of SEQ ID NO: 208.

In some embodiments, the CD16a binding agent provided herein (e.g., an antibody) comprises a VH region comprising a VH CDR1 comprising the amino acid sequence of SEQ ID NO: 209, a VH CDR2 comprising the amino acid sequence of SEQ ID NO: 210, and a VH CDR3 comprising the amino acid sequence of SEQ ID NO: 211; and a VL region comprising a VL CDR1 comprising the amino acid sequence of SEQ ID NO: 212, a VL CDR2 comprising the amino acid sequence of SEQ ID NO: 213, and a VL CDR3 comprising the amino acid sequence of SEQ ID NO: 214.

In some embodiments, the CD16a binding agent provided herein (e.g., an antibody) comprises a VH region comprising a VH CDR1 comprising the amino acid sequence of SEQ ID NO: 215, a VH CDR2 comprising the amino acid sequence of SEQ ID NO: 216, and a VH CDR3 comprising the amino acid sequence of SEQ ID NO: 217; and a VL region comprising a VL CDR1 comprising the amino acid sequence of SEQ ID NO: 218, a VL CDR2 comprising the amino acid sequence of SEQ ID NO: 219, and a VL CDR3 comprising the amino acid sequence of SEQ ID NO: 220.

In some embodiments, the CD16a binding agent provided herein (e.g., an antibody) comprises a VH region comprising a VH CDR1 comprising the amino acid sequence of SEQ ID NO: 221, a VH CDR2 comprising the amino acid sequence of SEQ ID NO: 222, and a VH CDR3 comprising the amino acid sequence of SEQ ID NO: 223; and a VL region comprising a VL CDR1 comprising the amino acid sequence of SEQ ID NO: 224, a VL CDR2 comprising the amino acid sequence of SEQ ID NO: 225, and a VL CDR3 comprising the amino acid sequence of SEQ ID NO: 226.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 265, and/or a VL CDR1, a VL CDR2, and/or a VL CDR3 as set forth in a VL comprising the amino acid sequence of SEQ ID NO: 266. In some embodiments, the CD16a binding agent provided herein (e.g., an antibody) comprises a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 229, 235, 241, 247, 253, and 259; a VH CDR2 having an amino acid sequence selected from the group consisting of SEQ ID NOs: 230, 236, 242, 248, 254, and 260; and a VH CDR3 having an amino acid sequence selected from the group consisting of SEQ ID NOs: 231, 237, 243, 249, 255, and 261; and (b) a VL region comprising a VL CDR1 having an amino acid sequence selected from the group consisting of SEQ ID NOs: 232, 238, 244, 250, 256, and 262; a VL CDR2 having an amino acid sequence selected from the group consisting of SEQ ID NOs: 233, 239, 245, 251, 257, and 263; and a VL CDR3 having an amino acid sequence selected from the group consisting of SEQ ID NOs: 234, 240, 246, 252, 258, and 264.

In some embodiments, the CD16a binding agent provided herein (e.g., an antibody) comprises a VH region comprising a VH CDR1 comprising the amino acid sequence of SEQ ID NO: 229, a VH CDR2 comprising the amino acid sequence of SEQ ID NO: 230, and a VH CDR3 comprising the amino acid sequence of SEQ ID NO: 231; and a VL region comprising a VL CDR1 comprising the amino acid sequence of SEQ ID NO: 232, a VL CDR2 comprising the amino acid sequence of SEQ ID NO: 233, and a VL CDR3 comprising the amino acid sequence of SEQ ID NO: 234.

In some embodiments, the CD16a binding agent provided herein (e.g., an antibody) comprises a VH region comprising a VH CDR1 comprising the amino acid sequence of SEQ ID NO: 235 a VH CDR2 comprising the amino acid sequence of SEQ ID NO: 236, and a VH CDR3 comprising the amino acid sequence of SEQ ID NO: 237; and a VL region comprising a VL CDR1 comprising the amino acid sequence of SEQ ID NO: 238, a VL CDR2 comprising the amino acid sequence of SEQ ID NO: 239, and a VL CDR3 comprising the amino acid sequence of SEQ ID NO: 240.

In some embodiments, the CD16a binding agent provided herein (e.g., an antibody) comprises a VH region comprising a VH CDR1 comprising the amino acid sequence of SEQ ID NO: 241, a VH CDR2 comprising the amino acid sequence of SEQ ID NO: 242, and a VH CDR3 comprising the amino acid sequence of SEQ ID NO: 243; and a VL region comprising a VL CDR1 comprising the amino acid sequence of SEQ ID NO: 244, a VL CDR2 comprising the amino acid sequence of SEQ ID NO: 245, and a VL CDR3 comprising the amino acid sequence of SEQ ID NO: 246.

In some embodiments, the CD16a binding agent provided herein (e.g., an antibody) comprises a VH region comprising a VH CDR1 comprising the amino acid sequence of SEQ ID NO: 247, a VH CDR2 comprising the amino acid sequence of SEQ ID NO: 248, and a VH CDR3 comprising the amino acid sequence of SEQ ID NO: 249; and a VL region comprising a VL CDR1 comprising the amino acid sequence of SEQ ID NO: 250, a VL CDR2 comprising the amino acid sequence of SEQ ID NO: 251, and a VL CDR3 comprising the amino acid sequence of SEQ ID NO: 252.

In some embodiments, the CD16a binding agent provided herein (e.g., an antibody) comprises a VH region comprising a VH CDR1 comprising the amino acid sequence of SEQ ID NO: 253, a VH CDR2 comprising the amino acid sequence of SEQ ID NO: 254, and a VH CDR3 comprising the amino acid sequence of SEQ ID NO: 255; and a VL region comprising a VL CDR1 comprising the amino acid sequence of SEQ ID NO: 256, a VL CDR2 comprising the amino acid sequence of SEQ ID NO: 257, and a VL CDR3 comprising the amino acid sequence of SEQ ID NO: 258.

In some embodiments, the CD16a binding agent provided herein (e.g., an antibody) comprises a VH region comprising a VH CDR1 comprising the amino acid sequence of SEQ ID NO: 259, a VH CDR2 comprising the amino acid sequence of SEQ ID NO: 260, and a VH CDR3 comprising the amino acid sequence of SEQ ID NO: 261; and a VL region comprising a VL CDR1 comprising the amino acid sequence of SEQ ID NO: 262, a VL CDR2 comprising the amino acid sequence of SEQ ID NO: 263, and a VL CDR3 comprising the amino acid sequence of SEQ ID NO: 264.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 303, and/or a VL CDR1, a VL CDR2, and/or a VL CDR3 as set forth in a VL comprising the amino acid sequence of SEQ ID NO: 304. In some embodiments, the CD16a binding agent provided herein (e.g., an antibody) comprises a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 267, 273, 279, 285, 291, and 297; a VH CDR2 having an amino acid sequence selected from the group consisting of SEQ ID NOs: 268, 274, 280, 286, 292, and 298; and a VH CDR3 having an amino acid sequence selected from the group consisting of SEQ ID NOs: 269, 275, 281, 287, 293, and 299; and (b) a VL region comprising a VL CDR1 having an amino acid sequence selected from the group consisting of SEQ ID NOs: 270, 276, 282, 288, 294, and 300; a VL CDR2 having an amino acid sequence selected from the group consisting of SEQ ID NOs: 271, 277, 283, 289, 295, and 301; and a VL CDR3 having an amino acid sequence selected from the group consisting of SEQ ID NOs: 272, 278, 284, 290, 296, and 302.

In some embodiments, the CD16a binding agent provided herein (e.g., an antibody) comprises a VH region comprising a VH CDR1 comprising the amino acid sequence of SEQ ID NO: 267, a VH CDR2 comprising the amino acid sequence of SEQ ID NO: 268, and a VH CDR3 comprising the amino acid sequence of SEQ ID NO: 269; and a VL region comprising a VL CDR1 comprising the amino acid sequence of SEQ ID NO: 270, a VL CDR2 comprising the amino acid sequence of SEQ ID NO: 271, and a VL CDR3 comprising the amino acid sequence of SEQ ID NO: 272.

In some embodiments, the CD16a binding agent provided herein (e.g., an antibody) comprises a VH region comprising a VH CDR1 comprising the amino acid sequence of SEQ ID NO: 273 a VH CDR2 comprising the amino acid sequence of SEQ ID NO: 274, and a VH CDR3 comprising the amino acid sequence of SEQ ID NO: 275; and a VL region comprising a VL CDR1 comprising the amino acid sequence of SEQ ID NO: 276, a VL CDR2 comprising the amino acid sequence of SEQ ID NO: 277, and a VL CDR3 comprising the amino acid sequence of SEQ ID NO: 278.

In some embodiments, the CD16a binding agent provided herein (e.g., an antibody) comprises a VH region comprising a VH CDR1 comprising the amino acid sequence of SEQ ID NO: 279, a VH CDR2 comprising the amino acid sequence of SEQ ID NO: 280, and a VH CDR3 comprising the amino acid sequence of SEQ ID NO: 281; and a VL region comprising a VL CDR1 comprising the amino acid sequence of SEQ ID NO: 282, a VL CDR2 comprising the amino acid sequence of SEQ ID NO: 283, and a VL CDR3 comprising the amino acid sequence of SEQ ID NO: 284.

In some embodiments, the CD16a binding agent provided herein (e.g., an antibody) comprises a VH region comprising a VH CDR1 comprising the amino acid sequence of SEQ ID NO: 285, a VH CDR2 comprising the amino acid sequence of SEQ ID NO: 286, and a VH CDR3 comprising the amino acid sequence of SEQ ID NO: 287; and a VL region comprising a VL CDR1 comprising the amino acid sequence of SEQ ID NO: 288, a VL CDR2 comprising the amino acid sequence of SEQ ID NO: 289, and a VL CDR3 comprising the amino acid sequence of SEQ ID NO: 290.

In some embodiments, the CD16a binding agent provided herein (e.g., an antibody) comprises a VH region comprising a VH CDR1 comprising the amino acid sequence of SEQ ID NO: 291, a VH CDR2 comprising the amino acid sequence of SEQ ID NO: 292, and a VH CDR3 comprising the amino acid sequence of SEQ ID NO: 293; and a VL region comprising a VL CDR1 comprising the amino acid sequence of SEQ ID NO: 294, a VL CDR2 comprising the amino acid sequence of SEQ ID NO: 295, and a VL CDR3 comprising the amino acid sequence of SEQ ID NO: 296.

In some embodiments, the CD16a binding agent provided herein (e.g., an antibody) comprises a VH region comprising a VH CDR1 comprising the amino acid sequence of SEQ ID NO: 297, a VH CDR2 comprising the amino acid sequence of SEQ ID NO: 298, and a VH CDR3 comprising the amino acid sequence of SEQ ID NO: 299; and a VL region comprising a VL CDR1 comprising the amino acid sequence of SEQ ID NO: 300, a VL CDR2 comprising the amino acid sequence of SEQ ID NO: 301, and a VL CDR3 comprising the amino acid sequence of SEQ ID NO: 302.

In some embodiments, the CD16a binding agent provided herein (e.g., an antibody) comprises a VH CDR1, a VH CDR2, and/or a VH CDR3 as set forth in a VH comprising the amino acid sequence of SEQ ID NO: 341, and/or a VL CDR1, a VL CDR2, and/or a VL CDR3 as set forth in a VL comprising the amino acid sequence of SEQ ID NO: 342. In some embodiments, the CD16a binding agent provided herein (e.g., an antibody) comprises a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia.

In some embodiments, the CD16a binding agent provided herein (e.g., an antibody) comprises (a) a VH region comprising a VH CDR1 having an amino acid sequence selected from the group consisting of SEQ ID NOs: 305, 311, 317, 323, 329, and 335; a VH CDR2 having an amino acid sequence selected from the group consisting of SEQ ID NOs: 306, 312, 318, 324, 330, and 336; and a VH CDR3 having an amino acid sequence selected from the group consisting of SEQ ID NOs: 307, 313, 319, 325, 331, and 337; and (b) a VL region comprising a VL CDR1 having an amino acid sequence selected from the group consisting of SEQ ID NOs: 308, 314, 320, 326, 332, and 338; a VL CDR2 having an amino acid sequence selected from the group consisting of SEQ ID NOs: 309, 315, 321, 327, 333, and 339; and a VL CDR3 having an amino acid sequence selected from the group consisting of SEQ ID NOs: 310, 316, 322, 328, 334, and 340.

In some embodiments, the CD16a binding agent provided herein (e.g., an antibody) comprises a VH region comprising a VH CDR1 comprising the amino acid sequence of SEQ ID NO: 305, a VH CDR2 comprising the amino acid sequence of SEQ ID NO: 306, and a VH CDR3 comprising the amino acid sequence of SEQ ID NO: 307; and a VL region comprising a VL CDR1 comprising the amino acid sequence of SEQ ID NO: 308, a VL CDR2 comprising the amino acid sequence of SEQ ID NO: 309, and a VL CDR3 comprising the amino acid sequence of SEQ ID NO: 310.

In some embodiments, the CD16a binding agent provided herein (e.g., an antibody) comprises a VH region comprising a VH CDR1 comprising the amino acid sequence of SEQ ID NO: 311 a VH CDR2 comprising the amino acid sequence of SEQ ID NO: 312, and a VH CDR3 comprising the amino acid sequence of SEQ ID NO: 313; and a VL region comprising a VL CDR1 comprising the amino acid sequence of SEQ ID NO: 314, a VL CDR2 comprising the amino acid sequence of SEQ ID NO: 315, and a VL CDR3 comprising the amino acid sequence of SEQ ID NO: 316.

In some embodiments, the CD16a binding agent provided herein (e.g., an antibody) comprises a VH region comprising a VH CDR1 comprising the amino acid sequence of SEQ ID NO: 317, a VH CDR2 comprising the amino acid sequence of SEQ ID NO: 318, and a VH CDR3 comprising the amino acid sequence of SEQ ID NO: 319; and a VL region comprising a VL CDR1 comprising the amino acid sequence of SEQ ID NO: 320, a VL CDR2 comprising the amino acid sequence of SEQ ID NO: 321, and a VL CDR3 comprising the amino acid sequence of SEQ ID NO: 322.

In some embodiments, the CD16a binding agent provided herein (e.g., an antibody) comprises a VH region comprising a VH CDR1 comprising the amino acid sequence of SEQ ID NO: 323, a VH CDR2 comprising the amino acid sequence of SEQ ID NO: 324, and a VH CDR3 comprising the amino acid sequence of SEQ ID NO: 325; and a VL region comprising a VL CDR1 comprising the amino acid sequence of SEQ ID NO: 326, a VL CDR2 comprising the amino acid sequence of SEQ ID NO: 327, and a VL CDR3 comprising the amino acid sequence of SEQ ID NO: 328.

In some embodiments, the CD16a binding agent provided herein (e.g., an antibody) comprises a VH region comprising a VH CDR1 comprising the amino acid sequence of SEQ ID NO: 329, a VH CDR2 comprising the amino acid sequence of SEQ ID NO: 330, and a VH CDR3 comprising the amino acid sequence of SEQ ID NO: 331; and a VL region comprising a VL CDR1 comprising the amino acid sequence of SEQ ID NO: 332, a VL CDR2 comprising the amino acid sequence of SEQ ID NO: 333, and a VL CDR3 comprising the amino acid sequence of SEQ ID NO: 334.

In some embodiments, the CD16a binding agent provided herein (e.g., an antibody) comprises a VH region comprising a VH CDR1 comprising the amino acid sequence of SEQ ID NO: 335, a VH CDR2 comprising the amino acid sequence of SEQ ID NO: 336, and a VH CDR3 comprising the amino acid sequence of SEQ ID NO: 337; and a VL region comprising a VL CDR1 comprising the amino acid sequence of SEQ ID NO: 338, a VL CDR2 comprising the amino acid sequence of SEQ ID NO: 339, and a VL CDR3 comprising the amino acid sequence of SEQ ID NO: 340.

In some embodiments, the antibody further comprises one or more framework regions of SEQ ID NOs: 37, 38, 75, 76, 113, 114, 151, 152, 189, 190, 227, 228, 265, 266, 303, 304, 341, and/or 342. In some embodiments, the antibody or fragment thereof further comprises a framework 1 (FR1), a framework 2 (FR2), a framework 3 (FR3) and/or a framework 4 (FR4) sequence as set forth in any one of SEQ ID NOs: 37, 38, 75, 76, 113, 114, 151, 152, 189, 190, 227, 228, 265, 266, 303, 304, 341, and/or 342. In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, CD16a binding agents (e.g., antibodies such as monospecific or bispecific antibodies), including human CD16a binding agents, described herein comprise a VH region or VH domain. Additionally or alternatively, in some embodiments, CD16a binding agents (e.g., antibodies such as monospecific or bispecific antibodies), including human CD16a binding agents, described herein comprise a VL region or VL domain. In some embodiments, CD16a binding agents (e.g., antibodies such as monospecific or bispecific antibodies), including human CD16a binding agents, described herein have a combination of (i) a VH domain or VH region; and (ii) a VL domain or VL region.

In some embodiments, the CD16a binding agent provided herein (e.g., an antibody) comprises a VH comprising the amino acid sequence of SEQ ID NO: 37. In some embodiments, the CD16a binding agent provided herein (e.g., an antibody) comprises a VL comprising the amino acid sequence of SEQ ID NO: 38. In some embodiments, the CD16a binding agent provided herein (e.g., an antibody) comprises a VH comprising the amino acid sequence of SEQ ID NO: 37, and a VL comprising the amino acid sequence of SEQ ID NO: 38.

In some embodiments, the CD16a binding agent provided herein (e.g., an antibody) comprises a VH comprising the amino acid sequence of SEQ ID NO: 75. In some embodiments, the CD16a binding agent provided herein (e.g., an antibody) comprises a VL comprising the amino acid sequence of SEQ ID NO: 76. In some embodiments, the CD16a binding agent provided herein (e.g., an antibody) comprises a VH comprising the amino acid sequence of SEQ ID NO: 75, and a VL comprising the amino acid sequence of SEQ ID NO: 76.

In some embodiments, the CD16a binding agent provided herein (e.g., an antibody) comprises a VH comprising the amino acid sequence of SEQ ID NO: 113. In some embodiments, the CD16a binding agent provided herein (e.g., an antibody) comprises a VL comprising the amino acid sequence of SEQ ID NO: 114. In some embodiments, the CD16a binding agent provided herein (e.g., an antibody) comprises a VH comprising the amino acid sequence of SEQ ID NO: 113 and a VL comprising the amino acid sequence of SEQ ID NO: 114.

In some embodiments, the CD16a binding agent provided herein (e.g., an antibody) comprises a VH comprising the amino acid sequence of SEQ ID NO: 151. In some embodiments, the CD16a binding agent provided herein (e.g., an antibody) comprises a VL comprising the amino acid sequence of SEQ ID NO: 152. In some embodiments, the CD16a binding agent provided herein (e.g., an antibody) comprises a VH comprising the amino acid sequence of SEQ ID NO: 151 and a VL comprising the amino acid sequence of SEQ ID NO: 152.

In some embodiments, the CD16a binding agent provided herein (e.g., an antibody) comprises a VH comprising the amino acid sequence of SEQ ID NO: 189. In some embodiments, the CD16a binding agent provided herein (e.g., an antibody) comprises a VL comprising the amino acid sequence of SEQ ID NO: 190. In some embodiments, the CD16a binding agent provided herein (e.g., an antibody) comprises a VH comprising the amino acid sequence of SEQ ID NO: 189 and a VL comprising the amino acid sequence of SEQ ID NO: 190.

In some embodiments, the CD16a binding agent provided herein (e.g., an antibody) comprises a VH comprising the amino acid sequence of SEQ ID NO: 227. In some embodiments, the CD16a binding agent provided herein (e.g., an antibody) comprises a VL comprising the amino acid sequence of SEQ ID NO: 228. In some embodiments, the CD16a binding agent provided herein (e.g., an antibody) comprises a VH comprising the amino acid sequence of SEQ ID NO: 227 and a VL comprising the amino acid sequence of SEQ ID NO: 228.

In some embodiments, the CD16a binding agent provided herein (e.g., an antibody) comprises a VH comprising the amino acid sequence of SEQ ID NO: 265. In some embodiments, the CD16a binding agent provided herein (e.g., an antibody) comprises a VL comprising the amino acid sequence of SEQ ID NO: 266. In some embodiments, the CD16a binding agent provided herein (e.g., an antibody) comprises a VH comprising the amino acid sequence of SEQ ID NO: 265 and a VL comprising the amino acid sequence of SEQ ID NO: 266.

In some embodiments, the CD16a binding agent provided herein (e.g., an antibody) comprises a VH comprising the amino acid sequence of SEQ ID NO: 303. In some embodiments, the CD16a binding agent provided herein (e.g., an antibody) comprises a VL comprising the amino acid sequence of SEQ ID NO: 304. In some embodiments, the CD16a binding agent provided herein (e.g., an antibody) comprises a VH comprising the amino acid sequence of SEQ ID NO: 303 and a VL comprising the amino acid sequence of SEQ ID NO: 304.

In some embodiments, the CD16a binding agent provided herein (e.g., an antibody) comprises a VH comprising the amino acid sequence of SEQ ID NO: 341. In some embodiments, the CD16a binding agent provided herein (e.g., an antibody) comprises a VL comprising the amino acid sequence of SEQ ID NO: 342. In some embodiments, the CD16a binding agent provided herein (e.g., an antibody) comprises a VH comprising the amino acid sequence of SEQ ID NO: 341 and a VL comprising the amino acid sequence of SEQ ID NO: 342.

In certain embodiments, the CD16a binding agent (e.g., an antibody of a fragment thereof) provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a CDR, VH or VL in Tables 1-9, or any full-length antibody chain as disclosed herein. In some embodiments, the CD16a binding agent (e.g., an antibody or a fragment thereof) provided herein comprises CDRs of any antibody or fragment thereof provided herein, for example in Tables 1-9. In further embodiments, the CD16a binding agent provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a VH or VL in Tables 1-9, or any full-length antibody chain as disclosed herein.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecule described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402(1997). In some embodiments, the percent identity between two sequences is calculated by dividing the number of residue(s) varied (excluding or including conservative amino acid substitution(s) or degenerate nucleotide substitution(s)) between the two sequences in the alignment with the residue number of any one of the following: (i) full length of the shorter sequence, (ii) full length of the longer sequence, (iii) mean length of the two sequences, (iv) total length of the non-gap portion of the alignment, (v) length of the alignment excluding overhangs, or (vi) length of the alignment including overhangs. Overhangs as used herein with respect to a sequence alignment refer to either or both ends of the alignment where residues of one sequence are considered as aligning to no residues (e.g., gap) in the other sequence. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17(1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the binding agent (e.g., an antibody) provided herein contains substitutions (e.g., conservative substitutions), insertions, or deletions relative to the reference sequence, but the binding agent comprising that sequence retains the ability to bind to CD16a. In some embodiments, a total of 1 to 10 amino acids have been substituted, inserted and/or deleted in a reference amino acid sequence. In some embodiments, substitutions, insertions, or deletions occur in regions outside the CDRs (e.g., in the FRs and/or constant regions).

In some embodiments, the position of one or more CDRs along the VH (e.g., CDR1, CDR2, or CDR3) and/or VL (e.g., CDR1, CDR2, or CDR3) region of a CD16a binding agent (e.g., an antibody), including a human CD16a binding agent, described herein may vary by one, two, three, four, five, or six amino acid positions so long as binding to CD16a(e.g., human CD16a) is maintained (e.g., substantially maintained, for example, at least 50%, at least 60%, at least 70%, at least 80%, at least 90%, or at least 95%). For example, in some embodiments, the position defining a CDR of any of Table 1, 2, 3, 4, 5, 6, 7, 8, or 9 may vary by shifting the N-terminal and/or C-terminal boundary of the CDR by one, two, three, four, five, or six amino acids, relative to the current CDR position, so long as binding to CD16a(e.g., human CD16a) is maintained (e.g., substantially maintained, for example, at least 50%, at least 60%, at least 70%, at least 80%, at least 90%, or at least 95%). Additionally or alternatively, in some embodiments, the length of one or more CDRs along the VH (e.g., CDR1, CDR2, or CDR3) and/or VL (e.g., CDR1, CDR2, or CDR3) region of an CD16a binding agent (e.g., an antibody), including a human CD16a binding agent, described herein may vary (e.g., be shorter or longer) by one, two, three, four, five, or more amino acids, so long as binding to CD16a(e.g., human CD16a) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOs: 1-36, 39-74, 77-112, 115-150, 153-188, 191-226, 229-264, 267-302, or 305-340, so long as binding to CD16a(e.g., human CD16a) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOs: 1-36, 39-74, 77-112, 115-150, 153-188, 191-226, 229-264, 267-302, or 305-340, so long as binding to CD16a (e.g., human CD16a) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 described herein may be extended or shortened by one, two, three, four, five or more amino acids compared to one or more of the CDRs described by SEQ ID NOs: 1-36, 39-74, 77-112, 115-150, 153-188, 191-226, 229-264, 267-302, or 305-340, so long as binding to CD16a(e.g., human CD16a) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 described herein may be extended or shortened by one, two, three, four, five or more amino acids compared to one or more of the CDRs described by SEQ ID NOs: 1-36, 39-74, 77-112, 115-150, 153-188, 191-226, 229-264, 267-302, or 305-340, so long as binding to CD16a(e.g., human CD16a) is maintained (e.g., substantially maintained, for example, at least 50%, at least 60%, at least 70%, at least 80%, at least 90%, or at least 95%). Any method known in the art can be used to ascertain whether binding to CD16a(e.g., human CD16a) is maintained, for example, the binding assays and conditions described in the “Examples” section described herein.

In other embodiments, the CD16a binding agents (e.g., antibodies), including human CD16a binding agents, presented herein that bind to CD16a, further comprise conservative sequence modifications. With respect to polypeptides that are CD16a binding agents (e.g., antibodies), such as human CD16a binding agents, conservative sequence modifications include conservative amino acid substitutions that include ones in which the amino acid residue is replaced with an amino acid residue having a similar side chain. Families of amino acid residues having similar side chains have been defined in the art. Thus, in some embodiments, a predicted nonessential amino acid residue in an CD16a is replaced with another amino acid residue from the same side chain family. Methods of identifying amino acid conservative substitutions which do not eliminate antigen binding and nucleotides encoding thereof are well-known in the art (see, e.g., Brummell et al., Biochem. 32:1180-1187(1993); Kobayashi et al. Protein Eng. 12(10): 879-884(1999); and Burks et al. Proc. Natl. Acad. Sci. USA 94:412-417(1997)). In some embodiments, the conservative sequence modifications described herein modify the amino acid sequences of the CD16a binding agents (e.g., antibodies), including human CD16a binding agents, by 50%, or 55%, or 60%, or 65%, or 70%, or 75%, or 80%, or 85%, or 90%, or 95%, or 98%, or 99%. In some embodiments, the amino acid sequence modifications refer to at most 1, 2, 3, 4, 5, or 6 amino acid substitutions to the CDRs, such as those described in any one of Tables 1-9. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, a CD16a binding agent (e.g., an antibody), including a human CD16a binding agent, contains one or more, including six, CDRs having at least 75%, 80%, 85%, 90%, 95%, 96%, 97%, 98%, or 99% identity to the CDRs of AvGn-CD16a-A2, AvGn-CD16a- #1A2, AvGn-CD16a- #2-1A2, AvGn-CD16a- #2-2A2, AvGn-CD16a- #2-3A2, AvGn-CD16a- #3A2, AvGn-CD16a- #6A2, AvGn-CD16a- #10A2, or AvGn-CD16a- #55 (see, e.g., Tables 1, 2, 3, 4, 5, 6, 7, 8, or 9).

In some embodiments, a CD16a binding agent (e.g., an antibody), including a human CD16a binding agent, contains a VH and a VL comprising CDRs identical to those of AvGn-CD16a-A2, AvGn-CD16a- #1A2, AvGn-CD16a- #2-1A2, AvGn-CD16a- #2-2A2, AvGn-CD16a- #2-3A2, AvGn-CD16a- #3A2, AvGn-CD16a- #6A2, AvGn-CD16a- #10A2, or AvGn-CD16a- #55 (see, e.g., Tables 1, 2, 3, 4, 5, 6, 7, 8, or 9). In some embodiments, the amino acid sequence modifications do not include any modification within an SDR. In some embodiments, the amino acid sequence modifications do not include any modification within a CDR (such as CDR1, CDR2, CDR3, or any combination thereof). Additionally or alternatively, the amino acid sequence modifications are in the framework, constant region, and/or fragment crystallizable region (Fc).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 37, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 38, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 75, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 76, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 113, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 114, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 151, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 152, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 189, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 190, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 227, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 228, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 265, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 266, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 303, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 304, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, the antibody or fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 341, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 342, and the binding of the antibody or fragment thereof to CD16a(e.g., human CD16a) is maintained (e.g., substantially maintained, for example, at least 50%, at least 60%, at least 70%, at least 80%, at least 90%, or at least 95%).

In some embodiments, functional epitopes can be mapped, e.g., by combinatorial alanine scanning, to identify amino acids in the CD16a protein (including CD16a-F and/or CD16a-V) that are necessary for interaction with CD16a binding agents (such as antibodies) provided herein. In some embodiments, conformational and crystal structure of CD16a binding agents (such as antibodies) bound to CD16a may be employed to identify the epitopes. In some embodiments, the present disclosure provides an antibody that specifically binds to the same epitope as any of the CD16a binding agents (such as antibodies or fragments thereof) provided herein.

For example, in some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38. In some embodiments, the CD16a binding agent provided herein (e.g., an antibody) binds to the same epitope as an anti-CD16a antibody comprising a VH comprising the amino acid sequence of SEQ ID NO: 37, and a VL comprising the amino acid sequence of SEQ ID NO: 38.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76. In some embodiments, the CD16a binding agent provided herein (e.g., an antibody) binds to the same epitope as an anti-CD16a antibody comprising a VH comprising the amino acid sequence of SEQ ID NO: 75, and a VL comprising the amino acid sequence of SEQ ID NO: 76.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114. In some embodiments, the CD16a binding agent provided herein (e.g., an antibody) binds to the same epitope as an anti-CD16a antibody comprising a VH comprising the amino acid sequence of SEQ ID NO: 113, and a VL comprising the amino acid sequence of SEQ ID NO: 114.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152. In some embodiments, the CD16a binding agent provided herein (e.g., an antibody) binds to the same epitope as an anti-CD16a antibody comprising a VH comprising the amino acid sequence of SEQ ID NO: 151, and a VL comprising the amino acid sequence of SEQ ID NO: 152.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190. In some embodiments, the CD16a binding agent provided herein (e.g., an antibody) binds to the same epitope as an anti-CD16a antibody comprising a VH comprising the amino acid sequence of SEQ ID NO: 189, and a VL comprising the amino acid sequence of SEQ ID NO: 190.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228. In some embodiments, the CD16a binding agent provided herein (e.g., an antibody) binds to the same epitope as an anti-CD16a antibody comprising a VH comprising the amino acid sequence of SEQ ID NO: 227, and a VL comprising the amino acid sequence of SEQ ID NO: 228.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266. In some embodiments, the CD16a binding agent provided herein (e.g., an antibody) binds to the same epitope as an anti-CD16a antibody comprising a VH comprising the amino acid sequence of SEQ ID NO: 265, and a VL comprising the amino acid sequence of SEQ ID NO: 266.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304. In some embodiments, the CD16a binding agent provided herein (e.g., an antibody) binds to the same epitope as an anti-CD16a antibody comprising a VH comprising the amino acid sequence of SEQ ID NO: 303, and a VL comprising the amino acid sequence of SEQ ID NO: 304.

In some embodiments, the CD16a binding agent provided herein (e.g., an antibody) binds to the same epitope as an anti-CD16a antibody comprising a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342. In some embodiments, the CD16a binding agent provided herein (e.g., an antibody) binds to the same epitope as an anti-CD16a antibody comprising a VH comprising the amino acid sequence of SEQ ID NO: 341, and a VL comprising the amino acid sequence of SEQ ID NO: 342.

In some embodiments, the CD16a binding agent provided herein recognizes an epitope comprising the residue Asp 82 of human CD16a(numbering based on UniProt propeptide sequence P08637). In some embodiments, the CD16a binding agent provided herein recognizes an epitope comprising the residue Asp 82 of human CD16a with an affinity for CD16a at least 10-fold higher than for CD16b. In some embodiments, the CD16a binding agent provided herein recognizes an epitope comprising the residue Tyr 158 of human CD16a (numbering based on UniProt propeptide sequence P08637). In some embodiments, the CD16a binding agent provided herein recognizes an epitope comprising the residue Tyr 158 of human CD16a with an affinity for CD16a at least 10-fold higher than for CD16b.

In some embodiments, the CD16a binding agent provided herein (e.g., an antibody) specifically binds to CD16a competitively with any one of the anti-CD16a antibodies or fragments thereof described herein.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38. In some embodiments, the CD16a binding agent provided herein (e.g., an antibody) specifically binds to CD16a competitively with an anti-CD16a antibody comprising a VH comprising the amino acid sequence of SEQ ID NO: 37, and a VL comprising the amino acid sequence of SEQ ID NO: 38.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76. In some embodiments, the CD16a binding agent provided herein (e.g., an antibody) specifically binds to CD16a competitively with an anti-CD16a antibody comprising a VH comprising the amino acid sequence of SEQ ID NO: 75, and a VL comprising the amino acid sequence of SEQ ID NO: 76.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114. In some embodiments, the CD16a binding agent provided herein (e.g., an antibody) specifically binds to CD16a competitively with an anti-CD16a antibody comprising a VH comprising the amino acid sequence of SEQ ID NO: 113, and a VL comprising the amino acid sequence of SEQ ID NO: 114.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152. In some embodiments, the CD16a binding agent provided herein (e.g., an antibody) specifically binds to CD16a competitively with an anti-CD16a antibody comprising a VH comprising the amino acid sequence of SEQ ID NO: 151, and a VL comprising the amino acid sequence of SEQ ID NO: 152.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190. In some embodiments, the CD16a binding agent provided herein (e.g., an antibody) specifically binds to CD16a competitively with an anti-CD16a antibody comprising a VH comprising the amino acid sequence of SEQ ID NO: 189, and a VL comprising the amino acid sequence of SEQ ID NO: 190.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228. In some embodiments, the CD16a binding agent provided herein (e.g., an antibody) specifically binds to CD16a competitively with an anti-CD16a antibody comprising a VH comprising the amino acid sequence of SEQ ID NO: 227, and a VL comprising the amino acid sequence of SEQ ID NO: 228.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266. In some embodiments, the CD16a binding agent provided herein (e.g., an antibody) specifically binds to CD16a competitively with an anti-CD16a antibody comprising a VH comprising the amino acid sequence of SEQ ID NO: 265, and a VL comprising the amino acid sequence of SEQ ID NO: 266.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304. In some embodiments, the CD16a binding agent provided herein (e.g., an antibody) specifically binds to CD16a competitively with an anti-CD16a antibody comprising a VH comprising the amino acid sequence of SEQ ID NO: 303, and a VL comprising the amino acid sequence of SEQ ID NO: 304.

In some embodiments, the CD16a binding agent provided herein (e.g., an antibody) specifically binds to CD16a competitively with an anti-CD16a antibody comprising a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342. In some embodiments, the CD16a binding agent provided herein (e.g., an antibody) specifically binds to CD16a competitively with an anti-CD16a antibody comprising a VH comprising the amino acid sequence of SEQ ID NO: 341, and a VL comprising the amino acid sequence of SEQ ID NO: 342.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a-A2. In yet further embodiments, the CD16a binding agent comprises six CDRs as listed in one column of Table 1. In some embodiments, the CD16a binding agent comprises three CDRs of the heavy chain variable region as set forth in SEQ ID NO: 37 and three CDRs of the light chain variable regions as set forth in SEQ ID NO: 38. In some embodiments, the CD16a binding agent comprises the heavy chain variable region as set forth in SEQ ID NO: 37 and the light chain variable regions as set forth in SEQ ID NO: 38.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #1A2. In yet further embodiments, the CD16a binding agent comprises six CDRs as listed in one column of Table 2. In some embodiments, the CD16a binding agent comprises three CDRs of the heavy chain variable region as set forth in SEQ ID NO: 75 and three CDRs of the light chain variable regions as set forth in SEQ ID NO: 76. In some embodiments, the CD16a binding agent comprises the heavy chain variable region as set forth in SEQ ID NO: 75 and the light chain variable regions as set forth in SEQ ID NO: 76.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #2-1A2. In yet further embodiments, the CD16a binding agent comprises six CDRs as listed in one column of Table 3. In some embodiments, the CD16a binding agent comprises three CDRs of the heavy chain variable region as set forth in SEQ ID NO: 113 and three CDRs of the light chain variable regions as set forth in SEQ ID NO: 114. In some embodiments, the CD16a binding agent comprises the heavy chain variable region as set forth in SEQ ID NO: 113 and the light chain variable regions as set forth in SEQ ID NO: 114.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #2-2A2. In yet further embodiments, the CD16a binding agent comprises six CDRs as listed in one column of Table 4. In some embodiments, the CD16a binding agent comprises three CDRs of the heavy chain variable region as set forth in SEQ ID NO: 151 and three CDRs of the light chain variable regions as set forth in SEQ ID NO: 152. In some embodiments, the CD16a binding agent comprises the heavy chain variable region as set forth in SEQ ID NO: 151 and the light chain variable regions as set forth in SEQ ID NO: 152.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #2-3A2. In yet further embodiments, the CD16a binding agent comprises six CDRs as listed in one column of Table 5. In some embodiments, the CD16a binding agent comprises three CDRs of the heavy chain variable region as set forth in SEQ ID NO: 189 and three CDRs of the light chain variable regions as set forth in SEQ ID NO: 190. In some embodiments, the CD16a binding agent comprises the heavy chain variable region as set forth in SEQ ID NO: 189 and the light chain variable regions as set forth in SEQ ID NO: 190.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #3A2. In yet further embodiments, the CD16a binding agent comprises six CDRs as listed in one column of Table 6. In some embodiments, the CD16a binding agent comprises three CDRs of the heavy chain variable region as set forth in SEQ ID NO: 227 and three CDRs of the light chain variable regions as set forth in SEQ ID NO: 228. In some embodiments, the CD16a binding agent comprises the heavy chain variable region as set forth in SEQ ID NO: 227 and the light chain variable regions as set forth in SEQ ID NO: 228.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #6A2. In yet further embodiments, the CD16a binding agent comprises six CDRs as listed in one column of Table 7. In some embodiments, the CD16a binding agent comprises three CDRs of the heavy chain variable region as set forth in SEQ ID NO: 265 and three CDRs of the light chain variable regions as set forth in SEQ ID NO: 266. In some embodiments, the CD16a binding agent comprises the heavy chain variable region as set forth in SEQ ID NO: 265 and the light chain variable regions as set forth in SEQ ID NO: 266.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #10A2. In yet further embodiments, the CD16a binding agent comprises six CDRs as listed in one column of Table 8. In some embodiments, the CD16a binding agent comprises three CDRs of the heavy chain variable region as set forth in SEQ ID NO: 303 and three CDRs of the light chain variable regions as set forth in SEQ ID NO: 304. In some embodiments, the CD16a binding agent comprises the heavy chain variable region as set forth in SEQ ID NO: 303 and the light chain variable regions as set forth in SEQ ID NO: 304.

In some embodiments, the CD16a binding agent binds to CD16a-F and CD16a-V, but not binds to CD16b-NA1 or CD16b-NA2. In further embodiments, the CD16a binding agent comprises six CDRs of the antibody designated AvGn-CD16a- #55. In yet further embodiments, the CD16a binding agent comprises six CDRs as listed in one column of Table 9. In some embodiments, the CD16a binding agent comprises three CDRs of the heavy chain variable region as set forth in SEQ ID NO: 341 and three CDRs of the light chain variable regions as set forth in SEQ ID NO: 342. In some embodiments, the CD16a binding agent comprises the heavy chain variable region as set forth in SEQ ID NO: 341 and the light chain variable regions as set forth in SEQ ID NO: 342.

In some embodiments, the CD16a binding agent provided herein is in a scFv format. In some embodiments, the CD16a binding agent comprises a scFv having a combination of (i) a VH described herein and (ii) a VL described herein. In some embodiments, the CD16a binding agent comprises an amino acid sequence of SEQ ID NO: 346.

In some embodiments, the CD16a binding agent provided herein is in a Fab format. In some embodiments, CD16a binding agents (e.g., antibodies such as monospecific or bispecific antibodies), including human CD16a binding agents, described herein comprise a heavy chain having a combination of (i) a VH described herein, such as in any one of Tables 1-9; and (ii) a heavy chain constant domain (e.g., CH1). In further embodiments, the carboxyl terminus (C terminus) of the VH is conjugated directly or indirectly to the amino terminus (N terminus) of the heavy chain constant domain (e.g., CH1). In some embodiments, CD16a binding agents (e.g., antibodies such as monospecific or bispecific antibodies), including human CD16a binding agents, described herein comprise a light chain having a combination of (i) a VL domain described herein, such as in any one of Tables 1-9; and (ii) a light chain constant domain (CL). In further embodiments, the C terminus of the VL is conjugated directly or indirectly to the N terminus of the CL. In some embodiments, CD16a binding agents (e.g., antibodies such as monospecific or bispecific antibodies), including human CD16a binding agents, described herein comprise (a) a heavy chain having a combination of (i) a VH described herein, such as in any one of Tables 1-9, and (ii) a heavy chain constant domain (e.g., CH1); and (b) a light chain having a combination of (i) a VL described herein, such as in any one of Tables 1-9, and (ii) a light chain constant domain (CL).

In some embodiments, the binding agents are superior developability based on a known assay in the art, for example, various chromatographic methods, including size exclusion chromatography (SEC), hydrophobic interaction chromatography (HIC), and standup monolayer adsorption chromatography (SMAC). In some embodiments, the binding agents are superior developability based on measurement of monomer percentage, solubility, and/or antibody aggregation or precipitation.

In some embodiments, CD16a binding agents (e.g., antibodies such as monospecific or bispecific antibodies), including human CD16a binding agents, described herein comprise a heavy chain having a combination of (i) a VH described herein, such as in any one of Tables 1-9; and (ii) one or more heavy chain constant domains (e.g., CH1, Hinge, CH2, and CH3). In further embodiments, the carboxyl terminus (C terminus) of the VH is conjugated directly or indirectly to the amino terminus (N terminus) of the one or more heavy chain constant domains.

In some embodiments, CD16a binding agents (e.g., antibodies such as monospecific or bispecific antibodies), including human CD16a binding agents, described herein comprise a light chain having a combination of (i) a VL domain described herein, such as in any one of Tables 1-9; and (ii) a light chain constant domain (CL). In further embodiments, the C terminus of the VL is conjugated directly or indirectly to the N terminus of the CL.

In some embodiments, CD16a binding agents (e.g., antibodies such as monospecific or bispecific antibodies), including human CD16a binding agents, described herein comprise (a) a heavy chain having a combination of (i) a VH described herein, such as in any one of Tables 1-9, and (ii) one or more heavy chain constant domains (e.g., CH1, Hinge, CH2, and CH3); and (b) a light chain having a combination of (i) a VL described herein, such as in any one of Tables 1-9, and (ii) a light chain constant domain in an IgG format (CL or CL1).

In some embodiments, the CD16a binding agent provided herein is in an IgG format. In some embodiments, the CD16a binding agent provided herein is in an IgG1 format, the CD16a binding agent provided herein is in an IgG4 format In some embodiments, the CD16a binding agent provided herein is in an IgG format with mutated Fcγ1 region to eliminate Fc binding activity to Fcγ receptors.

In some embodiments, the CD16a binding agent provided herein is in an IgG format comprising Leu234Ala, Leu235Ala and/or Pro329Gly mutations (Fc silencing mutations) to remove the Fc binding site for CD16a. In some embodiments, the CD16a binding properties of the CD16a binding agents are not significantly impacted by the Fc silencing mutations (e.g. Leu234Ala, Leu235Ala and/or Pro329Gly). In some embodiments, the CD16a binding agent the Fc silencing mutations provided herein binds to CD16a(e.g., human CD16a) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

In some embodiments, the CD16a binding properties of the CD16a binding agents are not significantly impacted by the presence of human IgG.

In some embodiments, provided herein is an CD16a binding protein comprising any one of the anti-CD16a antibodies described herein. In some embodiments, the CD16a binding protein is an antibody comprising two heavy chains and two light chains. In some embodiments, the CD16a binding protein is an antibody comprising two heavy chains comprising a same VH region and two light chains comprising a same VL region.

In some embodiments, the CD16a binding protein provided herein is a monoclonal antibody, including a mouse, chimeric, humanized, or human antibody. In some embodiments, the anti-CD16a antibody is an antibody fragment, e.g., an scFv. In some embodiments, the CD16a binding protein is a fusion protein comprising the anti-CD16a antibody provided herein. In other embodiments, the CD16a binding protein is a multispecific antibody comprising the anti-CD16a antibody or fragment thereof provided herein.

In some embodiments, the CD16a binding agent provided herein is a monoclonal antibody, including a mouse, chimeric, humanized, or human antibody. In some embodiments, the anti-CD16a antibody is an antibody fragment, e.g., an scFv. In some embodiments, the CD16a binding agent is a fusion protein comprising the anti-CD16a antibody provided herein. In other embodiments, the CD16a binding agent is a multispecific antibody comprising the anti-CD16a antibody or fragment thereof provided herein. In some embodiments, the CD16a binding agent described herein is a multispecific antibody comprising a first binding domain comprising any one of the anti-CD16a antibodies described herein, and a second binding domain that binds to a second antigen. In some embodiments, the second antigen is expressed on a tumor cell. In some embodiments, the second antigen is CD19.

In some embodiments, the CD16a binding agents (e.g. antibodies) can activate CD16a mediated cell signaling pathway. In some embodiments, the antibodies described herein activate CD16a mediated cell signaling pathway by at least about 10%. In some embodiments, the antibodies described herein activate CD16a mediated cell signaling pathway by at least about 20%. In some embodiments, the antibodies described herein activate CD16a mediated cell signaling pathway by at least about 30%. In some embodiments, the antibodies described herein activate CD16a mediated cell signaling pathway by at least about 40%. In some embodiments, the antibodies described herein activate CD16a mediated cell signaling pathway by at least about 50%. In some embodiments, the antibodies described herein activate CD16a mediated cell signaling pathway by at least about 60%. In some embodiments, the antibodies described herein activate CD16a mediated cell signaling pathway by at least about 70%. In some embodiments, the antibodies described herein activate CD16a mediated cell signaling pathway by at least about 80%. In some embodiments, the antibodies described herein activate CD16a mediated cell signaling pathway by at least about 90%. In some embodiments, the antibodies described herein activate CD16a mediated cell signaling pathway by at least about 95%. In certain embodiments, the antibodies described herein activate CD16a mediated cell signaling pathway by at least about 15% to about 65%. In certain embodiments, the antibodies described herein activate CD16a mediated cell signaling pathway by at least about 20% to about 65%. In certain embodiments, the antibodies described herein activate CD16a mediated cell signaling pathway by at least about 30% to about 65%. In some embodiments, the CD16a binding agent does not activate CD16b mediated cell signaling pathway.

In some embodiments, the CD16a binding agents (e.g. antibodies) can modulate NK cell activity. In some embodiments, the antibodies described herein can activate an NK cell. In some embodiments, the antibodies described herein activate an NK cell activity by at least about 10%. In some embodiments, the antibodies described herein activate an NK cell activity by at least about 20%. In some embodiments, the antibodies described herein activate an NK cell activity by at least about 30%. In some embodiments, the antibodies described herein activate an NK cell activity by at least about 40%. In some embodiments, the antibodies described herein activate an NK cell activity by at least about 50%. In some embodiments, the antibodies described herein activate an NK cell activity by at least about 60%. In some embodiments, the antibodies described herein activate an NK cell activity by at least about 70%. In some embodiments, the antibodies described herein activate an NK cell activity by at least about 80%. In some embodiments, the antibodies described herein activate an NK cell activity by at least about 90%. In some embodiments, the antibodies described herein activate an NK cell activity by at least about 95%. In certain embodiments, the antibodies described herein activate an NK cell activity by at least about 15% to about 65%. In certain embodiments, the antibodies described herein activate an NK cell activity by at least about 20% to about 65%. In certain embodiments, the antibodies described herein activate an NK cell activity by at least about 30% to about 65%. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a-F/F genotype. In specific embodiments, the NK cells are NK cells from a subject with a CD16a-V/F genotype. In specific embodiments, the NK cells are NK cells from a subject with a CD16a-V/V genotype.

In some embodiments, the CD16a binding agents (e.g. antibodies) described herein can induce ADCC activity of an NK cell. In some embodiments, the antibodies described herein induce ADCC activity of an NK cell by at least about 10%. In some embodiments, the antibodies described herein induce ADCC activity of an NK cell by at least about 20%. In some embodiments, the antibodies described herein induce ADCC activity of an NK cell by at least about 30%. In some embodiments, the antibodies described herein induce ADCC activity of an NK cell by at least about 40%. In some embodiments, the antibodies described herein induce ADCC activity of an NK cell by at least about 50%. In some embodiments, the antibodies described herein induce ADCC activity of an NK cell at least about 60%. In some embodiments, the antibodies described herein induce ADCC activity of an NK cell by at least about 70%. In some embodiments, the antibodies described herein induce ADCC activity of an NK cell by at least about 80%. In some embodiments, the antibodies described herein induce ADCC activity of an NK cell by at least about 90%. In some embodiments, the antibodies described herein induce ADCC activity of an NK cell by at least about 95%. In certain embodiments, the antibodies described herein induce ADCC activity of an NK cell by at least about 15% to about 65%. In certain embodiments, the antibodies described herein induce ADCC activity of an NK cell by at least about 20% to about 65%. In certain embodiments, the antibodies described herein induce ADCC activity of an NK cell by at least about 30% to about 65%. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a F/F genotype. In specific embodiments, the NK cells are NK cells from a subject with a CD16a-V/F genotype. In specific embodiments, the NK cells are NK cells from a subject with a CD16a-V/V genotype.

In some embodiments, the CD16a binding agents (e.g. antibodies) described herein can induce cytokine secretion of an NK cell. In some embodiments, the antibodies described herein induce cytokine secretion of an NK cell by at least about 10%. In some embodiments, the antibodies described herein induce cytokine secretion of an NK cell by at least about 20%. In some embodiments, the antibodies described herein induce cytokine secretion of an NK cell by at least about 30%. In some embodiments, the antibodies described herein induce cytokine secretion of an NK cell by at least about 40%. In some embodiments, the antibodies described herein induce cytokine secretion of an NK cell by at least about 50%. In some embodiments, the antibodies described herein induce cytokine secretion of an NK cell at least about 60%. In some embodiments, the antibodies described herein induce cytokine secretion of an NK cell by at least about 70%. In some embodiments, the antibodies described herein induce cytokine secretion of an NK cell by at least about 80%. In some embodiments, the antibodies described herein induce cytokine secretion of an NK cell by at least about 90%. In some embodiments, the antibodies described herein induce cytokine secretion of an NK cell by at least about 95%. In certain embodiments, the antibodies described herein induce cytokine secretion of an NK cell by at least about 15% to about 65%. In certain embodiments, the antibodies described herein induce cytokine secretion of an NK cell by at least about 20% to about 65%. In certain embodiments, the antibodies described herein induce cytokine secretion of an NK cell by at least about 30% to about 65%. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a-F/F genotype. In specific embodiments, the NK cells are NK cells from a subject with a CD16a-V/F genotype. In specific embodiments, the NK cells are NK cells from a subject with a CD16a-V/V genotype.

In some embodiments, the CD16a binding agents (e.g. antibodies) in an IgG format described herein can activate NK cell activity even with one or more Fc silencing mutations (e.g. Leu234Ala, Leu235Ala and/or Pro329Gly). In some embodiments, the antibodies in an IgG format described herein can activate NK cell activity even in an Fc silenced format with Leu234Ala, Leu235Ala and/or Pro329Gly mutations. In some embodiments, the antibodies in an IgG format described herein can activate NK cell activity with mutated Fcγ1 region to eliminate Fc binding activity to Fcγ receptors.

In some embodiments, the CD16a binding agents (e.g. antibodies) described herein do not bind to cells expressing CD16b. In some embodiments, the antibodies described herein do not bind to cells expressing CD16b-NA1. In some embodiments, the antibodies described herein do not bind to cells expressing CD16b-NA2. In some embodiments, the antibodies described herein do not bind to cells derived from a subject with CD16b-NA1/CD16b-NA1 genotype. In some embodiments, the antibodies described herein do not bind to cells derived from a subject with CD16b-NA1/CD16b-NA2 genotype. In some embodiments, the antibodies described herein do not bind to cells derived from a subject with CD16b-NA2/CD16b-NA2 genotype. In some embodiments, the antibodies described herein do not bind to neutrophils. In some embodiments, the antibodies described herein do not bind to neutrophils derived from a subject with CD16b-NA1/CD16b-NA1 genotype. In some embodiments, the antibodies described herein do not bind to neutrophils derived from a subject with CD16b-NA1/CD16b-NA2 genotype. In some embodiments, the antibodies described herein do not bind to neutrophils derived from a subject with CD16b-NA2/CD16b-NA2 genotype. In some embodiments, the antibodies described herein do not induce activation of neutrophils. In some embodiments, the antibodies described herein induce activation of NK cells and do not induce activation of neutrophils. In some embodiments, the antibodies described herein do not induce activation of neutrophils and other granulocytes. In some embodiments, the antibodies described herein induce activation of NK cells and do not induce activation of neutrophils and other granulocytes.

Other exemplary CD16a binding molecules are described in more detail in the following sections. In some embodiments, the anti-CD16a antibody or antigen-binding protein according to any of the above embodiments may incorporate any of the features, singly or in combination, as described in Sections 5.2.4 to 5.2.7 below.

TABLE 1
AvGn-CD16a-A2

| | | Exemplary | IMGT | Kabat | Chothia | Contact | AbM |
|---|---|---|---|---|---|---|---|
| VH | VH | GGSISRGSYYWG | GGSISRGSYY | RGSYYWG | GGSISRGSY | SRGSYYWG | GGSISRGSYYWG |
| CDR | CDR1 | (SEQ ID NO: | (SEQ ID NO: | (SEQ ID NO: 13) | (SEQ ID NO: 19) | (SEQ ID NO: 25) | (SEQ ID NO: 31) |
| Seq. | | 1) | 7) | | | | |
| | VH | TIDYSGRTYYNPS | TIDYSGRT | TIDYSGRTYYNPS | DYSGR | WIGTIDYSGRTY | TIDYSGRTY |
| | CDR2 | LKS (SEQ ID | (SEQ ID NO: | LKS | (SEQ ID NO: 20) | (SEQ ID NO: 26) | (SEQ ID NO: 32) |
| | | NO: 2) | 8) | (SEQ ID NO: 14) | | | |
| | VH | AFPGEYFGVFDH | ARAFPGEYFGVF | AFPGEYFGVFDH | AFPGEYFGVFDH | ARAFPGEYFGVF | AFPGEYFGVFDH |
| | CDR3 | (SEQ ID NO: | DH (SEQ ID | (SEQ ID NO: 15) | (SEQ ID NO: 21) | D | (SEQ ID NO: 33) |
| | | 3) | NO: 9) | | | (SEQ ID NO: 27) | |
| VL | VL | RASQSVSSSYLA | QSVSSSY | RASQSVSSSYLA | RASQSVSSSYLA | SSSYLAWY | RASQSVSSSYLA |
| CDR | CDR1 | (SEQ ID NO: | (SEQ ID NO: | (SEQ ID NO: 16) | (SEQ ID NO: 22) | (SEQ ID NO: 28) | (SEQ ID NO: 34) |
| Seq. | | 4) | 10) | | | | |
| | VL | DASSRAT | DAS | DASSRAT | DASSRAT | LLIYDASSRA | DASSRAT |
| | CDR2 | (SEQ ID NO: | (SEQ ID NO: | (SEQ ID NO: 17) | (SEQ ID NO: 23) | (SEQ ID NO: 29) | (SEQ ID NO: 35) |
| | | 5) | 11) | | | | |
| | VL | QQYGSSSLIT | QQYGSSSLIT | QQYGSSSLIT | QQYGSSSLIT | QQYGSSSLI | QQYGSSSLIT |
| | CDR3 | (SEQ ID NO: | (SEQ ID NO: | (SEQ ID NO: 18) | (SEQ ID NO: 24) | (SEQ ID NO: 30) | (SEQ ID NO: 36) |
| | | 6) | 12) | | | | |

VH Sequence:
QLQLQESGPGLVKPSETLSLTCTVSGGSISRGSYYWGWIRQPPGKGLEWIGTIDYSGRTYYNPSLKSRVTISVDTSKNQFSLKLSSVTAADTAVYYC
ARAFPGEYFGVFDHWGQGTLVTVSS (SEQ ID NO: 37)
VL Sequence:
EIVLTQSPGTLSLSPGERATLSCRASQSVSSSYLAWYQQKPGQAPRLLIYDASSRATGIPDRFSGSGSGTDFTLTISRLEPEDFAVYYCQQYGSSSL
ITFGQGTKVEIK (SEQ ID NO: 38)
scFv:
EIVLTQSPGTLSLSPGERATLSCRASQSVSSSYLAWYQQKPGQAPRLLIYDASSRATGIPDRFSGSGSGTDFTLTISRLEPEDFAVYYCQQYGSSSL
ITFGQGTKVEIKGGGGSGGGGSGGGGSQLQLQESGPGLVKPSETLSLTCTVSGGSISRGSYYWGWIRQPPGKGLEWIGTIDYSGRTYYNPSLKSRVT
ISVDTSKNQFSLKLSSVTAADTAVYYCARAFPGEYFGVFDHWGQGTLVTVSS (SEQ ID NO: 346)

TABLE 2
AvGn-CD16a-#1A2

| | | Exemplary | IMGT | Kabat | Chothia | Contact | AbM |
|---|---|---|---|---|---|---|---|
| VH | VH | GGSIGRGSYYWG | GGSIGRGSYY | RGSYYWG | GGSIGRGSY | GRGSYYWG | GGSIGRGSYYWG |
| CDR | CDR1 | (SEQ ID NO: | (SEQ ID NO: 45) | (SEQ ID NO: | (SEQ ID NO: 57) | (SEQ ID NO: 63) | (SEQ ID NO: |
| Seq. | | 39) | | 51) | | | 69) |
| | VH | TIDYSGRTYYNPS | TIDYSGRT | TIDYSGRTYYNPS | DYSGR | WIGTIDYSGRTY | TIDYSGRTY |
| | CDR2 | LKS (SEQ ID | (SEQ ID NO: 46) | LKS (SEQ ID | (SEQ ID NO: 58) | (SEQ ID NO: 64) | (SEQ ID NO: |
| | | NO: 40) | | NO: 52) | | | 70) |
| | VH | AFPGEYFAVFDH | ARAFPGEYFAVF | AFPGEYFAVFDH | AFPGEYFAVFDH | ARAFPGEYFAVF | AFPGEYFAVFDH |
| | CDR3 | (SEQ ID NO: | DH | (SEQ ID NO: | (SEQ ID NO: 59) | D | (SEQ ID NO: |
| | | 41) | (SEQ ID NO: 47) | 53) | | (SEQ ID NO: 65) | 71) |
| VL | VL | RASQSVSSIYLA | QSVSSIY | RASQSVSSIYLA | RASQSVSSIYLA | SSIYLAWY | RASQSVSSIYLA |
| CDR | CDR1 | (SEQ ID NO: | (SEQ ID NO: 48) | (SEQ ID NO: | (SEQ ID NO: 60) | (SEQ ID NO: 66) | (SEQ ID NO: |
| Seq. | | 42) | | 54) | | | 72) |
| | VL | DASSRAT | DAS | DASSRAT | DASSRAT | LLIYDASSRA | DASSRAT |
| | CDR2 | (SEQ ID NO: | (SEQ ID NO: 49) | (SEQ ID NO: | (SEQ ID NO: 61) | (SEQ ID NO: 67) | (SEQ ID NO: |
| | | 43) | | 55) | | | 73) |
| | VL | QQYGGSSLIT | QQYGGSSLIT | QQYGGSSLIT | QQYGGSSLIT | QQYGGSSLI | QQYGGSSLIT |
| | CDR3 | (SEQ ID NO: | (SEQ ID NO: 50) | (SEQ ID NO: | (SEQ ID NO: 62) | (SEQ ID NO: 68) | (SEQ ID NO: |
| | | 44) | | 56) | | | 74) |

VH Sequence:
QLQLQESGPGLVKPSETLSLTCTVSGGSIGRGSYYWGWIRQPPGKGLEWIGTIDYSGRTYYNPSLKSRVTISVDTSKNQFSLKLSSVTAADTAVYYC
ARAFPGEYFAVFDHWGQGTLVTVSS (SEQ ID NO: 75)
VL Sequence:
EIVLTQSPGTLSLSPGERAILSCRASQSVSSIYLAWYQQKPGQAPRLLIYDASSRATGIPDRFSGSGSGTDFTLTISRLEPEDFAVYYCQQYGGSSL
ITFGQGTKVEIK (SEQ ID NO: 76)

TABLE 3
AvGn-CD16a-#2-1A2

| | | Exemplary | IMGT | Kabat | Chothia | Contact | AbM |
|---|---|---|---|---|---|---|---|
| VH | VH | GGSISRGDYYWG | GGSISRGDYY | RGDYYWG | GGSISRGDY | SRGDYYWG | GGSISRGDYYWG |
| CDR | CDR1 | (SEQ ID NO: | (SEQ ID NO: 83) | (SEQ ID NO: 89) | (SEQ ID NO: 95) | (SEQ ID NO: | (SEQ ID NO: |
| Seq. | | 77) | | | | 101) | 107) |
| | VH | TVDYSGRTYYNP | TVDYSGRT | TVDYSGRTYYNP | DYSGR | WIGTVDYSGRTY | TVDYSGRTY |
| | CDR2 | SLKS (SEQ ID | (SEQ ID NO: 84) | SLKS | (SEQ ID NO: 96) | (SEQ ID NO: | (SEQ ID NO: |
| | | NO: 78) | | (SEQ ID NO: 90) | | 102) | 108) |
| | VH | AFPGEYFAVFDH | ARAFPGEYFAVF | AFPGEYFAVFDH | AFPGEYFAVFDH | ARAFPGEYFAVF | AFPGEYFAVFDH |
| | CDR3 | (SEQ ID NO: | DH | (SEQ ID NO: 91) | (SEQ ID NO: 97) | D (SEQ ID | (SEQ ID NO: |
| | | 79) | (SEQ ID NO: 85) | | | NO: 103) | 109) |
| VL | VL | RASQSLSSSYLA | QSLSSSY | RASQSLSSSYLA | RASQSLSSSYLA | SSSYLAWY | RASQSLSSSYLA |
| CDR | CDR1 | (SEQ ID NO: | (SEQ ID NO: 86) | (SEQ ID NO: 92) | (SEQ ID NO: 98) | (SEQ ID NO: | (SEQ ID NO: |
| Seq. | | 80) | | | | 104) | 110) |
| | VL | DAISRAT | DAI | DAISRAT | DAISRAT | LLIYDAISRA | DAISRAT |
| | CDR2 | (SEQ ID NO: | (SEQ ID NO: 87) | (SEQ ID NO: 93) | (SEQ ID NO: 99) | (SEQ ID NO: | (SEQ ID NO: |
| | | 81) | | | | 105) | 111) |
| | VL | QQYGSSRLIT | QQYGSSRLIT | QQYGSSRLIT | QQYGSSRLIT | QQYGSSRLI | QQYGSSRLIT |
| | CDR3 | (SEQ ID NO: | (SEQ ID NO: 88) | (SEQ ID NO: 94) | (SEQ ID NO: 100) | (SEQ ID NO: | (SEQ ID NO: |
| | | 82) | | | | 106) | 112) |

VH Sequence:
QLQLQESGPGLVKPSETLSLTCTVSGGSISRGDYYWGWIRQPPGKGLEWIGTVDYSGRTYYNPSLKSRVTISVDTSKNQFSLKLSSVTAADTAVYY
CARAFPGEYFAVFDHWGQGTLVTVSS (SEQ ID NO: 113)
VL Sequence:
EIVLTQSPGTLSLSPGERATLSCRASQSLSSSYLAWYQQKPGQAPRLLIYDAISRATGIPDRFSGSGSGTDFTLTISRLEPEDFAVYYCQQYGSSR
LITFGQGTKVEIK (SEQ ID NO: 114)

TABLE 4
AvGn-CD16a-#2-2A2

| | | Exemplary | IMGT | Kabat | Chothia | Contact | AbM |
|---|---|---|---|---|---|---|---|
| VH | VH | GGSISRGDYYWG | GGSISRGDYY | RGDYYWG | GGSISRGDY | SRGDYYWG | GGSISRGDYYWG |
| CDR | CDR1 | (SEQ ID NO: | (SEQ ID NO: | (SEQ ID NO: 127) | (SEQ ID NO: 133) | (SEQ ID NO: | (SEQ ID NO: |
| Seq. | | 115) | 121) | | | 139) | 145) |
| | VH | TVDYSGRTYYNP | TVDYSGRT | TVDYSGRTYYNP | DYSGR | WIGTVDYSGRTY | TVDYSGRTY |
| | CDR2 | SLKS (SEQ ID | (SEQ ID NO: | SLKS | (SEQ ID NO: 134) | (SEQ ID NO: | (SEQ ID NO: |
| | | NO: 116) | 122) | (SEQ ID NO: 128) | | 140) | 146) |
| | VH | AFPGEYFAVFDH | ARAFPGEYFAVF | AFPGEYFAVFDH | AFPGEYFAVFDH | ARAFPGEYFAVF | AFPGEYFAVFDH |
| | CDR3 | (SEQ ID NO: | DH (SEQ ID | (SEQ ID NO: 129) | (SEQ ID NO: 135) | D (SEQ ID | (SEQ ID NO: |
| | | 117) | NO: 123) | | | NO: 141) | 147) |
| VL | VL | RASQSVSSSYLA | QSVSSSY | RASQSVSSSYLA | RASQSVSSSYLA | SSSYLAWY | RASQSVSSSYLA |
| CDR | CDR1 | (SEQ ID NO: | (SEQ ID NO: | (SEQ ID NO: 130) | (SEQ ID NO: 136) | (SEQ ID NO: | (SEQ ID NO: |
| Seq. | | 118) | 124) | | | 142) | 148) |
| | VL | GASSRAT | GAS | GASSRAT | GASSRAT | LLIYGASSRA | GASSRAT |
| | CDR2 | (SEQ ID NO: | (SEQ ID NO: | (SEQ ID NO: 131) | (SEQ ID NO: 137) | (SEQ ID NO: | (SEQ ID NO: |
| | | 119) | 125) | | | 143) | 149) |
| | VL | QQYGASSLIT | QQYGASSLIT | QQYGASSLIT | QQYGASSLIT | QQYGASSLI | QQYGASSLIT |
| | CDR3 | (SEQ ID NO: | (SEQ ID NO: | (SEQ ID NO: 132) | (SEQ ID NO: 138) | (SEQ ID NO: | (SEQ ID NO: |
| | | 120) | 126) | | | 144) | 150) |

VH Sequence:
QLQLQESGPGLVKPSETLSLTCTVSGGSISRGDYYWGWIRQPPGKGLEWIGTVDYSGRTYYNPSLKSRVTISVDTSKNQFSLKLSSVTAADTAVYY
CARAFPGEYFAVFDHWGQGTLVTVSS (SEQ ID NO: 151)
VL Sequence:
EIVLTQSPGTLSLSPGERATLSCRASQSVSSSYLAWYQQKPGQAPRLLIYGASSRATGIPDRFSGSGSGTDFTLTISRLEPEDFAVYYCQQYGASS
LITFGQGTKVEIK (SEQ ID NO: 152)

TABLE 5
AvGn-CD16a-#2-3A2

| | | Exemplary | IMGT | Kabat | Chothia | Contact | AbM |
|---|---|---|---|---|---|---|---|
| VH | VH | GGSISRGDYYWG | GGSISRGDYY | RGDYYWG | GGSISRGDY | SRGDYYWG | GGSISRGDYYWG |
| CDR | CDR1 | (SEQ ID NO: | (SEQ ID NO: | (SEQ ID NO: 165) | (SEQ ID NO: 171) | (SEQ ID NO: | (SEQ ID NO: |
| Seq. | | 153) | 159) | | | 177) | 183) |
| | VH | TVDYSGRTYYNP | TVDYSGRT | TVDYSGRTYYNP | DYSGR | WIGTVDYSGRTY | TVDYSGRTY |
| | CDR2 | SLKS (SEQ ID | (SEQ ID NO: | SLKS | (SEQ ID NO: 172) | (SEQ ID NO: | (SEQ ID NO: |
| | | NO: 154) | 160) | (SEQ ID NO: 166) | | 178) | 184) |
| | VH | AFPGEYFAVFDH | ARAFPGEYFAVF | AFPGEYFAVFDH | AFPGEYFAVFDH | ARAFPGEYFAVF | AFPGEYFAVFDH |
| | CDR3 | (SEQ ID NO: | DH (SEQ ID | (SEQ ID NO: 167) | (SEQ ID NO: 173) | D (SEQ ID | (SEQ ID NO: |
| | | 155) | NO: 161) | | | NO: 179) | 185) |
| VL | VL | RASQSVRSSYLA | QSVRSSY | RASQSVRSSYLA | RASQSVRSSYLA | RSSYLAWY | RASQSVRSSYLA |
| CDR | CDR1 | (SEQ ID NO: | (SEQ ID NO: | (SEQ ID NO: 168) | (SEQ ID NO: 174) | (SEQ ID NO: | (SEQ ID NO: |
| Seq. | | 156) | 162) | | | 180) | 186) |
| | VL | DAFSRAT | DAF | DAFSRAT | DAFSRAT | LLIYDAFSRA | DAFSRAT |
| | CDR2 | (SEQ ID NO: | (SEQ ID NO: | (SEQ ID NO: 169) | (SEQ ID NO: 175) | (SEQ ID NO: | (SEQ ID NO: |
| | | 157) | 163) | | | 181) | 187) |
| | VL | QQYGSASLIT | QQYGSASLIT | QQYGSASLIT | QQYGSASLIT | QQYGSASLI | QQYGSASLIT |
| | CDR3 | (SEQ ID NO: | (SEQ ID NO: | (SEQ ID NO: 170) | (SEQ ID NO: 176) | (SEQ ID NO: | (SEQ ID NO: |
| | | 158) | 164) | | | 182) | 188) |

VH Sequence:
QLQLQESGPGLVKPSETLSLTCTVSGGSISRGDYYWGWIRQPPGKGLEWIGTVDYSGRTYYNPSLKSRVTISVDTSKNQFSLKLSSVTAADTAVYY
CARAFPGEYFAVFDHWGQGTLVTVSS (SEQ ID NO: 189)
VL Sequence:
EIVLTQSPGTLSLSPGERATLSCRASQSVRSSYLAWYQQKPGQAPRLLIYDAFSRATGIPDRFSGSGSGTDFTLTISRLEPEDFAVYYCQQYGSAS
LITFGQGTKVEIK (SEQ ID NO: 190)

TABLE 6
AvGn-CD16a-#3A2

| | | Exemplary | IMGT | Kabat | Chothia | Contact | AbM |
|---|---|---|---|---|---|---|---|
| VH | VH | GGSIGRGDYYWG | GGSIGRGDYY | RGDYYWG | GGSIGRGDY | GRGDYYWG | GGSIGRGDYYWG |
| CDR | CDR1 | (SEQ ID NO: | (SEQ ID NO: | (SEQ ID NO: 203) | (SEQ ID NO: 209) | (SEQ ID NO: | (SEQ ID NO: |
| Seq. | | 191) | 197) | | | 215) | 221) |
| | VH | TIDYSGRTYYNPS | TIDYSGRT | TIDYSGRTYYNPS | DYSGR | WIGTIDYSGRTY | TIDYSGRTY |
| | CDR2 | LKS (SEQ ID | (SEQ ID NO: | LKS | (SEQ ID NO: 210) | (SEQ ID NO: | (SEQ ID NO: |
| | | NO: 192) | 198) | (SEQ ID NO: 204) | | 216) | 222) |
| | VH | AFPGEYFGVFDH | ARAFPGEYFGVF | AFPGEYFGVFDH | AFPGEYFGVFDH | ARAFPGEYFGVF | AFPGEYFGVFDH |
| | CDR3 | (SEQ ID NO: | DH (SEQ ID | (SEQ ID NO: 205) | (SEQ ID NO: 211) | D (SEQ ID | (SEQ ID NO: |
| | | 193) | NO: 199) | | | NO: 217) | 223) |
| VL | VL | RASQSVSSSYLA | QSVSSSY | RASQSVSSSYLA | RASQSVSSSYLA | SSSYLAWY | RASQSVSSSYLA |
| CDR | CDR1 | (SEQ ID NO: | (SEQ ID NO: | (SEQ ID NO: 206) | (SEQ ID NO: 212) | (SEQ ID NO: | (SEQ ID NO: |
| Seq. | | 194) | 200) | | | 218) | 224) |
| | VL | DATSRAT | DAT | DATSRAT | DATSRAT | LLIYDATSRA | DATSRAT |
| | CDR2 | (SEQ ID NO: | (SEQ ID NO: | (SEQ ID NO: 207) | (SEQ ID NO: 213) | (SEQ ID NO: | (SEQ ID NO: |
| | | 195) | 201) | | | 219) | 225) |
| | VL | QQYDSSSLIT | QQYDSSSLIT | QQYDSSSLIT | QQYDSSSLIT | QQYDSSSLI | QQYDSSSLIT |
| | CDR3 | (SEQ ID NO: | (SEQ ID NO: | (SEQ ID NO: 208) | (SEQ ID NO: 214) | (SEQ ID NO: | (SEQ ID NO: |
| | | 196) | 202) | | | 220) | 226) |

VH Sequence:
QLQLQESGPGLVKPSETLSLTCTVSGGSIGRGDYYWGWIRQPPGKGLEWIGTIDYSGRTYYNPSLKSRVTISVDTSKNQFSLKLSSVTAADTAVYY
CARAFPGEYFGVFDHWGQGTLVTVSS (SEQ ID NO: 227)
VL Sequence:
EIVLTQSPGTLSLSPGERATLSCRASQSVSSSYLAWYQQKPGQAPRLLIYDATSRATGIPDRFSGSGSGTDFTLTISRLEPEDFAVYYCQQYDSSS
LITFGQGTKVEIK (SEQ ID NO: 228)

TABLE 7
AvGn-CD16a-#6A2

| | | Exemplary | IMGT | Kabat | Chothia | Contact | AbM |
|---|---|---|---|---|---|---|---|
| VH | VH | GGSIGRGDYYWG | GGSIGRGDYY | RGDYYWG | GGSIGRGDY | GRGDYYWG | GGSIGRGDYYWG |
| CDR | CDR1 | (SEQ ID NO: | (SEQ ID NO: | (SEQ ID NO: 241) | (SEQ ID NO: 247) | (SEQ ID NO: | (SEQ ID NO: |
| Seq. | | 229) | 235) | | | 253) | 259) |
| | VH | TIDYSGRTYYNPS | TIDYSGRT | TIDYSGRTYYNPS | DYSGR | WIGTIDYSGRTY | TIDYSGRTY |
| | CDR2 | LKS (SEQ ID | (SEQ ID NO: | LKS | (SEQ ID NO: 248) | (SEQ ID NO: | (SEQ ID NO: |
| | | NO: 230) | 236) | (SEQ ID NO: 242) | | 254) | 260) |
| | VH | AFPGEYFGVFDH | ARAFPGEYFGVF | AFPGEYFGVFDH | AFPGEYFGVFDH | ARAFPGEYFGVF | AFPGEYFGVFDH |
| | CDR3 | (SEQ ID NO: | DH (SEQ ID | (SEQ ID NO: 243) | (SEQ ID NO: 249) | D (SEQ ID | (SEQ ID NO: |
| | | 231) | NO: 237) | | | NO: 255) | 261) |
| VL | VL | RASQSVSSSYLA | QSVSSSY | RASQSVSSSYLA | RASQSVSSSYLA | SSSYLAWY | RASQSVSSSYLA |
| CDR | CDR1 | (SEQ ID NO: | (SEQ ID NO: | (SEQ ID NO: 244) | (SEQ ID NO: 250) | (SEQ ID NO: | (SEQ ID NO: |
| Seq. | | 232) | 238) | | | 256) | 262) |
| | VL | GASSRAT | GAS | GASSRAT | GASSRAT | LLIYGASSRA | GASSRAT |
| | CDR2 | (SEQ ID NO: | (SEQ ID NO: | (SEQ ID NO: 245) | (SEQ ID NO: 251) | (SEQ ID NO: | (SEQ ID NO: |
| | | 233) | 239) | | | 257) | 263) |
| | VL | QQYGSSPLIT | QQYGSSPLIT | QQYGSSPLIT | QQYGSSPLIT | QQYGSSPLI | QQYGSSPLIT |
| | CDR3 | (SEQ ID NO: | (SEQ ID NO: | (SEQ ID NO: 246) | (SEQ ID NO: 252) | (SEQ ID NO: | (SEQ ID NO: |
| | | 234) | 240) | | | 258) | 264) |

VH Sequence:
QLQLQESGPGLVKPSETLSLTCTVSGGSIGRGDYYWGWIRQPPGKGLEWIGTIDYSGRTYYNPSLKSRVTISVDTSKNQFSLKLSSVTAADTAVYY
CARAFPGEYFGVFDHWGQGTLVTVSS (SEQ ID NO: 265)
VL Sequence:
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP
LITFGQGTKVEIK (SEQ ID NO: 266)

TABLE 8
AvGn-CD16a-#10A2

| | | Exemplary | IMGT | Kabat | Chothia | Contact | AbM |
|---|---|---|---|---|---|---|---|
| VH | VH | GGSISRGDYYWG | GGSISRGDYY | RGDYYWG | GGSISRGDY | SRGDYYWG | GGSISRGDYYWG |
| CDR | CDR1 | (SEQ ID NO: | (SEQ ID NO: | (SEQ ID NO: 279) | (SEQ ID NO: 285) | (SEQ ID NO: | (SEQ ID NO: |
| Seq. | | 267) | 273) | | | 291) | 297) |
| | VH | TIDYSGRSYYNPS | TIDYSGRS | TIDYSGRSYYNPS | DYSGR | WIGTIDYSGRSY | TIDYSGRSY |
| | CDR2 | LKS (SEQ ID | (SEQ ID NO: | LKS | (SEQ ID NO: 286) | (SEQ ID NO: | (SEQ ID NO: |
| | | NO: 268) | 274) | (SEQ ID NO: 280) | | 292) | 298) |
| | VH | AFPGEYFGVFDH | ARAFPGEYFGVF | AFPGEYFGVFDH | AFPGEYFGVFDH | ARAFPGEYFGVF | AFPGEYFGVFDH |
| | CDR3 | (SEQ ID NO: | DH (SEQ ID | (SEQ ID NO: 281) | (SEQ ID NO: 287) | D (SEQ ID | (SEQ ID NO: |
| | | 269) | NO: 275) | | | NO: 293) | 299) |
| VL | VL | RASQRVSSSYLA | QRVSSSY | RASQRVSSSYLA | RASQRVSSSYLA | SSSYLAWY | RASQRVSSSYLA |
| CDR | CDR1 | (SEQ ID NO: | (SEQ ID NO: | (SEQ ID NO: 282) | (SEQ ID NO: 288) | (SEQ ID NO: | (SEQ ID NO: |
| Seq. | | 270) | 276) | | | 294) | 300) |
| | VL | DAISRAT | DAI | DAISRAT | DAISRAT | LLIYDAISRA | DAISRAT |
| | CDR2 | (SEQ ID NO: | (SEQ ID NO: | (SEQ ID NO: 283) | (SEQ ID NO: 289) | (SEQ ID NO: | (SEQ ID NO: |
| | | 271) | 277) | | | 295) | 301) |
| | VL | QQYGSSSLIT | QQYGSSSLIT | QQYGSSSLIT | QQYGSSSLIT | QQYGSSSLI | QQYGSSSLIT |
| | CDR3 | (SEQ ID NO: | (SEQ ID NO: | (SEQ ID NO: 284) | (SEQ ID NO: 290) | (SEQ ID NO: | (SEQ ID NO: |
| | | 272) | 278) | | | 296) | 302) |

VH Sequence:
QLQLQESGPGLVKPSETLSLTCTVSGGSISRGDYYWGWIRQPPGKGLEWIGTIDYSGRSYYNPSLKSRVTISVDTSKNQFSLKLSSVTAADTAVYY
CARAFPGEYFGVFDHWGQGTLVTVSS (SEQ ID NO: 303)
VL Sequence:
EIVLTQSPGTLSLSPGERATLSCRASQRVSSSYLAWYQQKPGQAPRLLIYDAISRATGIPDRFSGSGSGTDFTLTISRLEPEDFAVYYCQQYGSSS
LITFGQGTKVEIK (SEQ ID NO: 304)

TABLE 9
AvGn-CD16a-#55

| | | Exemplary | IMGT | Kabat | Chothia | Contact | AbM |
|---|---|---|---|---|---|---|---|
| VH | VH | GFTFSNYAMS | GFTFSNYA | NYAMS | GFTFSNY | SNYAMS | GFTFSNYAMS |
| CDR | CDR1 | (SEQ ID NO: | (SEQ ID NO: | (SEQ ID NO: 317) | (SEQ ID NO: 323) | (SEQ ID NO: | (SEQ ID NO: |
| Seq. | | 305) | 311) | | | 329) | 335) |
| | VH | AISGGGSYTYYA | ISGGGSYT | AISGGGSYTYYA | SGGGSY | WVSAISGGGSYT | AISGGGSYTY |
| | CDR2 | DSVKG (SEQ ID | (SEQ ID NO: | DSVKG | (SEQ ID NO: 324) | Y (SEQ ID | (SEQ ID NO: |
| | | NO: 306) | 312) | (SEQ ID NO: 318) | | NO: 330) | 336) |
| | VH | TYLRRWYYFDY | ARTYLRRWYYFD | TYLRRWYYFDY | TYLRRWYYFDY | ARTYLRRWYYFD | TYLRRWYYFDY |
| | CDR3 | (SEQ ID NO: | Y (SEQ ID | (SEQ ID NO: 319) | (SEQ ID NO: 325) | (SEQ ID NO: | (SEQ ID NO: |
| | | 307) | NO: 313) | | | 331) | 337) |
| VL | VL | RASQTITSYLN | QTITSY | RASQTITSYLN | RASQTITSYLN | TSYLNWY | RASQTITSYLN |
| CDR | CDR1 | (SEQ ID NO: | (SEQ ID NO: | (SEQ ID NO: 320) | (SEQ ID NO: 326) | (SEQ ID NO: | (SEQ ID NO: |
| Seq. | | 308) | 314) | | | 332) | 338) |
| | VL | AASSLQS | AAS | AASSLQS | AASSLQS | LLIYAASSLQ | AASSLQS |
| | CDR2 | (SEQ ID NO: | (SEQ ID NO: | (SEQ ID NO: 321) | (SEQ ID NO: 327) | (SEQ ID NO: | (SEQ ID NO: |
| | | 309) | 315) | | | 333) | 339) |
| | VL | QQSYTSPVT | QQSYTSPVT | QQSYTSPVT | QQSYTSPVT | QQSYTSPV | QQSYTSPVT |
| | CDR3 | (SEQ ID NO: | (SEQ ID NO: | (SEQ ID NO: 322) | (SEQ ID NO: 328) | (SEQ ID NO: | (SEQ ID NO: |
| | | 310) | 316) | | | 334) | 340) |

VH Sequence:
EVQLLESGGGLVQPGGSLRLSCAASGFTFSNYAMSWVRQAPGKGLEWVSAISGGGSYTYYADSVKGRFTISRDNSKNTLYLQMNSLRAEDTAVY
YCARTYLRRWYYFDYWGQGTLVTVSS (SEQ ID NO: 341)
VL Sequence:
DIQMTQSPSSLSASVGDRVTITCRASQTITSYLNWYQQKPGKAPKLLIYAASSLQSGVPSRFSGSGSGTDFTLTISSLQPEDFATYYCQQSYTS
PVTFGQGTKVEIK (SEQ ID NO: 342)

### 5.2.2. CD19 Binding Domains

Also provided herein is a binding agent that binds to CD19. In some embodiments, the binding agent comprises one or more CD19 binding domains. In some embodiments, the multispecific binding agents provided herein further comprise one or more CD19 binding domains. In some embodiments, described herein are multispecific binding agents (e.g., bispecific antibodies) that bind to CD19. As used herein, CD19 refers to a CD19 polypeptide, a CD19 polypeptide fragment, a CD19 peptide or a CD19 epitope. In some embodiments, the CD19 binding domains in the present binding agents, such as multispecific binding agents, are derived from human or humanized antibodies (e.g., comprising human framework regions) that bind CD19, including a CD19 polypeptide, a CD19 polypeptide fragment, a CD19 peptide or a CD19 epitope. In some embodiments, the binding agent (e.g., an antibody, a multispecific antibody, or antibody fragment) can bind to CD19 expressed on the surface of a mammalian (e.g., human) cell, including a CD19 expressing cancer or tumor cell. In some embodiments, the binding agent (e.g., an antibody, a multispecific antibody, or antibody fragment) provided herein binds a CD19 extracellular epitope exposed on a cell such as a cancer or tumor cell. In some embodiments, described herein is a binding agent (e.g., an antibody, a multispecific antibody, or antibody fragment) that binds to CD19, such as human CD19 or portions thereof. In some embodiments, CD19 is a human CD19. In some embodiments, the binding agent (such as an antibody, a multispecific antibody, or antibody fragment) provided herein is a human CD19 binding agent (e.g., an antibody that binds to human CD19). In some embodiments, the binding agent provided herein binds to both human CD19 and cyno CD19. In other embodiments, the binding agent provided herein binds to human CD19 but not cyno CD19.

In some embodiments, the binding agent (e.g., an antibody, a multispecific antibody, or antibody fragment) provided herein binds to CD19 (e.g., human CD19) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including those described herein, e.g., in Section 5.2.1 above.

In some embodiments, the binding agent provided herein is obtained from Drug Bank accession number DB09052. In some embodiments, the binding agents (e.g., an antibody, a multispecific antibody, or antibody fragment) described herein comprise a VH region, VL region, VH CDR1, VH CDR2, VH CDR3, VL CDR1, VL CDR2, and/or VL CDR3 of any anti-CD19 antibody, such as an amino acid sequence of a VH region, VL region, VH CDR1, VH CDR2, VH CDR3, VL CDR1, VL CDR2, and/or VL CDR3 depicted in Table 10. Accordingly, in some embodiments, a binding agent (e.g., an antibody, a multispecific antibody, or antibody fragment) described herein comprises any one, any two, and/or all three heavy chain CDRs and/or any one, any two, and/or all three light chain CDRs from the antibody as shown in Table 10. In some embodiments, a binding agent (e.g., an antibody, a multispecific antibody, or antibody fragment) described herein comprises any one, any two, and/or all three heavy chain CDRs and any one, any two, and/or all three light chain CDRs from the antibody as shown in Table 10.

In some embodiments, a binding agent (e.g., an antibody, a multispecific antibody, or antibody fragment) comprises a VH region, which comprises a VH CDR1, a VH CDR2, and/or a VH CDR3, and/or a VL region, which comprises a VL CDR1, a VL CDR2, and/or a VL CDR3, of any one of the binding agents described herein (see, e.g., Table 10). Accordingly, in some embodiments, a binding agent (e.g., an antibody, a multispecific antibody, or antibody fragment) described herein comprises any one, any two, and/or all three heavy chain CDRs and/or any one, any two, and/or all three light chain CDRs from Table 10.

In some embodiments, the binding agent provided herein (e.g., an antibody, a multispecific antibody, or antibody fragment) comprises (i) a VH CDR1, a VH CDR2, and a VH CDR3 as set forth in a VH comprising the amino acid sequence of SEQ ID NO: 343 and/or (ii) a VL CDR1, a VL CDR2, and a VL CDR3 as set forth in a VL comprising the amino acid sequence of SEQ ID NO: 344. In some embodiments, the binding agent provided herein (e.g., an antibody, a multispecific antibody, or antibody fragment) comprises a VH CDR1, a VH CDR2, and a VH CDR3 as set forth in a VH comprising the amino acid sequence of SEQ ID NO: 343 and/or a VL CDR1, a VL CDR2, and a VL CDR3 as set forth in a VL comprising the amino acid sequence of SEQ ID NO: 344. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the CDR sequences are determined according to a combination of any two or more of the above-mentioned numbering systems, for example, a combination of Kabat and Chothia. Various exemplary CDR numbering systems are described and illustrated above in Section 5.1.

In some embodiments, the CD19 binding domain in the binding agent (e.g., an antibody, a multispecific antibody, or antibody fragment) provided herein further comprises one or more framework regions of SEQ ID NOs: 343 and 344. In some embodiments, the CD19 binding domain further comprises a framework 1 (FR1), a framework 2 (FR2), a framework 3 (FR3) and/or a framework 4 (FR4) sequence as set forth in any one of SEQ ID NOs: 343 and 344. In some embodiments, the CD19 binding domain provided herein is derived from a humanized antibody. Framework regions described herein are determined based upon the boundaries of the CDR numbering system as described in Section 5.2.1 above.

In some embodiments, the CD19 binding domains described herein comprise a VH region or VH domain. Additionally or alternatively, in some embodiments, the CD19 binding domains described herein comprise a VL region or VL domain. In some embodiments, the CD19 binding domains described herein have a combination of (i) a VH domain or VH region; and (ii) a VL domain or VL region.

In some embodiments, the binding agent provided herein (e.g., an antibody, a multispecific antibody, or antibody fragment) comprises a VH comprising the amino acid sequence of SEQ ID NO: 343. In some embodiments, the binding agent provided herein (e.g., an antibody, a multispecific antibody, or antibody fragment) comprises a VL comprising the amino acid sequence of SEQ ID NO: 344. In some embodiments, the binding agent provided herein (e.g., an antibody, a multispecific antibody, or antibody fragment) comprises a VH comprising the amino acid sequence of SEQ ID NO: 343 and a VL comprising the amino acid sequence of SEQ ID NO: 344.

In certain embodiments, the binding agent (e.g., an antibody, a multispecific antibody, or antibody fragment) provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a CDR, VH or VL in Table 10, or a full-length antibody chain as disclosed herein. In some embodiments, the binding agent (e.g., an antibody, a multispecific antibody, or antibody fragment) provided herein comprises CDRs of any antibody or fragment thereof provided herein, for example in Table 10. In further embodiments, the binding agent (e.g., an antibody, a multispecific antibody, or antibody fragment) provided herein comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a VH or VL in Table 10, or a full-length antibody chain as disclosed herein. The determination of percent identity between two sequences (e.g., amino acid sequences or nucleic acid sequences) can be accomplished using a method as described in Section 5.2.1 above.

In some embodiments, the binding agent (e.g., an antibody, a multispecific antibody, or antibody fragment) provided herein contains substitutions (e.g., conservative substitutions), insertions, or deletions relative to the reference sequence, but the binding agent (e.g., an antibody, a multispecific antibody, or antibody fragment) comprising that sequence retains the ability to bind to CD19. In some embodiments, a total of 1 to 10 amino acids have been substituted, inserted and/or deleted in a reference amino acid sequence. In some embodiments, substitutions, insertions, or deletions occur in regions outside the CDRs (e.g., in the FRs, constant regions, and/or Fc regions).

In some embodiments, the position of one or more CDRs along the VH (e.g., CDR1, CDR2, or CDR3) and/or VL (e.g., CDR1, CDR2, or CDR3) region of a CD19 binding domain described herein may vary by one, two, three, four, five, or six amino acid positions so long as binding to CD19 (e.g., human CD19) is maintained (e.g., substantially maintained, for example, at least 50%, at least 60%, at least 70%, at least 80%, at least 90%, or at least 95%). For example, in some embodiments, the position defining a CDR of Table 10 may vary by shifting the N-terminal and/or C-terminal boundary of the CDR by one, two, three, four, five, or six amino acids, relative to the current CDR position, so long as binding to CD19 (e.g., human CD19) is maintained (e.g., substantially maintained, for example, at least 50%, at least 60%, at least 70%, at least 80%, at least 90%, or at least 95%). Additionally or alternatively, in some embodiments, the length of one or more CDRs along the VH (e.g., CDR1, CDR2, or CDR3) and/or VL (e.g., CDR1, CDR2, or CDR3) region of a CD19 binding domain described herein may vary (e.g., be shorter or longer) by one, two, three, four, five, or more amino acids, so long as binding to CD19 (e.g., human CD19) is maintained (e.g., substantially maintained, for example, at least 50%, at least 60%, at least 70%, at least 80%, at least 90%, or at least 95%). Any method known in the art can be used to ascertain whether binding to CD19 (e.g., human CD19) is maintained, for example, the binding assays and conditions described in the “Examples” section described herein.

In other embodiments, the binding agents (e.g., an antibody, a multispecific antibody, or antibody fragment) further comprise conservative sequence modifications (e.g., in a CD19 binding domain). Conservative sequence modifications are described in more detail in Section 5.2.1 above. In some embodiments, the conservative sequence modifications described herein modify the amino acid sequences of the binding agents (e.g., an antibody, a multispecific antibody, or antibody fragment), including human CD19 binding agents, by 50%, or 55%, or 60%, or 65%, or 70%, or 75%, or 80%, or 85%, or 90%, or 95%, or 98%, or 99%. In some embodiments, the amino acid sequence modifications refer to at most 1, 2, 3, 4, 5, or 6 amino acid substitutions to the CDRs, such as those described in Table 10. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, the CD19 binding domain contains one or more, including six, CDRs having at least 75%, 80%, 85%, 90%, 95%, 96%, 97%, 98%, or 99% identity to the CDRs of the antibody disclosed in Table 10.

In some embodiments, a CD19 binding domain contains a VH and a VL comprising CDRs identical to those of Table 10. In some embodiments, the amino acid sequence modifications do not include any modification within an SDR. In some embodiments, the amino acid sequence modifications do not include any modification within a CDR (such as CDR1, CDR2, CDR3, or any combination thereof). In further embodiments, the amino acid sequence modifications are in the framework, constant region, and/or Fc region.

In some embodiments, the binding agent (e.g., an antibody, a multispecific antibody, or antibody fragment) provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 343, and/or a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 344, and the binding of the binding agent to CD19 (e.g., human CD19) is maintained (e.g., substantially maintained, for example, at least 50%, at least 60%, at least 70%, at least 80%, at least 90%, or at least 95%).

In some embodiments, functional epitopes can be mapped, e.g., by combinatorial alanine scanning, to identify amino acids in the CD19 protein that are necessary for interaction with a multispecific binding agent, a CD19 binding domain thereof, and/or an anti-CD19 antibody provided herein (e.g., in the following paragraphs). In some embodiments, conformational and crystal structure of a multispecific binding agent, a CD19 binding domain thereof, and/or an anti-CD19 antibody (e.g., provided in the following paragraphs) bound to CD19 may be employed to identify the epitopes. In some embodiments, the present disclosure provides a binding agent (e.g., an antibody, a multispecific antibody, or antibody fragment) comprising a CD19 binding domain that specifically binds to the same epitope as any of the multispecific binding agents as disclosed herein, CD19 binding domains thereof, and/or the anti-CD19 antibodies or fragments thereof provided herein (e.g., provided in the following paragraphs).

For example, in some embodiments, the CD19 binding domain provided herein binds to the same epitope as an anti-CD19 antibody comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the CD19 binding domain provided herein binds to the same epitope as an anti-CD19 antibody comprising a VH comprising the amino acid sequence of SEQ ID NO: 343, and a VL comprising the amino acid sequence of SEQ ID NO: 344.

In some embodiments, the binding agent provided herein (e.g., an antibody, a multispecific antibody, or antibody fragment) specifically binds to CD19 competitively with any one of the anti-CD19 antibodies or fragments thereof described herein.

In some embodiments, the binding agent provided herein (e.g., an antibody, a multispecific antibody, or antibody fragment) specifically binds to CD19 competitively with an anti-CD19 antibody comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the binding agent provided herein (e.g., an antibody, a multispecific antibody, or antibody fragment) specifically binds to CD19 competitively with an anti-CD19 antibody comprising a VH comprising the amino acid sequence of SEQ ID NO: 343, and a VL comprising the amino acid sequence of SEQ ID NO: 344.

In yet another aspect, provided herein is a binding agent (e.g., an antibody or fragment thereof) that binds to CD19 comprising one or more CDR(s) described above. In some embodiments, provided herein is a binding agent (e.g., an antibody or fragment thereof) that binds to CD19 comprising the VH and/or VL described above. In some embodiments, the binding agent comprises a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344.

In some embodiments, the binding agent provided herein comprises a VH region comprising the amino acid sequence of SEQ ID NO: 343 and a VL region comprising the amino acid sequence of SEQ ID NO: 344.

In some embodiments, the CD19 binding agent comprises a scFv having a combination of (i) a VH described herein and (ii) a VL described herein. In some embodiments, the CD19 binding agent comprises an amino acid sequence of SEQ ID NO: 345 obtained from Drug Bank accession number DB09052.

TABLE 10
Anti-CD19 antibody

| VH | QVQLQQSGAELVRPGSSVKISCKASGYAFSSYWMNWVKQRPGQG |
|---|---|
| | LEWIGQIWPGDGDTNYNGKFKGKATLTADESSSTAYMQLSSLAS |
| | EDSAVYFCARRETTTVGRYYYAMDYWGQGTTVTVSS (SEQ ID |
| | NO: 343) |
| VL | DIQLTQSPASLAVSLGQRATISCKASQSVDYDGDSYLNWYQQIP |
| | GQPPKLLIYDASNLVSGIPPRFSGSGSGTDFTLNIHPVEKVDAA |
| | TYHCQQSTEDPWTFGGGTKLEIK (SEQ ID NO: 344) |
| scFv | DIQLTQSPASLAVSLGQRATISCKASQSVDYDGDSYLNWYQQIP |
| | GQPPKLLIYDASNLVSGIPPRFSGSGSGTDFTLNIHPVEKVDAA |
| | TYHCQQSTEDPWTFGGGTKLEIKGGGGSGGGGSGGGGSQVQLQQ |
| | SGAELVRPGSSVKISCKASGYAFSSYWMNWVKQRPGQGLEWIGQ |
| | IWPGDGDTNYNGKFKGKATLTADESSSTAYMQLSSLASEDSAVY |
| | FCARRETTTVGRYYYAMDYWGQGTTVTVSS (SEQ ID NO: |
| | 345) |

### 5.2.3. Multispecific Antibodies

Provided herein is a multispecific binding agent that binds to CD16a and a non-CD16a antigen (such as a tumor antigen). In some embodiments, the multispecific binding agent comprises one or more CD16a binding domains and one or more non-CD16a antigen binding domains (such as one or more tumor antigen binding domains). In certain embodiments, the present multispecific binding agent is a multispecific antibody comprising one or more CD16a binding domains each independently selected from the CD16a binding domains described in Section 5.2.1 above and one or more tumor antigen binding domain. In some embodiments, the tumor antigen is CD19. In some embodiments, the tumor antigen is BCMA. In certain embodiments, the present multispecific binding agent is a multispecific antibody comprising one or more CD16a binding domains each independently selected from the CD16a binding domains described in Section 5.2.1 above and one or more CD19 binding domains each independently selected from the CD19 binding domains described in Section 5.2.2 above.

In certain embodiments, the present multispecific binding agent is a multispecific antibody comprising a first binding domain that binds to CD16a and a second binding domain that binds to a second antigen. In some embodiments, the second antigen is a tumor antigen. In some embodiments, the second antigen is CD19.

In some embodiments of the multispecific antibody provided herein, the first binding domain that binds to CD16a comprises any antibody or antigen binding fragment described in Section 5.2.1 above. In some embodiments, the second antigen is a tumor antigen. In some embodiments, the second antigen is CD19. In some embodiments of the multispecific antibody provided herein, the second binding domain that binds to CD19 comprises any antibody or antigen binding fragment described in Section 5.2.2 above.

In some embodiments of the multispecific antibody provided herein, the first binding domain comprises six CDRs of any anti-CD16a antibody or antigen binding fragment described in Section 5.2.1 above. In some embodiments, the second antigen is a tumor antigen. In some embodiments, the second antigen is CD19. In some embodiments of the multispecific antibody provided herein, the second binding domain comprises six CDRs of any anti-CD19 antibody or antigen binding fragment described in Section 5.2.2 above.

In some embodiments of the multispecific antibody provided herein, the first binding domain comprises a VH and a VL of any anti-CD16a antibody or antigen binding fragment described in Section 5.2.1 above. In some embodiments, the second antigen is a tumor antigen. In some embodiments, the second antigen is CD19. In some embodiments of the multispecific antibody provided herein, the second binding domain comprises a VH and a VL of any anti-CD19 antibody or antigen binding fragments as described in Section 5.2.2 above.

In some embodiments of the multispecific antibody provided herein, the first binding domain comprises six CDRs of any anti-CD16a antibody or antigen binding fragment described in Tables 1-9. In some embodiments, the second antigen is a tumor antigen. In some embodiments, the second antigen is CD19. In some embodiments of the multispecific antibody provided herein, the second binding domain comprises six CDRs of any anti-CD19 antibody or antigen binding fragment described in Table 10.

In some embodiments of the multispecific antibody provided herein, the first binding domain comprises a VH and a VL of any anti-CD16a antibody or antigen binding fragment described in Tables 1-9. In some embodiments, the second antigen is a tumor antigen. In some embodiments, the second antigen is CD19. In some embodiments of the multispecific antibody provided herein, the second binding domain comprises a VH and a VL of any anti-CD19 antibody or antigen binding fragments as described in Table 10.

In some specific embodiments, provided herein is a multispecific binding agent comprising one or more CD16a binding domains each independently selected from CD16a binding domains comprising: (i) a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38; (ii) a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76; (iii) a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114; (iv) a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152; (v) a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190; (vi) a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228; (vii) a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266; (viii) a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304; or (ix) a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342; and one or more CD19 binding domains comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344.

In some embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some specific embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some specific embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some specific embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some specific embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some specific embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some specific embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some specific embodiments, provided herein is a multispecific binding agent comprising one or more CD16a binding domains, each comprising a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342; and one or more CD19 binding domains, each comprising a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344. In some embodiments, the multispecific binding agent provided herein comprises one CD16a binding domain (e.g., with a pair of VH and VL regions that bind to CD16a) and one CD19 binding domain (e.g., with a pair of VH and VL regions that bind to CD19). In some embodiments, the multispecific binding agent provided herein comprises two CD16a binding domains (e.g., with two identical pairs of VH and VL regions that bind to CD16a) and one CD19 binding domain (e.g., with a pair of VH and VL regions that bind to CD19). In some embodiments, the multispecific binding agent provided herein comprises one CD16a binding domain (e.g., with a pair of VH and VL regions that bind to CD16a) and two CD19 binding domains (e.g., with two identical pairs of VH and VL regions that bind to CD19). In some embodiments, the multispecific binding agent provided herein comprises two CD16a binding domains (e.g., with two identical pairs of VH and VL regions that bind to CD16a) and two CD19 binding domains (e.g., with two identical pairs of VH and VL regions that bind to CD19).

In some embodiments, provided herein is a multispecific binding agent comprising a first binding domain that binds to CD16a comprising a VH comprising the amino acid sequence of SEQ ID NO: 37 and a VL comprising the amino acid sequence of SEQ ID NO: 38; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 37 and a VL having at least 90% sequence identity to SEQ ID NO: 38; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 75 and a VL comprising the amino acid sequence of SEQ ID NO: 76; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 75 and a VL having at least 90% sequence identity to SEQ ID NO: 76; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 113 and a VL comprising the amino acid sequence of SEQ ID NO: 114; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 113 and a VL having at least 90% sequence identity to SEQ ID NO: 114; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 151 and a VL comprising the amino acid sequence of SEQ ID NO: 152; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 151 and a VL having at least 90% sequence identity to SEQ ID NO: 152; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 189 and a VL comprising the amino acid sequence of SEQ ID NO: 190; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 189 and a VL having at least 90% sequence identity to SEQ ID NO: 190; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 227 and a VL comprising the amino acid sequence of SEQ ID NO: 228; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 227 and a VL having at least 90% sequence identity to SEQ ID NO: 228; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 265 and a VL comprising the amino acid sequence of SEQ ID NO: 266; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 265 and a VL having at least 90% sequence identity to SEQ ID NO: 266; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 303 and a VL comprising the amino acid sequence of SEQ ID NO: 304; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 303 and a VL having at least 90% sequence identity to SEQ ID NO: 304; and a second binding domain that binds to CD19 comprising a VH having at least 90% sequence identity to SEQ ID NO: 343 and a VL having at least 90% sequence identity to SEQ ID NO: 344.

In some embodiments, provided herein is a multispecific binding agent comprising a first binding domain that binds to CD16a comprising a VH comprising the amino acid sequence of SEQ ID NO: 341 and a VL comprising the amino acid sequence of SEQ ID NO: 342; and a second binding domain that binds to CD19 comprising a VH comprising the amino acid sequence of SEQ ID NO: 343 and a VL comprising the amino acid sequence of SEQ ID NO: 344. In some embodiments, provided herein is a multispecific binding agent comprising a first binding domain that binds to CD16a comprising a VH having at least 90% sequence identity to SEQ ID NO: 341 and a VL having at least 90% sequence identity to SEQ ID NO: 342; and a second binding domain that binds to CD19 comprising a VH having at least 90% sequence identity to SEQ ID NO: 343 and a VL having at least 90% sequence identity to SEQ ID NO: 344.

In some embodiments, the multispecific binding agent provided herein comprises one CD16a binding domains (e.g., with a pair of VH and VL regions that bind to CD16a) and one tumor antigen binding domain (e.g., with a pair of VH and VL regions that bind to a tumor antigen). In some embodiments, the multispecific binding agent provided herein comprises two CD16a binding domains (e.g., with two identical or different pairs of VH and VL regions that bind to CD16a) and one tumor antigen binding domain (e.g., with a pair of VH and VL regions that bind to a tumor antigen). In some embodiments, the multispecific binding agent provided herein comprises one CD16a binding domain (e.g., with a pair of VH and VL regions that bind to CD16a) and two tumor antigen binding domains (e.g., with two identical or different pairs of VH and VL regions that bind to one or more tumor antigen). In some embodiments, the multispecific binding agent provided herein comprises two CD16a binding domains (e.g., with two identical or different pair of VH and VL regions that bind to CD16a) and two tumor antigen binding domains (e.g., with two identical or different pairs of VH and VL regions that bind to one or more tumor antigen). In some embodiments, the multispecific binding agent further comprises one or more heavy chain constant regions (such as CH1, CH2, and/or CH3) and/or one or more light chain constant regions (such as CL). In some embodiments, the tumor antigen is CD19.

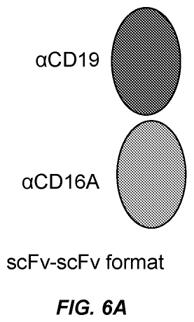

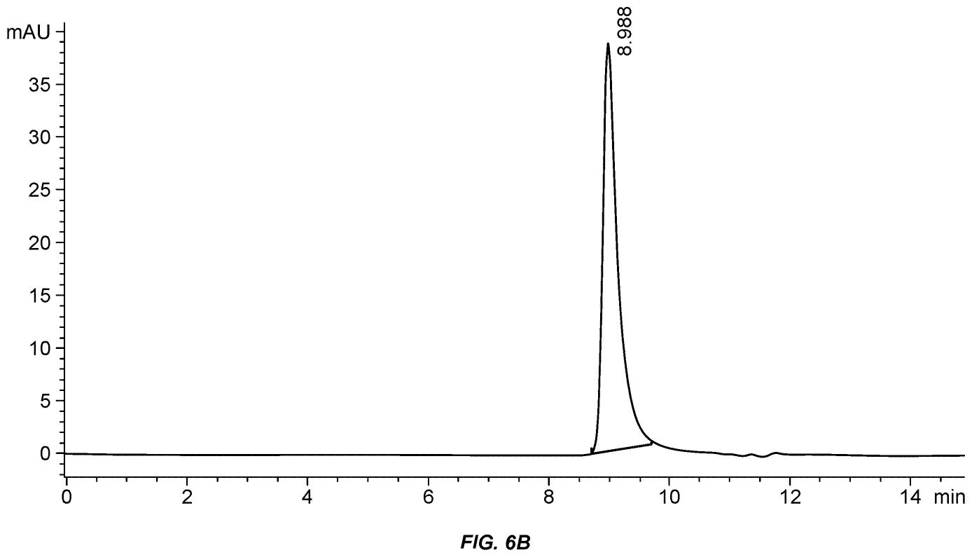

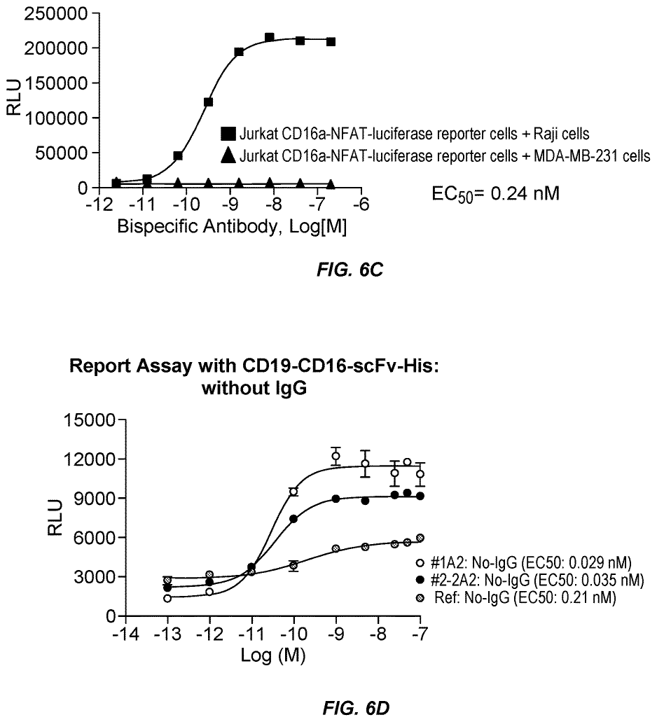

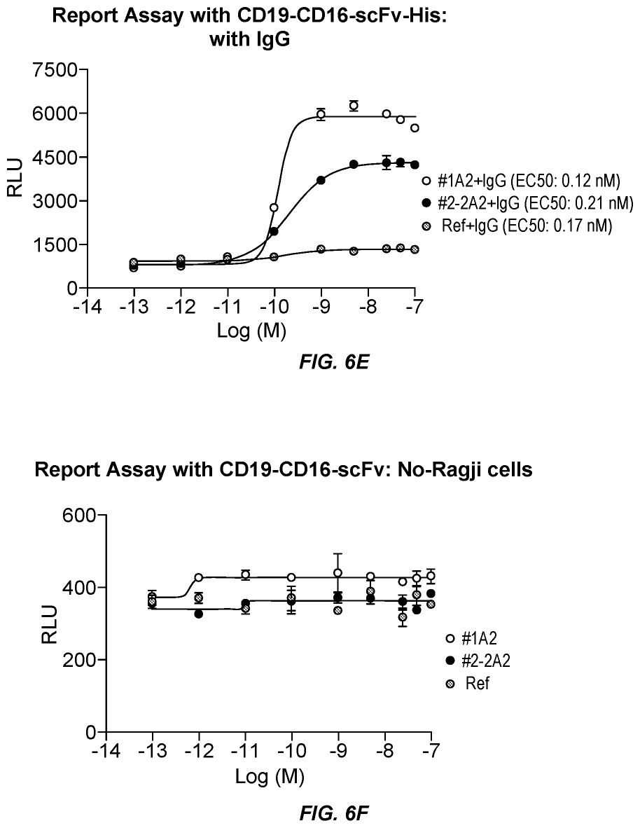

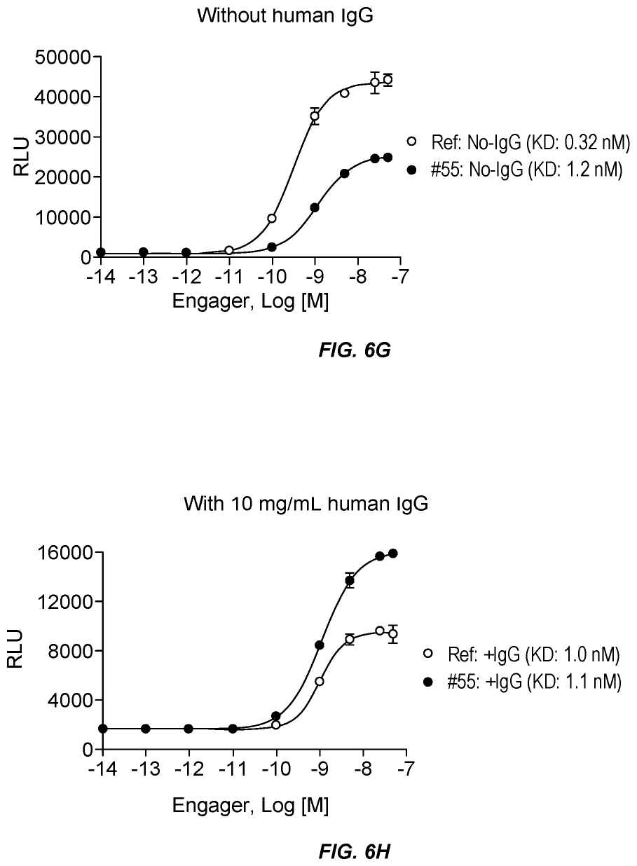

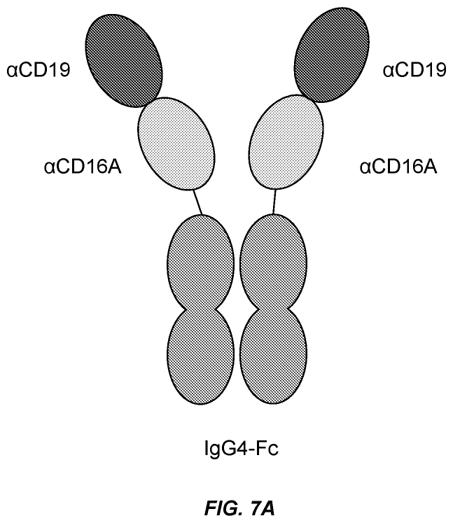

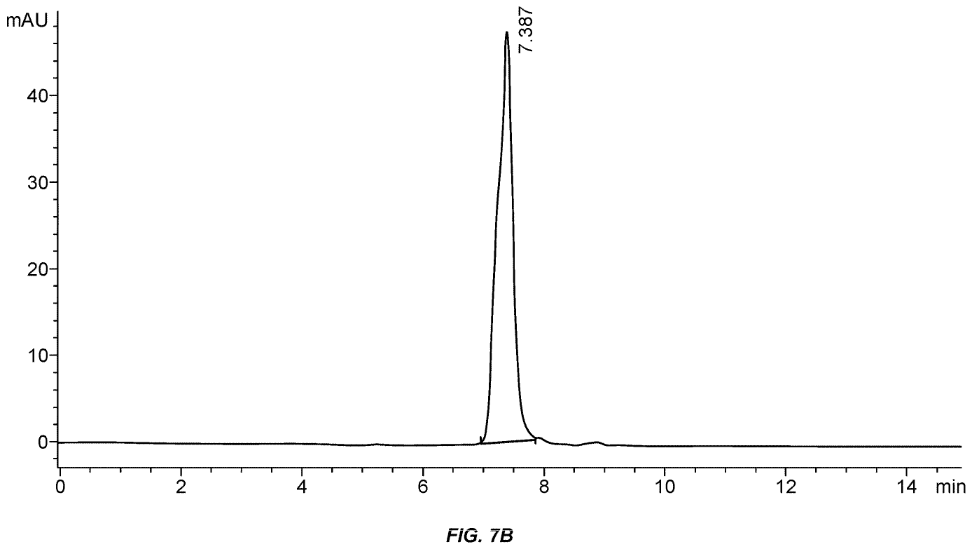

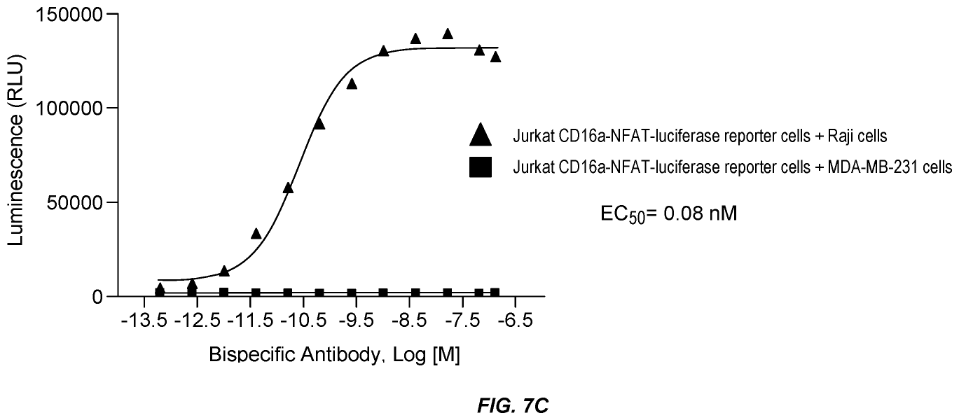

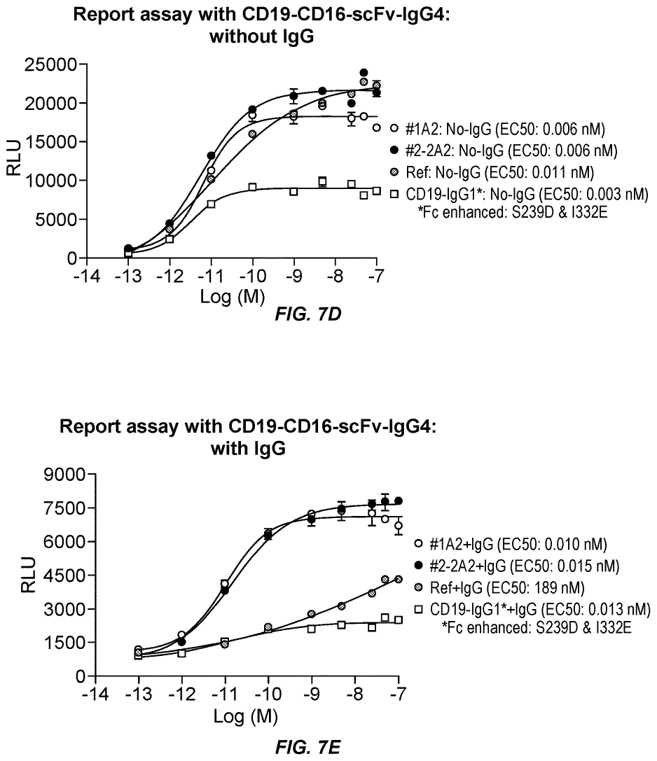

In some embodiments, the multispecific binding agent provided herein has an antibody format shown in FIG. 6A. In some embodiments, the multispecific binding agent provided herein has an antibody format shown FIG. 6A, but its CD19 binding arm is switched with its CD16a binding arm. In some embodiments, the multispecific binding agent provided herein has an antibody format shown in FIG. 7A. In some embodiments, the multispecific binding agent provided herein has an antibody format shown FIG. 7A, but its CD19 binding arm is switched with its CD16a binding arm.

Although specific exemplary multispecific antibody formats are described in the Example section below and in any of FIG. 6A or FIG. 7A, it is contemplated that any multispecific antibody formats known in the art can be used and are included in the present disclosure.

Multispecific binding agents (e.g., bispecific antibodies) described herein may be bispecific, trispecific or of greater multispecificity. Such agents may include multispecific antibodies. In some embodiments, multispecific antibodies, such as bispecific antibodies, are monoclonal antibodies that have binding specificities for at least two different targets (e.g., antigens) or two different epitopes on the same target (e.g., a bispecific antibody directed to CD16a with a first binding domain for a first epitope of an CD16a, and a second binding domain for a second epitope of CD16a). In some embodiments, the multispecific (e.g., bispecific) antibodies can be constructed based on the sequences of the antibodies described herein, e.g., the CDR sequences listed in Tables 1-9. In some embodiments, the multispecific antibodies described herein are bispecific antibodies. In some embodiments, the multispecific antibody described herein (e.g., a bispecific antibody) has binding specificities for two targets, for example, CD16a and a tumor antigen (such as CD19). In some embodiments, bispecific antibodies are mouse, chimeric, human or humanized antibodies.

In some embodiments, one of the binding specificities of the multispecific antibody provided herein is for CD16a, a second binding specificity of the multispecific antibody provided herein is a tumor antigen (such as CD19), and yet one or more additional binding specificities are for any other target(s) (e.g., antigen). In some embodiments, a multispecific antibody can comprise more than one target (e.g., antigen) binding domain, in which different binding domains are specific for different targets. In some embodiments, the additional target is an immune checkpoint regulator (e.g., a negative checkpoint regulator). In some embodiments, the additional target is expressed on an immune cell. In some embodiments, the additional target is expressed on a tumor or cancer cell.

In some embodiments, multispecific (e.g., bispecific) antibody molecules can bind more than one (e.g., two or more) epitopes on the same target (e.g., antigen). In some embodiments, a multispecific (e.g., bispecific) binder as disclosed herein can bind to one or more epitopes on a first target (such as CD16a) and one or more epitopes on a second target (such as a tumor antigen).

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Exemplary structures of multispecific antibodies are known in the art and are further described in Weidle et al., 2013, Cancer Genomics & Proteomics 10: 1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, KA-body, and/or orthogonal Fab.

In some embodiments, BslgG comprises heavy chains that are engineered for heterodimerization. For example, heavy chains can be engineered for heterodimerization using a “knobs-into-holes” strategy, a SEED platform, a common heavy chain (e.g., in KA-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015): 95-106. In some embodiments, an exemplary antibody format is a B-Body format for monospecific or multispecific (e.g., bispecific antibodies) as described in e.g. WO 2018/075692, U.S. Pat. Publ. No. 2018/0118811, and U.S. Pat. Publ. No. 2021/0155692.

Bispecific (Bs) antibody (BsAb) fragments are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Non-limiting examples of bispecific antibody fragments include, but are not limited to, nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)₂, F(ab′)₂-scFv2, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example, bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be constructed to extend the serum half-life of antibody fragments. In some embodiments, chemical conjugation, e.g., chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, multispecific binding agents (e.g., bispecific antibodies) disclosed herein can be provided in any antibody format disclosed herein or known in the art. As a non-limiting example, in some embodiments, the multispecific binding agents (e.g., bispecific antibodies) can be selected from Fabs-in-tandem-lg (FIT-lg); DVD-lg; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (Bites; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; Humanized bispecific IgG antibody (REGN1979) (Regeneron); Mab2 bispecific antibodies (F-Star); DVD-lg=dual variable domain immunoglobulin (Abbott); kappa-lambda bodies; TBTI=tetravalent bispecific tandem Ig; B-Body; and CrossMab (Roche).

In some specific embodiments, the bispecific antibody provided herein is in a format depicted in any of FIG. 6A or FIG. 7A. In some embodiments, the bispecific antibody provided herein is in the 1×1 format as depicted in FIG. 6A. In some embodiments, the bispecific antibody provided herein is in the 2×2 format as depicted in FIG. 7A.

In some embodiments, the multispecific antibody provided herein comprises one polypeptide chain comprising the first binding domain and the second binding domain. In some embodiment, the first binding domain comprises a first scFv that binds to CD16a, and the second binding domain comprises a second scFv that binds to a tumor antigen (such as CD19). In some embodiment, the multispecific antibody provided comprises an antibody format of ScFv-ScFv. “ScFv” are antibody fragments that comprise the VH and VL antibody domains connected into a single polypeptide chain. Preferably, the scFv polypeptide further comprises a polypeptide linker between the VH and VL domains which enables the sFv to form the desired structure for antigen binding. In some embodiments, the linker comprises amino acid sequence of GGGGSGGGGSGGGGS (SEQ ID NO: 358). In some embodiment, the first scFv is linked to the N terminal of the second scFv. In some embodiment, the first scFv is linked to the C terminal of the second scFv. In some embodiments, the scFv-scFv construct comprises amino acid sequence of SEQ ID NO: 357.

In some specific embodiments, the multispecific antibody provided herein comprises one polypeptide chain from N-terminus to C-terminus: a first VL, a first VH, a second VL, a second VH. In some specific embodiments, the multispecific antibody provided herein comprises one polypeptide from N-terminus to C-terminus: a first VH, a first VL, a second VL, a second VH. In some specific embodiments, the multispecific antibody provided herein comprises one polypeptide from N-terminus to C-terminus: a first VL, a first VH, a second VH, a second VL. In some specific embodiments, the multispecific antibody provided herein comprises one polypeptide from N-terminus to C-terminus: a first VH, a first VL, a second VH, a second VL. In some embodiments, the first VL and the first VH form a first scFv binding domain that binds to CD16a, and the second VL and the second VH form a second scFv binding domain that binds to a tumor antigen (such as CD19). In some embodiments, the first VL and the first VH form a first scFv binding domain that binds to a tumor antigen (such as CD19), and the second VL and the second VH form a second scFv binding domain that binds to CD16a. In some embodiments, the first scFv and the second scFv is linked through a polypeptide linker. In some embodiments, the polypeptide linker comprises GGSGGSGGS (SEQ ID NO: 359).

In some specific embodiments, the polypeptide chain described above further comprises a first CH2 and a first CH3 of an IgG Fc region. In some specific embodiments, the multispecific antibody provided herein comprises two identical polypeptide chains as described herein. In some specific embodiments, the first polypeptide and the second polypeptide form a bivalent format through dimerization of the CH2 and/or CH3 regions (e.g. the first CH2 is paired with the second CH2, and/or the first CH3 is paired with the second CH2). In some specific embodiments, the two polypeptide chains are not identical, wherein amino acid sequences of the first CH2 and the second CH2 are different from each other. In some specific embodiments, the two polypeptide chains are not identical, wherein amino acid sequences of the first CH3 and the second CH3 are different from each other. In some specific embodiments, the two polypeptide chains are not identical, wherein amino acid sequences of the first CH2 and the second CH2, and the first CH3 and the second CH3 are different from each other.

In some embodiments of the multispecific antibody provided herein, one or more scFvs are linked to an Fc region of an IgG. In some embodiments, one or more scFvs are linked to N terminal of the Fc region of the IgG. In some embodiments, one or more scFvs are linked to C terminal of the Fc region of the IgG. In some embodiments, one or more scFvs are linked to both N terminal and C terminal of the Fc region of the IgG. In some embodiments, at least one scFv binds to CD16a, and at least one scFv binds to a tumor antigen (such as CD19). In some embodiments, the IgG is IgG4. In some embodiments, the IgG is IgG1.

In some specific embodiments, the multispecific antibody provided herein comprises two polypeptides: a first polypeptide comprising from N-terminus to C-terminus: a first scFv, a second scFv, a first CH2, and a first CH3; a second polypeptide comprising from N-terminus to C-terminus: a third scFv, a forth scFv, a second CH2, and a second CH3. In some specific embodiments, the multispecific antibody provided herein comprises two polypeptides: a first polypeptide comprising from N-terminus to C-terminus: a first scFv, a second scFv, a first CH2, and a first CH3; a second polypeptide comprising from N-terminus to C-terminus: a second CH2, a second CH3, a third scFv, and a forth scFv. In some specific embodiments, the multispecific antibody provided herein comprises two polypeptides: a first polypeptide comprising from N-terminus to C-terminus: a first scFv, a second scFv, a first CH2, and a first CH3; a second polypeptide comprising from N-terminus to C-terminus: a third scFv, a second CH2, a second CH3, and a forth scFv. In some specific embodiments, the multispecific antibody provided herein comprises two polypeptides: a first polypeptide comprising from N-terminus to C-terminus: a first CH2, a first CH3, a first scFv, and a second scFv; a second polypeptide comprising from N-terminus to C-terminus: a second CH2, a second CH3, a third scFv, and a forth scFv. In some specific embodiments, the multispecific antibody provided herein comprises two polypeptides: a first polypeptide comprising from N-terminus to C-terminus: a first CH2, a first CH3, a first scFv, and a second scFv; a second polypeptide comprising from N-terminus to C-terminus: a third scFv, a forth scFv, a second CH2, and a second CH3. In some specific embodiments, the multispecific antibody provided herein comprises two polypeptides: a first polypeptide comprising from N-terminus to C-terminus: a first CH2, a first CH3, a first scFv, and a second scFv; a second polypeptide comprising from N-terminus to C-terminus: a third scFv, a second CH2, a second CH3, and a forth scFv. In some specific embodiments, the multispecific antibody provided herein comprises two polypeptides: a first polypeptide comprising from N-terminus to C-terminus: a first scFv, a first CH2, a first CH3, and a second scFv; a second polypeptide comprising from N-terminus to C-terminus: a third scFv, a second CH2, a second CH3, and a forth scFv. In some specific embodiments, the multispecific antibody provided herein comprises two polypeptides: a first polypeptide comprising from N-terminus to C-terminus: a first scFv, a first CH2, a first CH3, and a second scFv; a second polypeptide comprising from N-terminus to C-terminus: a third scFv, a forth scFv, a second CH2, and a second CH3. In some specific embodiments, the multispecific antibody provided herein comprises two polypeptides: a first polypeptide comprising from N-terminus to C-terminus: a first scFv, a first CH2, a first CH3, and a second scFv; a second polypeptide comprising from N-terminus to C-terminus: a second CH2, a second CH3, a third scFv, and a forth scFv. In some embodiments, the scFv domain is in the VL-VH orientation from N terminal to C terminal. In some embodiments the scFv domain is in the VH-VL orientation from N terminal to C terminal. In some embodiments, one scFv binds to CD16a, three scFvs bind to one or more tumor antigen. In some embodiments, one scFv binds to a tumor antigen, three scFvs bind CD16a. In some embodiments, two scFvs bind to CD16a, two scFvs bind to one or more tumor antigen (such as CD19). In some embodiments, the first scFv and the second scFv bind to CD16a, and the third scFv and the forth scFv bind to one or more tumor antigen (such as CD19). In some embodiments, the first scFv and the third scFv bind to CD16a, and the second scFv and the forth scFv bind to a tumor antigen (such as CD19). In some embodiments, the first scFv and the forth scFv bind to CD16a, and the second scFv and the third scFv bind to one or more tumor antigen (such as CD19). In some embodiments, the second scFv and the third scFv bind to CD16a, and the first scFv and the forth scFv bind to one or more tumor antigen (such as CD19). In some embodiments, the second scFv and the forth scFv bind to CD16a, and the first scFv and the third scFv bind to one or more tumor antigen (such as CD19). In some embodiments, the second scFv and the third scFv bind to CD16a, and the first scFv and the forth scFv bind to one or more tumor antigen (such as CD19). In some embodiments, the third scFv and the forth scFv bind to CD16a, and the first scFv and the second scFv bind to one or more tumor antigen (such as CD19). In some specific embodiments, amino acid sequences of the first CH2 is paired with the second CH2. In some specific embodiments, amino acid sequences of the first CH3 is paired with the second CH3. In some specific embodiments, amino acid sequences of the first CH2 and the second CH2 are different from each other. In some specific embodiments, amino acid sequences of the first CH3 and the second CH3 are different from each other. In some specific embodiments, amino acid sequences of the first CH2 and the second CH2 are identical to each other. In some specific embodiments, amino acid sequences of the first CH3 and the second CH3 are identical to each other.

In some embodiments, the multispecific binding agent as disclosed herein is in an IgG format. In some embodiments, the multispecific binding agent as disclosed herein is in an IgG format with mutated Fcγ1 region to eliminate Fc binding activity to Fcγ receptors. In some embodiments, the IgG is IgG4. In some embodiments, the IgG is IgG1.

In some embodiments, the multispecific binding agent as disclosed herein is in an IgG format comprising Leu234Ala, Leu235Ala and/or Pro329Gly mutations (Fc silencing mutations) to remove the Fc binding site for CD16a. In some embodiments, the CD16a binding properties of the multispecific binding agent as disclosed herein are not significantly impacted by the Fc silencing mutations (e.g. Leu234Ala, Leu235Ala and/or Pro329Gly). In some embodiments, the multispecific binding agent with the Fc silencing mutations provided herein binds to CD16a(e.g., human CD16a) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

In some embodiments, the multispecific binding agent as disclosed herein are modified by one or more insertions, one or more deletions, or one or more substitutions in the amino acid sequence. In further embodiments, these insertions, deletions, or substitutions are not within a CDR. In some embodiments, the modified polypeptide is at least 80%, or at least 85%, or at least 90%, or at least 91%, or at least 92%, or at least 93%, or at least 94%, or at least 95%, or at least 96%, or at least 97%, or at least 98%, or at least 99% identical to the polypeptide from which it is modified.

In some embodiments, the multispecific antibody described herein activates an NK cell. In certain embodiments, the multispecific antibody described herein contacts or directs NK cells to a target cell. In certain embodiments, the multispecific antibody described herein contacts or directs NK cells to a target cell, wherein the multispecific antibody comprises a first binding domain that binds to CD16a on an NK cell and a second binding domain that binds to a tumor antigen (such as CD19) on a target cell. In specific embodiments, the target cells are tumor cells. In certain embodiments, the multispecific antibody described herein induces NK cell dependent cytotoxicity of the tumor cell, wherein the multispecific antibody comprises a first binding domain that binds to CD16a on an NK cell and a second binding domain that binds to a tumor antigen (such as CD19) on a target cell. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a-F/F genotype. In specific embodiments, the NK cells are NK cells from a subject with a CD16a-V/F genotype. In specific embodiments, the NK cells are NK cells from a subject with a CD16a-V/V genotype.

In some embodiments, the multispecific antibodies can modulate NK cell activity. In some embodiments, the multispecific antibodies described herein can activate an NK cell. In some embodiments, the multispecific antibodies described herein activate an NK cell activity by at least about 10%. In some embodiments, the multispecific antibodies described herein activate an NK cell activity by at least about 20%. In some embodiments, the multispecific antibodies described herein activate an NK cell activity by at least about 30%. In some embodiments, the multispecific antibodies described herein activate an NK cell activity by at least about 40%. In some embodiments, the multispecific antibodies described herein activate an NK cell activity by at least about 50%. In some embodiments, the multispecific antibodies described herein activate an NK cell activity by at least about 60%. In some embodiments, the multispecific antibodies described herein activate an NK cell activity by at least about 70%. In some embodiments, the multispecific antibodies described herein activate an NK cell activity by at least about 80%. In some embodiments, the multispecific antibodies described herein activate an NK cell activity by at least about 90%. In some embodiments, the multispecific antibodies described herein activate an NK cell activity by at least about 95%. In certain embodiments, the multispecific antibodies described herein activate an NK cell activity by at least about 15% to about 65%. In certain embodiments, the multispecific antibodies described herein activate an NK cell activity by at least about 20% to about 65%. In certain embodiments, the multispecific antibodies described herein activate an NK cell activity by at least about 30% to about 65%. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a-F/F genotype. In specific embodiments, the NK cells are NK cells from a subject with a CD16a-V/F genotype. In specific embodiments, the NK cells are NK cells from a subject with a CD16a-V/V genotype.

In some embodiments, the multispecific antibodies described herein can induce ADCC activity of an NK cell. In some embodiments, the multispecific antibodies described herein induce ADCC activity of an NK cell by at least about 10%. In some embodiments, the multispecific antibodies described herein induce ADCC activity of an NK cell by at least about 20%. In some embodiments, the multispecific antibodies described herein induce ADCC activity of an NK cell by at least about 30%. In some embodiments, the multispecific antibodies described herein induce ADCC activity of an NK cell by at least about 40%. In some embodiments, the multispecific antibodies described herein induce ADCC activity of an NK cell by at least about 50%. In some embodiments, the multispecific antibodies described herein induce ADCC activity of an NK cell at least about 60%. In some embodiments, the multispecific antibodies described herein induce ADCC activity of an NK cell by at least about 70%. In some embodiments, the multispecific antibodies described herein induce ADCC activity of an NK cell by at least about 80%. In some embodiments, the multispecific antibodies described herein induce ADCC activity of an NK cell by at least about 90%. In some embodiments, the multispecific antibodies described herein induce ADCC activity of an NK cell by at least about 95%. In certain embodiments, the multispecific antibodies described herein induce ADCC activity of an NK cell by at least about 15% to about 65%. In certain embodiments, the multispecific antibodies described herein induce ADCC activity of an NK cell by at least about 20% to about 65%. In certain embodiments, the multispecific antibodies described herein induce ADCC activity of an NK cell by at least about 30% to about 65%. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a F/F genotype. In specific embodiments, the NK cells are NK cells from a subject with a CD16a-V/F genotype. In specific embodiments, the NK cells are NK cells from a subject with a CD16a-V/V genotype.

In some embodiments, the multispecific antibodies described herein can induce cytokine secretion of an NK cell. In some embodiments, the multispecific antibodies described herein induce cytokine secretion of an NK cell by at least about 10%. In some embodiments, the multispecific antibodies described herein induce cytokine secretion of an NK cell by at least about 20%. In some embodiments, the multispecific antibodies described herein induce cytokine secretion of an NK cell by at least about 30%. In some embodiments, the multispecific antibodies described herein induce cytokine secretion of an NK cell by at least about 40%. In some embodiments, the multispecific antibodies described herein induce cytokine secretion of an NK cell by at least about 50%. In some embodiments, the multispecific antibodies described herein induce cytokine secretion of an NK cell at least about 60%. In some embodiments, the multispecific antibodies described herein induce cytokine secretion of an NK cell by at least about 70%. In some embodiments, the multispecific antibodies described herein induce cytokine secretion of an NK cell by at least about 80%. In some embodiments, the multispecific antibodies described herein induce cytokine secretion of an NK cell by at least about 90%. In some embodiments, the multispecific antibodies described herein induce cytokine secretion of an NK cell by at least about 95%. In certain embodiments, the multispecific antibodies described herein induce cytokine secretion of an NK cell by at least about 15% to about 65%. In certain embodiments, the multispecific antibodies described herein induce cytokine secretion of an NK cell by at least about 20% to about 65%. In certain embodiments, the multispecific antibodies described herein induce cytokine secretion of an NK cell by at least about 30% to about 65%. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a-F/F genotype. In specific embodiments, the NK cells are NK cells from a subject with a CD16a-V/F genotype. In specific embodiments, the NK cells are NK cells from a subject with a CD16a-V/V genotype.

In certain embodiments, the multispecific antibody described herein induces NK cell dependent cytotoxicity of a tumor cell, wherein the multispecific antibody comprises a first binding domain that binds to CD16a on an NK cell and a second binding domain that binds to a tumor antigen (such as CD19) on the tumor cell. In specific embodiments, the NK cells are human NK cells. In some embodiments of the multispecific antibody provided herein, the multispecific antibody induces NK cell dependent cytotoxicity of the tumor cell by at least 10%. In some embodiments of the multispecific antibody provided herein, the multispecific antibody induces NK cell dependent cytotoxicity of the tumor cell by at least 20%. In some embodiments of the multispecific antibody provided herein, the multispecific antibody induces NK cell dependent cytotoxicity of the tumor cell by at least 30%. In some embodiments of the multispecific antibody provided herein, the multispecific antibody induces NK cell dependent cytotoxicity of the tumor cell by at least 40%. In some embodiments of the multispecific antibody provided herein, the multispecific antibody induces NK cell dependent cytotoxicity of the tumor cell by at least 50%. In some embodiments of the multispecific antibody provided herein, the multispecific antibody induces NK cell dependent cytotoxicity of the tumor cell by at least 60%. In some embodiments of the multispecific antibody provided herein, the multispecific antibody induces NK cell dependent cytotoxicity of the tumor cell by at least 70%. In some embodiments of the multispecific antibody provided herein, the multispecific antibody induces NK cell dependent cytotoxicity of the tumor cell by at least 80%. In some embodiments of the multispecific antibody provided herein, the multispecific antibody induces NK cell dependent cytotoxicity of the tumor cell by at least 90%. In some embodiments of the multispecific antibody provided herein, the multispecific antibody induces NK cell dependent cytotoxicity of the tumor cell by at least 95%. In certain embodiments of the multispecific antibody provided herein, the multispecific antibody induces NK cell dependent cytotoxicity of the tumor cell by at least about 15% to about 65%. In certain embodiments of the multispecific antibody provided herein, the multispecific antibody induces NK cell dependent cytotoxicity of the tumor cell by at least about 20% to about 65%. In certain embodiments of the multispecific antibody provided herein, the multispecific antibody induces NK cell dependent cytotoxicity of the tumor cell by at least about 30% to about 65%. In specific embodiments, the NK cells are human NK cells. In specific embodiments, the NK cells express CD16a-F. In specific embodiments, the NK cells express CD16a-V. In specific embodiments, the NK cells are NK cells from a subject with a CD16a-F/F genotype. In specific embodiments, the NK cells are NK cells from a subject with a CD16a-V/F genotype. In specific embodiments, the NK cells are NK cells from a subject with a CD16a-V/V genotype.

In some embodiments, the multispecific antibodies in an IgG format described herein can activate NK cell activity even with mutated Fcγ1 region to eliminate Fc binding activity to Fcγ receptors.

In some embodiments, the multispecific antibodies in an IgG format described herein can activate NK cell activity even with one or more Fc silencing mutations. In some embodiments, the multispecific antibodies in an IgG format described herein can activate NK cell activity even in an Fc silenced format with Leu234Ala, Leu235Ala and Pro329Gly mutations.

In some embodiments, the multispecific antibodies described herein do not bind to cells expressing CD16b. In some embodiments, the multispecific antibodies described herein do not bind to cells expressing CD16b-NA1. In some embodiments, the multispecific antibodies described herein do not bind to cells expressing CD16b-NA2. In some embodiments, the multispecific antibodies described herein do not bind to cells derived from a subject with CD16b-NA1/CD16b-NA1 genotype. In some embodiments, the multispecific antibodies described herein do not bind to cells derived from a subject with CD16b-NA1/CD16b-NA2 genotype. In some embodiments, the multispecific antibodies described herein do not bind to cells derived from a subject with CD16b-NA2/CD16b-NA2 genotype. In some embodiments, the multispecific antibodies described herein do not bind to neutrophils. In some embodiments, the multispecific antibodies described herein do not bind to neutrophils derived from a subject with CD16b-NA1/CD16b-NA1 genotype. In some embodiments, the multispecific antibodies described herein do not bind to neutrophils derived from a subject with CD16b-NA1/CD16b-NA2 genotype. In some embodiments, the multispecific antibodies described herein do not bind to neutrophils derived from a subject with CD16b-NA2/CD16b-NA2 genotype. In some embodiments, the multispecific antibodies described herein do not induce activation of neutrophils. In some embodiments, the multispecific antibodies described herein induce activation of NK cells and do not induce activation of neutrophils In some embodiments, the multispecific antibodies described herein induce activation of NK cells and do not induce activation of neutrophils and other granulocytes.

Other exemplary binding molecules are described in more detail in the following sections. In some embodiments, the multispecific binding agents according to any of the above embodiments may incorporate any of the features, singly or in combination, as described in Sections 5.2.4 to 5.2.7 below.

### 5.2.4. Antibody Fragments

Even though the term “antibody” is sometimes used in a phrase of “antibody or fragment thereof” herein, it should be understood the term “antibody” as used herein also includes various antibody fragments, such as an antigen-binding fragment or epitope-binding fragment. Thus, when the term “antibody” is used alone without being followed by “fragment thereof” or similar terms, it should be understood the term “antibody” includes an antibody fragment, such as an antigen-binding fragment or epitope-binding fragment. Antibodies provided herein include, but are not limited to, immunoglobulin molecules and immunologically active portions of immunoglobulin molecules. In some embodiments, the multispecific binding agents provided herein comprise one or more antibody fragments.

Variants and derivatives of antibodies include antibody functional fragments that retain the ability to bind to an antigen. Antibody fragments include but are not limited to those described in Section 5.1 above. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen-binding domains, each of which may be directed to a different epitope); a single chain comprising a variable region, also known as, scFv (e.g., the variable, antigen-binding determinative region of a single light and heavy chain of an antibody linked together by a chain of, e.g., 10-25 amino acids); a disulfide-linked Fv, or dsFv (e.g., the variable, antigen-binding determinative region of a single light and heavy chain of an antibody linked together by a disulfide bond); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen-binding domains may be directed towards the same or different epitopes); and a tetrabody (e.g., a tetramerized scFv, formed in a manner similar to a diabody, but in which four antigen-binding domains are created in a single complex; the four antigen-binding domains may be directed towards the same or different epitopes).

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. For example, Fab, Fv, and scFv antibody fragments can all be expressed in and secreted from E. coli, yeast or insect cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragments with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

### 5.2.5. Humanized Antibodies

The present disclosure provides humanized antibodies that bind to CD16a and/or the tumor target antigen, such as CD19, including human CD16a and/or human CD19. Humanized antibodies of the present disclosure may comprise one or more CDRs from a VH and/or VL disclosed herein, such as those as shown in Tables 1-10. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind CD16a and/or the tumor target antigen, such as CD19 may be produced using techniques known to those skilled in the art (Zhang et al., Molecular Immunology, 42(12): 1445-1451, 2005; Hwang et al., Methods, 36(1): 35-42, 2005; Dall'Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567).

In some cases, the humanized antibodies are constructed by CDR grafting, in which the amino acid sequences of the six CDRs of a VH and a VL of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36:25-34, 2005).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (Sims et al. (1993) J. Immunol. 151:2296; Chothia et al. (1987) J. Mol. Biol. 196:901). Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623). In some cases, the framework is derived from the consensus sequences of the most abundant human subclasses, V_{L}6 subgroup I (V_{L}6I) and V_{H} subgroup III (V_{H}III). In another method, human germline genes are used at the source of the framework regions.

In an alternative paradigm based on comparison of CDRs, called Superhumanization, framework homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as framework donors. Finally, the non-human CDRs are grafted onto these frameworks (see, e.g., Tan et al., J. Immunol. 169:1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13: 819-824, 2000), Modeller (Sali and Blundell, J. Mol. Biol. 234: 779-815, 1993), and Swiss PDB Viewer (Guex and Peitsch, Electrophoresis 18: 2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, framework residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. See, e.g., Lazar et al., Mol. Immunol. 44: 1986-1998, 2007.

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23: 1105-1116, 2005; Dufner et al., Trends Biotechnol. 24: 523-529, 2006; Feldhaus et al., Nat. Biotechnol. 21: 163-70, 2003; Schlapschy et al., Protein Eng. Des. Sel. 17: 847-60, 2004).

In the framework library approach, a collection of residue variants are introduced at specific positions in the framework followed by selection of the library to select the framework that best supports the grafted CDR. The residues to be substituted may include some or all of the “Vernier” residues identified as potentially contributing to CDR structure (see, e.g., Foote and Winter, J. Mol. Biol. 224: 487-499, 1992), or from the more limited set of target residues identified by Baca et al. (J. Biol. Chem. 272: 10678-10684, 1997).

In framework shuffling, whole frameworks are combined with the non-human CDRs instead of creating combinatorial libraries of selected residue variants (see, e.g., Dall'Acqua et al., Methods 36: 43-60, 2005). The libraries may be screened for binding in a two-step selection process, first humanizing VL, followed by VH. Alternatively, a one-step framework shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44: 3049-60, 2007).

The “humaneering” method is based on experimental identification of essential minimum specificity determinants (MSDs) and is based on sequential replacement of non-human fragments into libraries of human frameworks and assessment of binding. It begins with regions of the CDR3 of non-human VH and VL chains and progressively replaces other regions of the non-human antibody into the human frameworks, including the CDR1 and CDR2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. See, e.g., Alfenito, Cambridge Healthtech Institute's Third Annual PEGS, The Protein Engineering Summit, 2007.

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making a particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 7:805-814(1994), U.S. Pat. Nos. 5,766,886, 5,770,196, 5,821,123, and 5,869,619, and WO 93/11794.

### 5.2.6. Antibody Variants

Modifications of the antibodies that bind to CD16a and/or the tumor target antigen (such as CD19) described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, it is contemplated that variants of the antibodies described herein can be prepared and are included in the present disclosure. In some embodiments, antibody variants are antibodies with amino acid sequence variations as compared with the original antibody, for example, substitution, deletion, or insertion of one or more amino acid(s), as described above. For example, variations may be a substitution, deletion, or insertion of one or more codons encoding the antibody or polypeptide that results in a change in the amino acid sequence (e.g., a conservative substitution) as compared with the original antibody or polypeptide. Sites of interest for substitutional mutagenesis include the CDRs, FRs and/or constant regions. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody.

### Chemical Modifications

Other exemplary modifications include chemical modifications, for example, by the covalent attachment of any type of molecule to the binding agent. Antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the antibody provided herein is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., U.S. Pat. Publ. Nos. 2003/0157108 and 2004/0093621. Examples of publications related to “defucosylated” or “fucose-deficient” antibody variants include: U.S. Pat. Publ. No. 2003/0157108; WO 2000/61739; WO 2001/29246; U.S. Pat. Publ. No. 2003/0115614; U.S. Pat. Publ. No. 2002/0164328; U.S. Pat. Publ. No. 2004/0093621; U.S. Pat. Publ. No. 2004/0132140; U.S. Pat. Publ. No. 2004/0110704; U.S. Pat. Publ. No. 2004/0110282; U.S. Pat. Publ. No. 2004/0109865; WO 2003/085119; WO 2003/084570; WO 2005/035586; WO 2005/035778; WO2005/053742; WO2002/031140; Okazaki et al. J. Mol. Biol. 336:1239-1249(2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249: 533-545(1986); U.S. Pat. Publ. No. 2003/0157108; and WO 2004/056312, and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4): 680-688(2006); and WO2003/085107).

The binding molecules comprising an antibody provided herein are further provided with bisected oligosaccharides, e.g., in which a biantennary oligosaccharide attached to the Fc region is bisected by GlcNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and U.S. Pat. Publ. No. 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

In molecules that comprise the present antibody and an Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions. In some embodiments, the Fc region comprises Leu234Ala, Leu235Ala and/or Pro329Gly mutations. In some embodiments, the Fc region comprises mutated Fcγ1 region to eliminate Fc binding activity to Fcγ receptors.

In some embodiments, the present application contemplates variants that possess some but not all effector functions, which make it a desirable candidate for applications in which the half-life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcγR binding (hence likely lacking ADCC activity), but retains FcRn binding ability. Non-limiting examples of in vitro assays to assess ADCC activity of a molecule of interest are described in U.S. Pat. No. 5,500,362 (see, e.g., Hellstrom, I. et al. Proc. Nat'l Acad. Sci. USA 83:7059-7063(1986)) and Hellstrom, I et al., Proc. Nat'l Acad. Sci. USA 82:1499-1502(1985); U.S. Pat. No. 5,821,337 (see Bruggemann, M. et al., J. Exp. Med. 166:1351-1361(1987)). Alternatively, non-radioactive assay methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052(2003); and Cragg, M. S. and M. J. Glennie, Blood 103:2738-2743(2004)). FcRn binding and in vivo clearance/half-life determinations can also be performed using methods known in the art (see, e.g., Petkova, S. B. et al., Int'l. Immunol. 18(12): 1759-1769(2006)).

Binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9(2): 6591-6604(2001).)

In some embodiments, a variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (e.g., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184(2000).

Binding molecules with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40(1988); U.S. Pat. Nos. 5,648,260; 5,624,821; and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

Other known covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86(1983)), acetylation of the N-terminal amine, and amidation of any C-terminal carboxyl group.

The antibody that binds to CD16a and/or the tumor target antigen (such as CD19) of the present disclosure may also be modified to form chimeric molecules comprising the antibody fused or conjugated to another, heterologous polypeptide or amino acid sequence or a small molecule compound, for example, an immune activator (such as a cytokine), an epitope tag (see, e.g., Terpe, Appl. Microbiol. Biotechnol. 60:523-33(2003)) or the Fc region of an IgG molecule (see, e.g., Aruffo, Antibody Fusion Proteins 221-42 (Chamow and Ashkenazi eds., 1999)).

Also provided herein are fusion proteins comprising the antibody that binds to CD16a and/or the tumor target antigen (such as CD19) of the disclosure and a heterologous polypeptide. In some embodiments, the heterologous polypeptide to which the antibody is genetically fused or chemically conjugated is useful for targeting the antibody to cells having cell surface-expressed CD16a and/or the tumor target antigen, such as CD19. Genetically fused or chemically conjugated antibodies are described in more detail in sections below.

### In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37(2002); and Bradbury and Marks, J. Immunol. Methods 290:29-49(2004).

In a yeast display system (see, e.g., Boder et al., Nat. Biotech. 15:553-57(1997); and Chao et al., Nat. Protocols 1:755-68(2006)), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single-chain antibody (e.g., scFv). Expression has been shown to correlate with the stability of the displayed protein, and thus antibodies can be selected for improved stability as well as affinity (see, e.g., Shusta et al., J. Mol. Biol. 292:949-56(1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publ. No. 2003/0186374; and Blaise et al., Gene 342:211-18(2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., Nucleic Acids Res. 34:e127(2006)). In mRNA display, a covalent bond between antibody and mRNA is established using puromycin as an adaptor molecule (Wilson et al., Proc. Natl. Acad. Sci. USA 98:3750-55(2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step. In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003); U.S. Pat. Nos. 5,565,332 and 6,989,250). Alternative techniques target hypervariable loops extending into framework-region residues (see, e.g., Bond et al., J. Mol. Biol. 348:699-709(2005)) employ loop deletions and insertions in CDRs or use hybridization-based diversification (see, e.g., U.S. Pat. Publ. No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which is incorporated herein by reference.

Screening of the libraries can be accomplished by various techniques known in the art. For example, antibodies can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For reviews of in vitro affinity maturation methods, see, e.g., Hoogenboom, Nature Biotechnology 23:1105-16(2005); Quiroz and Sinclair, Revista Ingeneria Biomedia 4:39-51(2010); and references therein.

An antibody internalization assay may be used to determine receptor-mediated endocytosis when binding to an antibody. In some embodiments, the efficacy of certain antibody-based therapeutics depends on antibody internalization process. In some embodiments, an antibody internalization assay examines the rate and extent of antibody internalization in order to evaluate the antibody's ability of delivering treatments to sites or cells of interest. A non-limiting exemplary assay is briefly described below. Target cells of interest are seeded at an appropriate seeding density (e.g., in a 96-well U-bottom plate), and a tested antibody is labelled with a signal reporting reagent, for example, fluorescent compounds, Horseradish peroxidase (HRP) reagent, radiolabeled compounds, or biotin. Then the tested antibody and the target cells are incubated at an appropriate molar ratio. Following the incubation, unbound antibodies are removed by wash. The cells can be left on ice or incubated at 37° C. for a period of time to facilitate internalization. The cells may then be incubated for a period of time in the presence of a stop reagent to inhibit internalization. Subsequently, the cells are washed and incubated with the signal developing reagent. The final signal can be studied using plate reader or imaging instrument and an analytical software. For example, mean fluorescence intensity (MFI) of the cells can be measured using a flow cytometer, and MFI reduction can represent antibody internalization, antibody dissociation or a combination of both. Cell imaging can be scanned and acquired to analyze the signal intensity, size and shape. Alternatively, the cells are lysed, releasing internalized antibody. This antibody is then captured in a microtiter well plate coated with specific antigen against which the antibody was raised. Bound antibody in the well is detected using an alkaline phosphatase or HRP-conjugated secondary antibody and a chromogenic substrate. Alternative detectable labels for the antibody and means for detecting internalized labeled antibody will be obvious to those skilled in the art upon this disclosure. Any methods known in the art to determine antibody internalization can be used in the present disclosure.

### 5.2.7. Other Binding Agents Comprising the Antibodies

In some embodiments, the antibody or fragment thereof provided herein is a part of a larger binding agent. Non-limiting exemplary binding agents comprising the antibody or fragment provided herein are described below.

The present disclosure provides binding agents (e.g., antibodies, multispecific antibodies, or antibody fragments) with a masking moiety and/or cleavable moiety in which one or more of the CD16a binding domains and/or the tumor target antigen (such as CD19) binding domains of the binding agent (e.g., multispecific antibody) are masked (e.g., via a masking moiety) and/or activatable (e.g., via a cleavable moiety). Technologies for masking of antibodies are well known in the art, including SAFEbody masking technology (see, e.g., U.S. Pat. Publ. No. 2019/0241886) and Probody masking technology (see, e.g., U.S. Pat. Publ. No. 2015/0079088). Such technologies can be used to generate a binding agent (e.g., an antibody, a multispecific antibody, or antibody fragment) that is masked and/or activatable. Such masked and/or activatable binding agents (e.g., antibodies, multispecific antibodies, or antibody fragments) are also useful for the preparation of conjugates, including immunoconjugates, antibody-drug conjugates (ADCs), masked ADCs and activatable ADCs (AADCs), comprising any one of the binding agents (e.g., antibodies, multispecific antibodies, or antibody fragments) of the present disclosure, including those directly or indirectly linked another agent such as a drug and/or an immune activator (such as a cytokine). For example, binding agents (e.g., antibodies, multispecific antibodies, or antibody fragments) of the present disclosure may be covalently bound by a synthetic linker to one or more agents such as drugs and/or immune activators.

If desired, a binding agent (e.g., an antibody, a multispecific antibody, or an antibody fragment) provided herein is linked or conjugated (directly or indirectly) to a moiety with effector function, such as cytotoxic activity (e.g., a chemotherapeutic moiety or a radioisotope), immune recruitment or modulating activity. Moieties that are linked or conjugated (directly or indirectly) include drugs that are cytotoxic (e.g., toxins such as aurostatins) or non-cytotoxic, e.g., signal transduction modulators such as kinases or masking moieties that mask one or more binding domains of the binding agent provided herein, or cleavable moieties that allow for activating a binding agent by cleaving off a cleavable moiety to unmask one or more binding domains in the tumor microenvironment in the form of masked conjugates. Moieties that promote immune recruitment can include other antigen-binding agents, such as viral proteins that bind selectively to cells of the innate and/or adaptive immune system. Alternatively or in addition, a binding agent (e.g., an antibody, a multispecific antibody, or an antibody fragment) provided herein is optionally linked or conjugated (directly or indirectly) to a moiety that facilitates isolation from a mixture (e.g., a tag) or a moiety with reporter activity (e.g., a detection label or reporter protein). It will be appreciated that the features of a binding agent described herein extend also to a polypeptide comprising a binding agent fragment.

In some embodiments, the binding agents (e.g., antibodies, multispecific antibodies, or antibody fragments thereof) described herein may be linked or conjugated (directly or indirectly) to a polypeptide, which can result in the generation of an activatable antibody. In some embodiments, the multispecific binding agent provided herein is linked or conjugated (directly or indirectly) to an additional agent. In some embodiments, the additional agent is a drug, resulting in an ADC or an AADC when the antibody of the ADC comprises a masking moiety and a cleavable moiety.

In some embodiments, the binding agents (e.g., antibodies, multispecific antibodies, or antibody fragments thereof), described herein are conjugated or recombinantly linked (directly or indirectly) to a therapeutic agent (e.g., a cytotoxic agent or a cytokine) or to a diagnostic or detectable agent. The conjugated or recombinantly linked antibodies, including masked or activatable conjugates, can be useful, for example, for treating or preventing a disease, disorder or condition. The conjugated or recombinantly linked binding agents (e.g., antibodies, multispecific antibodies, or antibody fragments thereof), including masked or activatable conjugates, can be useful, for example, for monitoring or prognosing the onset, development, progression, and/or severity of a disease, disorder or condition.

Such diagnosis and detection can be accomplished, for example, by coupling a binding agent (e.g., an antibody, a multispecific antibody, or an antibody fragment) to detectable substances including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

Also described herein are binding agents (e.g., antibodies, multispecific antibodies, or antibody fragments thereof) that are recombinantly linked or conjugated (covalent or non-covalent conjugations, directly or indirectly) to a heterologous protein or polypeptide or fragment thereof, for example, to a polypeptide (e.g., of about 10, about 20, about 30, about 40, about 50, about 60, about 70, about 80, about 90, or about 100 amino acids) to generate fusion proteins, as well as uses thereof. In some embodiments, the heterologous protein, polypeptide, or peptide that a binding agent (e.g., an antibody, a multispecific antibody, or an antibody fragment) is linked to is useful for targeting the binding agent to a particular cell (e.g., an CD16a-expressing cell, such as, an immune cell; and/or CD19 expressing cell, including an immune cell, and/or a cancer or tumor cell). Other non-limiting heterologous protein, polypeptide, or peptide that a binding agent (e.g., an antibody, a multispecific antibody, or antibody fragment) is linked to can be useful as an internalization signal or engaging a tumor cell with an immune cell.

Moreover, binding agents (e.g., antibodies, multispecific antibodies, or antibody fragments thereof) described herein can be linked (directly or indirectly) to marker or “tag” sequences, such as a peptide, to facilitate purification. In some embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In some embodiments, a binding agent (e.g., an antibody, a multispecific antibody, or an antibody fragment) described herein is a fusion protein. The term “fusion protein” as used herein refers to a polypeptide that comprises an amino acid sequence of a binding agent (e.g., an antibody) and an amino acid sequence of a heterologous polypeptide or protein (e.g., a polypeptide or protein not normally a part of the antibody). In certain embodiments, the fusion protein retains the biological activity of a binding agent.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of binding agents (e.g., an antibody, a multispecific antibody, or antibody fragment) as described herein with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2): 76-82; Hansson et al., 1999, J. Mol. Biol. 287:265-76; and Lorenzo and Blasco, 1998, Biotechniques 24(2): 308-13). In some embodiments, the binding agents provided herein may be altered by being subjected to random mutagenesis by error-prone PCR, random nucleotide insertion, or other methods prior to recombination. A polynucleotide encoding a binding agent described herein may be recombined with one or more components, motifs, sections, parts, domains, fragments, etc. of one or more heterologous molecules.

The binding agents (e.g., antibodies, multispecific antibodies, or antibody fragments thereof) described herein may also be attached to solid supports, which are useful for immunoassays or purification of the target antigen. Such solid supports include, but are not limited to, glass, cellulose, polyacrylamide, nylon, polystyrene, polyvinyl chloride, or polypropylene.

The binding agents (e.g., antibodies, multispecific antibodies, or antibody fragments thereof) described herein can also be linked or conjugated (directly or indirectly) to a second antibody to form an antibody heteroconjugate.

The linker may be a “cleavable moiety” facilitating release of the linked or conjugated agent in a cell, but non-cleavable linkers are also contemplated herein. Linkers for use in conjugates (e.g., ADCs or AADCs) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and agent, including wherein the agent is a drug for the preparation of ADC or an AADC, may be made using a variety of bifunctional protein coupling agents such as N-(β-maleimidopropyloxy) succinimide ester (BMPS); N-ε-malemidocaproyl-oxysuccinimide ester (ECMS); N-γ-malemidocaproyl-oxysuccinimide ester (GMBS); 1,6-hexane-bis-vinylsulfone (HBVS); succinimidyl 4(-N-maleimidomethyl)cyclohexane-1-carboxy-(6-amidocaproate)) (LC-SMCC); m-maleimidobenzoyl-N-hydroxysuccinimide ester (MBS); 4-(4-N-maleimidophenyl)butyric acid hydrazide (MPBH); succinimidil 3-(bromoacetamido) propionate (SBAP); succinimidyl iodoacetate (SIA); succinimidyl (4-iodoacetyl)aminobenzoate (SIAB); succinimidyl-4-(N-maleimidomethyl)cyclohexane-1-carboxylate (SMCC); succinimidyl 4-(p-maleimido-phenyl)butyrate (SMPB); succinimidyl-6-(β-maleimidopropionamido)hexanoate (SMPH); N-(ε-maleimidocaproyloxy)sulfosuccinimide ester (sulfo-ECMS); N-(γ-maleimidobutryloxy)sulfosuccinimide ester (sulfo-GMBS); N-(κ-maleimidoundecanoyloxy)sulfosuccinimide ester (sulfo-KMUS); m-maleimidobenzoyl-N-hydroxysulfosuccinimide ester (sulfo-MBS); sulfosuccinimidyl (4-iodo-acetyl)aminobenzoate (sulfo-SIAB); sulfosuccinimidyl 4-(N-maleimido-methyl)cyclohexane-1-carboxylate (sulfo-SMCC); sulfosuccinimidyl 4-(p-maleimidophenyl)butyrate (sulfo-SMPB); and succinimidyl-(4-vinylsulfone)benzoate) (SVSB).

The present disclosure further contemplates conjugates of antibodies and agents, including wherein the agent is a drug for the preparation of ADC or AADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of ADC or AADC, have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332:41-52; and Junutula et al., 2008, Nature Biotechnol. 26:925-32). In another method, selenocysteine is co-translationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50): 12047-57).

In some embodiments, the binding agent (the antibody, the multispecific antibody, or the antibody fragment) described herein is conjugated to an agent, for example, an immune activator (such as a cytokine) or a cytotoxic agent. In some embodiments, a binding agent (an antibody, a multispecific antibody, or antibody fragment) described herein can be optionally conjugated with one or more cytotoxic agent(s) disclosed herein or known in the art in order to generate an ADC or AADC. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In other embodiments, the agent is a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, such as camptothecin or an analog thereof, and a tyrosine kinase inhibitor. In some embodiments, the mitotic inhibitor is a dolastatin, an auristatin, a maytansinoid, and a plant alkaloid. In some embodiments, the agent is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In some embodiments, the anti-tumor antibiotic is selected from the group consisting of an actinomycine, an anthracycline, a calicheamicin, and a duocarmycin. An example of an actinomycine is a pyrrolobenzodiazepine (PBD). An example of an anthracycline is a PNU-anthracycline such as PNU-159682 or derivative.

### 5.3. Nucleic Acids, Vectors and Cells

Additionally provided are a nucleic acid encoding a binding agent (e.g., an antibody, a multispecific antibody, or an antibody fragment thereof) or a fusion polypeptide as disclosed herein, a nucleic acid complementary thereto; a vector comprising a nucleic acid as disclosed herein; and a cell comprising any one or more of: a binding agent as disclosed herein, a nucleic acid as disclosed herein, or a vector as disclosed herein. In some embodiments, the cell expresses the binding agent provided herein. In some embodiments, the cell replicates the nucleic acid or the vector. In some embodiments, provided are the materials for generating binding agents and fragments thereof. For example, an isolated cell may produce a binding agent (e.g., an antibody or fragment thereof, a multispecific antibody or antibody fragment). In this regard, a cell (e.g., an isolated cell) may produce an antibody or fragment thereof comprising a VH and a VL as disclosed herein. In some embodiments, polynucleotides described herein may comprise one or more nucleic acid sequences encoding a binding agent (e.g., an antibody or fragment thereof, a multispecific antibody or antibody fragment). In some embodiments, the polynucleotide is an isolated and/or recombinant polynucleotide. In various aspects, the isolated polynucleotide comprises a nucleotide sequence that encodes a VH and/or a VL, wherein the VH and the VL comprise complementarity determining regions (CDRs) identical to CDRs as disclosed herein.

As used herein, the term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present. The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term “stringent assay conditions” generally refers to the combination of hybridization and wash conditions.

In some embodiments, one or more vectors (e.g., expression vectors) may comprise one or more polynucleotides for expression of the one or more polynucleotides in a suitable host cell. Such vectors are useful, for example, for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind CD16a(including CD16a-V and/or CD16a-F) and/or CD19 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Expression constructs may also include sequences that facilitate, and preferably promote, homologous recombination in a host cell. In some embodiments, expression constructs of the can also include sequences necessary for replication in a host cell.

Exemplary expression control sequences include promoter/enhancer sequences, e.g., cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132: 2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences whose only intended function is to facilitate large scale production of the vector. One can manufacture and administer polynucleotides for gene therapy using procedures that have been described in the literature for a variety of transgenes. See, e.g., Isner et al., Circulation, 91: 2687-2692, 1995; and Isner et al., Human Gene Therapy, 7: 989-1011, 1996.

In some embodiments, polynucleotides may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the binding molecules of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the binding molecule of the disclosure. As used herein, the phrase “a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Any suitable vectors may be used to introduce one or more polynucleotides that encode an antibody or fragment thereof into the host. Exemplary vectors that have been described include replication deficient retroviral vectors, including but not limited to lentivirus vectors (Kim et al., J. Virol., 72(1): 811-816, 1998; Kingsman & Johnson, Scrip Magazine, October 1998, pp. 43-46); parvoviral vectors, such as adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricaudet et al., J. Clin. Invest., 90: 626-630, 1992; and Rosenfeld et al., Cell, 68: 143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

Other non-viral delivery mechanisms contemplated include calcium phosphate precipitation (Graham and Van Der Eb, Virology, 52: 456-467, 1973; Chen and Okayama, Mol. Cell Biol., 7: 2745-2752, 1987; Rippe et al., Mol. Cell Biol., 10: 689-695, 1990) DEAE-dextran (Gopal, Mol. Cell Biol., 5: 1188-1190, 1985), electroporation (Tur-Kaspa et al., Mol. Cell Biol., 6: 716-718, 1986; Potter et al., Proc. Nat. Acad. Sci. USA, 81: 7161-7165, 1984), direct microinjection (Harland and Weintraub, J. Cell Biol., 101: 1094-1099, 1985), DNA-loaded liposomes (Nicolau and Sene, Biochim. Biophys. Acta, 721: 185-190, 1982; Fraley et al., Proc. Natl. Acad. Sci. USA, 76: 3348-3352, 1979; Felgner, Sci Am., 276(6): 102-6, 1997; Felgner, Hum Gene Ther., 7(15): 1791-3, 1996), cell sonication (Fechheimer et al., Proc. Natl. Acad. Sci. USA, 84: 8463-8467, 1987), gene bombardment using high velocity microprojectiles (Yang et al., Proc. Natl. Acad. Sci USA, 87: 9568-9572, 1990), and receptor-mediated transfection (Wu and Wu, J. Biol. Chem., 262: 4429-4432, 1987; Wu and Wu, Biochemistry, 27: 887-892, 1988; Wu and Wu, Adv. Drug Delivery Rev., 12: 159-167, 1993).

A vector (or the antibody or fragment thereof or a nucleic acid as discussed herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814, (1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (Kaneda et al., Science, 243: 375-378, 1989). In some embodiments, the liposome is complexed or employed in conjunction with nuclear nonhistone chromosomal proteins (HMG-1) (Kato et al., J. Biol. Chem., 266: 3361-3364, 1991). In some embodiments, the liposome is complexed or employed in conjunction with both HVJ and HMG-1. Such expression constructs have been successfully employed in transfer and expression of nucleic acid in vitro and in vivo. In some embodiments, a binding agent (e.g., an antibody) is included in the liposome to target the liposome to cells (such as an immune cell) expressing CD16a(including CD16a-V and/or CD16a-F) and/or cells (such as cancer cell) expressing a tumor antigen (such as CD19) on their surface.

A cell may comprise one or more polynucleotides or one or more vectors, e.g., the cell is transformed or transfected with one or more polynucleotides encoding a binding agent (e.g., an antibody) or the one or more vectors comprising the one or more polynucleotides. In some embodiments, cells express a binding agent (e.g., an antibody) containing one or more, including six CDRs having at least 75%, 80%, 85%, 90%, 95%, 96%, 97%, 98%, or 99% identity to the CDRs of the antibodies disclosed in Tables 1, 2, 3, 4, 5, 6, 7, 8, 9, or 10. In some embodiments, the cell expresses a binding agent (e.g., an antibody) containing the VH and the VL comprising CDRs identical to those of the antibodies disclosed in Tables 1, 2, 3, 4, 5, 6, 7, 8, 9, or 10. The cells may be prokaryotic cells, such as Escherichia coli (see, e.g., Pluckthun et al., Methods Enzymol., 178:497-515, 1989), or eukaryotic cells, such as an animal cell (e.g., a myeloma cell, Chinese Hamster Ovary (CHO) cell, or hybridoma cell), yeast (e.g., Saccharomyces cerevisiae), an insect cell, or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., multispecific binding agents (e.g., bispecific antibodies) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of a binding agent (e.g., a multispecific binding agent or a bispecific antibody) may comprise culturing a host cell and isolating the binding agent. Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327: 70-73, 1987). Several devices for accelerating small particles have been developed. One such device relies on a high voltage discharge to generate an electrical current, which in turn provides the motive force (Yang et al., Proc. Natl. Acad. Sci USA, 87: 9568-9572, 1990). The microprojectiles used have consisted of biologically inert substances such as tungsten or gold beads. A host cell may be isolated and/or purified. A host cell also may be a cell transformed in vivo to cause transient or permanent expression of the polypeptide in vivo. A host cell may also be an isolated cell transformed ex vivo and introduced post-transformation, e.g., to produce the polypeptide in vivo for therapeutic purposes. The definition of host cell explicitly excludes a transgenic human being.

### 5.4. Methods of Making

Antibodies that bind to CD16a and/or a tumor antigen (such as CD19) may be obtained by any suitable method. Each CD16a and tumor antigen (such as CD19) binding domain can be derived from an anti-CD16a antibody or tumor antigen (such as CD19) binding antibody, which may be obtained by suitable methods, such as (but not limited to) immunization with whole cells comprising CD16a(including CD16a-V and/or CD16a-F) or the tumor antigen (such as CD19) and collection of antibodies, recombinant, or screening libraries of antibodies or antibody fragments using CD16a(including CD16a-V and/or CD16a-F) or the tumor antigen (such as CD19) extracellular domain epitopes. Monoclonal antibodies may be generated using a variety of known techniques (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). One exemplary technique for generating monoclonal antibodies comprises immunizing an animal with a human CD16a antigen (including CD16a-F and/or CD16-V) or a human CD19 antigen and generating a hybridoma from spleen cells taken from the animal. A hybridoma may produce a monoclonal antibody or antibody fragment that binds CD16a (including CD16a-F and/or CD16-V) or CD19.

In a further embodiment, monoclonal antibodies or antibody fragments can be isolated from antibody phage libraries generated using the techniques described in, for example, Antibody Phage Display: Methods and Protocols, P. M. O'Brien and R. Aitken, eds, Humana Press, Totawa N.J., 2002. In principle, synthetic antibody clones are selected by screening phage libraries containing phage that display various fragments of antibody variable region (Fv) fused to phage coat protein. Such phage libraries are screened for against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734(1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227: 381-388(1992).

Screening of the libraries can be accomplished by various techniques known in the art. For example, CD16a(e.g., a CD16a polypeptide, fragment or epitope) or a tumor target antigen, such as CD19 (e.g., a CD19 polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8: 309-314(1990) and in WO 92/09690, and a low coating density of antigen as described in Marks et al., Biotechnol., 10: 779-783(1992).

Multispecific binding agents (e.g., antibodies) can be obtained by designing a suitable antigen screening procedure to select for the phage clone of interest followed by construction of a full length binding agent (e.g., an antibody) clone using VH and/or VL sequences (e.g., the Fv sequences), or various CDR sequences from VH and VL sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

Likewise, human antibodies that bind CD16a and/or a tumor target antigen (such as CD19) may be generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8: 455 58, 1997; Jakobovits et al., Ann. N. Y. Acad. Sci., 764: 525 35, 1995; Green et al., Nature Genet., 7: 13-21, 1994; Lonberg et al., Nature, 368: 856-859, 1994; Taylor et al., Int. Immun. 6: 579-591, 1994; and U.S. Pat. No. 5,877,397.

For example, human antibodies that bind CD16a and/or a tumor target antigen (such as CD19) may be obtained from transgenic animals that have been engineered to produce specific human antibodies in response to antigenic challenge. For example, WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

In some embodiments, a binding agent described herein comprises a non-antibody protein scaffold. Non-limiting examples of such a non-antibody protein scaffold include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate an CD16a and/or a tumor target such as CD19 binding agent comprising a non-antibody protein scaffold (see, e.g., Simeon and Chen, Protein Cell, 9(1): 3-14(2018); Yang et al., Annu Rev Anal Chem (Palo Alto Calif). 10(1):293-320(2017)).

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al., 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, U K, 1992; and Current Protocols in Molecular Biology, Ausubel ed., Wiley Interscience, New York, 1999).

A binding agent (e.g., an antibody) is produced using any suitable method, e.g., isolated from an immunized animal, recombinantly or synthetically generated, or genetically-engineered, including as described above. Antibody fragments derived from an antibody are obtained by, e.g., proteolytic hydrolysis of an antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1: 422 Academic Press (1967); Nisonoff et al., Arch. Biochem. Biophys., 89: 230-244, 1960; Porter, Biochem. J., 73: 119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1 2.8.10 and 2.10A.1 2.10A.5.

A binding agent (e.g., an antibody) can be genetically engineered. For example, a binding agent (e.g., an antibody) comprises, for example, a variable region domain generated by recombinant DNA engineering techniques. In this regard, a variable region is optionally modified by insertions, deletions, or changes in the amino acid sequence of the antibody to produce an antibody of interest, including as described above. Polynucleotides encoding CDRs of interest are prepared, for example, by using polymerase chain reaction to synthesize variable regions using mRNA of antibody producing cells as a template (see, for example, Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2: 106-110, 1991). Current antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target. Exemplary methods for generating humanized antibodies are described in sections above too.

The invention provides polypeptides that can be used as immunogens to generate anti-CD16a antibodies or antigen-binding fragments thereof. The polypeptides also can be used in screening for such antibodies and antigen-binding fragments. In some embodiments, provided herein is a polypeptide comprising a contiguous amino acid sequence of human CD16a-F (e.g. SEQ ID NO: 352) or a variant of said contiguous amino acid sequence. In some embodiments, provided herein is a polypeptide comprising a contiguous amino acid sequence of human CD16a-V (e.g. SEQ ID NO: 353) or a variant of said contiguous amino acid sequence. In some embodiments, Y158 of SEQ ID NO: 352 is critical for the binding of the CD16a binding agent provided herein to CD16a-F. In some embodiments, Y158 of SEQ ID NO: 353 is critical for the binding of the CD16a binding agent provided herein to CD16a-V. In some embodiments, Y158 of SEQ ID NO: 352 is not critical for the binding of the CD16a binding agent provided herein to CD16a-F. In some embodiments, a Y158 of SEQ ID NO: 353 is not critical for the binding of the CD16a binding agent provided herein to CD16a-V. In some embodiments, glycosylation at position 82 of SEQ ID NO: 352 is critical for the binding of the CD16a binding agent provided herein to CD16a-F. In some embodiments, glycosylation at position 82 of SEQ ID NO: 353 is critical for the binding of the CD16a binding agent provided herein to CD16a-V. In some embodiments, glycosylation at position 82 of SEQ ID NO: 352 is not critical for the binding of the CD16a binding agent provided herein to CD16a-F. In some embodiments, glycosylation at position 82 of SEQ ID NO: 353 is not critical for the binding of the CD16a binding agent provided herein to CD16a-V. In some embodiments, deglycosylation at position 82 of CD16b does not change the binding affinity of the CD16a binding agent provided herein to CD16b. In some embodiments, deglycosylation at position 82 of CD16b does not increases the binding affinity of the CD16a binding agent provided herein to CD16b. In some embodiments, deglycosylation at position 82 of CD16b changes the binding of the CD16a binding agent provided herein to CD16b. In some embodiments, deglycosylation at position 82 of CD16b increases the binding of the CD16a binding agent provided herein to CD16b.

### 5.5. Pharmaceutical Compositions

In one aspect, the present disclosure further provides a composition, such as a pharmaceutical composition, comprising at least one of the following: a binding agent provided herein (e.g., an antibody, a multispecific antibody, or an antigen-binding fragment thereof of the present disclosure), a nucleic acid provided herein, a vector provided herein, or a cell provided herein. In some embodiments, a pharmaceutical composition comprises a therapeutically effective amount of a binding agent provided herein (e.g., an antibody, a multispecific antibody, or an antigen-binding fragment thereof of the present disclosure) and a pharmaceutically acceptable excipient. In some embodiments, a pharmaceutical composition comprises a therapeutically effective amount of a nucleic acid provided herein (such as a nucleic acid encoding an antibody, a multispecific antibody, or antigen-binding fragment thereof of the present disclosure) and a pharmaceutically acceptable excipient. In some embodiments, a pharmaceutical composition comprises a therapeutically effective amount of a vector provided herein (such as a vector comprising a nucleic acid as disclosed herein and expressing a binding agent as disclosed herein) and a pharmaceutically acceptable excipient. In some embodiments, a pharmaceutical composition comprises a therapeutically effective amount of a cell provided herein (such as a cell comprising a nucleic acid encoding an antibody, a multispecific antibody, or antigen-binding fragment thereof of the present disclosure, and/or expressing an antibody, a multispecific antibody, or antigen-binding fragment thereof of the present disclosure) and a pharmaceutically acceptable excipient.

In some embodiments, pharmaceutical compositions provided herein are prepared for storage by mixing the binding agents, nucleic acids, vectors, or cells provided herein having the desired degree of purity with optional physiologically acceptable excipients (see, e.g., Remington, Remington's Pharmaceutical Sciences (18th ed. 1980)) in the form of aqueous solutions or lyophilized or other dried forms.

The binding agents, nucleic acids, vectors, or cells of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

The binding agents, nucleic acids, vectors, or cells provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with the binding agents, nucleic acids, vectors or cells as described herein, including, but not limited to, encapsulation in liposomes, microparticles, microcapsules, recombinant cells capable of expressing the antibody or antigen-binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen-binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38(1984)). Controlled release systems are discussed, for example, by Langer, 1990, Science 249:1527-33. Any technique known to one of skill in the art can be used to produce sustained release formulations comprising one or more antibody or antigen-binding fragment thereof as described herein (see, e.g., U.S. Pat. No. 4,526,938, WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60)

### 5.6. Methods of Uses

In another aspect, provided herein are methods for using the binding agents such as an antibody, a multispecific antibody, or antigen-binding fragment thereof of the present disclosure or the composition provided herein. In a further aspect, provided herein are an antibody, a multispecific antibody, or antigen-binding fragment or the composition as disclosed herein for use as a medicament. In yet a further aspect, provided herein are an antibody, a multispecific antibody, or antigen-binding fragment thereof or compositions as disclosed herein for use in the treatment of a disease or condition, such as those disclosed herein. In one aspect, provided herein are an antibody, a multispecific antibody, or antigen-binding fragment thereof or compositions as disclosed herein for use in a method as disclosed herein.

In some embodiments, the binding agents bind to CD16a. In some embodiments, the binding agents bind to CD16a-V. In some embodiments, the binding agents bind to CD16a-F. In some embodiments, the binding agents bind to CD16a-V and CD16a-F.

In some embodiments, the CD16a binding agent (e.g., an antibody) provided herein does not bind to CD16b(e.g., human CD16b and/or cyno CD16b). In some embodiments, the CD16a binding agent (e.g., an antibody) provided herein does not bind to CD16b-NA1. In some embodiments, the CD16a binding agent (e.g., an antibody) provided herein does not bind to CD16b-NA2. In some embodiments, the CD16a binding agent (e.g., an antibody) provided herein does not bind to CD16b-NA1 and/or CD16b-NA2.

In some embodiments, provided herein is a method of enriching, isolating, separating, purifying, sorting, selecting, capturing, detecting, or activating cells expressing CD16a comprising providing a sample comprising the cells expressing CD16a; contacting the sample with the CD16a antibody provided herein; and enriching, isolating, separating, purifying, sorting, selecting, capturing, detecting, or activating the cells expressing CD16a and bound to the CD16a antibody. In some embodiments, the cells are NK cells. In some embodiments, the sample is a blood sample. In other embodiments, the sample is a tissue sample.

In yet another aspect, provided herein is a method of directing an NK cell to a target cell, comprising contacting the NK cell with a multispecific antibody provided herein, thereby directing the NK cell to the target cell, wherein the multispecific antibody comprises a CD16a binding domain that binds to a first antigen on an NK cell and a second binding domain that binds to a second antigen on a target cell. In some embodiments, the target cell is a tumor cell. In some embodiments, the second antigen is CD19.

In yet another aspect, provided herein is a method of activating an NK cell, comprising contacting the NK cell with a CD16a antibody provided herein.

In yet another aspect, provided herein is a method of activating an NK cell, comprising contacting the NK cell with a multispecific antibody provided herein, wherein the multispecific antibody comprises a first binding domain that binds to CD16a on the NK cell and a second binding domain that binds to a second antigen on a target cell. In some embodiments, the target cell is a tumor cell. In certain embodiments, the second antigen is CD19.

In some embodiments, provided herein is a method of inducing ADCC activity of an NK cell, comprising contacting the NK cell with a CD16a antibody provided herein. In yet another aspect, provided herein is a method of inducing ADCC activity of an NK cell, comprising contacting the NK cell with a multispecific antibody provided herein, wherein the multispecific antibody comprises a first binding domain that binds to CD16a on the NK cell and a second binding domain that binds to a second antigen on a target cell. In some embodiments, the target cell is a tumor cell. In certain embodiments, the second antigen is CD19.

In some embodiments, provided herein is a method of inducing cytokine secretion of an NK cell, comprising contacting the NK cell with a CD16a antibody provided herein. In yet another aspect, provided herein is a method of inducing cytokine secretion of an NK cell, comprising contacting the NK cell with a multispecific antibody provided herein, wherein the multispecific antibody comprises a first binding domain that binds to CD16a on the NK cell and a second binding domain that binds to a second antigen on a target cell. In some embodiments, the target cell is a tumor cell. In certain embodiments, the second antigen is CD19.

In yet another aspect, provided herein is a method of inhibiting growth or proliferation of target cells expressing a second antigen on the cell surface, the method comprising contacting the target cells with a multispecific antibody provided herein, wherein the multispecific antibody comprises a first binding domain that binds to CD16a on an NK cell and a second binding domain that binds to the second antigen. In some embodiments, the second antigen express on a tumor cell. In certain embodiments, the second antigen is CD19.

In yet another aspect, provided herein is a method for eliminating target cells expressing a second antigen or treating a disease or disorder caused all or in part by target cells expressing the second antigen in a subject, comprising administering an effective amount of a multispecific antibody provided herein to the subject, wherein the multispecific antibody comprises a first binding domain that binds to CD16a on an NK cell and a second binding domain that binds to the second antigen. In some embodiments, the second antigen express on a tumor cell. In certain embodiments, the second antigen is CD19.

In some embodiments, a method as disclosed herein is an in vitro method or an ex vivo method. In other embodiments, a method as disclosed herein is an in vivo method. In some embodiments, a use as disclosed herein is an in vitro use or an ex vivo use. In other embodiments, a use as disclosed herein is an in vivo use. In some embodiments, an in vivo method or an in vivo use as disclosed herein comprises administering a binding agent as disclosed herein to a subject having a cell expressing CD19, such as a tumor cell expressing CD19.

### Therapeutic Uses and Methods of Treatment

In some embodiments, binding agents (e.g., antibodies, multispecific antibodies, or antigen-binding fragments thereof) described herein are useful in compositions and in methods of treating a disease or disorder. Thus, in some embodiments, provided herein is a method for treating a disease or disorder in a subject comprising administering the binding agent or the pharmaceutical composition provided herein to the subject. In other embodiments, provided herein is a use of the binding agent or pharmaceutical composition provided herein for treating a disease or disorder in a subject. In other embodiments, provided herein is a binding agent or pharmaceutical composition provided herein for use in the manufacture of a medicament for the treatment of a disease or disorder.

In some embodiments, the treatment provided herein includes alleviating one or more symptoms associated with a disease or disorder (e.g., a cancer or a tumor).

Thus, in some embodiments, provided herein is a method for alleviating one or more symptoms associated with a cancer or a tumor in a subject comprising administering to the subject a binding agent (e.g., an antibody, a multispecific antibody, or an antigen-binding fragment thereof) described herein or a pharmaceutical composition described herein. In some embodiments, provided herein is a binding agent (e.g., an antibody, a multispecific antibody, or an antigen-binding fragment thereof) described herein or a pharmaceutical composition described herein for use in alleviating one or more symptoms associated with a cancer or a tumor in a subject. In some embodiments, provided herein is a use of a binding agent (e.g., an antibody, a multispecific antibody, or an antigen-binding fragment thereof) described herein or a pharmaceutical composition described herein in the manufacture of a medicament for alleviating one or more symptoms associated with a cancer or a tumor in a subject.

In some embodiments, the treatment provided herein includes decreasing tumor size in a subject with a tumor. Thus, in some embodiments, described herein is a method for decreasing tumor size in a subject with a tumor comprising administering to the subject a binding agent (e.g., an antibody, a multispecific antibody, or an antigen-binding fragment thereof) described herein or a pharmaceutical composition described herein. In some embodiments, provided herein is a binding agent (e.g., an antibody, a multispecific antibody, or an antigen-binding fragment thereof) described herein or a pharmaceutical composition described herein for use in decreasing tumor size in a subject with a tumor. In some embodiments, provided herein is a use of a binding agent (e.g., an antibody, a multispecific antibody, or an antigen-binding fragment thereof) described herein or a pharmaceutical composition described herein in the manufacture of a medicament for decreasing tumor size in a subject with a tumor.

In some embodiments, described herein is a method for enhancing tumor cell removal in a subject with a tumor comprising administering to the subject a binding agent (e.g., an antibody, a multispecific antibody, or an antigen-binding fragment thereof) described herein or a pharmaceutical composition described herein. In some embodiments, provided herein is a binding agent (e.g., an antibody, a multispecific antibody, or an antigen-binding fragment thereof) described herein or a pharmaceutical composition described herein for use in enhancing tumor cell removal in a subject with a tumor. In some embodiments, provided herein is a use of a binding agent (e.g., an antibody, a multispecific antibody, or an antigen-binding fragment thereof) described herein or a pharmaceutical composition described herein in the manufacture of a medicament for enhancing tumor cell removal in a subject with a tumor.

“Enhancing” tumor cell removal includes, yet does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is included but not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a binding agent (e.g., a multispecific binding agent, such as a bispecific antibody) or a pharmaceutical composition of the method.

A method of modulating (e.g., inhibiting, reducing, preventing) tumor growth in a subject also is provided. For example, the method comprises administering to the subject a composition comprising a binding agent (e.g., an antibody, a multispecific antibody, or antigen-binding fragment) or another composition as disclosed herein in an amount effective to modulate tumor growth in the subject.

In some embodiments, the disease or disorder is a cancer or a tumor. In further embodiments, the cancer or tumor expresses CD19 and the cancer is a B-cell leukemia or lymphoma. In further embodiments, the cancer or a tumor expresses BCMA and the tumor or cancer is multiple myeloma. In some embodiments, the subject is a human subject.

As used herein, “tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated cell growth.

In some embodiments, the tumor is a solid tumor. In some embodiments, the tumor or cancer is not a solid tumor. In further embodiments, the cancer is a B-cell leukemia cancer. In further embodiments, the cancer is a B-cell lymphoma cancer. In further embodiments, the cancer is multiple myeloma. In some embodiments, the tumor or cancer is a relapsed tumor or cancer. In some embodiments, the tumor or cancer is a metastatic tumor or cancer. In some embodiments, the tumor or cancer is a primary tumor or cancer. In some embodiments, the tumor or cancer reaches a remission, but can relapse. In some embodiments, the tumor or cancer is unresectable. Additionally or alternatively, the tumor or cancer is resistant to a chemotherapy or other anti-cancer therapy. In further embodiments, the cancer or tumor expresses CD19. In further embodiments, the cancer or a tumor expresses BCMA.

Additionally, binding agents (e.g., antibodies, multispecific antibodies, or antigen-binding fragments thereof) may be used to alleviate or reduce side effects associated with cancer, for example, bone deterioration, vertebral collapse, and paralysis. In one aspect, the subject suffers from or is at risk of suffering from bone metastases and a binding agent (e.g., an antibody, a multispecific antibody, or an antigen-binding fragment thereof) is administered in an amount to reduce deterioration of surrounding bone. Accordingly, in some aspects, a binding agent provided herein prevents bone deterioration due to bone metastases, wherein tumor cell proliferation is or is not reduced. In some aspects, a binding agent (e.g., an antibody, a multispecific antibody, or an antigen-binding fragment thereof) both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen may be inhibited. In contrast, proliferation of metastases located in microenvironments lacking substantial amounts of type 1 collagen may not be inhibited, yet bone deterioration in the vicinity of the metastasis is reduced or prevented.

Also provided is a method of treating a disease or disorder (e.g., a cancer) by administering a binding agent (e.g., an antibody, a multispecific antibody, or an antigen-binding fragment thereof) provided herein, or a pharmaceutical composition as disclosed herein, to a subject in need thereof, alone or in combination with another agent.

The subject of a method described herein can be administered one or more additional therapeutic agents in combination with a binding agent (e.g., an antibody, a multispecific antibody, or an antigen-binding fragment thereof) described herein or fragment thereof or a pharmaceutical composition described herein. An additional agent can be an agent that targets a tumor or a cancer cell. An additional agent can also be an agent that targets an immune cell (e.g., an NK cell or a T cell). In some embodiments, the binding agent or pharmaceutical composition provided herein increases a therapeutic effect of the additional agent by about 10%-90% or about 2-100 folds. In some embodiments, the binding agent or pharmaceutical composition provided herein increases a therapeutic effect of the additional agent by at least 10%. In some embodiments, the binding agent or pharmaceutical composition provided herein increases a therapeutic effect of the additional agent by at least 20%. In some embodiments, the binding agent or pharmaceutical composition provided herein increases a therapeutic effect of the additional agent by at least 30%. In some embodiments, the binding agent or pharmaceutical composition provided herein increases a therapeutic effect of the additional agent by at least 40%. In some embodiments, the binding agent or pharmaceutical composition provided herein increases a therapeutic effect of the additional agent by at least 50%. In some embodiments, the binding agent or pharmaceutical composition provided herein increases a therapeutic effect of the additional agent by at least 60%. In some embodiments, the binding agent or pharmaceutical composition provided herein increases a therapeutic effect of the additional agent by at least 70%. In some embodiments, the binding agent or pharmaceutical composition provided herein increases a therapeutic effect of the additional agent by at least 80%. In some embodiments, the binding agent or pharmaceutical composition provided herein increases a therapeutic effect of the additional agent by at least 90%. In some embodiments, the binding agent or pharmaceutical composition provided herein increases a therapeutic effect of the additional agent by at least 2 fold. In some embodiments, the binding agent or pharmaceutical composition provided herein increases a therapeutic effect of the additional agent by at least 5 fold. In some embodiments, the binding agent or pharmaceutical composition provided herein increases a therapeutic effect of the additional agent by at least 10 fold. In some embodiments, the binding agent or pharmaceutical composition provided herein increases a therapeutic effect of the additional agent by at least 20 fold. In some embodiments, the binding agent or pharmaceutical composition provided herein increases a therapeutic effect of the additional agent by more than 50 fold.

A particular administration regimen of a binding agent (e.g., an antibody, a multispecific antibody, or an antigen-binding fragment thereof) or pharmaceutical composition as disclosed herein for a particular subject will depend, in part, upon the agent used, the amount of agent administered, the route of administration, and the cause and extent of any side effects. The amount of the binding agent (e.g., an antibody, a multispecific antibody, or an antigen-binding fragment thereof) administered to a subject (e.g., a mammal, such as a human) should be sufficient to effect the desired response over a reasonable time frame. According, in some embodiments, the amount of a binding agent (e.g., an antibody, a multispecific antibody, or an antigen-binding fragment thereof) or pharmaceutical composition described herein administered to a subject is an effective amount.

Suitable routes of administering a binding agent (e.g., an antibody, a multispecific antibody, or an antigen-binding fragment thereof), or a composition described herein, are well known in the art, such as intravenous injection (such as intravenous infusion), intratumoral injection, or injection adjacent to a tumor or cancer. Although more than one route can be used to administer an agent (e.g., an antibody, a multispecific antibody, or an antigen-binding fragment thereof), a particular route can provide a more immediate and more effective reaction than another route.

### Gene and Cellular Therapies

In some embodiments, the composition for use according to the present disclosure comprises one or more nucleic acids encoding a binding agent provided herein (e.g., an antibody, a multispecific antibody, or an antigen-binding fragment thereof) or complementary nucleic acids thereto. In a specific embodiment, the nucleic acids are administered to a subject for use in a method provided herein, for example, to prevent, manage, treat and/or ameliorate a disease or disorder (e.g., a cancer, for example a cancer expressing CD19) by way of gene and/or cell therapy. Such therapy encompasses that performed by the administration to a subject of an expressed or expressible nucleic acid. In an embodiment, the nucleic acids produce their encoded antibody, and the antibody mediates a prophylactic or therapeutic effect.

Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIBTECH 11(5): 155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, a composition comprises nucleic acids encoding a binding agent (e.g., an antibody, a multispecific antibody, or an antigen-binding fragment thereof) or a fusion protein provided herein, the nucleic acids being part of an expression vector that expresses the binding agent or fusion proteins or heavy or light chains thereof in a suitable host. In particular, such nucleic acids have promoters, such as heterologous promoters, operably linked to the coding region, the promoter being inducible or constitutive, and, optionally, tissue-specific and/or tumor/cancer-specific. In another particular embodiment, nucleic acid molecules are used in which the coding sequences and any other desired sequences are flanked by regions that promote homologous recombination at a desired site in the genome, thus providing for intrachromosomal expression of the antibody or fusion protein encoding nucleic acids (Koller and Smithies, 1989, Proc. Natl. Acad. Sci. USA 86:8932-8935; Zijlstra et al., 1989, Nature 342:435-438).

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., WO 92/06180; WO 92/22635; WO 92/20316; WO 93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express a bispecific antibody provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

In some embodiments, oncolytic viruses may be used in the recombinant production of antibodies provided herein. An oncolytic virus may preferentially infect and kill cancer cells. As the infected cancer cells are destroyed by oncolysis, they may release new infectious virus particles or virions to help destroy the remaining tumor. In a specific embodiment, an oncolytic virus is a virus that when injected into a tumor results in tumor regression. In another specific embodiment, an oncolytic virus is a virus that selectively replicates in and kills cancer cells, and spreads within the tumor. In another specific embodiment, an oncolytic virus is a virus that selectively replicates in and kills cancer cells, and spreads within the tumor without causing any significant damage to normal tissue. In some embodiments, an in vitro or ex vivo assay known to one skilled in the art is used to determine the selectively of a virus to replicate in cancer cells versus non-cancerous cells (e.g., healthy cells). In one embodiment, a virus selectively replicates in cancer cells if a statistically significant increase in the number of virus particles is detected in cancer cells in an in vitro assay or ex vivo assay relative to the number of virus particles detected in non-cancerous cells (e.g., healthy cells) in the same assay after incubation with the virus. In another embodiment, a virus selectively kills cancer cells if a statistically significant amount of the cancer cells are killed in an in vitro or ex vivo assay relative to the amount of non-cancerous cells (e.g., healthy cells) killed in the same assay. In one embodiment, an oncolytic virus naturally preferentially replicates in cancer cells and is non-pathogenic in humans. An oncolytic virus may be non-pathogenic in humans due to elevated sensitivity to innate antiviral signal or dependence on oncogenic signaling pathways. In some embodiments, an oncolytic virus is a parovirus (e.g., an autonomous parvovirus), a myxoma virus, an avian paramyxovirus (e.g., Newcastle disease virus), a reovirus, or Seneca valley virus. In one embodiment, an oncolytic virus is wild-type parvovirus H1 (ParvOryx). In another embodiment, an oncolytic virus is Vesicular stomatitis virus. In another embodiment, an oncolytic virus is an avian paramyxovirus. In some embodiments, an oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In some embodiments, such viruses are attenuated.

Another approach to gene and cell therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92(1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene and/or cell therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for cell and/or gene therapy is autologous to the subject. In other embodiments, the cell used for cell and/or gene therapy is allogeneic to the subject, such as an NK cell.

In an embodiment in which recombinant cells are used in gene and/or therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., WO 94/08598; Stemple and Anderson, 1992, Cell 7 1:973-985; Rheinwald, 1980, Meth. Cell Bio. 21A: 229; and Pittelkow and Scott, 1986, Mayo Clinic Proc. 61:771).

In a specific embodiment, the nucleic acid to be introduced for purposes of gene and/or therapy comprises an inducible promoter operably linked to the coding region, such that expression of the nucleic acid is controllable by controlling the presence or absence of the appropriate inducer of transcription.

### 6. Embodiments

The present disclosure encompasses the following non-limiting embodiments:

Embodiment 1. An antibody or fragment thereof that binds to CD16a, wherein the antibody or antigen binding fragment comprises:
(i) a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38;
(ii) a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76;
(iii) a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114;
(iv) a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152;
(v) a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190;
(vi) a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228;
(vii) a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266;
(vii) a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304; or
(ix) a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342.

The antibody or fragment thereof of embodiment 1, wherein the antibody or fragment thereof comprises:
(a) a VH region comprising:
(1) a VH CDR1 having an amino acid sequence selected from the group consisting of SEQ ID NOs: 1, 7, 13, 19, 25, and 31;
(2) a VH CDR2 having an amino acid sequence selected from the group consisting of SEQ ID NOs: 2, 8, 14, 20, 26, and 32; and
(3) a VH CDR3 having an amino acid sequence selected from the group consisting of SEQ ID NOs: 3, 9, 15, 21, 27, and 33;
   and
(b) a VL region comprising:
(1) a VL CDR1 having an amino acid sequence selected from the group consisting of SEQ ID NOs: 4, 10, 16, 22, 28, and 34;
(2) a VL CDR2 having an amino acid sequence selected from the group consisting of SEQ ID NOs: 5, 11, 17, 23, 29, and 35; and
(3) a VL CDR3 having an amino acid sequence selected from the group consisting of SEQ ID NOs: 6, 12, 18, 24, 30, and 36.

Embodiment 3. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 1, a VH CDR2 comprising the amino acid sequence of SEQ ID NO: 2, a VH CDR3 comprising the amino acid sequence of SEQ ID NO: 3; and a VL region comprising a VL CDR1 comprising the amino acid sequence of SEQ ID NO: 4, a VL CDR2 comprising the amino acid sequence of SEQ ID NO: 5, and a VL CDR3 comprising the amino acid sequence of SEQ ID NO: 6;
(ii) a VH region comprising a VH CDR1 comprising the amino acid sequence of SEQ ID NO: 7, a VH CDR2 comprising the amino acid sequence of SEQ ID NO: 8, a VH CDR3 comprising the amino acid sequence of SEQ ID NO: 9; and a VL region comprising a VL CDR1 comprising the amino acid sequence of SEQ ID NO: 10, a VL CDR2 comprising the amino acid sequence of SEQ ID NO: 11, and a VL CDR3 comprising the amino acid sequence of SEQ ID NO: 12;
(iii) a VH region comprising a VH CDR1 comprising the amino acid sequence of SEQ ID NO: 13, a VH CDR2 comprising the amino acid sequence of SEQ ID NO: 14, a VH CDR3 comprising the amino acid sequence of SEQ ID NO: 15; and a VL region comprising a VL CDR1 comprising the amino acid sequence of SEQ ID NO: 16, a VL CDR2 comprising the amino acid sequence of SEQ ID NO: 17, and a VL CDR3 comprising the amino acid sequence of SEQ ID NO: 18;
(iv) a VH region comprising a VH CDR1 comprising the amino acid sequence of SEQ ID NO: 19, a VH CDR2 comprising the amino acid sequence of SEQ ID NO: 20, a VH CDR3 comprising the amino acid sequence of SEQ ID NO: 21; and a VL region comprising a VL CDR1 comprising the amino acid sequence of SEQ ID NO: 22, a VL CDR2 comprising the amino acid sequence of SEQ ID NO: 23, and a VL CDR3 comprising the amino acid sequence of SEQ ID NO: 24;
(v) a VH region comprising a VH CDR1 comprising the amino acid sequence of SEQ ID NO: 25, a VH CDR2 comprising the amino acid sequence of SEQ ID NO: 26, a VH CDR3 comprising the amino acid sequence of SEQ ID NO: 27; and a VL region comprising a VL CDR1 comprising the amino acid sequence of SEQ ID NO: 28, a VL CDR2 comprising the amino acid sequence of SEQ ID NO: 29, and a VL CDR3 comprising the amino acid sequence of SEQ ID NO: 30; or
(vi) a VH region comprising a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33; and a VL region comprising a VL CDR1 comprising the amino acid sequence of SEQ ID NO: 34, a VL CDR2 comprising the amino acid sequence of SEQ ID NO: 35, and a VL CDR3 comprising the amino acid sequence of SEQ ID NO: 36.

Embodiment 4. The antibody or fragment thereof of embodiment 1, wherein the antibody or fragment thereof comprises
(a) a VH region comprising:
(1) a VH CDR1 having an amino acid sequence selected from the group consisting of SEQ ID NOs: 39, 45, 51, 57, 63, and 69;
(2) a VH CDR2 having an amino acid sequence selected from the group consisting of SEQ ID NOs: 40, 46, 52, 58, 64, and 70; and
(3) a VH CDR3 having an amino acid sequence selected from the group consisting of SEQ ID NOs: 41, 47, 53, 59, 65, and 71;
   and
(b) a VL region comprising:
(1) a VL CDR1 having an amino acid sequence selected from the group consisting of SEQ ID NOs: 42, 48, 54, 60, 66, and 72;
(2) a VL CDR2 having an amino acid sequence selected from the group consisting of SEQ ID NOs: 43, 49, 55, 61, 67, and 73; and
(3) a VL CDR3 having an amino acid sequence selected from the group consisting of SEQ ID NOs: 44, 50, 56, 62, 68, and 74.

Embodiment 5. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 39, a VH CDR2 comprising the amino acid sequence of SEQ ID NO: 40, and a VH CDR3 comprising the amino acid sequence of SEQ ID NO: 41; and a VL region comprising a VL CDR1 comprising the amino acid sequence of SEQ ID NO: 42, a VL CDR2 comprising the amino acid sequence of SEQ ID NO: 43, and a VL CDR3 comprising the amino acid sequence of SEQ ID NO: 44;
(ii) a VH region comprising a VH CDR1 comprising the amino acid sequence of SEQ ID NO: 45, a VH CDR2 comprising the amino acid sequence of SEQ ID NO: 46, and a VH CDR3 comprising the amino acid sequence of SEQ ID NO: 47; and a VL region comprising a VL CDR1 comprising the amino acid sequence of SEQ ID NO: 48, a VL CDR2 comprising the amino acid sequence of SEQ ID NO: 49, and a VL CDR3 comprising the amino acid sequence of SEQ ID NO: 50;
(iii) a VH region comprising a VH CDR1 comprising the amino acid sequence of SEQ ID NO: 51, a VH CDR2 comprising the amino acid sequence of SEQ ID NO: 52, and a VH CDR3 comprising the amino acid sequence of SEQ ID NO: 53; and a VL region comprising a VL CDR1 comprising the amino acid sequence of SEQ ID NO: 54, a VL CDR2 comprising the amino acid sequence of SEQ ID NO: 55, and a VL CDR3 comprising the amino acid sequence of SEQ ID NO: 56;
(iv) a VH region comprising a VH CDR1 comprising the amino acid sequence of SEQ ID NO: 57, a VH CDR2 comprising the amino acid sequence of SEQ ID NO: 58, and a VH CDR3 comprising the amino acid sequence of SEQ ID NO: 59; and a VL region comprising a VL CDR1 comprising the amino acid sequence of SEQ ID NO: 60, a VL CDR2 comprising the amino acid sequence of SEQ ID NO: 61, and a VL CDR3 comprising the amino acid sequence of SEQ ID NO: 62;
(v) a VH region comprising a VH CDR1 comprising the amino acid sequence of SEQ ID NO: 63, a VH CDR2 comprising the amino acid sequence of SEQ ID NO: 64, and a VH CDR3 comprising the amino acid sequence of SEQ ID NO: 65; and a VL region comprising a VL CDR1 comprising the amino acid sequence of SEQ ID NO: 66, a VL CDR2 comprising the amino acid sequence of SEQ ID NO: 67, and a VL CDR3 comprising the amino acid sequence of SEQ ID NO: 68; or
(vi) a VH region comprising a VH CDR1 comprising the amino acid sequence of SEQ ID NO: 69, a VH CDR2 comprising the amino acid sequence of SEQ ID NO: 70, and a VH CDR3 comprising the amino acid sequence of SEQ ID NO: 71; and a VL region comprising a VL CDR1 comprising the amino acid sequence of SEQ ID NO: 72, a VL CDR2 comprising the amino acid sequence of SEQ ID NO: 73, and a VL CDR3 comprising the amino acid sequence of SEQ ID NO: 74.

Embodiment 6. The antibody or fragment thereof of embodiment 1, wherein the antibody or fragment thereof comprises:
(a) a VH region comprising:
(1) a VH CDR1 having an amino acid sequence selected from the group consisting of SEQ ID NOs: 77, 83, 89, 95, 101, and 107;
(2) a VH CDR2 having an amino acid sequence selected from the group consisting of SEQ ID NOs: 78, 84, 90, 96, 102, and 108; and
(3) a VH CDR3 having an amino acid sequence selected from the group consisting of SEQ ID NOs: 79, 85, 91, 97, 103, and 109;
   and
(b) a VL region comprising:
(1) a VL CDR1 having an amino acid sequence selected from the group consisting of SEQ ID NOs: 80, 86, 92, 98, 104, and 110;
(2) a VL CDR2 having an amino acid sequence selected from the group consisting of SEQ ID NOs: 81, 87, 93, 99, 105, and 111; and
(3) a VL CDR3 having an amino acid sequence selected from the group consisting of SEQ ID NOs: 82, 88, 94, 100, 106, and 112.

Embodiment 7. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 77, a VH CDR2 comprising the amino acid sequence of SEQ ID NO: 78, and a VH CDR3 comprising the amino acid sequence of SEQ ID NO: 79; and a VL region comprising a VL CDR1 comprising the amino acid sequence of SEQ ID NO: 80, a VL CDR2 comprising the amino acid sequence of SEQ ID NO: 81, and a VL CDR3 comprising the amino acid sequence of SEQ ID NO: 82;
(ii) a VH region comprising a VH CDR1 comprising the amino acid sequence of SEQ ID NO: 83, a VH CDR2 comprising the amino acid sequence of SEQ ID NO: 84, and a VH CDR3 comprising the amino acid sequence of SEQ ID NO: 85; and a VL region comprising a VL CDR1 comprising the amino acid sequence of SEQ ID NO: 86, a VL CDR2 comprising the amino acid sequence of SEQ ID NO: 87, and a VL CDR3 comprising the amino acid sequence of SEQ ID NO: 88;
(iii) a VH region comprising a VH CDR1 comprising the amino acid sequence of SEQ ID NO: 89, a VH CDR2 comprising the amino acid sequence of SEQ ID NO: 90, and a VH CDR3 comprising the amino acid sequence of SEQ ID NO: 91; and a VL region comprising a VL CDR1 comprising the amino acid sequence of SEQ ID NO: 92, a VL CDR2 comprising the amino acid sequence of SEQ ID NO: 93, and a VL CDR3 comprising the amino acid sequence of SEQ ID NO: 94;
(iv) a VH region comprising a VH CDR1 comprising the amino acid sequence of SEQ ID NO: 95, a VH CDR2 comprising the amino acid sequence of SEQ ID NO: 96, and a VH CDR3 comprising the amino acid sequence of SEQ ID NO: 97; and a VL region comprising a VL CDR1 comprising the amino acid sequence of SEQ ID NO: 98, a VL CDR2 comprising the amino acid sequence of SEQ ID NO: 99, and a VL CDR3 comprising the amino acid sequence of SEQ ID NO: 100;
(v) a VH region comprising a VH CDR1 comprising the amino acid sequence of SEQ ID NO: 101, a VH CDR2 comprising the amino acid sequence of SEQ ID NO: 102, and a VH CDR3 comprising the amino acid sequence of SEQ ID NO: 103; and a VL region comprising a VL CDR1 comprising the amino acid sequence of SEQ ID NO: 104, a VL CDR2 comprising the amino acid sequence of SEQ ID NO: 105, and a VL CDR3 comprising the amino acid sequence of SEQ ID NO: 106; or
(vi) a VH region comprising a VH CDR1 comprising the amino acid sequence of SEQ ID NO: 107, a VH CDR2 comprising the amino acid sequence of SEQ ID NO: 108, and a VH CDR3 comprising the amino acid sequence of SEQ ID NO: 109; and a VL region comprising a VL CDR1 comprising the amino acid sequence of SEQ ID NO: 110, a VL CDR2 comprising the amino acid sequence of SEQ ID NO: 111, and a VL CDR3 comprising the amino acid sequence of SEQ ID NO: 112.

Embodiment 8. The antibody or fragment thereof of embodiment 1, wherein the antibody or fragment thereof comprises:
(a) a VH region comprising:
(1) a VH CDR1 having an amino acid sequence selected from the group consisting of SEQ ID NOs: 115, 121, 127, 133, 139, and 145;
(2) a VH CDR2 having an amino acid sequence selected from the group consisting of SEQ ID NOs: 116, 122, 128, 134, 140, and 146; and
(3) a VH CDR3 having an amino acid sequence selected from the group consisting of SEQ ID NOs: 117, 123, 129, 135, 141, and 147;
   and
(b) a VL region comprising:
(1) a VL CDR1 having an amino acid sequence selected from the group consisting of SEQ ID NOs: 118, 124, 130, 136, 142, and 148;
(2) a VL CDR2 having an amino acid sequence selected from the group consisting of SEQ ID NOs: 119, 125, 131, 137, 143, and 149; and
(3) a VL CDR3 having an amino acid sequence selected from the group consisting of SEQ ID NOs: 120, 126, 132, 138, 144, and 150.

Embodiment 9. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 115, a VH CDR2 comprising the amino acid sequence of SEQ ID NO: 116, and a VH CDR3 comprising the amino acid sequence of SEQ ID NO: 117; and a VL region comprising a VL CDR1 comprising the amino acid sequence of SEQ ID NO: 118, a VL CDR2 comprising the amino acid sequence of SEQ ID NO: 119, and a VL CDR3 comprising the amino acid sequence of SEQ ID NO: 120;
(ii) a VH region comprising a VH CDR1 comprising the amino acid sequence of SEQ ID NO: 121, a VH CDR2 comprising the amino acid sequence of SEQ ID NO: 122, and a VH CDR3 comprising the amino acid sequence of SEQ ID NO: 123; and a VL region comprising a VL CDR1 comprising the amino acid sequence of SEQ ID NO: 124, a VL CDR2 comprising the amino acid sequence of SEQ ID NO: 125, and a VL CDR3 comprising the amino acid sequence of SEQ ID NO: 126;
(iii) a VH region comprising a VH CDR1 comprising the amino acid sequence of SEQ ID NO: 127, a VH CDR2 comprising the amino acid sequence of SEQ ID NO: 128, and a VH CDR3 comprising the amino acid sequence of SEQ ID NO: 129; and a VL region comprising a VL CDR1 comprising the amino acid sequence of SEQ ID NO: 130, a VL CDR2 comprising the amino acid sequence of SEQ ID NO: 131, and a VL CDR3 comprising the amino acid sequence of SEQ ID NO: 132;
(iv) a VH region comprising a VH CDR1 comprising the amino acid sequence of SEQ ID NO: 133, a VH CDR2 comprising the amino acid sequence of SEQ ID NO: 134, and a VH CDR3 comprising the amino acid sequence of SEQ ID NO: 135; and a VL region comprising a VL CDR1 comprising the amino acid sequence of SEQ ID NO: 136, a VL CDR2 comprising the amino acid sequence of SEQ ID NO: 137, and a VL CDR3 comprising the amino acid sequence of SEQ ID NO: 138;
(v) a VH region comprising a VH CDR1 comprising the amino acid sequence of SEQ ID NO: 139, a VH CDR2 comprising the amino acid sequence of SEQ ID NO: 140, and a VH CDR3 comprising the amino acid sequence of SEQ ID NO: 141; and a VL region comprising a VL CDR1 comprising the amino acid sequence of SEQ ID NO: 142, a VL CDR2 comprising the amino acid sequence of SEQ ID NO: 143, and a VL CDR3 comprising the amino acid sequence of SEQ ID NO: 144; or
(vi) a VH region comprising a VH CDR1 comprising the amino acid sequence of SEQ ID NO: 145, a VH CDR2 comprising the amino acid sequence of SEQ ID NO: 146, and a VH CDR3 comprising the amino acid sequence of SEQ ID NO: 147; and a VL region comprising a VL CDR1 comprising the amino acid sequence of SEQ ID NO: 148, a VL CDR2 comprising the amino acid sequence of SEQ ID NO: 149, and a VL CDR3 comprising the amino acid sequence of SEQ ID NO: 150.

Embodiment 10. The antibody or fragment thereof of embodiment 1, wherein the antibody or fragment thereof comprises
(a) a VH region comprising:
(1) a VH CDR1 having an amino acid sequence selected from the group consisting of SEQ ID NOs: 153, 159, 165, 171, 177, and 183;
(2) a VH CDR2 having an amino acid sequence selected from the group consisting of SEQ ID NOs: 154, 160, 166, 172, 178, and 184; and
(3) a VH CDR3 having an amino acid sequence selected from the group consisting of SEQ ID NOs: 155, 161, 167, 173, 179, and 185;
   and
(b) a VL region comprising:
(1) a VL CDR1 having an amino acid sequence selected from the group consisting of SEQ ID NOs: 156, 162, 168, 174, 180, and 186;
(2) a VL CDR2 having an amino acid sequence selected from the group consisting of SEQ ID NOs: 157, 163, 169, 175, 181, and 187; and
(3) a VL CDR3 having an amino acid sequence selected from the group consisting of SEQ ID NOs: 158, 164, 170, 176, 182, and 188.

Embodiment 11. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 153, a VH CDR2 comprising the amino acid sequence of SEQ ID NO: 154, and a VH CDR3 comprising the amino acid sequence of SEQ ID NO: 155; and a VL region comprising a VL CDR1 comprising the amino acid sequence of SEQ ID NO: 156, a VL CDR2 comprising the amino acid sequence of SEQ ID NO: 157, and a VL CDR3 comprising the amino acid sequence of SEQ ID NO: 158;
(ii) a VH region comprising a VH CDR1 comprising the amino acid sequence of SEQ ID NO: 159, a VH CDR2 comprising the amino acid sequence of SEQ ID NO: 160, and a VH CDR3 comprising the amino acid sequence of SEQ ID NO: 161; and a VL region comprising a VL CDR1 comprising the amino acid sequence of SEQ ID NO: 162, a VL CDR2 comprising the amino acid sequence of SEQ ID NO: 163, and a VL CDR3 comprising the amino acid sequence of SEQ ID NO: 164;
(iii) a VH region comprising a VH CDR1 comprising the amino acid sequence of SEQ ID NO: 165, a VH CDR2 comprising the amino acid sequence of SEQ ID NO: 166, and a VH CDR3 comprising the amino acid sequence of SEQ ID NO: 167; and a VL region comprising a VL CDR1 comprising the amino acid sequence of SEQ ID NO: 168, a VL CDR2 comprising the amino acid sequence of SEQ ID NO: 169, and a VL CDR3 comprising the amino acid sequence of SEQ ID NO: 170;
(iv) a VH region comprising a VH CDR1 comprising the amino acid sequence of SEQ ID NO: 171, a VH CDR2 comprising the amino acid sequence of SEQ ID NO: 172, and a VH CDR3 comprising the amino acid sequence of SEQ ID NO: 173; and a VL region comprising a VL CDR1 comprising the amino acid sequence of SEQ ID NO: 174, a VL CDR2 comprising the amino acid sequence of SEQ ID NO: 175, and a VL CDR3 comprising the amino acid sequence of SEQ ID NO: 176;
(v) a VH region comprising a VH CDR1 comprising the amino acid sequence of SEQ ID NO: 177, a VH CDR2 comprising the amino acid sequence of SEQ ID NO: 178, and a VH CDR3 comprising the amino acid sequence of SEQ ID NO: 179; and a VL region comprising a VL CDR1 comprising the amino acid sequence of SEQ ID NO: 180, a VL CDR2 comprising the amino acid sequence of SEQ ID NO: 181, and a VL CDR3 comprising the amino acid sequence of SEQ ID NO: 182; or
(vi) a VH region comprising a VH CDR1 comprising the amino acid sequence of SEQ ID NO: 183, a VH CDR2 comprising the amino acid sequence of SEQ ID NO: 184, and a VH CDR3 comprising the amino acid sequence of SEQ ID NO: 185; and a VL region comprising a VL CDR1 comprising the amino acid sequence of SEQ ID NO: 186, a VL CDR2 comprising the amino acid sequence of SEQ ID NO: 187, and a VL CDR3 comprising the amino acid sequence of SEQ ID NO: 188.

Embodiment 12. The antibody or fragment thereof of embodiment 1, wherein the antibody or fragment thereof comprises:
(a) a VH region comprising:
(1) a VH CDR1 having an amino acid sequence selected from the group consisting of SEQ ID NOs: 191, 197, 203, 209, 215, and 221;
(2) a VH CDR2 having an amino acid sequence selected from the group consisting of SEQ ID NOs: 192, 198, 204, 210, 216, and 222; and
(3) a VH CDR3 having an amino acid sequence selected from the group consisting of SEQ ID NOs: 193, 199, 205, 211, 217, and 223;
   and
(b) a VL region comprising:
(1) a VL CDR1 having an amino acid sequence selected from the group consisting of SEQ ID NOs: 194, 200, 206, 212, 218, and 224;
(2) a VL CDR2 having an amino acid sequence selected from the group consisting of SEQ ID NOs: 195, 201, 207, 213, 219, and 225; and
(3) a VL CDR3 having an amino acid sequence selected from the group consisting of SEQ ID NOs: 196, 202, 208, 214, 220, and 226.

Embodiment 13. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 191, a VH CDR2 comprising the amino acid sequence of SEQ ID NO: 192, and a VH CDR3 comprising the amino acid sequence of SEQ ID NO: 193; and a VL region comprising a VL CDR1 comprising the amino acid sequence of SEQ ID NO: 194, a VL CDR2 comprising the amino acid sequence of SEQ ID NO: 195, and a VL CDR3 comprising the amino acid sequence of SEQ ID NO: 196;
(ii) a VH region comprising a VH CDR1 comprising the amino acid sequence of SEQ ID NO: 197, a VH CDR2 comprising the amino acid sequence of SEQ ID NO: 198, and a VH CDR3 comprising the amino acid sequence of SEQ ID NO: 199; and a VL region comprising a VL CDR1 comprising the amino acid sequence of SEQ ID NO: 200, a VL CDR2 comprising the amino acid sequence of SEQ ID NO: 201, and a VL CDR3 comprising the amino acid sequence of SEQ ID NO: 202;
(iii) a VH region comprising a VH CDR1 comprising the amino acid sequence of SEQ ID NO: 203, a VH CDR2 comprising the amino acid sequence of SEQ ID NO: 204, and a VH CDR3 comprising the amino acid sequence of SEQ ID NO: 205; and a VL region comprising a VL CDR1 comprising the amino acid sequence of SEQ ID NO: 206, a VL CDR2 comprising the amino acid sequence of SEQ ID NO: 207, and a VL CDR3 comprising the amino acid sequence of SEQ ID NO: 208;
(iv) a VH region comprising a VH CDR1 comprising the amino acid sequence of SEQ ID NO: 209, a VH CDR2 comprising the amino acid sequence of SEQ ID NO: 210, and a VH CDR3 comprising the amino acid sequence of SEQ ID NO: 211; and a VL region comprising a VL CDR1 comprising the amino acid sequence of SEQ ID NO: 212, a VL CDR2 comprising the amino acid sequence of SEQ ID NO: 213, and a VL CDR3 comprising the amino acid sequence of SEQ ID NO: 214;
(v) a VH region comprising a VH CDR1 comprising the amino acid sequence of SEQ ID NO: 215, a VH CDR2 comprising the amino acid sequence of SEQ ID NO: 216, and a VH CDR3 comprising the amino acid sequence of SEQ ID NO: 217; and a VL region comprising a VL CDR1 comprising the amino acid sequence of SEQ ID NO: 218, a VL CDR2 comprising the amino acid sequence of SEQ ID NO: 219, and a VL CDR3 comprising the amino acid sequence of SEQ ID NO: 220; or
(vi) a VH region comprising a VH CDR1 comprising the amino acid sequence of SEQ ID NO: 221, a VH CDR2 comprising the amino acid sequence of SEQ ID NO: 222, and a VH CDR3 comprising the amino acid sequence of SEQ ID NO: 223; and a VL region comprising a VL CDR1 comprising the amino acid sequence of SEQ ID NO: 224, a VL CDR2 comprising the amino acid sequence of SEQ ID NO: 225, and a VL CDR3 comprising the amino acid sequence of SEQ ID NO: 226.

Embodiment 14. The antibody or fragment thereof of embodiment 1, wherein the antibody or fragment thereof comprises:
(a) a VH region comprising:
(1) a VH CDR1 having an amino acid sequence selected from the group consisting of SEQ ID NOs: 229, 235, 241, 247, 253, and 259;
(2) a VH CDR2 having an amino acid sequence selected from the group consisting of SEQ ID NOs: 230, 236, 242, 248, 254, and 260; and
(3) a VH CDR3 having an amino acid sequence selected from the group consisting of SEQ ID NOs: 231, 237, 243, 249, 255, and 261;
   and
(b) a VL region comprising:
(1) a VL CDR1 having an amino acid sequence selected from the group consisting of SEQ ID NOs: 232, 238, 244, 250, 256, and 262;
(2) a VL CDR2 having an amino acid sequence selected from the group consisting of SEQ ID NOs: 233, 239, 245, 251, 257, and 263; and
(3) a VL CDR3 having an amino acid sequence selected from the group consisting of SEQ ID NOs: 234, 240, 246, 252, 258, and 264.

Embodiment 15. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 229, a VH CDR2 comprising the amino acid sequence of SEQ ID NO: 230, and a VH CDR3 comprising the amino acid sequence of SEQ ID NO: 231; and a VL region comprising a VL CDR1 comprising the amino acid sequence of SEQ ID NO: 232, a VL CDR2 comprising the amino acid sequence of SEQ ID NO: 233, and a VL CDR3 comprising the amino acid sequence of SEQ ID NO: 234;
(ii) a VH region comprising a VH CDR1 comprising the amino acid sequence of SEQ ID NO: 235 a VH CDR2 comprising the amino acid sequence of SEQ ID NO: 236, and a VH CDR3 comprising the amino acid sequence of SEQ ID NO: 237; and a VL region comprising a VL CDR1 comprising the amino acid sequence of SEQ ID NO: 238, a VL CDR2 comprising the amino acid sequence of SEQ ID NO: 239, and a VL CDR3 comprising the amino acid sequence of SEQ ID NO: 240;
(iii) a VH region comprising a VH CDR1 comprising the amino acid sequence of SEQ ID NO: 241, a VH CDR2 comprising the amino acid sequence of SEQ ID NO: 242, and a VH CDR3 comprising the amino acid sequence of SEQ ID NO: 243; and a VL region comprising a VL CDR1 comprising the amino acid sequence of SEQ ID NO: 244, a VL CDR2 comprising the amino acid sequence of SEQ ID NO: 245, and a VL CDR3 comprising the amino acid sequence of SEQ ID NO: 246;
(iv) a VH region comprising a VH CDR1 comprising the amino acid sequence of SEQ ID NO: 247, a VH CDR2 comprising the amino acid sequence of SEQ ID NO: 248, and a VH CDR3 comprising the amino acid sequence of SEQ ID NO: 249; and a VL region comprising a VL CDR1 comprising the amino acid sequence of SEQ ID NO: 250, a VL CDR2 comprising the amino acid sequence of SEQ ID NO: 251, and a VL CDR3 comprising the amino acid sequence of SEQ ID NO: 252;
(v) a VH region comprising a VH CDR1 comprising the amino acid sequence of SEQ ID NO: 253, a VH CDR2 comprising the amino acid sequence of SEQ ID NO: 254, and a VH CDR3 comprising the amino acid sequence of SEQ ID NO: 255; and a VL region comprising a VL CDR1 comprising the amino acid sequence of SEQ ID NO: 256, a VL CDR2 comprising the amino acid sequence of SEQ ID NO: 257, and a VL CDR3 comprising the amino acid sequence of SEQ ID NO: 258; or
(vi) a VH region comprising a VH CDR1 comprising the amino acid sequence of SEQ ID NO: 259, a VH CDR2 comprising the amino acid sequence of SEQ ID NO: 260, and a VH CDR3 comprising the amino acid sequence of SEQ ID NO: 261; and a VL region comprising a VL CDR1 comprising the amino acid sequence of SEQ ID NO: 262, a VL CDR2 comprising the amino acid sequence of SEQ ID NO: 263, and a VL CDR3 comprising the amino acid sequence of SEQ ID NO: 264.

Embodiment 16. The antibody or fragment thereof of embodiment 1, wherein the antibody or fragment thereof comprises:
(a) a VH region comprising:
(1) a VH CDR1 having an amino acid sequence selected from the group consisting of SEQ ID NOs: 267, 273, 279, 285, 291, and 297;
(2) a VH CDR2 having an amino acid sequence selected from the group consisting of SEQ ID NOs: 268, 274, 280, 286, 292, and 298; and
(3) a VH CDR3 having an amino acid sequence selected from the group consisting of SEQ ID NOs: 269, 275, 281, 287, 293, and 299;
   and
(b) a VL region comprising:
(1) a VL CDR1 having an amino acid sequence selected from the group consisting of SEQ ID NOs: 270, 276, 282, 288, 294, and 300;
(2) a VL CDR2 having an amino acid sequence selected from the group consisting of SEQ ID NOs: 271, 277, 283, 289, 295, and 301; and
(3) a VL CDR3 having an amino acid sequence selected from the group consisting of SEQ ID NOs: 272, 278, 284, 290, 296, and 302.

Embodiment 17. The antibody or fragment thereof of embodiment 1, wherein the antibody or fragment thereof comprises any one or more of (i)-(ix):
(i) a VH region comprising a VH CDR1 comprising the amino acid sequence of SEQ ID NO: 267, a VH CDR2 comprising the amino acid sequence of SEQ ID NO: 268, and a VH CDR3 comprising the amino acid sequence of SEQ ID NO: 269; and a VL region comprising a VL CDR1 comprising the amino acid sequence of SEQ ID NO: 270, a VL CDR2 comprising the amino acid sequence of SEQ ID NO: 271, and a VL CDR3 comprising the amino acid sequence of SEQ ID NO: 272;
(ii) a VH region comprising a VH CDR1 comprising the amino acid sequence of SEQ ID NO: 273 a VH CDR2 comprising the amino acid sequence of SEQ ID NO: 274, and a VH CDR3 comprising the amino acid sequence of SEQ ID NO: 275; and a VL region comprising a VL CDR1 comprising the amino acid sequence of SEQ ID NO: 276, a VL CDR2 comprising the amino acid sequence of SEQ ID NO: 277, and a VL CDR3 comprising the amino acid sequence of SEQ ID NO: 278;
(iii) a VH region comprising a VH CDR1 comprising the amino acid sequence of SEQ ID NO: 279, a VH CDR2 comprising the amino acid sequence of SEQ ID NO: 280, and a VH CDR3 comprising the amino acid sequence of SEQ ID NO: 281; and a VL region comprising a VL CDR1 comprising the amino acid sequence of SEQ ID NO: 282, a VL CDR2 comprising the amino acid sequence of SEQ ID NO: 283, and a VL CDR3 comprising the amino acid sequence of SEQ ID NO: 284;
(iv) a VH region comprising a VH CDR1 comprising the amino acid sequence of SEQ ID NO: 285, a VH CDR2 comprising the amino acid sequence of SEQ ID NO: 286, and a VH CDR3 comprising the amino acid sequence of SEQ ID NO: 287; and a VL region comprising a VL CDR1 comprising the amino acid sequence of SEQ ID NO: 288, a VL CDR2 comprising the amino acid sequence of SEQ ID NO: 289, and a VL CDR3 comprising the amino acid sequence of SEQ ID NO: 290;
(v) a VH region comprising a VH CDR1 comprising the amino acid sequence of SEQ ID NO: 291, a VH CDR2 comprising the amino acid sequence of SEQ ID NO: 292, and a VH CDR3 comprising the amino acid sequence of SEQ ID NO: 293; and a VL region comprising a VL CDR1 comprising the amino acid sequence of SEQ ID NO: 294, a VL CDR2 comprising the amino acid sequence of SEQ ID NO: 295, and a VL CDR3 comprising the amino acid sequence of SEQ ID NO: 296; or
(vi) a VH region comprising a VH CDR1 comprising the amino acid sequence of SEQ ID NO: 297, a VH CDR2 comprising the amino acid sequence of SEQ ID NO: 298, and a VH CDR3 comprising the amino acid sequence of SEQ ID NO: 299; and a VL region comprising a VL CDR1 comprising the amino acid sequence of SEQ ID NO: 300, a VL CDR2 comprising the amino acid sequence of SEQ ID NO: 301, and a VL CDR3 comprising the amino acid sequence of SEQ ID NO: 302.

Embodiment 18. The antibody or fragment thereof of embodiment 1, wherein the antibody or fragment thereof comprises:
(a) a VH region comprising:
(1) a VH CDR1 having an amino acid sequence selected from the group consisting of SEQ ID NOs: 305, 311, 317, 323, 329, and 335;
(2) a VH CDR2 having an amino acid sequence selected from the group consisting of SEQ ID NOs: 306, 312, 318, 324, 330, and 336; and
(3) a VH CDR3 having an amino acid sequence selected from the group consisting of SEQ ID NOs: 307, 313, 319, 325, 331, and 337;
   and
(b) a VL region comprising:
(1) a VL CDR1 having an amino acid sequence selected from the group consisting of SEQ ID NOs: 308, 314, 320, 326, 332, and 338;
(2) a VL CDR2 having an amino acid sequence selected from the group consisting of SEQ ID NOs: 309, 315, 321, 327, 333, and 339; and
(3) a VL CDR3 having an amino acid sequence selected from the group consisting of SEQ ID NOs: 310, 316, 322, 328, 334, and 340 . . .

Embodiment 19. The antibody or fragment thereof of embodiment 1, wherein the antibody or fragment thereof comprises any one or more of (i)-(vi):
(i) a VH region comprising a VH CDR1 comprising the amino acid sequence of SEQ ID NO: 305, a VH CDR2 comprising the amino acid sequence of SEQ ID NO: 306, and a VH CDR3 comprising the amino acid sequence of SEQ ID NO: 307; and a VL region comprising a VL CDR1 comprising the amino acid sequence of SEQ ID NO: 308, a VL CDR2 comprising the amino acid sequence of SEQ ID NO: 309, and a VL CDR3 comprising the amino acid sequence of SEQ ID NO: 310;
(ii) a VH region comprising a VH CDR1 comprising the amino acid sequence of SEQ ID NO: 311 a VH CDR2 comprising the amino acid sequence of SEQ ID NO: 312, and a VH CDR3 comprising the amino acid sequence of SEQ ID NO: 313; and a VL region comprising a VL CDR1 comprising the amino acid sequence of SEQ ID NO: 314, a VL CDR2 comprising the amino acid sequence of SEQ ID NO: 315, and a VL CDR3 comprising the amino acid sequence of SEQ ID NO: 316;
(iii) a VH region comprising a VH CDR1 comprising the amino acid sequence of SEQ ID NO: 317, a VH CDR2 comprising the amino acid sequence of SEQ ID NO: 318, and a VH CDR3 comprising the amino acid sequence of SEQ ID NO: 319; and a VL region comprising a VL CDR1 comprising the amino acid sequence of SEQ ID NO: 320, a VL CDR2 comprising the amino acid sequence of SEQ ID NO: 321, and a VL CDR3 comprising the amino acid sequence of SEQ ID NO: 322;
(iv) a VH region comprising a VH CDR1 comprising the amino acid sequence of SEQ ID NO: 323, a VH CDR2 comprising the amino acid sequence of SEQ ID NO: 324, and a VH CDR3 comprising the amino acid sequence of SEQ ID NO: 325; and a VL region comprising a VL CDR1 comprising the amino acid sequence of SEQ ID NO: 326, a VL CDR2 comprising the amino acid sequence of SEQ ID NO: 327, and a VL CDR3 comprising the amino acid sequence of SEQ ID NO: 328;
(v) a VH region comprising a VH CDR1 comprising the amino acid sequence of SEQ ID NO: 329, a VH CDR2 comprising the amino acid sequence of SEQ ID NO: 330, and a VH CDR3 comprising the amino acid sequence of SEQ ID NO: 331; and a VL region comprising a VL CDR1 comprising the amino acid sequence of SEQ ID NO: 332, a VL CDR2 comprising the amino acid sequence of SEQ ID NO: 333, and a VL CDR3 comprising the amino acid sequence of SEQ ID NO: 334; or
(vi) a VH region comprising a VH CDR1 comprising the amino acid sequence of SEQ ID NO: 335, a VH CDR2 comprising the amino acid sequence of SEQ ID NO: 336, and a VH CDR3 comprising the amino acid sequence of SEQ ID NO: 337; and a VL region comprising a VL CDR1 comprising the amino acid sequence of SEQ ID NO: 338, a VL CDR2 comprising the amino acid sequence of SEQ ID NO: 339, and a VL CDR3 comprising the amino acid sequence of SEQ ID NO: 340.

Embodiment 20. The antibody or fragment thereof of embodiment 1, wherein the antibody or fragment thereof comprises:
(i) a VH comprising the amino acid sequence of SEQ ID NO: 37 and a VL comprising the amino acid sequence of SEQ ID NO: 38;
(ii) a VH comprising the amino acid sequence of SEQ ID NO: 75 and a VL comprising the amino acid sequence of SEQ ID NO: 76;
iii) a VH comprising the amino acid sequence of SEQ ID NO: 113 and a VL comprising the amino acid sequence of SEQ ID NO: 114;
(iv) a VH comprising the amino acid sequence of SEQ ID NO: 151 and a VL comprising the amino acid sequence of SEQ ID NO: 152;
(v) a VH comprising the amino acid sequence of SEQ ID NO: 189 and a VL comprising the amino acid sequence of SEQ ID NO: 190;
(vi) a VH comprising the amino acid sequence of SEQ ID NO: 227 and a VL comprising the amino acid sequence of SEQ ID NO: 228.
(vii) a VH comprising the amino acid sequence of SEQ ID NO: 265 and a VL comprising the amino acid sequence of SEQ ID NO: 266;
(viii) a VH comprising the amino acid sequence of SEQ ID NO: 303 and a VL comprising the amino acid sequence of SEQ ID NO: 304; or
(ix) a VH comprising the amino acid sequence of SEQ ID NO: 341 and a VL comprising the amino acid sequence of SEQ ID NO: 342.

Embodiment 21. An antibody or fragment thereof that binds to CD16a, wherein the antibody or fragment thereof binds to both CD16a-F and CD16a-V.

Embodiment 22. The antibody or antigen binding fragment of embodiment 21, wherein the antibody or fragment thereof does not bind to CD16b.

Embodiment 23. The antibody or antigen binding fragment of embodiment 21 or 22, wherein the antibody or fragment thereof does not bind to CD16b-NA1 and/or CD16b-NA2.

Embodiment 24. A multispecific antibody or fragment thereof, comprising a first binding domain that binds to CD16a and a second binding domain that binds to a second antigen, wherein the first binding domain comprises an antibody or fragment thereof according to any one of embodiments 1 to 23.

Embodiment 25. The multispecific antibody or fragment thereof of embodiment 24, wherein the second antigen is expressed on a tumor cell.

Embodiment 26. The multispecific antibody or fragment thereof of embodiment 24 or 25, wherein the second antigen is CD19.

Embodiment 27. The multispecific antibody or fragment thereof of any one of embodiments 24 to 26, wherein the first binding domain comprises any one or more of (i)-(ix):
(i) a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38;
(ii) a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76;
(iii) a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114;
(iv) a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152;
(v) a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190;
(vi) a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228;
(vii) a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266;
(vii) a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304; or
(ix) a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342; and
   wherein the second binding domain comprises a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344.

Embodiment 28. The multispecific antibody or fragment thereof of embodiment 27, wherein the first binding domain comprises any one or more of (i)-(ix):
(i) a VH comprising the amino acid sequence of SEQ ID NO: 37 and a VL comprising the amino acid sequence of SEQ ID NO: 38;
(ii) a VH comprising the amino acid sequence of SEQ ID NO: 75 and a VL comprising the amino acid sequence of SEQ ID NO: 76;
(iii) a VH comprising the amino acid sequence of SEQ ID NO: 113 and a VL comprising the amino acid sequence of SEQ ID NO: 114;
(iv) a VH comprising the amino acid sequence of SEQ ID NO: 151 and a VL comprising the amino acid sequence of SEQ ID NO: 152;
(v) a VH comprising the amino acid sequence of SEQ ID NO: 189 and a VL comprising the amino acid sequence of SEQ ID NO: 190;
(vi) a VH comprising the amino acid sequence of SEQ ID NO: 227 and a VL comprising the amino acid sequence of SEQ ID NO: 228.
(vii) a VH comprising the amino acid sequence of SEQ ID NO: 265 and a VL comprising the amino acid sequence of SEQ ID NO: 266;
(viii) a VH comprising the amino acid sequence of SEQ ID NO: 303 and a VL comprising the amino acid sequence of SEQ ID NO: 304; or
(ix) a VH comprising the amino acid sequence of SEQ ID NO: 341 and a VL comprising the amino acid sequence of SEQ ID NO: 342; and
   wherein the second binding domain comprises a VH comprising the amino acid sequence of SEQ ID NO: 343 and a VL comprising the amino acid sequence of SEQ ID NO: 344;

Embodiment 29. The multispecific antibody or fragment thereof of any embodiment of embodiments 24-28, wherein the first binding domain and the second binding domain can be linked through a peptide bond or a peptide linker.

Embodiment 30. The multispecific antibody or fragment thereof of any one of embodiments 26-29, wherein the multispecific antibody or fragment thereof comprises one polypeptide chain comprising the first binding domain and the second binding domain, wherein the first binding domain comprises a first scFv that binds to CD16, and the second binding domain comprises a second scFv that binds to CD19.

Embodiment 31. The multispecific antibody or fragment thereof of embodiment 30, wherein the first scFv is linked to the N terminal of the second scFv.

Embodiment 32. The multispecific antibody or fragment thereof of embodiment 30, wherein the first scFv is linked to the C terminal of the second scFv.

Embodiment 33. The multispecific antibody or fragment thereof of any one of embodiments 30-32, wherein the polypeptide chain further comprises a CH2 and a CH3 of an IgG Fc region.

Embodiment 34. The multispecific antibody or fragment thereof of any one of embodiments 30-33, the multispecific antibody or fragment comprises two identical polypeptide chains of embodiment 33.

Embodiment 35. The multispecific antibody or fragment thereof of embodiment 34, wherein the first polypeptide and the second polypeptide form a bivalent format through dimerization of the CH2 and/or CH3 regions.

Embodiment 36. An isolated nucleic acid comprising a nucleic acid sequence encoding the antibody of any one of embodiments 1-23, or the multispecific antibody of any one of embodiments 24-35.

Embodiment 37. A vector comprising the isolated nucleic acid of embodiment 36.

Embodiment 38. A pharmaceutical composition, comprising the antibody of any one of embodiments 1-23, the multispecific antibody of any one of embodiments 24-35, or the vector of embodiment 37, and a pharmaceutically acceptable excipient.

Embodiment 39. A method of treating a disease or disorder in a subject, comprising administering to the subject an effective amount of the antibody of any one of embodiments 1-23, the multispecific antibody of any one of embodiments 24-35, or the vector of embodiment 37, or the pharmaceutical composition of embodiment 38.

Embodiment 40. The method of embodiment 39, wherein the disease or disorder is a cancer.

Embodiment 41. The method of embodiment 39, wherein the disease or disorder is leukemia or lymphoma.

Embodiment 42. A method of activating NK cell activity in a subject, comprising administering to the subject an effective amount of the antibody of any one of embodiments 1-23, the multispecific antibody of any one of embodiments 24-35, or the vector of embodiment 37, or the pharmaceutical composition of embodiment 38.

Embodiment 43. A method of activating NK cell activity without activating neutrophils in a subject, comprising administering to the subject an effective amount of the antibody of any one of embodiments 1-23, the multispecific antibody of any one of embodiments 24-35, or the vector of embodiment 37, or the pharmaceutical composition of embodiment 38.

Embodiment 44. The method of any one of embodiments 39 to 43, wherein the subject has CD16a-F/F, CD16a-F/V, CD16a-V/V genotype.

Embodiment 45. The method of any one of embodiments 39 to 44, wherein the subject has CD16b-NA1/CD16b-NA2, CD16b-NA1/CD16b-NA1, CD16b-NA2/CD16b-NA2genotype.

Embodiment 46. An antibody or fragment thereof which specifically recognizes an epitope comprising the residue Asp 82 of human CD16a with an affinity for CD16a at least 10-fold higher than for CD16b.

### 7. Examples

### 7.1 Example 1. Generation of Antibody Clones.

The following biotinylated recombinant extracellular domains (ECDs) were purchased from Acrobiosystems. CD16a-F176 and CD16a-V176 ECDs encompassing residues Gly 17 to Gln 208 followed by a His and an Avi tag at the C-terminus with a M. Wt. of 25.5 kDa (hereon referred to as biotin-CD16a-F and biotin-CD16a-V, respectively) (catalogue numbers CDA-H82E8 and CDA-H82E9, respectively). CD16b-NA1 and NA2 ECDs encompassing residues Gly 17 to Ser 200 followed by an Avi tag and His tag at the C terminus with M. Wt. of 24.6 kDa (hereon referred to as biotin-CD16b-NA1 and biotin-CD16b-NA2, respectively) (catalogue numbers CDB-H82E4 and CDB-H82Ea, respectively).

Library screening: The human antibody libraries used for screening were based on five different VH families each combined with four V libraries and one V library which together in total consist of 100 billion independent antibody clones. Consequently, libraries were split into 6 pools: 1) the VH1-69 combined with all 4 V libraries (V1-39, 3-15, 3-20 [9 aa in CDR3], 3-20 [10 aa in CDR3]), 2) the VH3-15 combined with all the V libraries, 3) the VH3-23 combined with the V libraries, 4) the VH3-30 combined with the V libraries, 5) the VH4-39 combined with the V libraries and 6) each of the VH libraries combined with the V 1-40 library. The six library pools were grown up to 3-fold of the original library titer in selective media (0.74 g amino acids without tryptophan or leucine for the Fab library, or 1.0 g of amino acids without tryptophan for the scFv library, 20 mg uracil, 10 mg adenine, 7 g yeast nitrogen base with ammonium sulfate, 20 g glucose, 10.19 g Na2HPO4-7H2O and 8.56 g NaH2PO4-H2O, per liter, pH 6.25), induced for antibody expression.

The screening process involved a preliminary enrichment for CD16a antigen-binding antibody clones using magnetic bead activated cell sorting (MACS) followed by FACS. For the initial magnetic bead sorting step, yeast cells were grown and induced to express antibodies by culturing in induction medium (0.74 g amino acids without tryptophan or leucine [1 μm amino acids minus tryptophan for the scFv libraries], 20 mg uracil, 10 mg adenine 7 g yeast nitrogen base with ammonium sulfate, 1 g glucose, 20 g raffinose, 20 g galactose, 10.19 g NaH2PO4-7H2O and 8.56 g Na2HPO4, pH 6.25 per liter) at 20° C. for 16 hours to induce the expression of the Fab. Biotinylated CD16a-F and CD16a-V were added in binding buffer (PBS plus 5 g/l BSA, but without Ca2+ or Mg2+) to a final concentration of 100 nM each. After incubation with the yeast libraries at room temperature for 1 hr, the cells were incubated on ice for 10 min, washed 3 times with 50 ml binding buffer, followed by incubation with streptavidin-magnetic beads for 10 min on ice. The cells were then washed once with binding buffer and resuspended in binding buffer before each library was passed through a magnetic sorting column. After all the cells had passed through the column, the column was washed 3 times with 7 ml binding buffer. Captured cells were eluted in 10 ml of binding buffer per column by turning off the magnetic field, then pelleted and resuspended in selective medium. The collected cells were grown up and induced for Fab expression as described above and used for a second round of magnetic bead sorting again using a concentration of 100 nM of each biotinylated CD16a antigen.

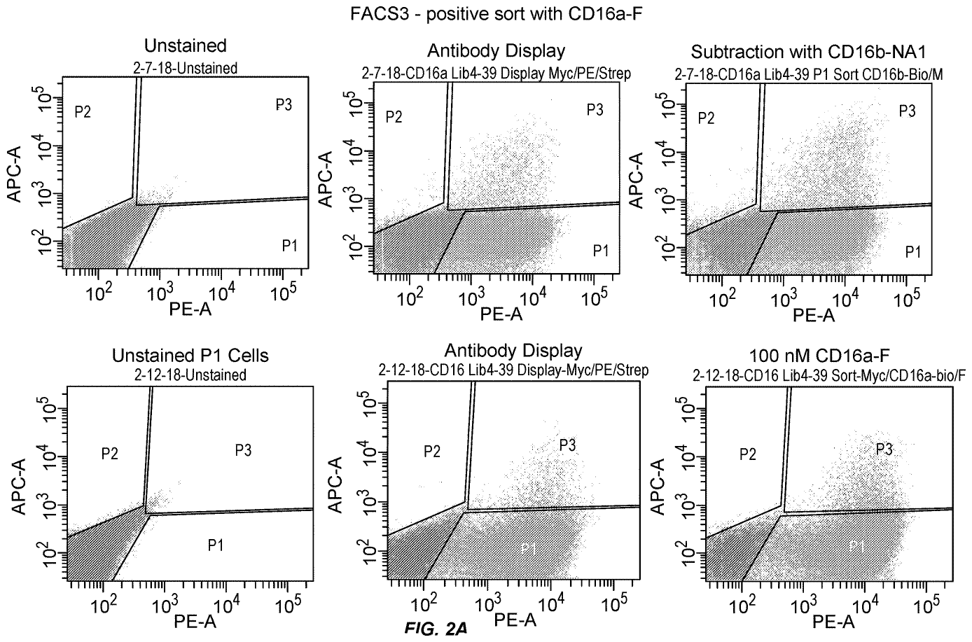

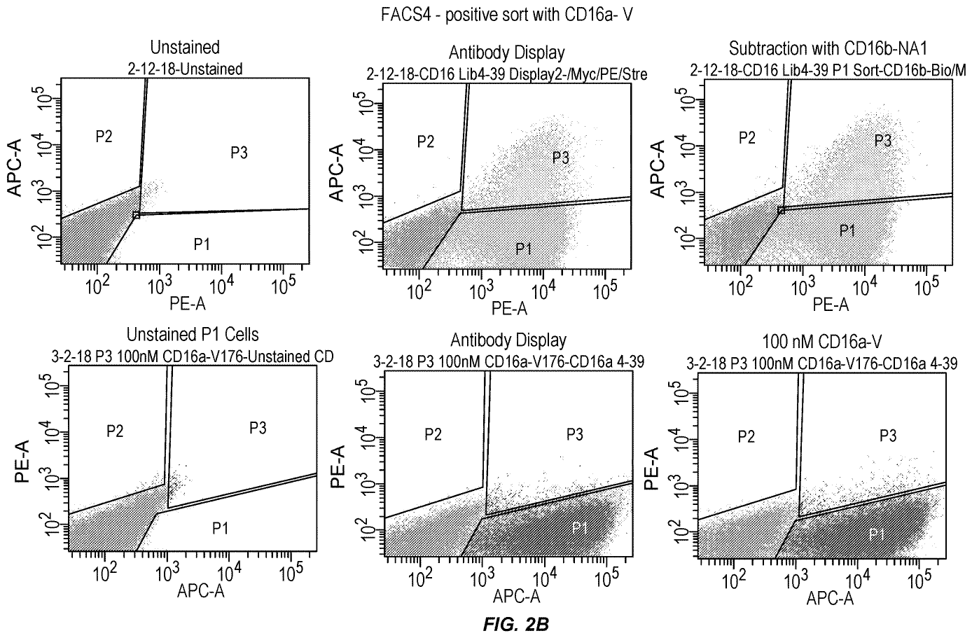

The selected cells were then grown up for further selection by FACS. Fab-expressing yeast cells were incubated with the presence of 100 nM each of biotinylated CD16a-F and CD16a-V and bound antigens were detected with phycoerythrin (PE)-conjugated streptavidin (Jackson Immunoresearch, catalogue #JAX-016-110-084), while the displayed Fab was detected with affinity purified goat anti-human kappa or lambda light chain antibody (Bethyl Laboratories, catalogue #A80-115A and #80-116A, respectively) followed by Alexa 647-conjugated AffiniPure donkey anti-goat IgG (H+L) (Jackson Immunoresearch, catalogue #JAX 705-605-003). Double positive cells were identified and collected using a FACSAria II cell sorter (Becton Dickenson) at a rate of 20,000 events per second. The harvested cells were expanded and used for the next round of sorting again performed in the presence of 100 nM of each biotinylated CD16a antigen that was detected with PE-labeled streptavidin. The antigen (PE) and Fab positive (Alexa-647) cells were collected and then used for a 3rd round of FACS (FACS3), where a subtractive pre-sort was performed using 200 nM biotinylated CD16b-NA1 and staining for antibody expression was performed using goat anti-cMyc (Bethyl Laboratories, catalogue #190-104A) that was detected by PE-labeled donkey-anti-goat IgG. About 25% of the antibody expressing yeast cells bound to the CD16b-NA1 antigen and expressed antibody (P3 gate in right upper panel in FIG. 2A). The antibody-expressing, APC-negative cells (the population found in the P1 gate; top right panel of FIG. 2A) were collected and used for a positive sort using 100 nM biotinylated CD16a-F antigen. The cells that were double-positive for CD16a-F binding (APC positive) and antibody expression (PE-positive), which represented about 20% of the post-subtraction sorted population (lower right panel of FIG. 2A), were harvested for FACS4 which was performed with a similar pre-sort step but this time using 100 nM of the biotinylated CD16a-V variant that was detected with APC-labeled streptavidin; this reduced the number of CD16a-antigen-positive, antibody-positive cells to less than 3% of the total population (FIG. 2B). This subtraction-presort followed by a positive sort was repeated with two more rounds of subtraction with CD16b followed by positive selection with CD16a-F but decreasing the CD16a-F antigen first to 75 nM biotinylated CD16a-F, then to 25 nM CD16a-F plus CD16a-V to select for higher affinity anti-CD16a-F and CD16a-V clones. The CD16a-positively stained cells from the final round of FACS were collected and plated on selective agar for individual clone analysis.

### 7.2 Example 2. Selection of Antibody Clones

The yeast antibody clones isolated from the final two rounds of FACS from each library were plated on selective media plates and incubated at 30° C. for 2 days. Individual colonies were randomly picked from the plates and grown in selective medium overnight at 30° C. Replica plates were seeded and after 24 hours the medium was replaced with induction medium and cells cultured at 20° C. overnight to induce the expression of Fab or scFv antibodies. The individual clones were then washed and incubated in the presence of 25 nM biotinylated CD16a-F, CD16a-V or CD16b-NA1. Bound antigen was detected with APC-labeled streptavidin. Clones that stained positive for both CD16a isoforms but were negative for CD16b were consolidated into a new plate that was then used for ELISA.

In this yeast display system, a significant amount of antibody is secreted into the culture medium in addition to those displayed antibodies on the yeast cell surface. The secreted Fab in the culture medium can be used directly for binding assays.

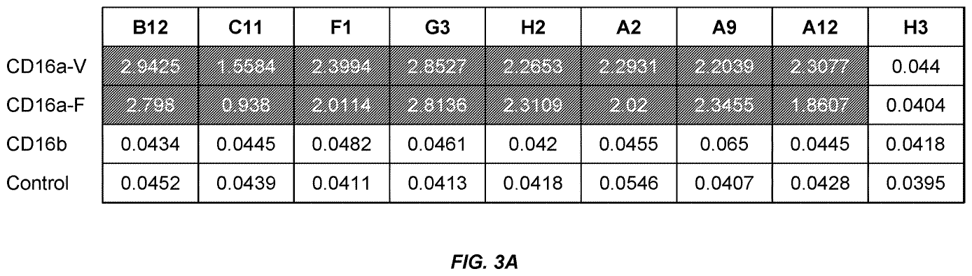

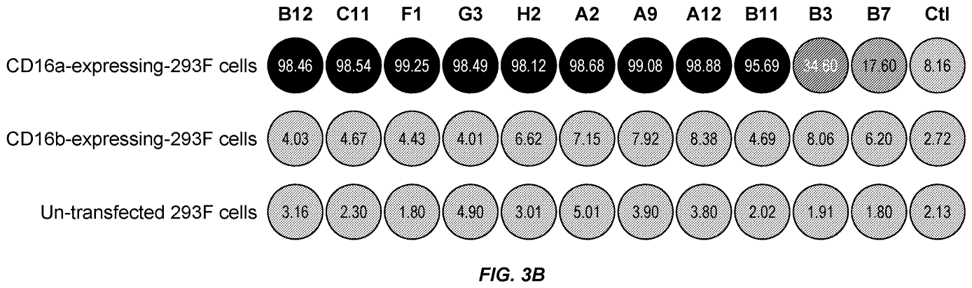

For the cells harvested from the library campaign, an ELISA was performed with the antibody-containing culture supernatants to determine binding activity of individual antibody clones to wells coated with either CD16a-F, CD16a-V, CD16b-NA1 or a control irrelevant protein. For this assay, 100 ng of antigen in 50 μl PBS was used to coat the wells of Immulon 2 HB ELISA plates at 4° C. overnight. The next day, wells were washed and were blocked with PBS containing 3% BSA for 1 hour at room temperature then washed again. The secreted Fab antibodies in 20 μl culture media were added to 30 μl 2× binding buffer (0.5 M HEPES, 0.5 M NaCl, 5% BSA, pH 7.5) and the plates incubated for 60 mins at room temperature. Bound Fab was detected with HRP-labeled goat anti-light chain. Clones that exhibited robust binding to the two isoforms of CD16a but exhibited no binding above background to CD16b-NA1 or the control protein (FIG. 3A) were selected for further analysis.

Separately, the clone supernatants were tested for their binding activity to transiently transfected 293 cells expressing full length CD16a-F or CD16b-NA2 to ensure the clones exhibited specific binding bound to native CD16a but not CD16b. For this, 293 cells were transiently transfected with a mammalian pCMV3-C expression vector encoding human CD16a-F or CD16b-NA2* (Sino Biologicals, Cat #HG10389-UT and HG11046-UT, respectively) (CD16b-NA2* is a variant of CD16b-NA2 in which amino acid #65 is an Asparagine, rather than a Serine). Two days after transfection, aliquots of 20,000 transfected or untransfected 293F cells were added to each well of 96 well plates. 20 μl of individual yeast clone supernatants were added to 80 μl PBS containing 3% BSA and the plates incubated on ice for 1 hour. The wells were washed by aspirating off the media, and then bound Fab detected with Dylight 650-labeled goat anti-cMyc to detect the c-Myc tag on the Fab. The cells were then analyzed using a 96-well IntelliCyt® iQue Screener PLUS and ForeCyt Software (Sartorius). All the supernatants from the CD16a-positive, CD16b-negative clones from the ELISA exhibited specific binding to native human CD16a expressed on the transfected 293 cells, confirming that they recognized native CD16a-F protein but not to CD16b-expressing 293 cells (FIG. 3B).

The DNA encoding the heavy and light chains of the 19 clones with the correct binding profile by ELISA and iQue analysis were sequenced: this indicated that these represented 10 unique sequences.

The light and heavy chains of the 10 unique sequences were separately cloned into different expression vectors carrying the constant regions of human IgG1 heavy chain and the human kappa or lambda light chain. The heavy and light chain plasmids were co-transfected into Expi293 suspension cells. Secreted antibody was then purified from the culture supernatants by Protein A chromatography, buffer-exchanged into PBS pH 7.4 and its concentration determined by Nanodrop A280 assay. The quality of each IgG was assessed by SDS-PAGE performed under both reducing and no-reducing conditions and by HPLC.

### 7.3 Example 3. Antibody Antigen Binding Properties and Activity

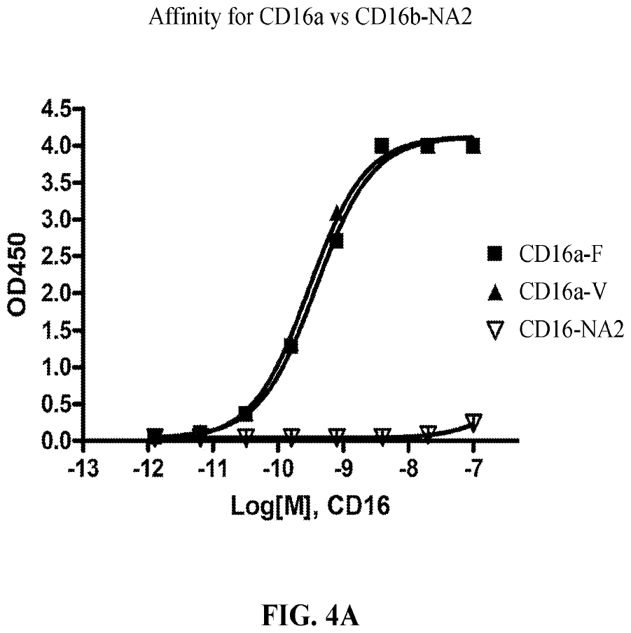

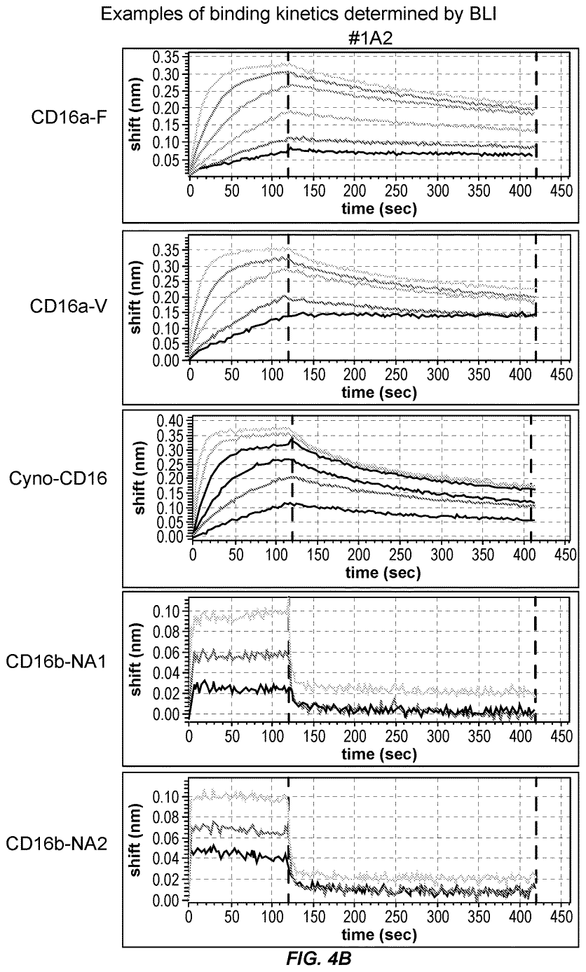

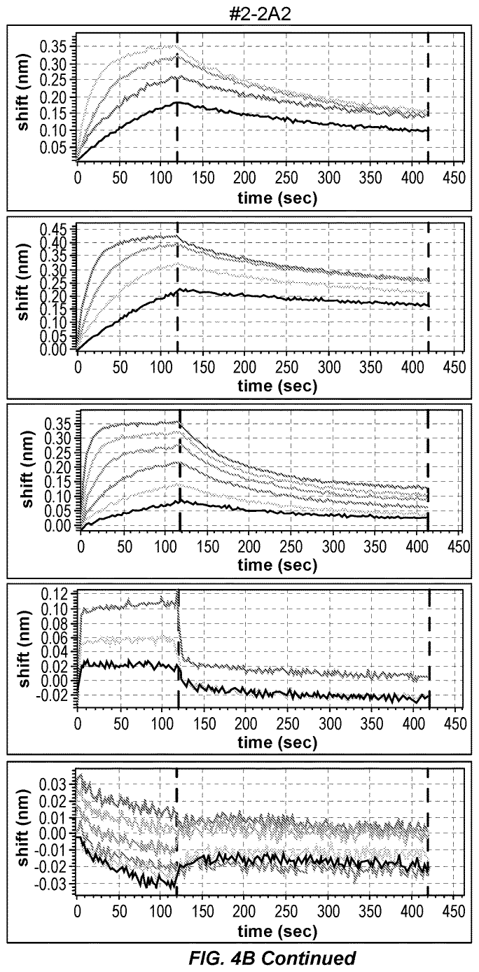

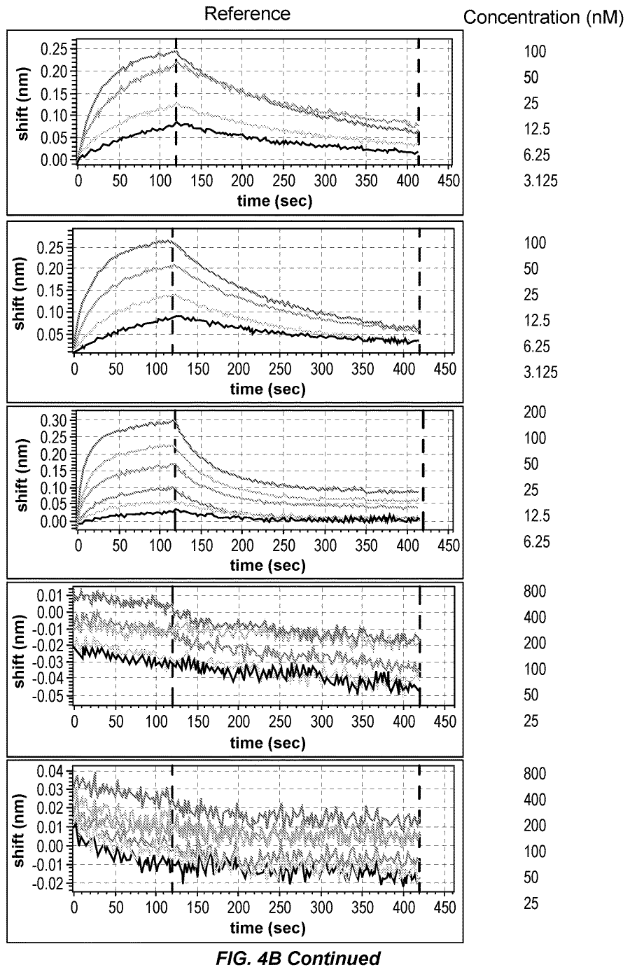

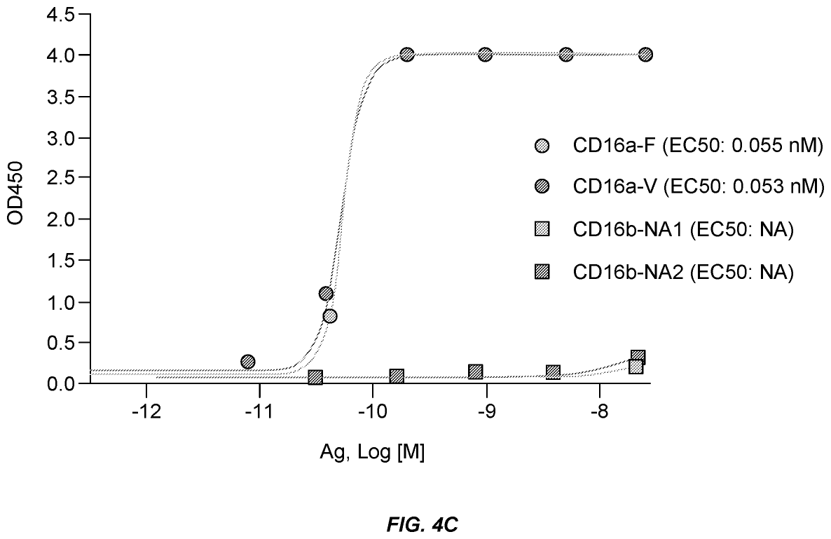

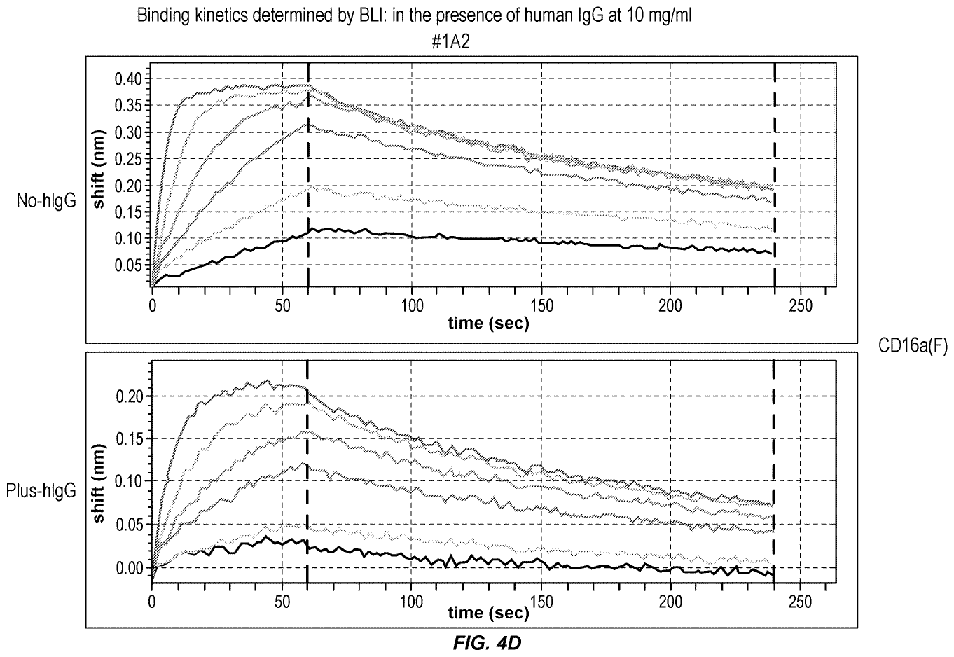

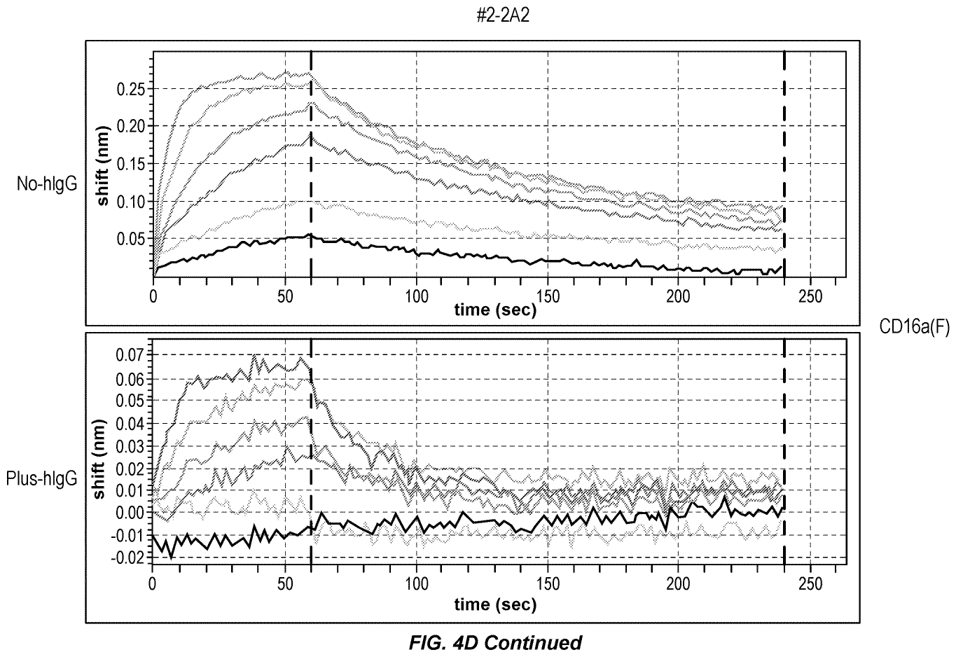

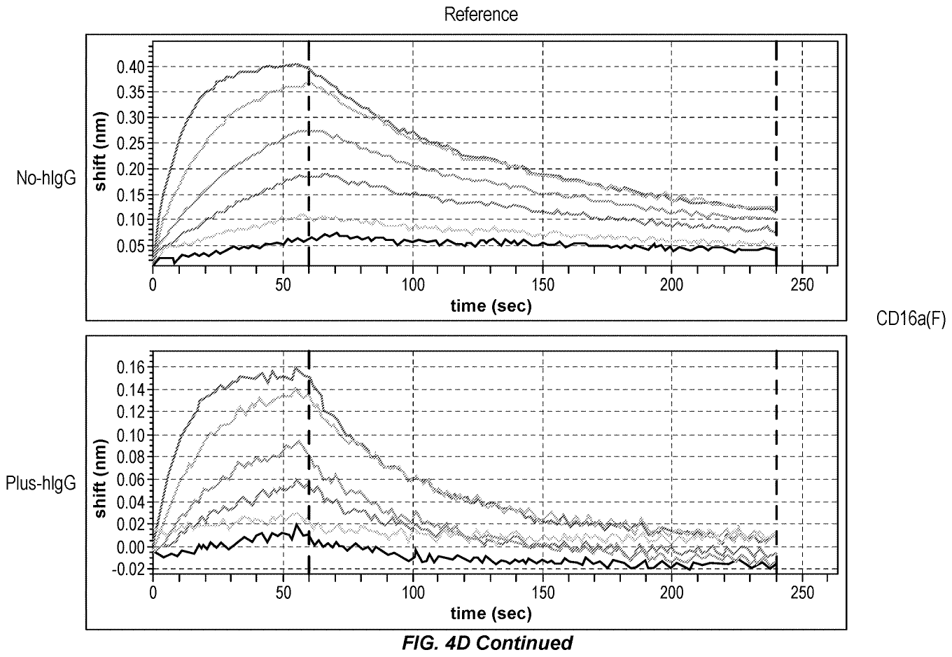

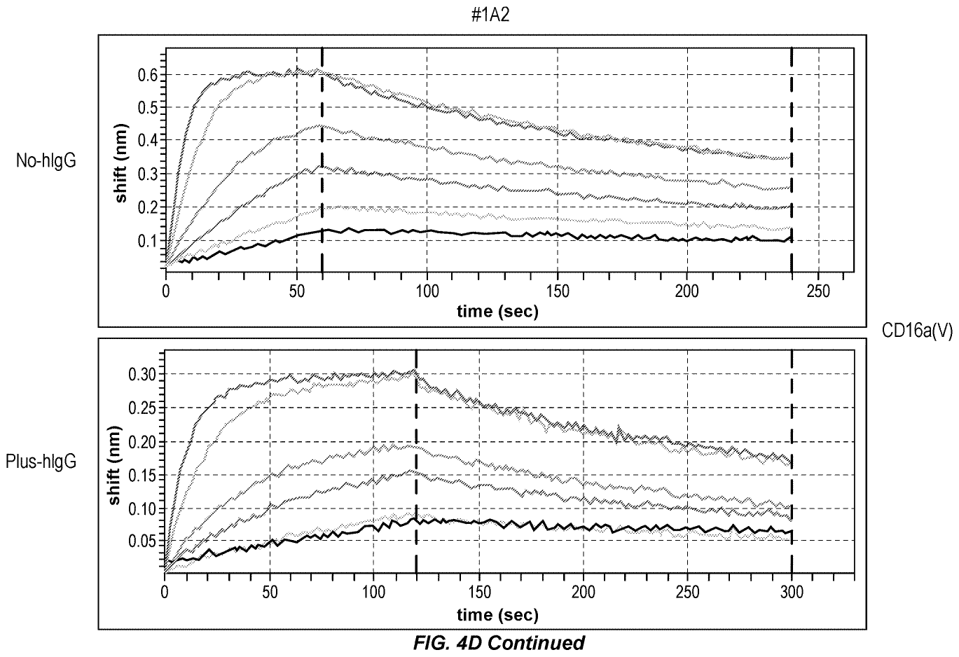

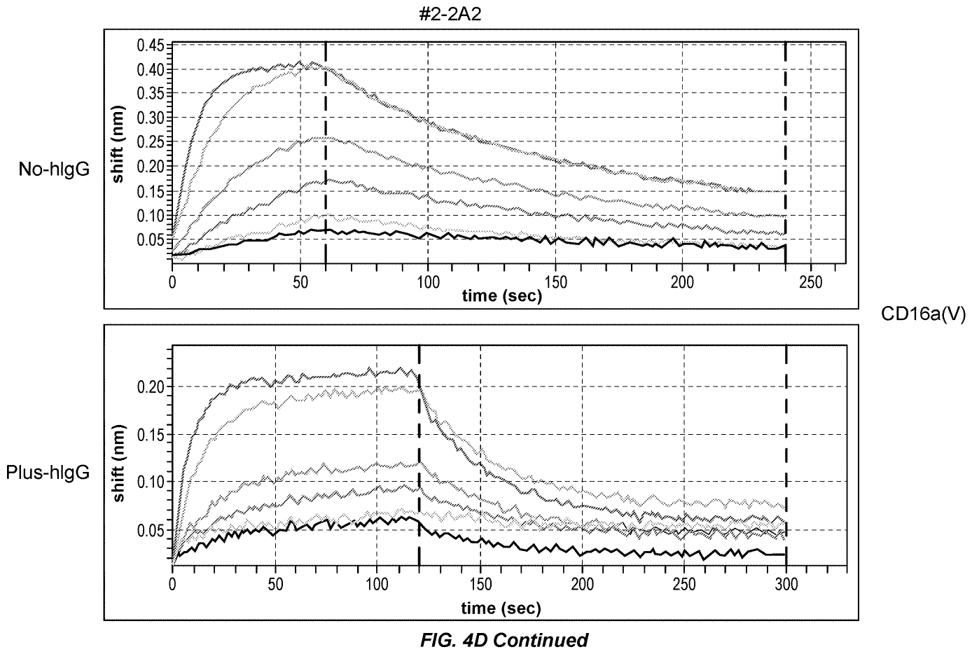

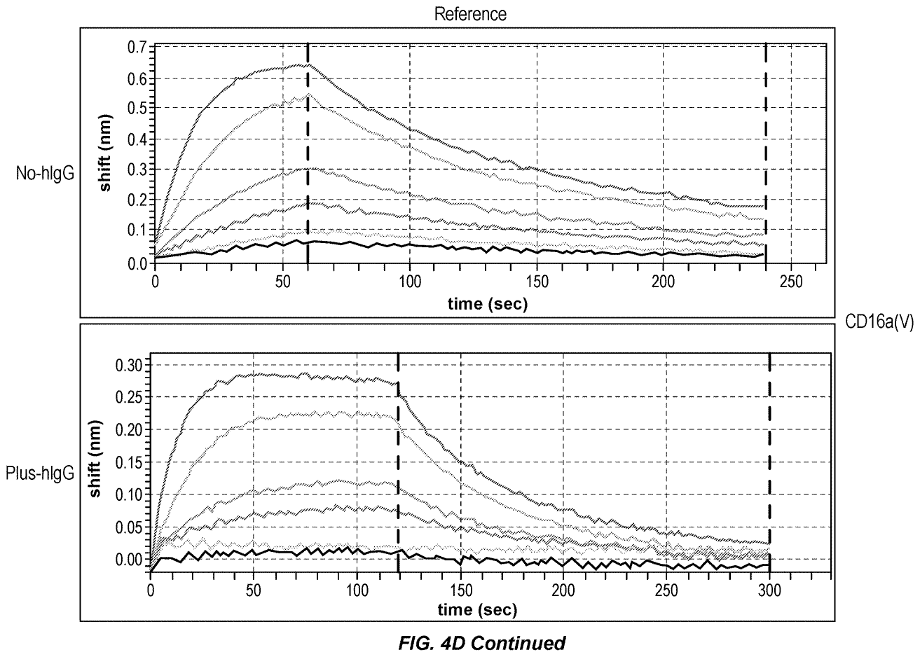

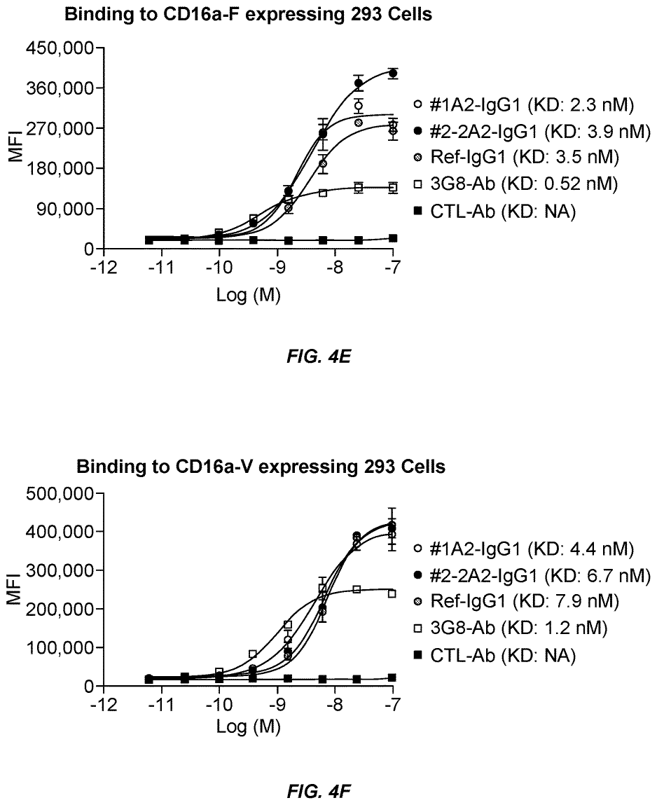

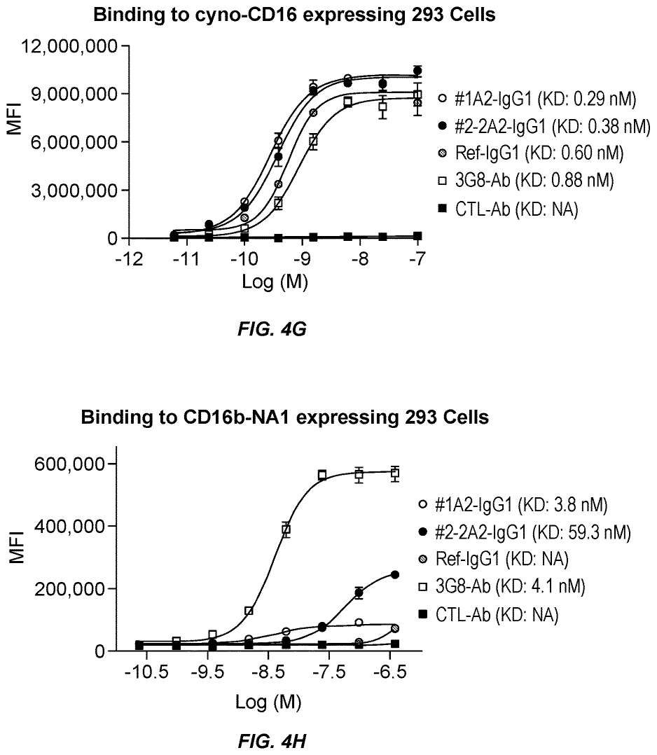

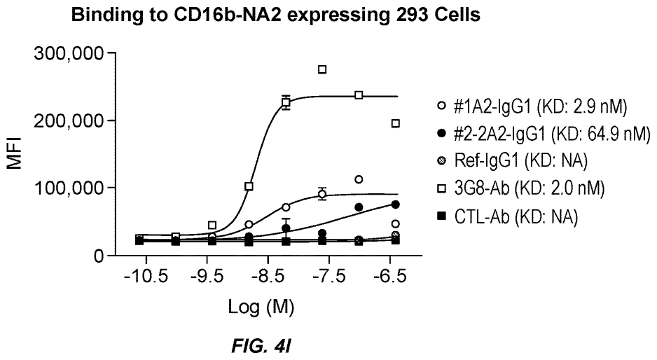

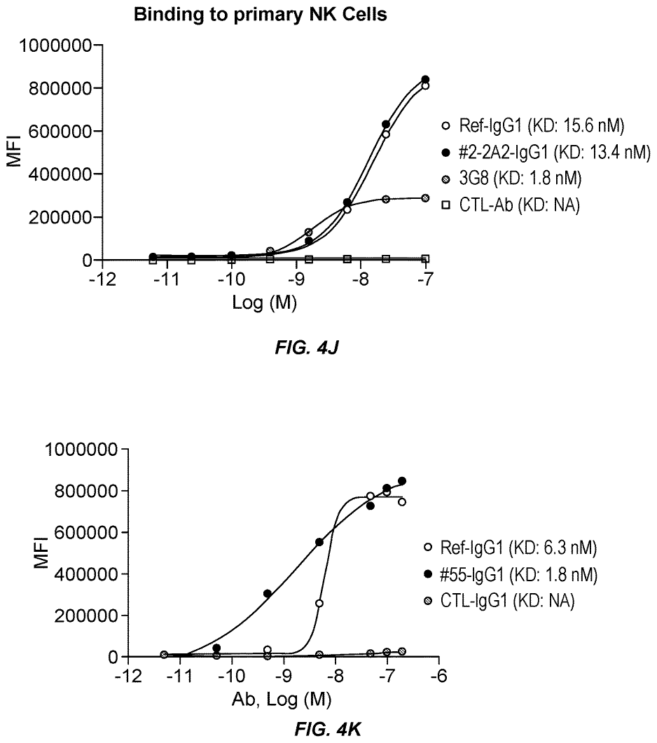

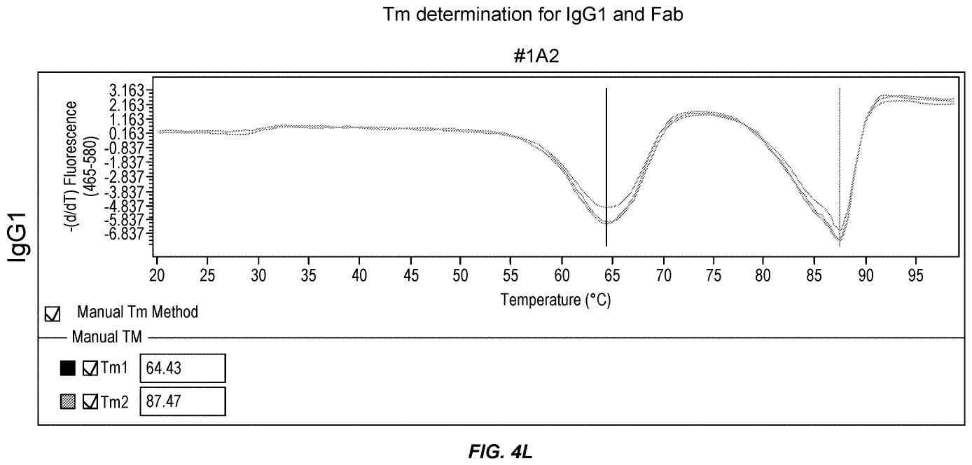

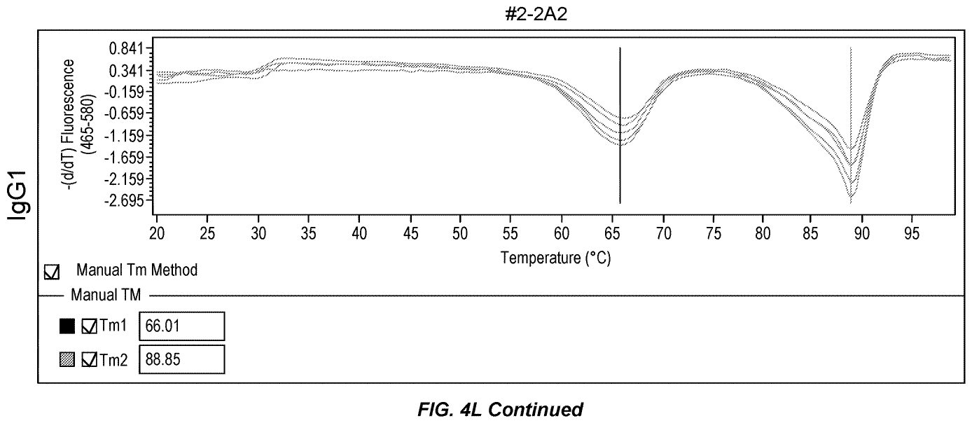

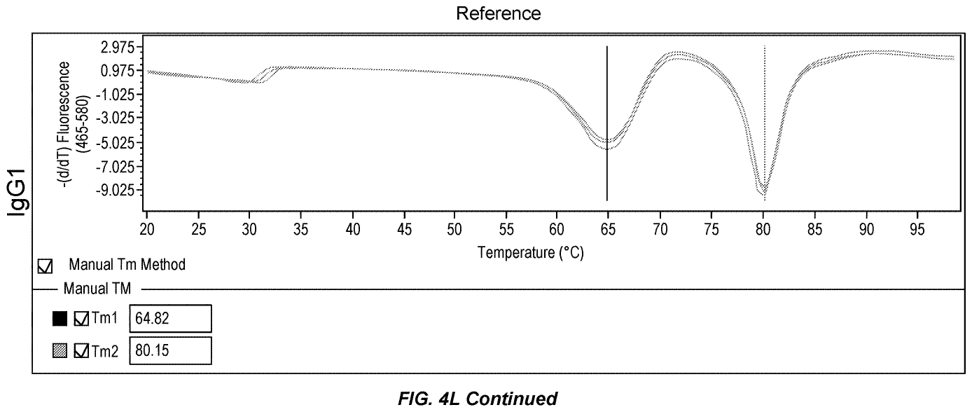

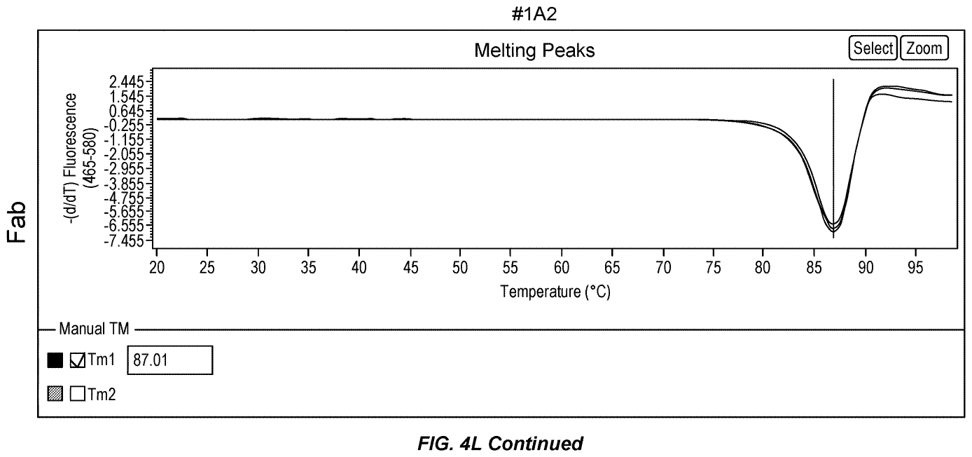

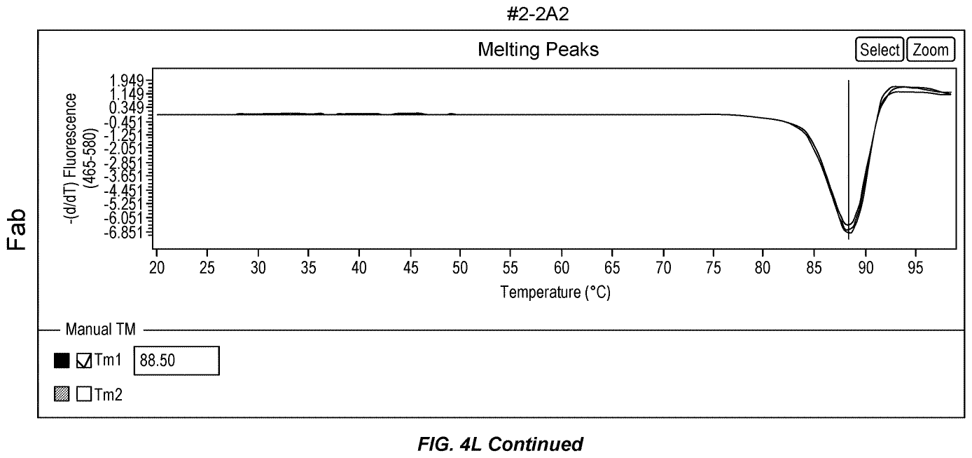

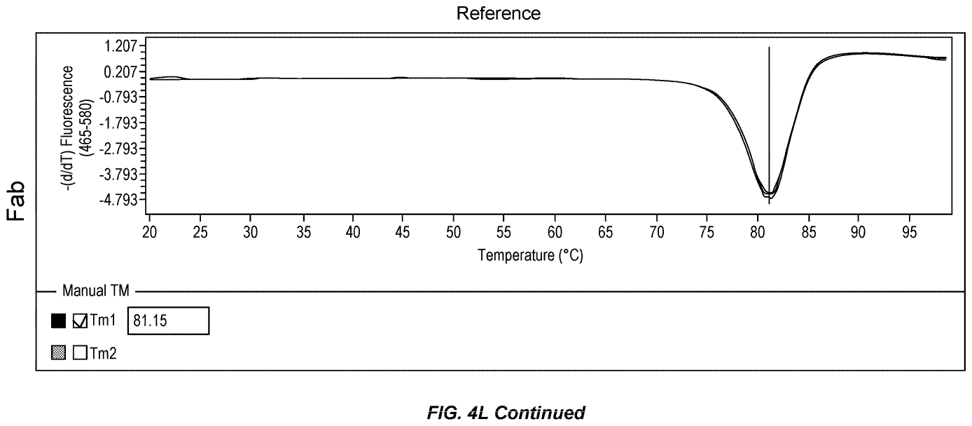

The apparent affinity of the 10 purified clones from the screening campaign was determined by ELISA. For the ELISA, wells were coated with 100 ng/well of the test antibody clones overnight at 4° C., washed then blocked in PBS plus 3% BSA. Five-fold serial dilutions of biotinylated CD16a or irrelevant negative control antigen starting at 100 nM were added to the relevant wells and incubated for 1 hr at room temperature. Wells were then washed and bound biotinylated antigen detected with HRP-labeled streptavidin. Specific binding (subtracting OD450 values for BSA from OD450 values for the control-His protein at the same concentration) was plotted and curve fitted using Prism software. The binding curve for an exemplary clone is shown in FIG. 4A. The apparent K_{D} values for the clones were determined to range from 0.16 to 6.6 nM for the two forms of CD16a(Table 11). Three of the clones, A2, D8 and E3, exhibited limited binding for CD16b-NA2 at the highest concentration used in the experiment.

TABLE 11

| | Apparent K_{D} (nM) | | |
|---|---|---|---|
| Clone | CD16a-V | CD16a-F | CD16b-NA2 |
| A2 | 0.38, 0.3 | 0.27 | >>100 |
| A12 | 0.36, 0.3 | 0.25 | 12.1 |
| B8 | 6.6, 2.4 | 1.50 | 114 |
| B11 | 0.41, 0.41 | | >100 |
| B12 | 0.19, 017 | 0.16 | 2.4 |
| C11 | 2.7, 2.7 | 0.26 | 42.1 |
| D8 | 1.2, 1.5 | 0.75, 1.2 | >>100 |
| E3 | 0.31 (0.31) | 0.32 (0.32) | >>100 |
| G3 | 0.17, 0.18 | 0.16 | 12.7 |
| G8 | 0.53, 0.28 | 0.32 | 1.1 |

Based on the ELISA data, four clones, namely A2, B11, D8 and E3, which show the lowest binding activity to CD16b-NA2, together with G8, which could serve as a non-selective CD16a and CD16b binder, were selected for further characterization. The clones were then tested for their ability to bind to Jurkat cells stably transfected with CD16a-V under control of a NFAT (Nuclear Factor of Activated T-cell response elements) luciferase reporter system (Promega). 25,000 cells were seeded per well and a serial dilution of these 5 selected clones added in duplicate in the presence or absence of human serum. After 1 hour incubation at 4° C., cells were washed and bound IgG detected with goat-anti-human-Fc-APC and fluorescence of each well measured using an iQue 96 well flow cytometer (Intellicyt). CD16a is the low affinity IgG Fc receptor, therefore binding activity of the clones was assessed by ELISA in the presence or absence of 10 mg/ml human IgG. Clone A2 and D8 were the least impacted by the presence of human IgG.

Next the ability of these 3 selected clones to activate CD16a when they are pre-complexed with goat anti-human Fc antibodies was assessed using the Jurkat CD16a-V NFAT (Nuclear Factor of Activated T-cell response elements) luciferase reporter system (Promega). For this, 75×10⁶ cells were plated per well of a 96-well plate in RPMI media containing 4% low IgG fetal calf serum and a 3-fold serial dilution of each antibody clone added in duplicate to designated wells. The plate was incubated for 20 hours at 37° C. in a 5% CO2 atmosphere. Then wells were washed and 100 μl Bio-Glo Luciferase reagent added per well and after 10 minutes incubation at room temperature, the luminescence signal was measured using a FilterMax F5 plate reader (Molecular Devices) in luminescence mode. Only clone A2 from this set of clones activated CD16a-mediated NFAT luminescence.

To generate variants of clone A2, an antibody engineering campaign was performed. For this, a focused library of clone A2 was built whereby changes were introduced at key CDR residues, 1 mutation per CDR per antibody clone, based on the CDR amino acid usage observed in a large database of naturally occurring human antibody sequences for the specific VH and VL subfamilies from which clone A2 is derived. While any one clone will have a maximum of 6 mutations, the focused library as a whole covers more than 2.5 billion variants. The library was displayed on yeast and screened by FACS.

The library was screened in FACS1 and FACS2 in the presence of 5 nM biotinylated human CD16a-F (ACRO Biosystems). Then for FACS3, the harvested cells were first subjected to a subtraction sort in the presence of CD16b-NA2 transfected 293 cell lysates, which was followed by a positive selection for the binders to CD16a-F cell lysates in the presence of normal human IgG. A total of 344 individual clone Fab-containing culture supernatants were tested for their binding to 5 nM recombinant human CD16a-V or CD16a-F in the presence and absence of 10 mg/ml of human IgG, and to 500 nM recombinant human CD16b-NA2. Clones that showed robust binding in the presence of 10 mg/ml human IgG and were negative for binding to CD16b-NA2 were selected for sequencing. Seven (7) unique clones were identified. These were subcloned into a mammalian expression vector carrying a mutated Fcγ1 with the LALA (Leu234Ala plus Leu235Ala) and Pro329Gly mutations to eliminate the Fcγ1 binding site for CD16. The vector was used to transfect ExpiCHO cells and the clones purified as full-length IgGs from the culture supernatants by Protein A chromatography. The purified IgGs were then assessed for their binding affinity and specificity in the presence and absence of 10 mg/mL normal human IgG by ELISA.

Out of these, all the A2 variants except #6A2 exhibited good binding properties, with a higher apparent affinity for CD16a isoforms compared to the parental A2 clone, with values still in the sub nM range in the presence of 10 mg/ml human non-immune IgG and with low affinity (>100 nM) for CD16b-NA2 (Table 12). The clones that appeared to lose the least change in affinity in the presence of high concentrations of normal human IgG and also exhibited low cross-reactivity with the two CD16b isoforms were #1A2 and #2-1A2 (Table 12).

TABLE 12

| | Apparent KD (nM) | | | | | |
|---|---|---|---|---|---|---|
| | CD16a-V | | CD16a-F | | CD16b- | CD16b- |
| Clone | No IgG | + hIgG | No IgG | + hlgG | NA1 | NA2 |
| A2 | 0.19 | 1.3 | 0.25 | 1.3 | 350 | 262 |
| #1A2 | 0.040 | 0.050 | 0.048 | 0.051 | 746 | 652 |
| #2-1A2 | 0.028 | 0.040 | 0.037 | 0.046 | 420 | 871 |
| #2-2A2 | 0.053 | 0.26 | 0.055 | 0.26 | >1 μM | 428 |
| #2-3A2 | 0.029 | 0.039 | 0.039 | 0.051 | 64 | 340 |
| #3A2 | 0.051 | 0.53 | 0.048 | 0.74 | >>1 μM | 546 |
| #6A2 | 0.16 | 0.61 | 0.14 | 0.67 | 1.3 μM | 298 |
| #10A2 | 0.043 | 0.13 | 0.044 | 0.15 | 530 | 357 |

Separately, a second de novo screening of the original library was performed where initial positive selection of binders to CD16a-F plus CD16a-V was followed by subtractive sorts using both CD16b-NA1 and CD16b-NA2 extracellular domains. Then additional final rounds of sorting were included that used cell lysates of Expi293 cells that had been transiently transfected with CD16a-F vector to collect the positive binders, and lysates from cells transiently transfected with CD16b-NA2* vector, which was purchased from SinoBiological (Cat. #HG11046-UT) to collect the negative binders.

For lysate preparation, Expi293 cells were transiently transfected with the relevant expression plasmid. Three days after transfection, the cells were harvested. The viable cells were counted, washed in ice-cold binding buffer (1×PBS, pH7.4 containing 0.5% BSA) and resuspended in binding buffer containing proteinase inhibitors. They were then lysed by passing through a 27G gauge needle, 7-10 times on ice. The lysate was then centrifuged at 1,000×g for 1 minute to collect the supernatant. The pellet was then lyzed two more times. The supernatants were combined to a final concentration equivalent to 20 million cells/ml. The lysates were added to be 50% of the cell binding assay volume, one aliquot in the presence and the other in the absence of 10 mg/mL human IgG to select for clones whose binding to CD16a would be less impacted by the presence of high concentrations of human IgG. Expressed CD16a-F and CD16b-NA2* captured by the yeast cells were detected using a pan anti-CD16 antibody, G8, which had been labeled with DyLight-650.

Out of the 112 individual yeast clones tested for their binding activity based on iQue analysis described above, clone #55 when purified as an IgG1 from transfected 293 cells, exhibited sub nM binding to both forms of CD16a and a three-log shift in the mid 200 nM range for binding to CD16b-NA2, similar to the parental A2 clone (Table 13).

TABLE 13

| | Apparent K_{D} (nM) | | |
|---|---|---|---|
| | CD16a-V | CD16a-F | CD16b-NA2 |
| A2 | 0.39 | 0.38 | 262 |
| #55 | 0.40 | 0.28 | 239 |

The binding kinetics of the top 5 affinity-optimized clones and clone #55 compared to the parental A2 clone were also assessed by Bio-Layer Interferometry (BLI) assays using a Gator™ instrument (Probe Life, Inc (Palo Alto, CA)). For this study the clones were produced as IgG1 in an Fc silenced format using an Fc region carrying the Leu234Ala, Leu235Ala and Pro329Gly mutations to remove the Fc binding site for CD16a and CD16b.

In this study, the following non-biotinylated recombinant extracellular domains (ECDs) were purchased from Acrobiosystems. Human CD16a-F176 and CD16a-V176 ECDs encompassing residues Gly 17 to Gln 208 followed by a His at the C-terminus with a M. Wt. of 23.7 kDa (hereon referred to as CD16a-F and CD16a-V, respectively) (catalogue numbers CDA-H5220 and CDA-H52H4, respectively); human CD16b-NA1 and NA2 ECDs encompassing residues Gly 17 to Ser 200 followed by an His tag at the C terminus with M. Wt. of 228 kDa (hereon referred to as CD16b-NA1 and CD16b-NA2) (catalogue numbers CDB-H5227 and CDB-H5222, respectively); Cynomolgus CD16a(Uniprot #Q8SPW2) ECD encompassing residues Gly 17 to Gln 208 followed by a His at the C-terminus with a M. Wt. of 23.8 kDa (hereon referred to as Cyno-CD16a) (Acrobiosystems, catalogue number FC6-C52H9).

In brief, the binding kinetics study was carried out with filtered Q-buffer (1×PBS with 0.2% BSA and 0.02% Tween-20) at a temperature of 30° C. After establishing a baseline with the Q-buffer, test IgG1 antibodies at 1 μg/ml (200 μl per well) were captured onto six ProtG probes until a wavelength shift of approximately 1 nm was observed on the Gator sensorgram. After reestablishing baseline, binding of captured antibody to the test CD16 analyte (200 μl per well) was measured via separate association (six concentrations (1 per well) consisting of a 2-fold serial dilution range of 100-3.125 nM for human CD16a, 200-6.25 nM for Cynomolgus CD16, and 800-25 nM for CD16b) and dissociation steps. Non-specific binding events, to probe or an irrelevant control antibody, were monitored in two separate wells and reference subtracted from each test antibody sensorgram. Antibody Kₒₙ, K_{off} and K_{D} values were determined using Gator software through global fitting of the data to a 1:1 binding model. Exemplary binding kinetics of clones #1A2 and #2-2A2, and reference IgG1 antibodies determined by BLI are shown in FIG. 4B. The results for binding kinetics to the different human CD16 extracellular domains are shown in Table 14 and to the cynomolgus CD16 and mouse FcγRIV (the homolog of human CD16a) in Table 15. ND indicates could Not be Determined because the binding activity was too low even at the highest concentration of antibody used, 800 nM. FIG. 4C shows the binding curves of clone #2-2A2 to recombinant CD16 a (F/V) and CD16b (NA1/NA2) determined by ELISA. As shown in the results, tested anti-CD16a antibody clones specifically recognized both isoforms of recombinant human CD16a. They also recognized cynomolgus CD16 with high affinity. These antibodies in general showed no or low binding to CD16b (NA1&NA2). They did not recognize mouse FcγRIV, the homolog of human CD16a.

TABLE 14

| | | | | | | | CD16b | CD16b |
|---|---|---|---|---|---|---|---|---|
| | Huma CD16a(F) | | | Huma CD16a(V) | | | NA1 | NA2 |
| | KD | Kon | Koff | KD | Kon | Koff | KD | KD |
| Antibody* | (nM) | (1/Ms) | (1/s) | (nM) | (1/Ms) | (1/s) | (nM) | (nM) |
| #A2-IgG1 | 16.5 | 6.41E+05 | 0.0106 | 14.9 | 7.50E+05 | 0.0112 | ND | ND |
| #1A2-IgG1 | 2.3 | 6.22E+05 | 0.0014 | 3.1 | 7.94E+05 | 0.0025 | 111.0 | ND |
| #2-1A2-IgG1 | 3.0 | 5.06E+05 | 0.0015 | 4.4 | 8.29E+05 | 0.0036 | ND | >>1 μM |
| #2-2A2-IgG1 | 7.7 | 5.10E+05 | 0.0039 | 3.6 | 6.22E+05 | 0.0023 | 446.0 | ND |
| #2-3A2-IgG1 | 1.9 | 5.90E+05 | 0.0011 | 3.0 | 8.65E+05 | 0.0026 | ND | >>1 μM |
| #10A2-IgG1 | 4.1 | 5.35E+05 | 0.0022 | 3.1 | 9.07E+05 | 0.0028 | ND | ND |
| #55-IgG1 | 18.0 | 9.21E+05 | 0.0166 | 6.6 | 1.51E+06 | 0.00995 | ND | 214 |
| Reference- | 15.1 | | | 13.3 | | | ND | ND |
| IgG1 | | | | | | | | |

*Abs in IgG1 format with silenced Fc.

TABLE 15

| | | | | Mouse |
|---|---|---|---|---|
| | Cyno CD16a | | | FcγRIV |
| Antibody * | KD (nM) | Kon (1/Ms) | Koff (1/s) | ND |
| A2 | 15.6 | 7.72E+05 | 0.012 | ND |
| #1A2 | 6.3 | 7.29E+05 | 0.0046 | ND |
| #2-1A2 | 3.5 | 7.77E+05 | 0.0028 | ND |
| #2-2A2 | 7.0 | 6.96E+05 | 0.0071 | ND |
| #2-3A2 | 4.9 | 8.38E+05 | 0.0037 | ND |
| #10A2 | 6.6 | 9.36E+05 | 0.0062 | ND |
| #55 | ND | | | ND |
| Reference- | 35.4 | | | ND |
| IgG1 | | | | |

* Abs in IgG1 format with silenced Fc.

BLI assays also confirmed that clone #55 bound to a distinct epitope on CD16a than clone A2 and its derivatives.

The binding kinetics of these clones compared to the parental A2 clone were also assessed by BLI in the presence of human IgG. Examples of binding kinetics of the clones #1A2 and #2-2A2 determined by BLI in presence of human IgG at 10 mg/ml are shown in FIG. 4D. For these experiments, anti-CD16a Fab-His clones were produced from transfected 293 cells, and purified from the culture supernatants with Nickle and SEC column chromatography. Biotinylated CD16a(F/V) (Acro Biosystems) was captured by Streptavidin biosensors (Gator). Serial dilutions of Fab was added to the captured CD16a, in the absence or presence of human IgG at 10 mg/ml (Athens Research & Technology). The results for binding kinetics to CD16a-F and CD16a-V in the absence or presence of human IgG are shown in Table 16. The results show that tested anti-CD16a clones are generally more resistant to the human IgG competition than the reference antibody.

TABLE 16

| | KD (nM) | | | | | |
|---|---|---|---|---|---|---|
| | CD16a(F) | | | CD16a(V) | | |
| | | | Fold | | | Fold |
| | | | Changed | | | Changed |
| | No- | Plus- | (+IgG/ | No- | Plus- | (+IgG/ |
| Fab | hIgGs | hIgGs | No-IgG) | hIgGs | hIgGs | No-IgG) |
| A2-Fab | 40.2 | 98.6 | 2.5 | 42.3 | 82.2 | 1.9 |
| #1A2-Fab | 3.6 | 9.9 | 2.8 | 4.0 | 6.5 | 1.6 |
| #2-1A2-Fab | 3.5 | 4.7 | 1.3 | 2.7 | 3.7 | 1.4 |
| #2-2A2-Fab | 6.4 | 24.6 | 3.8 | 9.3 | 11.0 | 1.2 |
| #2-3A2-Fab | 2.8 | 1.9 | 0.7 | 2.9 | 5.4 | 1.9 |
| #10A2-Fab | 3.5 | 2.3 | 0.7 | 3.8 | 6.0 | 1.6 |
| Reference- | 11.7 | 66.4 | 5.7 | 22.1 | 47.9 | 2.2 |
| Fab | | | | | | |

These A2 and A2 variant clones in IgG1 (with silenced Fc) format were further tested for their binding affinity to CD16 expressing cells. Examples of the tested clones binding to CD16 expressing 293 cells are shown in FIGS. 4E-4I. Expi293 cells were transfected with CD16 expression plasmids. Serial dilutions of anti-CD16-IgG1 (with silenced Fc) antibodies were added to transfected cells. Bound antibodies were detected with FTIC-Fab-anti-Fc antibodies. FITC-labeled 3G8 (a pan CD16 clone) antibody was used as positive binding control. The results for binding kinetics to the different human CD16 extracellular domains are shown in Table 17. The tested anti-CD16a clones recognized both isoforms of human CD16a expressed on 293 cells. They also recognized cynomolgus CD16 expressed on 293 cells.

TABLE 17
Binding to CD16-transfected 293 cells:

| | KD (nM) | | | | |
|---|---|---|---|---|---|
| | | | Cyno- | CD16b | CD16b |
| Antibody* | CD16a(F) | CD16a(V) | CD16 | (NA1) | (NA2) |
| A2 | 1.2 | 9.0 | 0.69 | NA | NA |
| #1A2 | 2.3 | 4.4 | 0.29 | 3.8 | 2.9 |
| #2-1A2 | 1.9 | 3.3 | 0.14 | 2.8 | 36.0 |
| #2-2A2 | 3.9 | 6.7 | 0.38 | 59.3 | 64.9 |
| #2-3A2 | 3.3 | 6.2 | 0.26 | 6.9 | 15.2 |
| #10A2 | 3.9 | 4.0 | 0.28 | 7.1 | 13.7 |
| Reference- | 3.5 | 7.9 | 0.60 | NA | NA |
| IgG1 | | | | | |
| 3G8-Ab | 0.52 | 1.2 | 0.88 | 4.1 | 2.0 |

*Antibody in IgG1 format with silenced Fc

An example of the #2-2A2 clone in IgG1 format (with silenced Fc) binding to primary NK cells is shown in FIG. 4J. Primary NK cells were purchased from AllCells. The anti-CD16a antibody, #2-2A2 clone in IgG1 format was added to the primary NK cells at serial dilution. Bound antibody was detected with FTIC-Fab-anti-Fc antibody. FITC-labeled 3G8 Ab was used as positive binding control. Clone #2-2A2 showed a similar binding EC50 to the Reference antibody.

An example of the #55 clone in IgG1 format (with silenced Fc) binding to primary NK cells is shown in FIG. 4K. Clone #55 showed high binding affinity to NK cells.

Antigen ECD sequences used for screening in the above experiments are listed as below:

CD16a-F
(SEQ ID NO: 347)
GMRTEDLPKAVVFLEPQWYRVLEKDSVTLKCQGAYSPEDNSTQWFHNESL
ISSQASSYFIDAATVDDSGEYRCQTNLSTLSDPVQLEVHIGWLLLQAPRW
VFKEEDPIHLRCHSWKNTALHKVTYLQNGKGRKYFHHNSDFYIPKATLKD
SGSYFCRGLFGSKNVSSETVNITITQGLAVSTISSFFPPGYQ
CD16a-V
(SEQ ID NO: 348)
GMRTEDLPKAVVFLEPQWYRVLEKDSVTLKCQGAYSPEDNSTQWFHNESL
ISSQASSYFIDAATVDDSGEYRCQTNLSTLSDPVQLEVHIGWLLLQAPRW
VFKEEDPIHLRCHSWKNTALHKVTYLQNGKGRKYFHHNSDFYIPKATLKD
SGSYFCRGLVGSKNVSSETVNITITQGLAVSTISSFFPPGYQ
CD16b-NA1
(SEQ ID NO: 349)
GMRTEDLPKAVVFLEPQWYRVLEKDSVTLKCQGAYSPEDNSTQWFHNENL
ISSQASSYFIDAATVDDSGEYRCQTNLSTLSDPVQLEVHVGWLLLQAPRW
VFKEEDPIHLRCHSWKNTALHKVTYLQNGKDRKYFHHNSDFHIPKATLKD
SGSYFCRGLVGSKNVSSETVNITITQGLAVSTIS
CD16b-NA2
(SEQ ID NO: 350)
GMRTEDLPKAVVFLEPQWYSVLEKDSVTLKCQGAYSPEDNSTQWFHNESL
ISSQASSYFIDAATVNDSGEYRCQTNLSTLSDPVQLEVHIGWLLLQAPRW
VFKEEDPIHLRCHSWKNTALHKVTYLQNGKDRKYFHHNSDFHIPKATLKD
SGSYFCRGLVGSKNVSSETVNITITQGLAVSTIS
Cynomolgus CD16a
(SEQ ID NO: 351)
GMRAEDLPKAVVFLEPQWYRVLEKDSVTLKCQGAYSPEDNSTRWFHNESL
ISSQTSSYFIAAARVNNSGEYRCQTSLSTLSDPVQLEVHIGWLLLQAPRW
VFKEEESIHLRCHSWKNTLLHKVTYLQNGKGRKYFHQNSDFYIPKATLKD
SGSYFCRGLIGSKNVSSETVNITITQDLAVSSISSFFPPGYQ

CD16a-F
(SEQ ID NO: 352)
MWQLLLPTALLLLVSAGMRTEDLPKAVVFLEPQWYRVLEKDSVTL
KCQGAYSPEDNSTQWFHNESLISSQASSYFIDAATVDDSGEYRCQ
TNLSTLSDPVQLEVHIGWLLLQAPRWVFKEEDPIHLRCHSWKNTA
LHKVTYLQNGKGRKYFHHNSDFYIPKATLKDSGSYFCRGLFGSKN
VSSETVNITITQGLAVSTISSFFPPGYQVSFCLVMVLLFAVDTGL
YFSVKTNIRSSTRDWKDHKFKWRKDPQDK
CD16a-V
(SEQ ID NO: 353)
MWQLLLPTALLLLVSAGMRTEDLPKAVVFLEPQWYRVLEKDSVTL
KCQGAYSPEDNSTQWFHNESLISSQASSYFIDAATVDDSGEYRCQ
TNLSTLSDPVQLEVHIGWLLLQAPRWVFKEEDPIHLRCHSWKNTA
LHKVTYLQNGKGRKYFHHNSDFYIPKATLKDSGSYFCRGLVGSKN
VSSETVNITITQGLAVSTISSFFPPGYQVSFCLVMVLLFAVDTGL
YFSVKTNIRSSTRDWKDHKFKWRKDPQDK
CD16b-NA1
(SEQ ID NO: 354)
MWQLLLPTALLLLVSAGMRTEDLPKAVVFLEPQWYRVLEKDSVTL
KCQGAYSPEDNSTQWFHNENLISSQASSYFIDAATVDDSGEYRCQ
TNLSTLSDPVQLEVHVGWLLLQAPRWVFKEEDPIHLRCHSWKNTA
LHKVTYLQNGKDRKYFHHNSDFHIPKATLKDSGSYFCRGLVGSKN
VSSETVNITITQGLAVSTISSFSPPGYQVSFCLVMVLLFAVDTGL
YFSVKTNI
CD16b-NA2
(SEQ ID NO: 355)
MWQLLLPTALLLLVSAGMRTEDLPKAVVFLEPQWYSVLEKDSVTL
KCQGAYSPEDNSTQWFHNESLISSQASSYFIDAATVNDSGEYRCQ
TNLSTLSDPVQLEVHIGWLLLQAPRWVFKEEDPIHLRCHSWKNTA
LHKVTYLQNGKDRKYFHHNSDFHIPKATLKDSGSYFCRGLVGSKN
VSSETVNITITQGLAVSTISSFSPPGYQVSFCLVMVLLFAVDTGL
YFSVKTNI
Cynomolgus CD16a
(SEQ ID NO: 356)
MWQLLLPTALLLLVSAGMRAEDLPKAVVFLEPQWYRVLEKDSVTL
KCQGAYSPEDNSTRWFHNESLISSQTSSYFIAAARVNNSGEYRCQ
TSLSTLSDPVQLEVHIGWLLLQAPRWVFKEEESIHLRCHSWKNTL
LHKVTYLQNGKGRKYFHQNSDFYIPKATLKDSGSYFCRGLIGSKN
VSSETVNITITQDLAVSSISSFFPPGYQVSFCLVMVLLFAVDTGL
YFSVKKSVPSSTRDWEDHKFKWSKDPQDK

Sequence liability information of these anti-CD16a antibody clones are shown in Table 18 below:

TABLE 18

| | | | Deamidation | | | |
|---|---|---|---|---|---|---|
| | N- | | (NS, NG, | | Protease | |
| | glycosylation | | NH, NT, NQ, | Isomerization | cleavage | |
| | (NX[S/T]), | Unpaired | NF, NW, | (DS, DG, DD, | site (DP, | Oxidation |
| Clone | XP | Cys | NY) | DN, DR) | DQ) | (M) |
| A2 | None | None | None | None | None | None |
| #1A2 | None | None | None | None | None | None |
| #2-1A2 | None | None | None | None | None | None |
| #2-2A2 | None | None | None | None | None | None |
| #2-3A2 | None | None | None | None | None | None |
| #10A2 | None | None | None | None | None | None |
| Reference | Potential site | None | None | Potential site | None | None |

These anti-CD16a antibody clones in IgG1 and Fab formats were also tested for their melting point temperature (Tm) by differential scanning fluorimetry (DSF) with SYPRO Orange Protein Gel Stain (Millipore Sigma) using a Roche LightCycler 480 Real-Time PCR instrument. The results show that the tested anti-CD16a clones generally have higher Tm (7-8° C.) than that of the Reference clone. Results of the Tm are shown in FIG. 4L, Tables 19 and 20 below:

TABLE 19

Tm (° C.)

| IgG* | Tm1 (CH2) | Tm2 |
|---|---|---|
| A2-IgG1 | 64.73 | 90.14 |
| #1A2-IgG1 | 64.43 | 87.47 |
| #2-1A2-IgG1 | 64.23 | 89.74 |
| #2-2A2-IgG1 | 66.01 | 88.85 |
| #2-3A2-IgG1 | 64.63 | 90.63 |
| #10A2-IgG1 | 65.02 | 90.14 |
| Reference-IgG1 | 64.82 | 80.15 |

*Ab with silenced Fc.

TABLE 20

| Fab | Tm (° C.) |
|---|---|
| A2-Fab | 88.80 |
| #1A2-Fab | 87.01 |
| #2-1A2-Fab | 88.31 |
| #2-2A2-Fab | 88.50 |
| #2-3A2-Fab | 89.20 |
| #10A2-Fab | 88.80 |
| Reference-Fab | 81.50 |

Anti-CD16a antibody clones in IgG1, Fab, or scFv-scFv-IgG4 bivalent antibody (see Section 7.5 below) formats were produced from Expi293 cells with transient transfection method using ExpiFectamine (Thermofisher). The purification was carried out 5 days post-transfection. Tested anti-CD16a clones showed high expression in Fab and bispecific formats. The results are shown in Tables 21-23 below.

TABLE 21
Production of IgG1-Abs from 25-ml of Expi293 cells

| Ab* | Yield (mg) |
|---|---|
| A2-IgG1 | 11.85 |
| #1A2-IgG1 | 6.93 |
| #2-1A2-IgG1 | 6.08 |
| #2-2A2-IgG1 | 3.80 |
| #2-3A2-IgG1 | 5.83 |
| #10A2-IgG1 | 5.08 |
| Reference-IgG1 | 5.75 |

*Ab with silenced Fc.

TABLE 22
Production of Fabs from 100-ml of Expi293 cells

| Fab | Yield (mg) |
|---|---|
| A2-Fab-His | 2.16 |
| #1A2-Fab-His | 0.91 |
| #2-1A2-Fab-His | 1.79 |
| #2-2A2-Fab-His | 1.47 |
| #2-3A2-Fab-His | 1.82 |
| #10A2-Fab-His | 0.74 |
| Reference-Fab-His | 0.61 |

TABLE 23
Production of CD19-CD16-scFv-IgG4 from 50 mL Expi293 cells

| BsAb | Total (mg) |
|---|---|
| CD19-A2-scFv-IgG4 | 0.858 |
| CD19-#1A2-scFv-IgG4 | 0.622 |
| CD19-#2-1A2-scFv-IgG4 | 0.92 |
| CD19-#2-2A2-scFv-IgG4 | 1.075 |
| CD19-#2-3A2-scFv-IgG4 | 1.258 |
| CD19-#10A2-scFv-IgG4 | 0.989 |
| CD19-Reference-scFv-IgG4 | 0.44 |

### 7.4 Example 4. Epitope Binding Sites

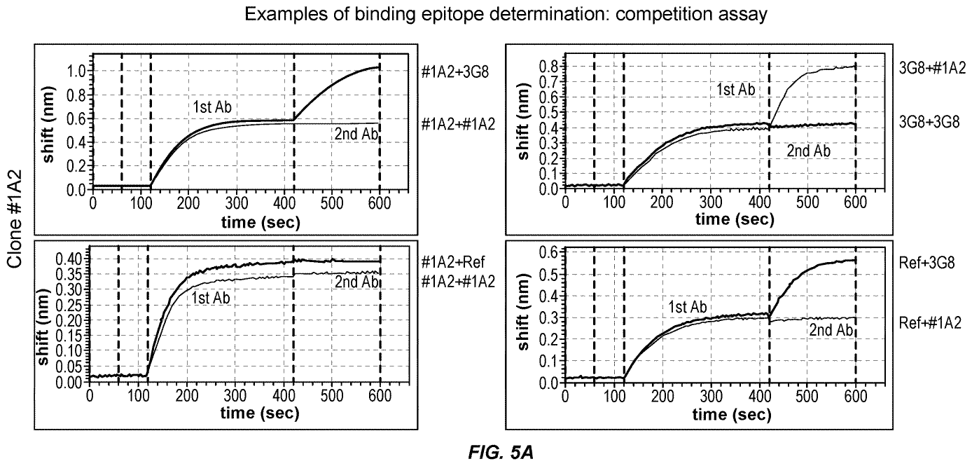

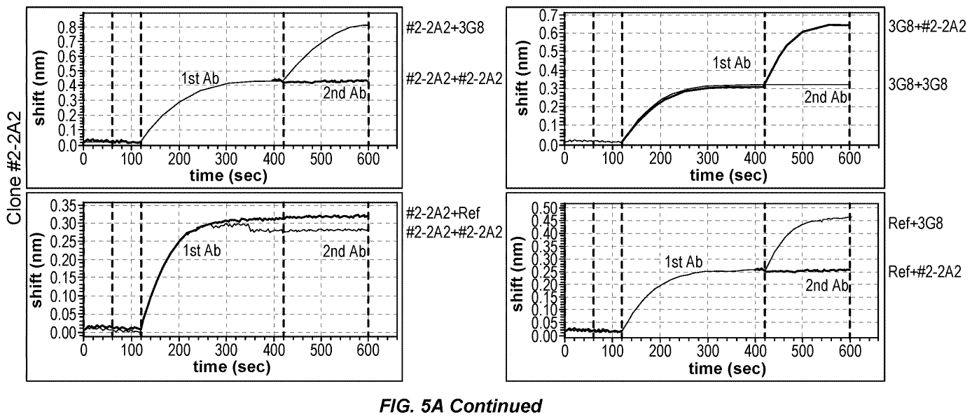

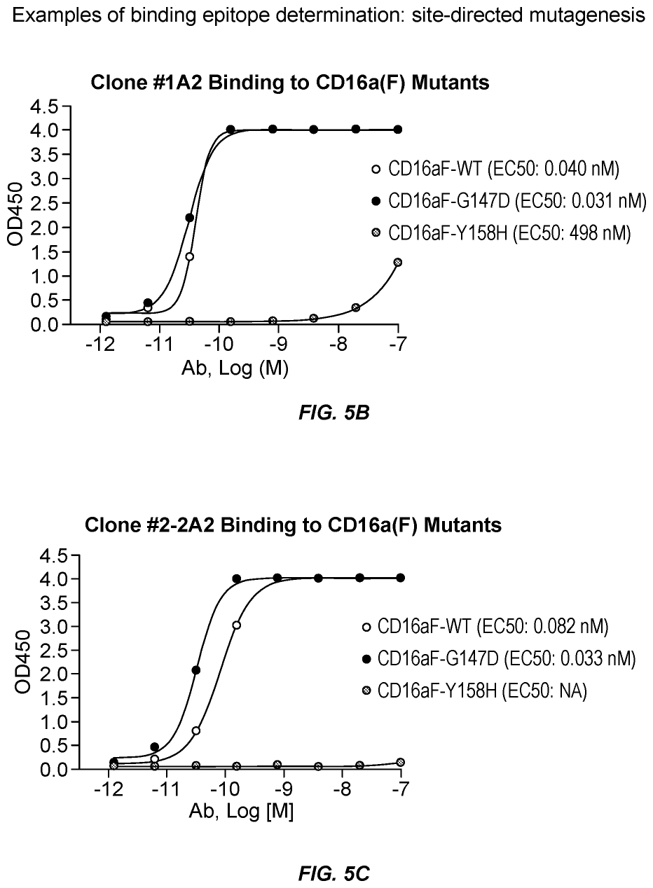

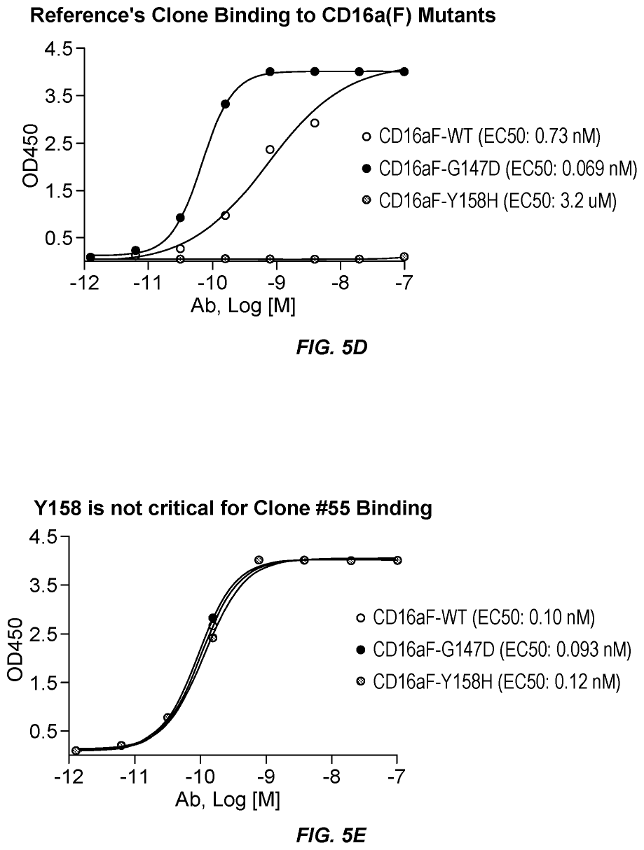

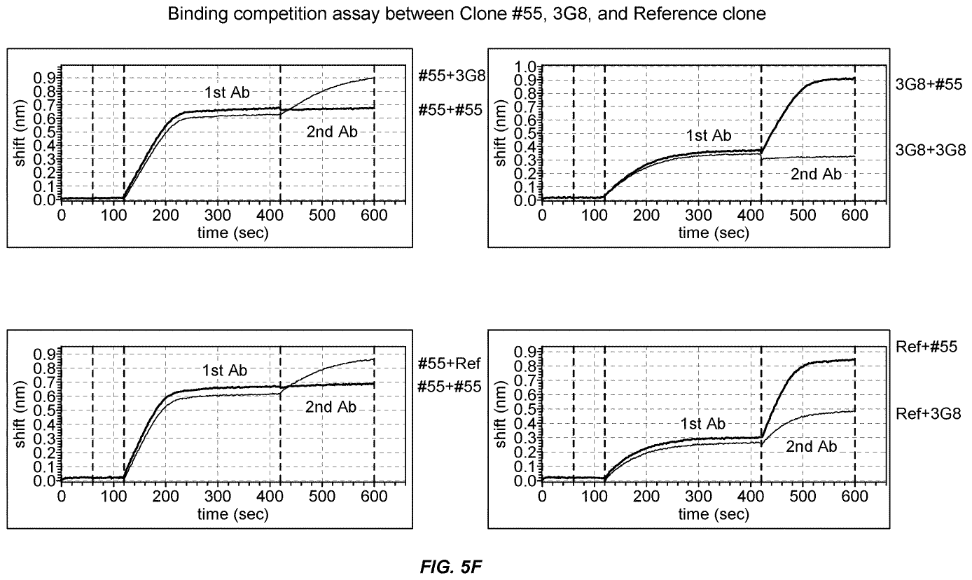

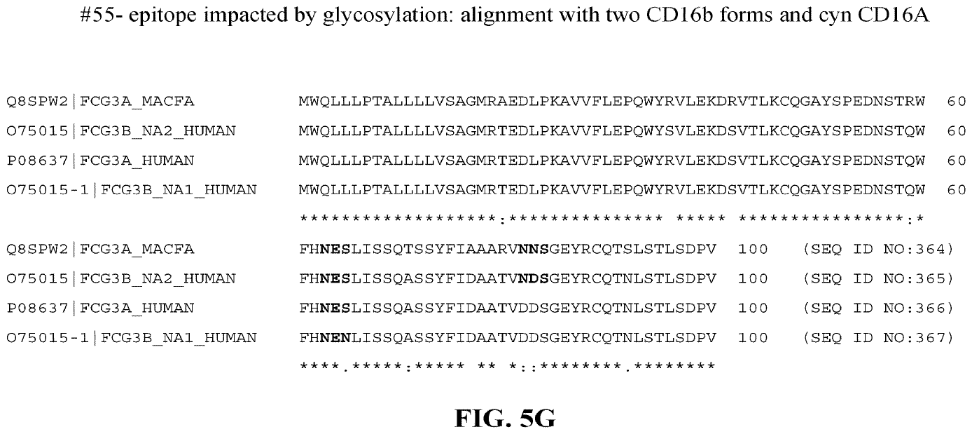

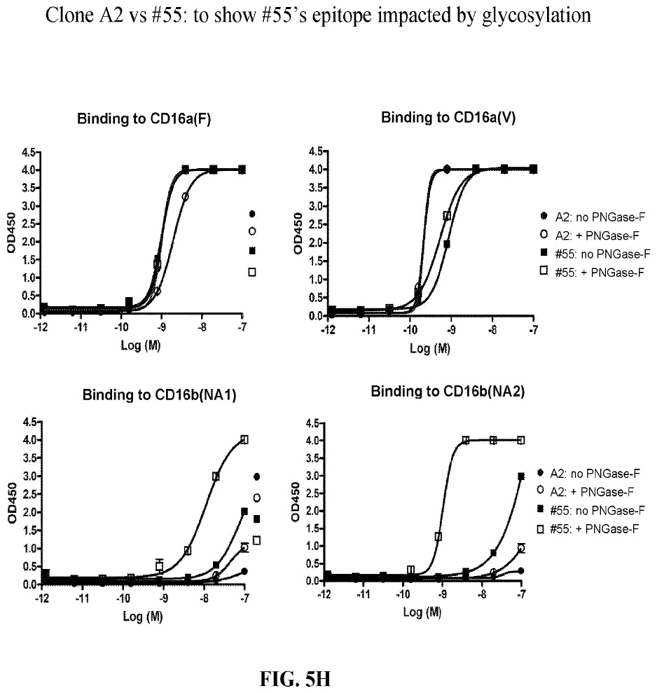

A competition assay was performed for binding epitope determination. Biotinylated CD16a(F) was captured by Streptavidin biosensors (Gator). The first antibody was then added and allowed to associate to reach saturation and then the second antibody added for further association. Additional association after addition of the second antibody indicates a separate binding site, lack of further association indicates identical or overlapping binding site to that of the first antibody. Binding competition by BLI between clones #1A2 #2-2A2, 3G8 and the reference clone are shown in FIG. 5A. Binding competition by BLI between clone #55, 3G8 and the reference clone are shown in FIG. 5F. Results of the competition assay are shown in Table 24. All tested anti-CD16a antibody clones except clone #55 competed with the Reference antibody, indicating that clone #55 recognizes a different epitope on CD16a from the reference clone, and clone A2 and its derivative clones. All the anti-CD16a antibodies did not compete with the 3G8 antibody.

TABLE 24

Competition with

| Antibody* | Reference-IgG1 | 3G8 |
|---|---|---|
| A2-IgG1 | Yes | No |
| #1A2-IgG1 | Yes | No |
| #2-1A2-IgG1 | Yes | No |
| #2-2A2-IgG1 | Yes | No |
| #2-3A2-IgG1 | Yes | No |
| #10A2-IgG1 | Yes | No |
| Reference-IgG1 | Yes | No |
| #55-IgG1 | No | No |

*Antibody with silenced Fc.

The only three residues that differ between the two CD16a and two CD16b allotypes are G147, Y158 and F203 (numbering based on UniProt propeptide sequence P08637). F203 is adjacent to the transmembrane sequence, therefore, the key residues for conferring CD16a specificity are likely to be G147 and/or Y158. Mutated versions of the extracellular domain of both CD16a-F and CD16a-V that we fused with human Fc region were generated and the binding of select clones was assessed by ELISA.

Wells of 96-well plated were coated with either WT-CD16a-F-ECD, the G147D variant or the Y158H variant and serial dilutions of each of the clones added. After 1 hour incubation at RT wells were washed and bound IgG detected with HRP labeled goat anti human kappa IgG. The data clearly indicated that Y158 was critical for the binding of the A2 family of clones to CD16a (Table 25). The binding curves of the exemplary clones #1A2, #2-2A2, and the reference clone binding to CD16(F) mutants are shown in FIGS. 5B-5D. In contrast, these mutations made no difference to the binding affinity of clone #55 (FIG. 5E), which indicates that clone #55 binds to a different epitope that is not dependent on Y158.

TABLE 25

| | K_{D} (nM) | | | |
|---|---|---|---|---|
| | WT | G147D | Y158H | |
| A2 | 0.25 | 0.034 | <10% at 100 | nM |
| #1 A2 | 0.040 | 0.031 | ~25% at 100 | nM |
| #2-1A2 | 0.041 | 0.036 | NB at 100 | nM |
| #2-2A2 | 0.082 | 0.033 | 10% at 100 | nM |
| #2-3A2 | 0.040 | 0.030 | ~25% at 100 | nM |
| #3-A2 | 0.11 | 0.040 | 10% at 100 | nM |
| #6-A2 | 0.13 | 0.040 | 10% at 100 | nM |
| #10-A2 | 0.041 | 0.034 | 10% at 100 | nM |

| | | | |
|---|---|---|---|
| #55 | 0.10 | 0.093 | 0.12 |

Since the binding properties of clone #55 to CD16a ECDs were not impacted by either the G147D or Y158H mutations and clone #55 does not bind to cynomolgus CD16, the ECD sequences were compared and a potential difference in the cynomolgus CD16 sequence compared to human CD16a was the presence of a glycosylation site at position 82, which was also present in CD16b(NA2) (FIG. 5G). A potential glycosylation site was also present at position 63, which was shared between human CD16a and CD16b(NA2). To determine whether glycosylation at Gln82 impacted clone #55 binding, the various CD16 ECDs were subjected to Peptide-N-Glycosidase F (PNGase F; Thermo Fisher, catalogue A39245) digestion following the manufacturer's protocol. After confirming de-glycosylation of aliquots by SDS-PAGE, these were then used to compare binding to the glycosylated and de-glycosylated PNGase F treated CD16 proteins by ELISA. As shown in FIG. 5H and Table 26, while de-glycosylation of CD16a(F) had no effect on the apparent affinity of either clone A2 or clone #55, clone #55 showed a marked increase in affinity for deglycosylated CD16b(NA2) (>200 nM for the untreated, glycosylated CD16b(NA2) versus 1 nM for the deglycosylated CD16b(NA2)). In contrast clone A2, showed poor binding to both glycosylated and deglycosylated CD16b(NA2). The relatively low affinity binding of clone #55 to CD16b(NA1) was also enhanced upon deglycosylation following PNGase-F treatment, albeit the difference was not so marked, suggesting that lack of glycosylation at position 82 was not the only factor influencing the ability of clone #55 to bind to CD16b and that the nearby glycosylation site at position 63 may also impact the ability of clone #55 to bind to both de-glycosylated CD16b(NA1) and CD16b(NA2).

TABLE 26

| | KD (nM) | | | | | | | |
|---|---|---|---|---|---|---|---|---|
| | CD16a(F) | | CD16a(V) | | CD16b(NA1) | | CD16b(NA2) | |
| Clone | Control | PNGaseF | Control | PNGaseF | Control | PNGaseF | Control | PNGaseF |
| A2 | 1.0 | 2.0 | 0.21 | 0.21 | NB | >>100 | NB | NB |
| #55 | 0.98 | 1.0 | 0.85 | 0.53 | 73 | 11.2 | ~200 | 1 |

To assess whether introducing a glycosylation site at position 82 by mutating the Asp82 to Asn (D82N), vectors encoding wild-type (WT) CD16a(F) or a variant with a Asp82Asn mutations were constructed and these were used to transiently transfect 293 cells. After two (2) days, a cell binding assay was performed using a 5-fold serial dilution of either clone A2 or clone #55. Clone A2 bound with equal affinity to both wtCD16a(F) and the Asp82Asn CD16a(F) expressing cells, whereas clone #55 exhibited high binding to wtCD16a(F) but no binding above background to the Asp82Asn CD16a(F) expressing cells.

### 7.5 Example 5. Activity of the Antibody Clones in a NK-Cell Engager Format

An scFv-scFv construct was generated whereby the variable regions of clone A2 were converted into an scFv and coupled with an scFv encoding an anti-CD19 clone, using the sequence for the VH (SEQ ID NO: 343) and VL (SEQ ID NO: 344) described in the DB09052 entry in drugbank.com. This was chosen as a known tumor target/targeting antibody for the purposes of exemplification of a tumor targeting moiety. The orientation was anti-CD19-(VL-VH)-GS linker-anti-CD16a-A2-(VL-VH) (SEQ ID NO: 345) (FIG. 6A) followed by a hexa-His-tag to facilitate purification.

CD-19 VH
(SEQ ID NO: 343)
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTTVTVSS
CD-19 VL
(SEQ ID NO: 344)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIK
CD19 scFv
(SEQ. ID NO: 345)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS
CD16a (A2) scFv
(SEQ. ID NO: 346)
EIVLTQSPGTLSLSPGERATLSCRASQSVSSSYLAWYQQKPGQAPRLLIY
DASSRATGIPDRFSGSGSGTDFTLTISRLEPEDFAVYYCQQYGSSSLITF
GQGTKVEIKGGGGSGGGGSGGGGSQLQLQESGPGLVKPSETLSLTCTVSG
GSISRGSYYWGWIRQPPGKGLEWIGTIDYSGRTYYNPSLKSRVTISVDTS
KNQFSLKLSSVTAADTAVYYCARAFPGEYFGVFDHWGQGTLVTVSS
CD19-CD16a (A2) scFv-scFv construct
(SEQ. ID NO: 357)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVSS
GGSGGSGGSEIVLTQSPGTLSLSPGERATLSCRASQSVSSSYLAWYQQKP
GQAPRLLIYDASSRATGIPDRFSGSGSGTDFTLTISRLEPEDFAVYYCQQ
YGSSSLITFGQGTKVEIKGGGGSGGGGSGGGGSQLQLQESGPGLVKPSET
LSLTCTVSGGSISRGSYYWGWIRQPPGKGLEWIGTIDYSGRTYYNPSLKS
RVTISVDTSKNQFSLKLSSVTAADTAVYYCARAFPGEYFGVFDHWGQGTL
VTVSS

The construct was cloned into a mammalian expression vector which was used to transiently transfect Expi293 cells. Six days later the scFv-scFv antibody was purified from the culture supernatant using a HisPur Nickel column (Thermo Fisher, catalogue #88221) according to the manufacturer's protocol, then further purified by FPLC-SEC using a HiLoad 16/600 Superdex 200 μg column (Sigma-Aldrich, Cat #28-9893-35). The sample was loaded onto the column, eluted with PBS buffer, pH 7.4, and the peak corresponding to the correct molecular weight collected. This yielded a single discrete peak by HPLC (FIG. 6B).

To assess the ability of the CD19-CD16a (A2) scFv-scFv antibody to function as an NK cell engager, 75,000 Jurkat-CD16a-V NFAT luciferase reporter cells were mixed with 25,000 CD19-positive Raji target cells or MDA-MB-231 CD19-negative cells per well of a 96-well plate. Serial dilutions of the CD19-CD16a (A2) scFv-scFv, with wells containing no added antibody serving as the negative control. In the absence of Raji target cells, the CD19-CD16a (A2) scFv-scFv antibody failed to activate luciferase but in the presence of Raji target cells, the construct was a potent activator of CD16a mediated signaling with an EC50 of 0.24 nM (FIG. 6C). In contrast, no activation of the Jurkat reporter cells was observed in the presence of MDA-MB-231 CD19-negative cells. In a second experiment, the effect of mixing Jurkat-CD16a reporter cells with Raji-CD19-positive cells in the presence of the CD19-CD16a (A2) scFv-scFv antibody was compared with the effect of an scFv-A2 coupled to an scFv against respiratory syncytial virus (RSV), i.e., an irrelevant non-Raji cell target. Similar dose-dependent activation of the Jurkat-CD16a reporter cells was observed in the presence of the CD19-CD16a (A2) scFv-scFv antibody, but no activation of the Jurkat-CD16a reporter cells was observed in the presence of the A2-RSV scFv-scFv antibody.

The A2 variant clones were also generated in scFv (CD19)-scFv (CD16a) format. These bispecific Abs were produced from 293 cells, and purified from the cell culture medium with nickel and SEC column chromatography. Activation of the reporter cell assay was carried out with Raji cells added to CD16a-V-expressing Jurkat NFAT luciferase reporter cells (Promega) at ratio of 1:3, in the absence or presence of human IgG at 10 mg/ml. The luciferase activity was measured with Bio-Glo Luciferase reagent (Promega). Results of the EC50 values for activation of CD16a of exemplary clones #1A2, #2-2A2, and the reference clone in scFv (CD19)-scFv (CD16a) format are shown in FIG. 6D (in absence of human IgG) and FIG. 6E (in presence of human IgG). Tested exemplary clones are more resistant to IgG competition than reference clone. Activation of the reporter cell assay in the absence of Raji cells was also performed (FIG. 6F). Results of the EC50 values of these bispecific Abs in the activating reporter cell assays are shown in Table 27 below.

TABLE 27

Report Assay (EC50, nM)

| BsAb | No-IgG | Plus-IgG |
|---|---|---|
| CD19-A2-scFv-His | 0.18 | 0.77 |
| CD19-#1A2-scFv-His | 0.029 | 0.12 |
| CD19-#2-1A2-scFv-His | 0.021 | 0.098 |
| CD19-#2-2A2-scFv-His | 0.035 | 0.21 |
| CD19-#2-3A2-scFv-His | 0.022 | 0.11 |
| CD19-#10A2-scFv-His | 0.018 | 0.15 |
| CD19-Reference-scFv-His | 0.21 | 0.17* |

*Very low maximal activation

Same reporter cell assay was also performed for clone #55. Results of the EC50 values for activation of CD16a of exemplary clone #55, and the reference clone in scFv (CD19)-scFv (CD16a) format are shown in FIG. 6G (in absence of human IgG) and FIG. 6H (in presence of human IgG). The study was also performed to compare the activity of clone #55 with clone A2. While the EC50 value was higher for clone #55 compared to clone A2 in the absence of IgG, the EC50 value was unaffected by the presence of 10 mg/mL human IgG (Table 28).

TABLE 28

EC₅₀ (nM)/Maximum Signal RLU

| Clone | No hIgG | Plus hIgG |
|---|---|---|
| A2 | 0.17/41,000 | 1.2/12,000 |
| #55 | 1.2/27,000 | 1.1/16,000 |

A second format was constructed whereby the scFv-scFv was fused to IgG4 Fc (FIG. 7A). The constructs were subcloned into mammalian expression vectors and transfected into 293 cells. The scFv (CD19)-scFv (CD16a-A2)-IgG4 antibody was purified by Protein A chromatography and assessed by FPLC-SEC. Approximately 85% was the desired monomeric peak occurring at an elution volume of 60 to 68 ml, which was purified by preparative SEC as described above (FIG. 7B). To assess the ability of the scFv (CD19)-scFv (CD16a-A2)-IgG4 bivalent antibody to function as an NK cell engager, the purified antibody was then evaluated in the Jurkat-CD16a-V NFAT luciferase reporter (effector) cells and CD19-positive Raji target cells as described above. In this bivalent format, the EC50 value for activation of CD16a was 0.08 nM (FIG. 7C).

The A2 variant clones were also generated in scFv (CD19)-scFv (CD16a)-IgG4 bivalent format. These bispecific Abs were produced from 293 cells, purified with Protein-A and SEC column chromatography. Activation of a reporter cell assay was carried out with and CD19-positive Raji target cells to CD16a-V-expressing Jurkat NFAT luciferase reporter cells (Promega) at ratio of 1:3 in the absence or presence of human IgG at 10 mg/ml. The luciferase activity was measured with Bio-Glo Luciferase reagent (Promega). Results of the EC50 values for activation of CD16a of exemplary clones #1A2, #2-2A2, and the reference clone in scFv (CD19)-scFv (CD16a)-IgG4 bivalent format, and CD19-IG1 with Fc enhanced mutations (S239D and I332E) are shown in FIG. 7D (in absence of human IgG) and FIG. 7E (in presence of human IgG). EC50 values of the bivalent BsAb in activating reporter cells are shown in Table 29 below.

The results show that the tested clones in bivalent format are more active than the reference antibody clone, especially in the presence of 10 mg/ml IgG. The tested clones are more active than the bivalent CD19-IgG1 with enhanced Fc in the absence and presence of 10 mg/mL human IgG.

TABLE 29

Report Assay (EC50, nM)

| BsAb | No-IgG | Plus-IgG |
|---|---|---|
| CD19-A2-scFv-IgG4 | 0.005 | 0.3 |
| CD19-#1A2-scFv-IgG4 | 0.006 | 0.01 |
| CD19-#2-1A2-scFv-IgG4 | 0.008 | 0.027 |
| CD19-#2-2A2-scFv-IgG4 | 0.006 | 0.015 |
| CD19-#2-3A2-scFv-IgG4 | 0.009 | 0.037 |
| CD19-#10A2-scFv-IgG4 | 0.011 | 0.12 |
| CD19-Reference-scFv-IgG4 | 0.11 | 189 |
| CD19-IgG1* | 0.003** | 0.013** |

*Fc enhanced: S239D and I332E;
**very low maximal activation

The ability of both construct formats to mediate lysis of Raji cells by NK cells from a healthy donor was then evaluated. To ensure that the clones bound to native CD16a expressed by human NK cells, the ability of the CD19-CD16aA2-IgG4 to bind to purified NK cells (Astarte Biologics) was first evaluated by flow cytometry. Aliquots of 50,000 NK cells were incubated in the presence of 100 nM CD19-CD16aA2-IgG4 followed by Alexa-488 labeled goat anti-human IgG, or secondary detection agent alone. The cells were then analyzed by flow cytometry using an iQue flow cytometer. The CD19-CD16aA2-IgG4 clone exhibited robust binding to NK cells (FIG. 8).

Normally, Raji cells are resistant to NK cell-mediated lysis since they express multiple ligands for inhibitory NK cell receptors including HLA-A*03 (ligand for KIR3DL2), and Cw*03 and Cw*04 (HLA-C ligands for KIR2DL2/3 and KIR2DL1). The first experiment was performed with the CD19-CD16a-A2 scFv-scFv format. NK cells from a healthy donor were obtained from Astarte Biologics. Raji cells were pre-loaded with calcein AM and seeded at 10,000 per well of a 96-well plate together with 100,000 per well PBMCs in 100 μl RPMI Media. Serial dilutions of the CD19-CD16a-A2 scFv-scFv or a control A2-CD16a-RSV-scFv were added, the mixtures incubated at 37° C. for 3.5 hours, then washed for 15 minutes. The percent non-fluorescent compared to fluorescent live cells was counted by iQue flow cytometry to determine the percent cell killing at each concentration. This CD19-CD16a-A2 scFv-scFv antibody induced potent NK-cell mediated cell killing of Raji cells with an EC50 value of approximately 0.5 nM whereas the A2-CD16a-RSV control had no ADCC mediated cytotoxicity effect.

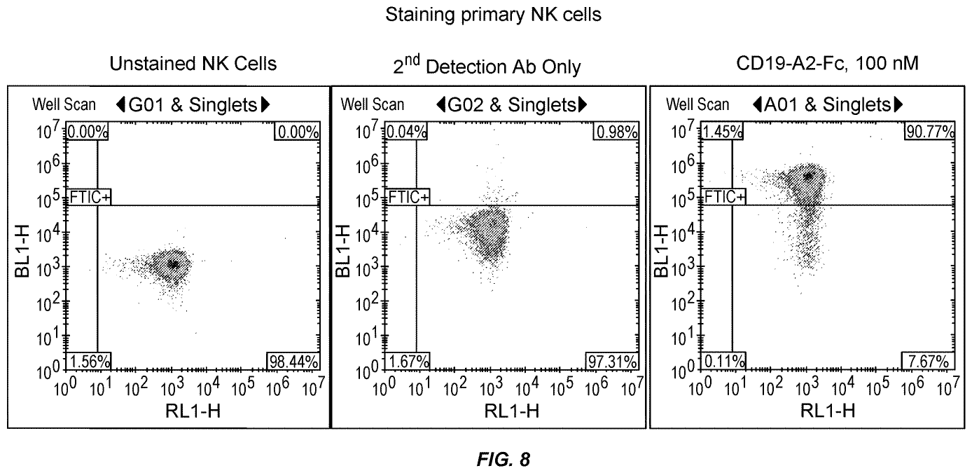

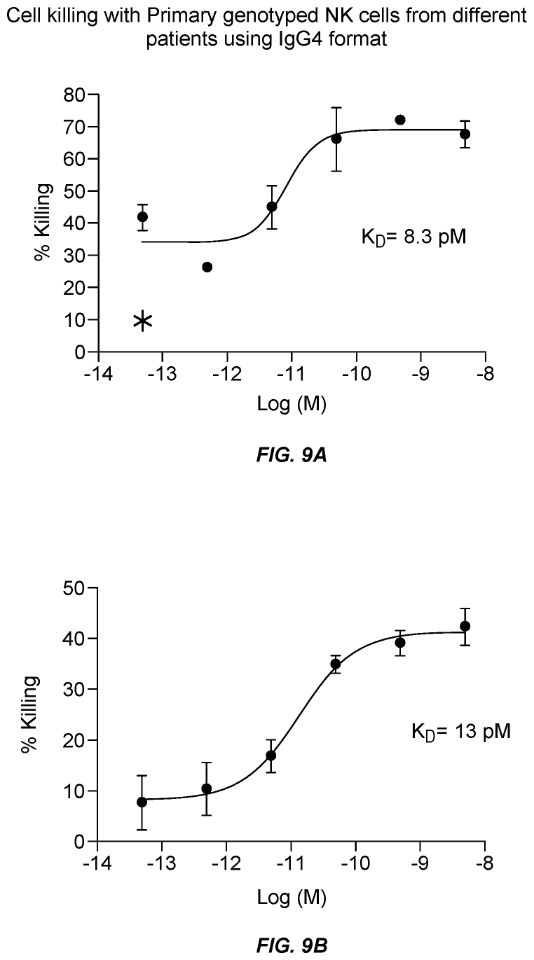

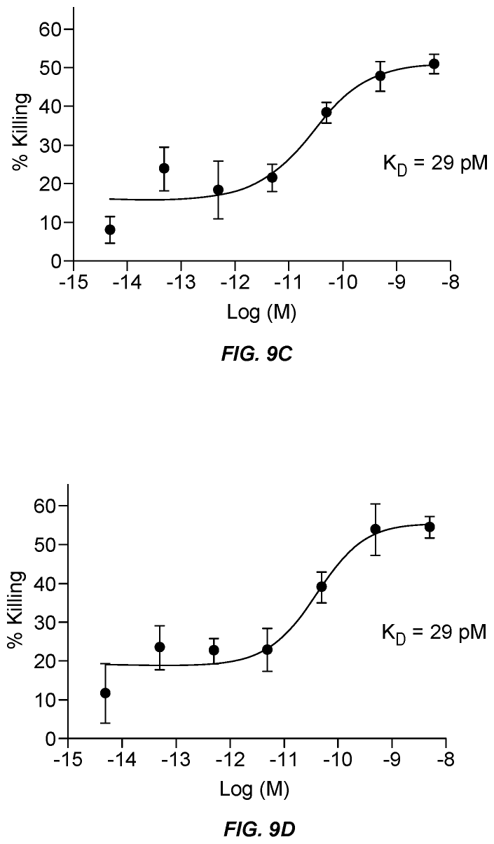

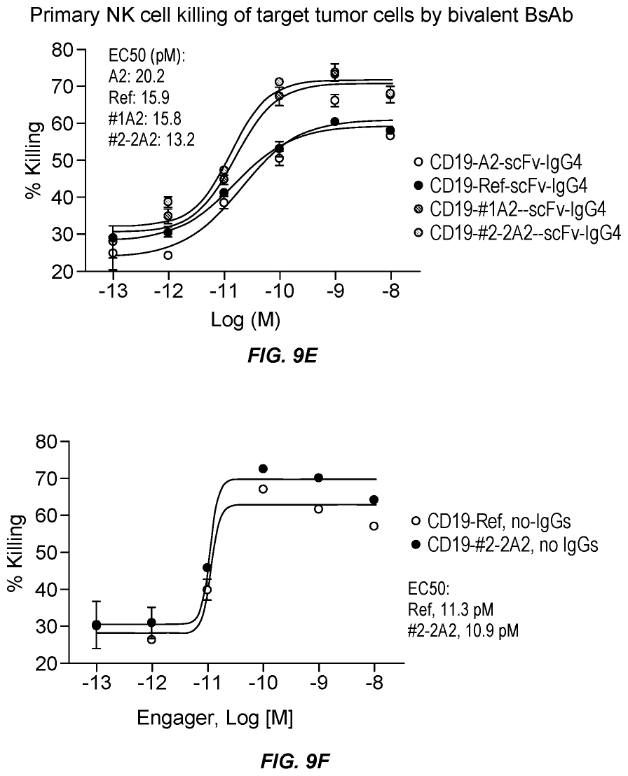

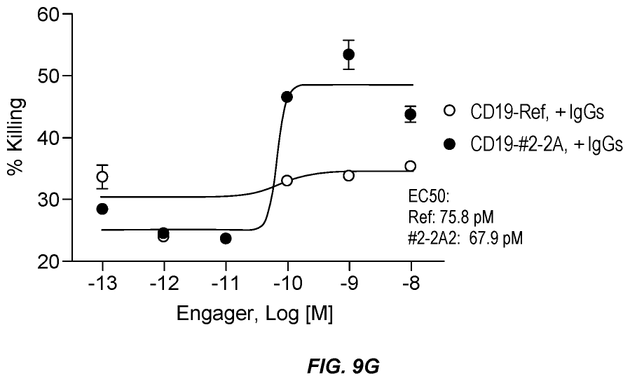

Next the scFv (CD19)-scFv (CD16a-A2)-IgG4 bivalent antibody construct was tested in the Raji cell cytotoxicity assay using three different patient NK samples with defined CD16a genotype (Astarte Biologics). Two patients had a 176 F/V genotype and one a 176 F/F genotype. Aliquots of 100,000 NK cells were mixed with 20,000 Raji cells pre-loaded with carboxyfluorescein succinimidyl ester (CFSE; Thermo Fisher, catalogue #C34554) per well of a 96-well plate and a 5-fold serial dilution of the CD19-CD16a bivalent IgG4 clones ranging from 5 nM down to 5×10⁻¹⁴ M added to the designated wells. The plates were incubated for 20 hours at 37° C. then viable cells were labeled with 7-amino-actinomycin D (7-ADD; Thermo Fisher, catalogue #00-6993-50), and both viable and dead cells were detected with iQue Screener Plus (Intellicyt). In all three cases the CD19-CD16a-A2 IgG4 antibody exhibited potent activity both in the presence and absence of 10 mg/ml human IgG (FIGS. 9A-9C and Table 30).

TABLE 30
(ND indicates not determined; PCF indicates poor curve fit)

Cytotoxicity: EC50

| | | A2 + 10 |
|---|---|---|
| | A2 No hIgG | mg/ml hIgG |
| Subject Genotype | A2 | A2 |
| Subject #1: 176F/V | 8.3 pM | PCF |
| Subject #2: 176F/F | 13.3 pM | 178 pM |
| Subject #3: 176F/V | 29.4 pM | ND |

Selected A2 variant clones (#1A2 and #2-2A2) in scFv (CD19)-scFv (CD16a)-IgG4 bivalent antibody format were also tested in the Raji cell cytotoxicity assay using primary NK cell samples. ScFv (CD19)-scFv (CD16a)-IgG4 bivalent Abs were produced from 293 cells, purified with protein A and SEC columns. Raji cells pre-stained with CFSE were added with primary NK cells (at 1:5 ratio) in the presence of serial dilution of the NK cell engagers in the absence or presence of human IgG. The live cells were detected with an iQue Screener Plus (Intellicyte). Exemplary results are shown in FIGS. 9E-9G. The tested anti-CD16a clones are more active than the reference antibody clone, especially in the presence of 10 mg/mL human IgG in primary NK cell killing assay.

### 7.6 Example 6. Lack of Binding Activity to Neutrophils

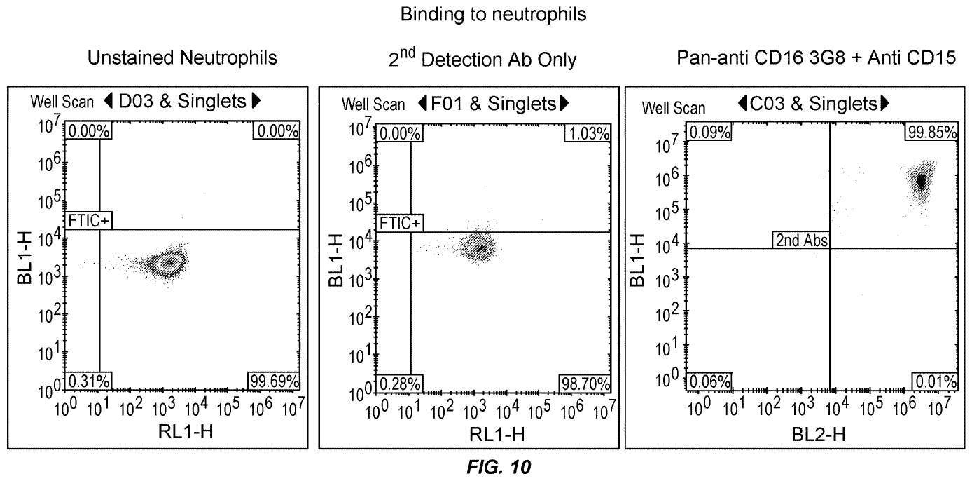

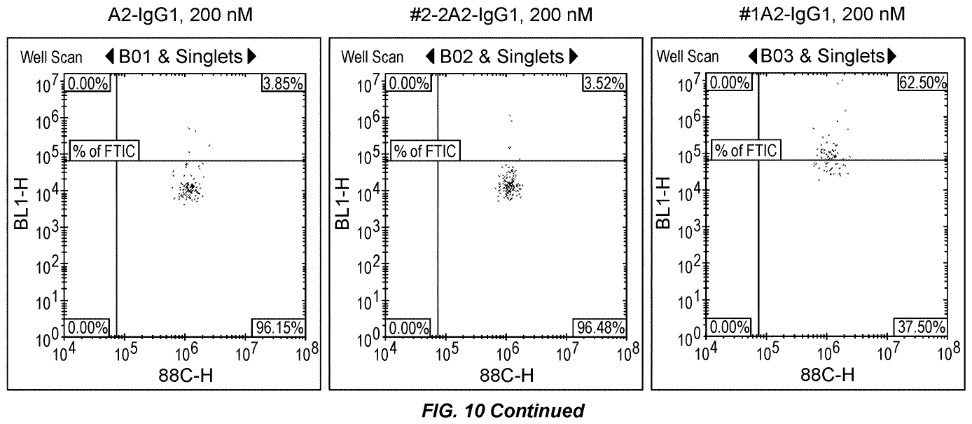

Since the low affinity binding to neutrophils may reflect binding activity of the Fc moiety, to ensure that the selected clones, did not bind neutrophils, a binding study with neutrophils was performed using antibody clones with mutated Fcγ1 region to eliminate Fc binding activity to Fcγ receptors. Human neutrophils were isolated from human blood samples obtained from the San Diego Blood Bank using an EasySep Neutrophil Isolation kit (Stemcell Technologies, catalogue #19666). Staining an aliquot with APC-labeled clone HI98, an anti-CD15 clone (the sialyl LewisX glycan marker; BioLegend catalogue #301908) and a PE-labeled CD16 clone, 3G8, raised against neutrophils that recognizes both CD16a and CD16b as a positive control (Biolegend catalogue #302056) confirmed that the neutrophil population expressed CD16b (FIG. 10). However, even at a concentration as high as 200 nM, the CD16a-A2 and CD16a- #2-2A2 antibody clones in IgG1 format with mutated Fcγ1 region to eliminate Fc binding activity to Fcγ receptors exhibited no binding above the background level of staining observed with the secondary detection antibody alone, while the CD16a- #1A2 clone did show some degree of staining at 200 nM albeit not as strong as the 3G8 fluorescence intensity (FIG. 10).

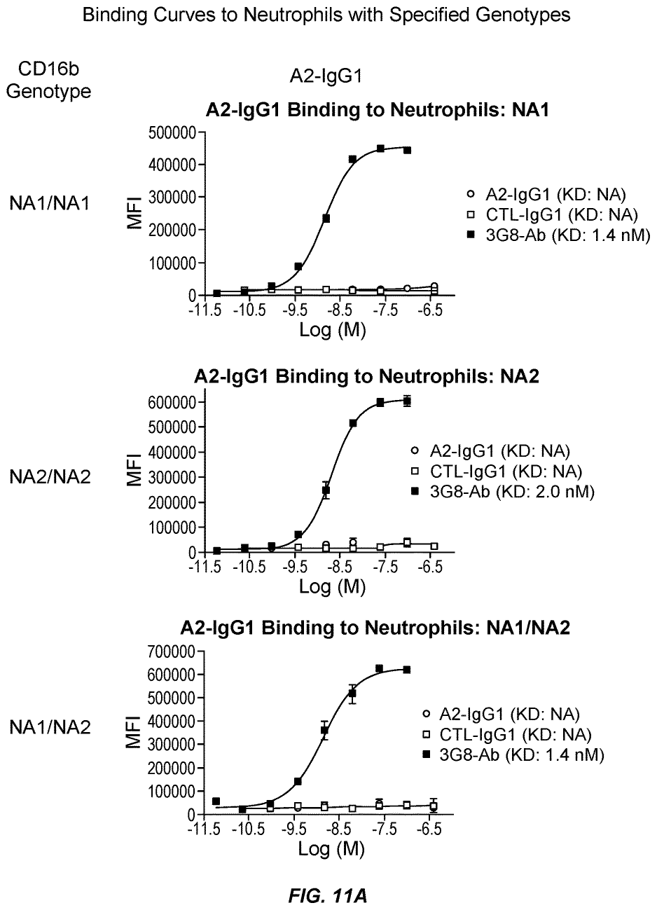

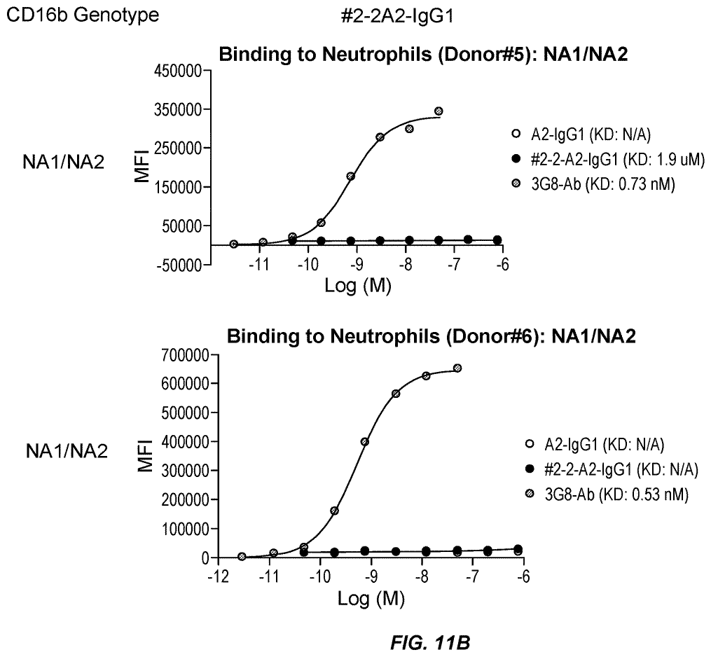

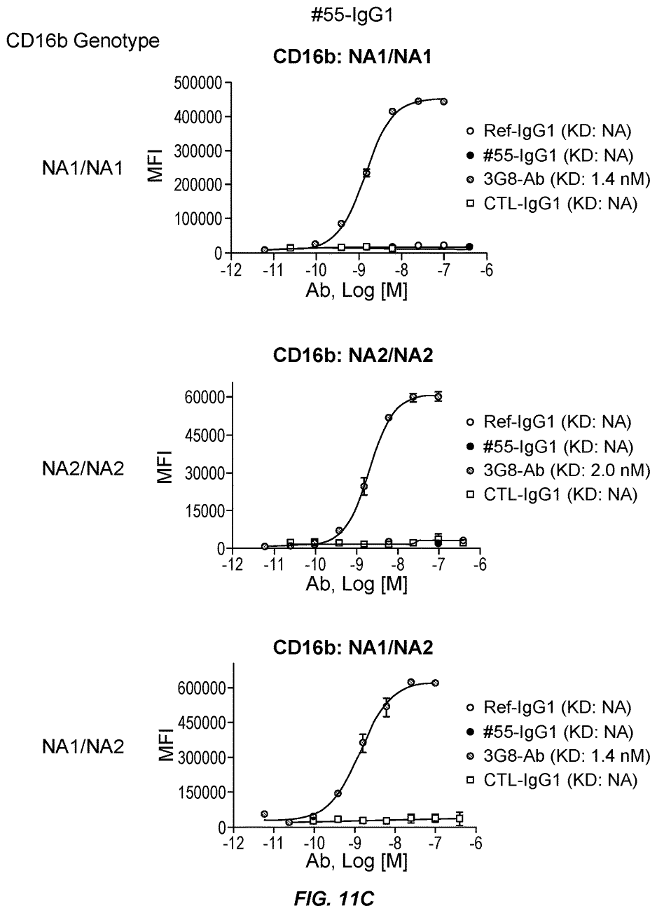

Three different neutrophil samples were obtained from three subjects who differed in their CD16b genotype, which was then used for the binding with CD16a-A2 with mutated Fcγ1 region to eliminate Fc binding activity to Fcγ receptors. The first subject was homozygous for CD16b-NA1, the second subject was homozygous for CD16b-NA2 and the third subject was heterozygous and expressed both CD16b-NA1 and NA2. As shown in FIG. 11A, clone A2 was compared to an irrelevant negative control clone (CTL) and the positive control clone, 3G8, binding to each set of neutrophils using a serial dilution of each antibody ranging from 5×10⁻⁶ M to 10⁻¹¹ M. Clone 3G8 exhibited robust binding to all three neutrophil samples, with an EC50 of 1.4 to 2 nM. Clone A2 and the negative control clone exhibited no significant binding above background even at the highest antibody concentrations. Clone #2-2A2 with mutated Fcγ1 region to eliminate Fc binding activity to Fcγ receptors, exhibits no binding to Neutrophils from 2 donors expressing both CD16b-NA1 and NA2 (FIG. 11B).

Similar binding experiments were performed for other clones including the pan anti-human CD16 3G8 mouse monoclonal antibody (ThermoFisher Scientific, catalogue number MHCD1601). Neutrophils from two genotyped donors both with a NA1/NA2 genotype were purified from blood samples and incubated in the presence of a 4-fold serial dilution of each clone ranging from 50 nM to 0.003 nM for the 3G8 positive control, and 800 nM to 0.049 nM for the A2 family of clones carrying the mutated Fcγ1. After incubation on ice for 30 minutes, cells were washed and bound anti-CD16a IgG detected using FITC-labeled goat anti-human Fc F(ab)2 fragment (Invitrogen, catalogue number A24477). Curve fitting of the MFI values vs concentration was performed using Prism software. The positive control clone 3G8 had an apparent K_{D} value of 0.73 nM and 0.53 nM, and a maximum mean fluorescence intensity (to the nearest 1,000 MFI units) of 344,000 and 651,000 for neutrophil sample 1 and 2, respectively. In contrast, clones A2 and #2-2A2 exhibited no binding above background up to 1 μM, with clone #1-2A2 exhibiting only slight binding above background at 1 μM. The remaining A2 variants exhibited background binding up to a concentration of 40 nM, and the maximum MFI at 1 μM fell below 25% of the peak MFI value exhibited by the 3G8 clone, therefore a meaningful K_{D} value could not be determined (Table 31).

TABLE 31

| | KD | KD | Max MFI | Max MFI |
|---|---|---|---|---|
| Clone | (nM) #1 | (nM) #2 | (×1,000) #1 | (×1,000) #2 |
| 3G8 | 0.73 | 0.53 | 344 | 650 |
| A2 | NB | NB | 5.4 | 20 |
| | | | (background = 8.9) | (background = 18.6) |
| #1-A2 | N/A | N/A | 81 | 128 |
| #2-1A2 | N/A | N/A | 83 | 115 |
| #2-2A2 | N/A | N/A | 14.6 | 30.6 |
| | | | (background = 8.9) | (background = 18.6) |
| #2-3A2 | N/A | N/A | 99 | 248 |
| #10-A2 | N/A | N/A | 55 | 97 |

Similar binding experiments were performed for clone #55. As shown in FIG. 11C, clone #55 in IgG1 format with silenced Fc region exhibited no binding to Neutrophils from 3 different donors (the first subject was homozygous for CD16b-NA1, the second subject was homozygous for CD16b-NA2 and the third subject was heterozygous and expressed both CD16b-NA1 and NA2).

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

What is claimed is:

1. An antibody or fragment thereof that binds to CD16a, wherein the antibody or antigen binding fragment comprises:
(i) a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38;
(ii) a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76;
(iii) a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114;
(iv) a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152;
(v) a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190;
(vi) a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228;
(vii) a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266;
(vii) a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304; or
(ix) a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342.

2. The antibody or fragment thereof of claim 1, wherein the antibody or fragment thereof comprises:
(a) a VH region comprising:
(1) a VH CDR1 having an amino acid sequence selected from the group consisting of SEQ ID NOs: 1, 7, 13, 19, 25, and 31;
(2) a VH CDR2 having an amino acid sequence selected from the group consisting of SEQ ID NOs: 2, 8, 14, 20, 26, and 32; and
(3) a VH CDR3 having an amino acid sequence selected from the group consisting of SEQ ID NOs: 3, 9, 15, 21, 27, and 33;
and
(b) a VL region comprising:
(1) a VL CDR1 having an amino acid sequence selected from the group consisting of SEQ ID NOs: 4, 10, 16, 22, 28, and 34;
(2) a VL CDR2 having an amino acid sequence selected from the group consisting of SEQ ID NOs: 5, 11, 17, 23, 29, and 35; and
(3) a VL CDR3 having an amino acid sequence selected from the group consisting of SEQ ID NOs: 6, 12, 18, 24, 30, and 36.

3. The antibody or fragment thereof of claim 1, wherein the antibody or fragment thereof comprises any one or more of (i)-(vi):
(i) a VH region comprising a VH CDR1 comprising the amino acid sequence of SEQ ID NO: 1, a VH CDR2 comprising the amino acid sequence of SEQ ID NO: 2, a VH CDR3 comprising the amino acid sequence of SEQ ID NO: 3; and a VL region comprising a VL CDR1 comprising the amino acid sequence of SEQ ID NO: 4, a VL CDR2 comprising the amino acid sequence of SEQ ID NO: 5, and a VL CDR3 comprising the amino acid sequence of SEQ ID NO: 6;
(ii) a VH region comprising a VH CDR1 comprising the amino acid sequence of SEQ ID NO: 7, a VH CDR2 comprising the amino acid sequence of SEQ ID NO: 8, a VH CDR3 comprising the amino acid sequence of SEQ ID NO: 9; and a VL region comprising a VL CDR1 comprising the amino acid sequence of SEQ ID NO: 10, a VL CDR2 comprising the amino acid sequence of SEQ ID NO: 11, and a VL CDR3 comprising the amino acid sequence of SEQ ID NO: 12;
(iii) a VH region comprising a VH CDR1 comprising the amino acid sequence of SEQ ID NO: 13, a VH CDR2 comprising the amino acid sequence of SEQ ID NO: 14, a VH CDR3 comprising the amino acid sequence of SEQ ID NO: 15; and a VL region comprising a VL CDR1 comprising the amino acid sequence of SEQ ID NO: 16, a VL CDR2 comprising the amino acid sequence of SEQ ID NO: 17, and a VL CDR3 comprising the amino acid sequence of SEQ ID NO: 18;
(iv) a VH region comprising a VH CDR1 comprising the amino acid sequence of SEQ ID NO: 19, a VH CDR2 comprising the amino acid sequence of SEQ ID NO: 20, a VH CDR3 comprising the amino acid sequence of SEQ ID NO: 21; and a VL region comprising a VL CDR1 comprising the amino acid sequence of SEQ ID NO: 22, a VL CDR2 comprising the amino acid sequence of SEQ ID NO: 23, and a VL CDR3 comprising the amino acid sequence of SEQ ID NO: 24;
(v) a VH region comprising a VH CDR1 comprising the amino acid sequence of SEQ ID NO: 25, a VH CDR2 comprising the amino acid sequence of SEQ ID NO: 26, a VH CDR3 comprising the amino acid sequence of SEQ ID NO: 27; and a VL region comprising a VL CDR1 comprising the amino acid sequence of SEQ ID NO: 28, a VL CDR2 comprising the amino acid sequence of SEQ ID NO: 29, and a VL CDR3 comprising the amino acid sequence of SEQ ID NO: 30; or
(vi) a VH region comprising a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33; and a VL region comprising a VL CDR1 comprising the amino acid sequence of SEQ ID NO: 34, a VL CDR2 comprising the amino acid sequence of SEQ ID NO: 35, and a VL CDR3 comprising the amino acid sequence of SEQ ID NO: 36.

4. The antibody or fragment thereof of claim 1, wherein the antibody or fragment thereof comprises
(a) a VH region comprising:
(1) a VH CDR1 having an amino acid sequence selected from the group consisting of SEQ ID NOs: 39, 45, 51, 57, 63, and 69;
(2) a VH CDR2 having an amino acid sequence selected from the group consisting of SEQ ID NOs: 40, 46, 52, 58, 64, and 70; and
(3) a VH CDR3 having an amino acid sequence selected from the group consisting of SEQ ID NOs: 41, 47, 53, 59, 65, and 71;
and
(b) a VL region comprising:
(1) a VL CDR1 having an amino acid sequence selected from the group consisting of SEQ ID NOs: 42, 48, 54, 60, 66, and 72;
(2) a VL CDR2 having an amino acid sequence selected from the group consisting of SEQ ID NOs: 43, 49, 55, 61, 67, and 73; and
(3) a VL CDR3 having an amino acid sequence selected from the group consisting of SEQ ID NOs: 44, 50, 56, 62, 68, and 74.

5. The antibody or fragment thereof of claim 1, wherein the antibody or fragment thereof comprises any one or more of (i)-(vi):
(i) a VH region comprising a VH CDR1 comprising the amino acid sequence of SEQ ID NO: 39, a VH CDR2 comprising the amino acid sequence of SEQ ID NO: 40, and a VH CDR3 comprising the amino acid sequence of SEQ ID NO: 41; and a VL region comprising a VL CDR1 comprising the amino acid sequence of SEQ ID NO: 42, a VL CDR2 comprising the amino acid sequence of SEQ ID NO: 43, and a VL CDR3 comprising the amino acid sequence of SEQ ID NO: 44;
(ii) a VH region comprising a VH CDR1 comprising the amino acid sequence of SEQ ID NO: 45, a VH CDR2 comprising the amino acid sequence of SEQ ID NO: 46, and a VH CDR3 comprising the amino acid sequence of SEQ ID NO: 47; and a VL region comprising a VL CDR1 comprising the amino acid sequence of SEQ ID NO: 48, a VL CDR2 comprising the amino acid sequence of SEQ ID NO: 49, and a VL CDR3 comprising the amino acid sequence of SEQ ID NO: 50;
(iii) a VH region comprising a VH CDR1 comprising the amino acid sequence of SEQ ID NO: 51, a VH CDR2 comprising the amino acid sequence of SEQ ID NO: 52, and a VH CDR3 comprising the amino acid sequence of SEQ ID NO: 53; and a VL region comprising a VL CDR1 comprising the amino acid sequence of SEQ ID NO: 54, a VL CDR2 comprising the amino acid sequence of SEQ ID NO: 55, and a VL CDR3 comprising the amino acid sequence of SEQ ID NO: 56;
(iv) a VH region comprising a VH CDR1 comprising the amino acid sequence of SEQ ID NO: 57, a VH CDR2 comprising the amino acid sequence of SEQ ID NO: 58, and a VH CDR3 comprising the amino acid sequence of SEQ ID NO: 59; and a VL region comprising a VL CDR1 comprising the amino acid sequence of SEQ ID NO: 60, a VL CDR2 comprising the amino acid sequence of SEQ ID NO: 61, and a VL CDR3 comprising the amino acid sequence of SEQ ID NO: 62;
(v) a VH region comprising a VH CDR1 comprising the amino acid sequence of SEQ ID NO: 63, a VH CDR2 comprising the amino acid sequence of SEQ ID NO: 64, and a VH CDR3 comprising the amino acid sequence of SEQ ID NO: 65; and a VL region comprising a VL CDR1 comprising the amino acid sequence of SEQ ID NO: 66, a VL CDR2 comprising the amino acid sequence of SEQ ID NO: 67, and a VL CDR3 comprising the amino acid sequence of SEQ ID NO: 68; or
(vi) a VH region comprising a VH CDR1 comprising the amino acid sequence of SEQ ID NO: 69, a VH CDR2 comprising the amino acid sequence of SEQ ID NO: 70, and a VH CDR3 comprising the amino acid sequence of SEQ ID NO: 71; and a VL region comprising a VL CDR1 comprising the amino acid sequence of SEQ ID NO: 72, a VL CDR2 comprising the amino acid sequence of SEQ ID NO: 73, and a VL CDR3 comprising the amino acid sequence of SEQ ID NO: 74.

6. The antibody or fragment thereof of claim 1, wherein the antibody or fragment thereof comprises:
(a) a VH region comprising:
(1) a VH CDR1 having an amino acid sequence selected from the group consisting of SEQ ID NOs: 77, 83, 89, 95, 101, and 107;
(2) a VH CDR2 having an amino acid sequence selected from the group consisting of SEQ ID NOs: 78, 84, 90, 96, 102, and 108; and
(3) a VH CDR3 having an amino acid sequence selected from the group consisting of SEQ ID NOs: 79, 85, 91, 97, 103, and 109;
and
(b) a VL region comprising:
(1) a VL CDR1 having an amino acid sequence selected from the group consisting of SEQ ID NOs: 80, 86, 92, 98, 104, and 110;
(2) a VL CDR2 having an amino acid sequence selected from the group consisting of SEQ ID NOs: 81, 87, 93, 99, 105, and 111; and
(3) a VL CDR3 having an amino acid sequence selected from the group consisting of SEQ ID NOs: 82, 88, 94, 100, 106, and 112.

7. The antibody or fragment thereof of claim 1, wherein the antibody or fragment thereof comprises any one or more of (i)-(vi):
(i) a VH region comprising a VH CDR1 comprising the amino acid sequence of SEQ ID NO: 77, a VH CDR2 comprising the amino acid sequence of SEQ ID NO: 78, and a VH CDR3 comprising the amino acid sequence of SEQ ID NO: 79; and a VL region comprising a VL CDR1 comprising the amino acid sequence of SEQ ID NO: 80, a VL CDR2 comprising the amino acid sequence of SEQ ID NO: 81, and a VL CDR3 comprising the amino acid sequence of SEQ ID NO: 82;
(ii) a VH region comprising a VH CDR1 comprising the amino acid sequence of SEQ ID NO: 83, a VH CDR2 comprising the amino acid sequence of SEQ ID NO: 84, and a VH CDR3 comprising the amino acid sequence of SEQ ID NO: 85; and a VL region comprising a VL CDR1 comprising the amino acid sequence of SEQ ID NO: 86, a VL CDR2 comprising the amino acid sequence of SEQ ID NO: 87, and a VL CDR3 comprising the amino acid sequence of SEQ ID NO: 88;
(iii) a VH region comprising a VH CDR1 comprising the amino acid sequence of SEQ ID NO: 89, a VH CDR2 comprising the amino acid sequence of SEQ ID NO: 90, and a VH CDR3 comprising the amino acid sequence of SEQ ID NO: 91; and a VL region comprising a VL CDR1 comprising the amino acid sequence of SEQ ID NO: 92, a VL CDR2 comprising the amino acid sequence of SEQ ID NO: 93, and a VL CDR3 comprising the amino acid sequence of SEQ ID NO: 94;
(iv) a VH region comprising a VH CDR1 comprising the amino acid sequence of SEQ ID NO: 95, a VH CDR2 comprising the amino acid sequence of SEQ ID NO: 96, and a VH CDR3 comprising the amino acid sequence of SEQ ID NO: 97; and a VL region comprising a VL CDR1 comprising the amino acid sequence of SEQ ID NO: 98, a VL CDR2 comprising the amino acid sequence of SEQ ID NO: 99, and a VL CDR3 comprising the amino acid sequence of SEQ ID NO: 100;
(v) a VH region comprising a VH CDR1 comprising the amino acid sequence of SEQ ID NO: 101, a VH CDR2 comprising the amino acid sequence of SEQ ID NO: 102, and a VH CDR3 comprising the amino acid sequence of SEQ ID NO: 103; and a VL region comprising a VL CDR1 comprising the amino acid sequence of SEQ ID NO: 104, a VL CDR2 comprising the amino acid sequence of SEQ ID NO: 105, and a VL CDR3 comprising the amino acid sequence of SEQ ID NO: 106; or
(vi) a VH region comprising a VH CDR1 comprising the amino acid sequence of SEQ ID NO: 107, a VH CDR2 comprising the amino acid sequence of SEQ ID NO: 108, and a VH CDR3 comprising the amino acid sequence of SEQ ID NO: 109; and a VL region comprising a VL CDR1 comprising the amino acid sequence of SEQ ID NO: 110, a VL CDR2 comprising the amino acid sequence of SEQ ID NO: 111, and a VL CDR3 comprising the amino acid sequence of SEQ ID NO: 112.

8. The antibody or fragment thereof of claim 1, wherein the antibody or fragment thereof comprises:
(a) a VH region comprising:
(1) a VH CDR1 having an amino acid sequence selected from the group consisting of SEQ ID NOs: 115, 121, 127, 133, 139, and 145;
(2) a VH CDR2 having an amino acid sequence selected from the group consisting of SEQ ID NOs: 116, 122, 128, 134, 140, and 146; and
(3) a VH CDR3 having an amino acid sequence selected from the group consisting of SEQ ID NOs: 117, 123, 129, 135, 141, and 147;
and
(b) a VL region comprising:
(1) a VL CDR1 having an amino acid sequence selected from the group consisting of SEQ ID NOs: 118, 124, 130, 136, 142, and 148;
(2) a VL CDR2 having an amino acid sequence selected from the group consisting of SEQ ID NOs: 119, 125, 131, 137, 143, and 149; and
(3) a VL CDR3 having an amino acid sequence selected from the group consisting of SEQ ID NOs: 120, 126, 132, 138, 144, and 150.

9. The antibody or fragment thereof of claim 1, wherein the antibody or fragment thereof comprises any one or more of (i)-(vi):
(i) a VH region comprising a VH CDR1 comprising the amino acid sequence of SEQ ID NO: 115, a VH CDR2 comprising the amino acid sequence of SEQ ID NO: 116, and a VH CDR3 comprising the amino acid sequence of SEQ ID NO: 117; and a VL region comprising a VL CDR1 comprising the amino acid sequence of SEQ ID NO: 118, a VL CDR2 comprising the amino acid sequence of SEQ ID NO: 119, and a VL CDR3 comprising the amino acid sequence of SEQ ID NO: 120;
(ii) a VH region comprising a VH CDR1 comprising the amino acid sequence of SEQ ID NO: 121, a VH CDR2 comprising the amino acid sequence of SEQ ID NO: 122, and a VH CDR3 comprising the amino acid sequence of SEQ ID NO: 123; and a VL region comprising a VL CDR1 comprising the amino acid sequence of SEQ ID NO: 124, a VL CDR2 comprising the amino acid sequence of SEQ ID NO: 125, and a VL CDR3 comprising the amino acid sequence of SEQ ID NO: 126;
(iii) a VH region comprising a VH CDR1 comprising the amino acid sequence of SEQ ID NO: 127, a VH CDR2 comprising the amino acid sequence of SEQ ID NO: 128, and a VH CDR3 comprising the amino acid sequence of SEQ ID NO: 129; and a VL region comprising a VL CDR1 comprising the amino acid sequence of SEQ ID NO: 130, a VL CDR2 comprising the amino acid sequence of SEQ ID NO: 131, and a VL CDR3 comprising the amino acid sequence of SEQ ID NO: 132;
(iv) a VH region comprising a VH CDR1 comprising the amino acid sequence of SEQ ID NO: 133, a VH CDR2 comprising the amino acid sequence of SEQ ID NO: 134, and a VH CDR3 comprising the amino acid sequence of SEQ ID NO: 135; and a VL region comprising a VL CDR1 comprising the amino acid sequence of SEQ ID NO: 136, a VL CDR2 comprising the amino acid sequence of SEQ ID NO: 137, and a VL CDR3 comprising the amino acid sequence of SEQ ID NO: 138;
(v) a VH region comprising a VH CDR1 comprising the amino acid sequence of SEQ ID NO: 139, a VH CDR2 comprising the amino acid sequence of SEQ ID NO: 140, and a VH CDR3 comprising the amino acid sequence of SEQ ID NO: 141; and a VL region comprising a VL CDR1 comprising the amino acid sequence of SEQ ID NO: 142, a VL CDR2 comprising the amino acid sequence of SEQ ID NO: 143, and a VL CDR3 comprising the amino acid sequence of SEQ ID NO: 144; or
(vi) a VH region comprising a VH CDR1 comprising the amino acid sequence of SEQ ID NO: 145, a VH CDR2 comprising the amino acid sequence of SEQ ID NO: 146, and a VH CDR3 comprising the amino acid sequence of SEQ ID NO: 147; and a VL region comprising a VL CDR1 comprising the amino acid sequence of SEQ ID NO: 148, a VL CDR2 comprising the amino acid sequence of SEQ ID NO: 149, and a VL CDR3 comprising the amino acid sequence of SEQ ID NO: 150.

10. The antibody or fragment thereof of claim 1, wherein the antibody or fragment thereof comprises
(a) a VH region comprising:
(1) a VH CDR1 having an amino acid sequence selected from the group consisting of SEQ ID NOs: 153, 159, 165, 171, 177, and 183;
(2) a VH CDR2 having an amino acid sequence selected from the group consisting of SEQ ID NOs: 154, 160, 166, 172, 178, and 184; and
(3) a VH CDR3 having an amino acid sequence selected from the group consisting of SEQ ID NOs: 155, 161, 167, 173, 179, and 185;
and
(b) a VL region comprising:
(1) a VL CDR1 having an amino acid sequence selected from the group consisting of SEQ ID NOs: 156, 162, 168, 174, 180, and 186;
(2) a VL CDR2 having an amino acid sequence selected from the group consisting of SEQ ID NOs: 157, 163, 169, 175, 181, and 187; and
(3) a VL CDR3 having an amino acid sequence selected from the group consisting of SEQ ID NOs: 158, 164, 170, 176, 182, and 188.

11. The antibody or fragment thereof of claim 1, wherein the antibody or fragment thereof comprises any one or more of (i)-(vi):
(i) a VH region comprising a VH CDR1 comprising the amino acid sequence of SEQ ID NO: 153, a VH CDR2 comprising the amino acid sequence of SEQ ID NO: 154, and a VH CDR3 comprising the amino acid sequence of SEQ ID NO: 155; and a VL region comprising a VL CDR1 comprising the amino acid sequence of SEQ ID NO: 156, a VL CDR2 comprising the amino acid sequence of SEQ ID NO: 157, and a VL CDR3 comprising the amino acid sequence of SEQ ID NO: 158;
(ii) a VH region comprising a VH CDR1 comprising the amino acid sequence of SEQ ID NO: 159, a VH CDR2 comprising the amino acid sequence of SEQ ID NO: 160, and a VH CDR3 comprising the amino acid sequence of SEQ ID NO: 161; and a VL region comprising a VL CDR1 comprising the amino acid sequence of SEQ ID NO: 162, a VL CDR2 comprising the amino acid sequence of SEQ ID NO: 163, and a VL CDR3 comprising the amino acid sequence of SEQ ID NO: 164;
(iii) a VH region comprising a VH CDR1 comprising the amino acid sequence of SEQ ID NO: 165, a VH CDR2 comprising the amino acid sequence of SEQ ID NO: 166, and a VH CDR3 comprising the amino acid sequence of SEQ ID NO: 167; and a VL region comprising a VL CDR1 comprising the amino acid sequence of SEQ ID NO: 168, a VL CDR2 comprising the amino acid sequence of SEQ ID NO: 169, and a VL CDR3 comprising the amino acid sequence of SEQ ID NO: 170;
(iv) a VH region comprising a VH CDR1 comprising the amino acid sequence of SEQ ID NO: 171, a VH CDR2 comprising the amino acid sequence of SEQ ID NO: 172, and a VH CDR3 comprising the amino acid sequence of SEQ ID NO: 173; and a VL region comprising a VL CDR1 comprising the amino acid sequence of SEQ ID NO: 174, a VL CDR2 comprising the amino acid sequence of SEQ ID NO: 175, and a VL CDR3 comprising the amino acid sequence of SEQ ID NO: 176;
(v) a VH region comprising a VH CDR1 comprising the amino acid sequence of SEQ ID NO: 177, a VH CDR2 comprising the amino acid sequence of SEQ ID NO: 178, and a VH CDR3 comprising the amino acid sequence of SEQ ID NO: 179; and a VL region comprising a VL CDR1 comprising the amino acid sequence of SEQ ID NO: 180, a VL CDR2 comprising the amino acid sequence of SEQ ID NO: 181, and a VL CDR3 comprising the amino acid sequence of SEQ ID NO: 182; or
(vi) a VH region comprising a VH CDR1 comprising the amino acid sequence of SEQ ID NO: 183, a VH CDR2 comprising the amino acid sequence of SEQ ID NO: 184, and a VH CDR3 comprising the amino acid sequence of SEQ ID NO: 185; and a VL region comprising a VL CDR1 comprising the amino acid sequence of SEQ ID NO: 186, a VL CDR2 comprising the amino acid sequence of SEQ ID NO: 187, and a VL CDR3 comprising the amino acid sequence of SEQ ID NO: 188.

12. The antibody or fragment thereof of claim 1, wherein the antibody or fragment thereof comprises:
(a) a VH region comprising:
(1) a VH CDR1 having an amino acid sequence selected from the group consisting of SEQ ID NOs: 191, 197, 203, 209, 215, and 221;
(2) a VH CDR2 having an amino acid sequence selected from the group consisting of SEQ ID NOs: 192, 198, 204, 210, 216, and 222; and
(3) a VH CDR3 having an amino acid sequence selected from the group consisting of SEQ ID NOs: 193, 199, 205, 211, 217, and 223;
and
(b) a VL region comprising:
(1) a VL CDR1 having an amino acid sequence selected from the group consisting of SEQ ID NOs: 194, 200, 206, 212, 218, and 224;
(2) a VL CDR2 having an amino acid sequence selected from the group consisting of SEQ ID NOs: 195, 201, 207, 213, 219, and 225; and
(3) a VL CDR3 having an amino acid sequence selected from the group consisting of SEQ ID NOs: 196, 202, 208, 214, 220, and 226.

13. The antibody or fragment thereof of claim 1, wherein the antibody or fragment thereof comprises any one or more of (i)-(vi):
(i) a VH region comprising a VH CDR1 comprising the amino acid sequence of SEQ ID NO: 191, a VH CDR2 comprising the amino acid sequence of SEQ ID NO: 192, and a VH CDR3 comprising the amino acid sequence of SEQ ID NO: 193; and a VL region comprising a VL CDR1 comprising the amino acid sequence of SEQ ID NO: 194, a VL CDR2 comprising the amino acid sequence of SEQ ID NO: 195, and a VL CDR3 comprising the amino acid sequence of SEQ ID NO: 196;
(ii) a VH region comprising a VH CDR1 comprising the amino acid sequence of SEQ ID NO: 197, a VH CDR2 comprising the amino acid sequence of SEQ ID NO: 198, and a VH CDR3 comprising the amino acid sequence of SEQ ID NO: 199; and a VL region comprising a VL CDR1 comprising the amino acid sequence of SEQ ID NO: 200, a VL CDR2 comprising the amino acid sequence of SEQ ID NO: 201, and a VL CDR3 comprising the amino acid sequence of SEQ ID NO: 202;
(iii) a VH region comprising a VH CDR1 comprising the amino acid sequence of SEQ ID NO: 203, a VH CDR2 comprising the amino acid sequence of SEQ ID NO: 204, and a VH CDR3 comprising the amino acid sequence of SEQ ID NO: 205; and a VL region comprising a VL CDR1 comprising the amino acid sequence of SEQ ID NO: 206, a VL CDR2 comprising the amino acid sequence of SEQ ID NO: 207, and a VL CDR3 comprising the amino acid sequence of SEQ ID NO: 208;
(iv) a VH region comprising a VH CDR1 comprising the amino acid sequence of SEQ ID NO: 209, a VH CDR2 comprising the amino acid sequence of SEQ ID NO: 210, and a VH CDR3 comprising the amino acid sequence of SEQ ID NO: 211; and a VL region comprising a VL CDR1 comprising the amino acid sequence of SEQ ID NO: 212, a VL CDR2 comprising the amino acid sequence of SEQ ID NO: 213, and a VL CDR3 comprising the amino acid sequence of SEQ ID NO: 214;
(v) a VH region comprising a VH CDR1 comprising the amino acid sequence of SEQ ID NO: 215, a VH CDR2 comprising the amino acid sequence of SEQ ID NO: 216, and a VH CDR3 comprising the amino acid sequence of SEQ ID NO: 217; and a VL region comprising a VL CDR1 comprising the amino acid sequence of SEQ ID NO: 218, a VL CDR2 comprising the amino acid sequence of SEQ ID NO: 219, and a VL CDR3 comprising the amino acid sequence of SEQ ID NO: 220; or
(vi) a VH region comprising a VH CDR1 comprising the amino acid sequence of SEQ ID NO: 221, a VH CDR2 comprising the amino acid sequence of SEQ ID NO: 222, and a VH CDR3 comprising the amino acid sequence of SEQ ID NO: 223; and a VL region comprising a VL CDR1 comprising the amino acid sequence of SEQ ID NO: 224, a VL CDR2 comprising the amino acid sequence of SEQ ID NO: 225, and a VL CDR3 comprising the amino acid sequence of SEQ ID NO: 226.

14. The antibody or fragment thereof of claim 1, wherein the antibody or fragment thereof comprises:
(a) a VH region comprising:
(1) a VH CDR1 having an amino acid sequence selected from the group consisting of SEQ ID NOs: 229, 235, 241, 247, 253, and 259;
(2) a VH CDR2 having an amino acid sequence selected from the group consisting of SEQ ID NOs: 230, 236, 242, 248, 254, and 260; and
(3) a VH CDR3 having an amino acid sequence selected from the group consisting of SEQ ID NOs: 231, 237, 243, 249, 255, and 261;
and
(b) a VL region comprising:
(1) a VL CDR1 having an amino acid sequence selected from the group consisting of SEQ ID NOs: 232, 238, 244, 250, 256, and 262;
(2) a VL CDR2 having an amino acid sequence selected from the group consisting of SEQ ID NOs: 233, 239, 245, 251, 257, and 263; and
(3) a VL CDR3 having an amino acid sequence selected from the group consisting of SEQ ID NOs: 234, 240, 246, 252, 258, and 264.

15. The antibody or fragment thereof of claim 1, wherein the antibody or fragment thereof comprises any one or more of (i)-(vi):
(i) a VH region comprising a VH CDR1 comprising the amino acid sequence of SEQ ID NO: 229, a VH CDR2 comprising the amino acid sequence of SEQ ID NO: 230, and a VH CDR3 comprising the amino acid sequence of SEQ ID NO: 231; and a VL region comprising a VL CDR1 comprising the amino acid sequence of SEQ ID NO: 232, a VL CDR2 comprising the amino acid sequence of SEQ ID NO: 233, and a VL CDR3 comprising the amino acid sequence of SEQ ID NO: 234;
(ii) a VH region comprising a VH CDR1 comprising the amino acid sequence of SEQ ID NO: 235 a VH CDR2 comprising the amino acid sequence of SEQ ID NO: 236, and a VH CDR3 comprising the amino acid sequence of SEQ ID NO: 237; and a VL region comprising a VL CDR1 comprising the amino acid sequence of SEQ ID NO: 238, a VL CDR2 comprising the amino acid sequence of SEQ ID NO: 239, and a VL CDR3 comprising the amino acid sequence of SEQ ID NO: 240;
(iii) a VH region comprising a VH CDR1 comprising the amino acid sequence of SEQ ID NO: 241, a VH CDR2 comprising the amino acid sequence of SEQ ID NO: 242, and a VH CDR3 comprising the amino acid sequence of SEQ ID NO: 243; and a VL region comprising a VL CDR1 comprising the amino acid sequence of SEQ ID NO: 244, a VL CDR2 comprising the amino acid sequence of SEQ ID NO: 245, and a VL CDR3 comprising the amino acid sequence of SEQ ID NO: 246;
(iv) a VH region comprising a VH CDR1 comprising the amino acid sequence of SEQ ID NO: 247, a VH CDR2 comprising the amino acid sequence of SEQ ID NO: 248, and a VH CDR3 comprising the amino acid sequence of SEQ ID NO: 249; and a VL region comprising a VL CDR1 comprising the amino acid sequence of SEQ ID NO: 250, a VL CDR2 comprising the amino acid sequence of SEQ ID NO: 251, and a VL CDR3 comprising the amino acid sequence of SEQ ID NO: 252;
(v) a VH region comprising a VH CDR1 comprising the amino acid sequence of SEQ ID NO: 253, a VH CDR2 comprising the amino acid sequence of SEQ ID NO: 254, and a VH CDR3 comprising the amino acid sequence of SEQ ID NO: 255; and a VL region comprising a VL CDR1 comprising the amino acid sequence of SEQ ID NO: 256, a VL CDR2 comprising the amino acid sequence of SEQ ID NO: 257, and a VL CDR3 comprising the amino acid sequence of SEQ ID NO: 258; or
(vi) a VH region comprising a VH CDR1 comprising the amino acid sequence of SEQ ID NO: 259, a VH CDR2 comprising the amino acid sequence of SEQ ID NO: 260, and a VH CDR3 comprising the amino acid sequence of SEQ ID NO: 261; and a VL region comprising a VL CDR1 comprising the amino acid sequence of SEQ ID NO: 262, a VL CDR2 comprising the amino acid sequence of SEQ ID NO: 263, and a VL CDR3 comprising the amino acid sequence of SEQ ID NO: 264.

16. The antibody or fragment thereof of claim 1, wherein the antibody or fragment thereof comprises:
(a) a VH region comprising:
(1) a VH CDR1 having an amino acid sequence selected from the group consisting of SEQ ID NOs: 267, 273, 279, 285, 291, and 297;
(2) a VH CDR2 having an amino acid sequence selected from the group consisting of SEQ ID NOs: 268, 274, 280, 286, 292, and 298; and
(3) a VH CDR3 having an amino acid sequence selected from the group consisting of SEQ ID NOs: 269, 275, 281, 287, 293, and 299;
and
(b) a VL region comprising:
(1) a VL CDR1 having an amino acid sequence selected from the group consisting of SEQ ID NOs: 270, 276, 282, 288, 294, and 300;
(2) a VL CDR2 having an amino acid sequence selected from the group consisting of SEQ ID NOs: 271, 277, 283, 289, 295, and 301; and
(3) a VL CDR3 having an amino acid sequence selected from the group consisting of SEQ ID NOs: 272, 278, 284, 290, 296, and 302.

17. The antibody or fragment thereof of claim 1, wherein the antibody or fragment thereof comprises any one or more of (i)-(ix):
(i) a VH region comprising a VH CDR1 comprising the amino acid sequence of SEQ ID NO: 267, a VH CDR2 comprising the amino acid sequence of SEQ ID NO: 268, and a VH CDR3 comprising the amino acid sequence of SEQ ID NO: 269; and a VL region comprising a VL CDR1 comprising the amino acid sequence of SEQ ID NO: 270, a VL CDR2 comprising the amino acid sequence of SEQ ID NO: 271, and a VL CDR3 comprising the amino acid sequence of SEQ ID NO: 272;
(ii) a VH region comprising a VH CDR1 comprising the amino acid sequence of SEQ ID NO: 273 a VH CDR2 comprising the amino acid sequence of SEQ ID NO: 274, and a VH CDR3 comprising the amino acid sequence of SEQ ID NO: 275; and a VL region comprising a VL CDR1 comprising the amino acid sequence of SEQ ID NO: 276, a VL CDR2 comprising the amino acid sequence of SEQ ID NO: 277, and a VL CDR3 comprising the amino acid sequence of SEQ ID NO: 278;
(iii) a VH region comprising a VH CDR1 comprising the amino acid sequence of SEQ ID NO: 279, a VH CDR2 comprising the amino acid sequence of SEQ ID NO: 280, and a VH CDR3 comprising the amino acid sequence of SEQ ID NO: 281; and a VL region comprising a VL CDR1 comprising the amino acid sequence of SEQ ID NO: 282, a VL CDR2 comprising the amino acid sequence of SEQ ID NO: 283, and a VL CDR3 comprising the amino acid sequence of SEQ ID NO: 284;
(iv) a VH region comprising a VH CDR1 comprising the amino acid sequence of SEQ ID NO: 285, a VH CDR2 comprising the amino acid sequence of SEQ ID NO: 286, and a VH CDR3 comprising the amino acid sequence of SEQ ID NO: 287; and a VL region comprising a VL CDR1 comprising the amino acid sequence of SEQ ID NO: 288, a VL CDR2 comprising the amino acid sequence of SEQ ID NO: 289, and a VL CDR3 comprising the amino acid sequence of SEQ ID NO: 290;
(v) a VH region comprising a VH CDR1 comprising the amino acid sequence of SEQ ID NO: 291, a VH CDR2 comprising the amino acid sequence of SEQ ID NO: 292, and a VH CDR3 comprising the amino acid sequence of SEQ ID NO: 293; and a VL region comprising a VL CDR1 comprising the amino acid sequence of SEQ ID NO: 294, a VL CDR2 comprising the amino acid sequence of SEQ ID NO: 295, and a VL CDR3 comprising the amino acid sequence of SEQ ID NO: 296; or
(vi) a VH region comprising a VH CDR1 comprising the amino acid sequence of SEQ ID NO: 297, a VH CDR2 comprising the amino acid sequence of SEQ ID NO: 298, and a VH CDR3 comprising the amino acid sequence of SEQ ID NO: 299; and a VL region comprising a VL CDR1 comprising the amino acid sequence of SEQ ID NO: 300, a VL CDR2 comprising the amino acid sequence of SEQ ID NO: 301, and a VL CDR3 comprising the amino acid sequence of SEQ ID NO: 302.

18. The antibody or fragment thereof of claim 1, wherein the antibody or fragment thereof comprises:
(a) a VH region comprising:
(1) a VH CDR1 having an amino acid sequence selected from the group consisting of SEQ ID NOs: 305, 311, 317, 323, 329, and 335;
(2) a VH CDR2 having an amino acid sequence selected from the group consisting of SEQ ID NOs: 306, 312, 318, 324, 330, and 336; and
(3) a VH CDR3 having an amino acid sequence selected from the group consisting of SEQ ID NOs: 307, 313, 319, 325, 331, and 337;
and
(b) a VL region comprising:
(1) a VL CDR1 having an amino acid sequence selected from the group consisting of SEQ ID NOs: 308, 314, 320, 326, 332, and 338;
(2) a VL CDR2 having an amino acid sequence selected from the group consisting of SEQ ID NOs: 309, 315, 321, 327, 333, and 339; and
(3) a VL CDR3 having an amino acid sequence selected from the group consisting of SEQ ID NOs: 310, 316, 322, 328, 334, and 340.

19. The antibody or fragment thereof of claim 1, wherein the antibody or fragment thereof comprises any one or more of (i)-(vi):
(i) a VH region comprising a VH CDR1 comprising the amino acid sequence of SEQ ID NO: 305, a VH CDR2 comprising the amino acid sequence of SEQ ID NO: 306, and a VH CDR3 comprising the amino acid sequence of SEQ ID NO: 307; and a VL region comprising a VL CDR1 comprising the amino acid sequence of SEQ ID NO: 308, a VL CDR2 comprising the amino acid sequence of SEQ ID NO: 309, and a VL CDR3 comprising the amino acid sequence of SEQ ID NO: 310;
(ii) a VH region comprising a VH CDR1 comprising the amino acid sequence of SEQ ID NO: 311 a VH CDR2 comprising the amino acid sequence of SEQ ID NO: 312, and a VH CDR3 comprising the amino acid sequence of SEQ ID NO: 313; and a VL region comprising a VL CDR1 comprising the amino acid sequence of SEQ ID NO: 314, a VL CDR2 comprising the amino acid sequence of SEQ ID NO: 315, and a VL CDR3 comprising the amino acid sequence of SEQ ID NO: 316;
(iii) a VH region comprising a VH CDR1 comprising the amino acid sequence of SEQ ID NO: 317, a VH CDR2 comprising the amino acid sequence of SEQ ID NO: 318, and a VH CDR3 comprising the amino acid sequence of SEQ ID NO: 319; and a VL region comprising a VL CDR1 comprising the amino acid sequence of SEQ ID NO: 320, a VL CDR2 comprising the amino acid sequence of SEQ ID NO: 321, and a VL CDR3 comprising the amino acid sequence of SEQ ID NO: 322;
(iv) a VH region comprising a VH CDR1 comprising the amino acid sequence of SEQ ID NO: 323, a VH CDR2 comprising the amino acid sequence of SEQ ID NO: 324, and a VH CDR3 comprising the amino acid sequence of SEQ ID NO: 325; and a VL region comprising a VL CDR1 comprising the amino acid sequence of SEQ ID NO: 326, a VL CDR2 comprising the amino acid sequence of SEQ ID NO: 327, and a VL CDR3 comprising the amino acid sequence of SEQ ID NO: 328;
(v) a VH region comprising a VH CDR1 comprising the amino acid sequence of SEQ ID NO: 329, a VH CDR2 comprising the amino acid sequence of SEQ ID NO: 330, and a VH CDR3 comprising the amino acid sequence of SEQ ID NO: 331; and a VL region comprising a VL CDR1 comprising the amino acid sequence of SEQ ID NO: 332, a VL CDR2 comprising the amino acid sequence of SEQ ID NO: 333, and a VL CDR3 comprising the amino acid sequence of SEQ ID NO: 334; or
(vi) a VH region comprising a VH CDR1 comprising the amino acid sequence of SEQ ID NO: 335, a VH CDR2 comprising the amino acid sequence of SEQ ID NO: 336, and a VH CDR3 comprising the amino acid sequence of SEQ ID NO: 337; and a VL region comprising a VL CDR1 comprising the amino acid sequence of SEQ ID NO: 338, a VL CDR2 comprising the amino acid sequence of SEQ ID NO: 339, and a VL CDR3 comprising the amino acid sequence of SEQ ID NO: 340.

20. The antibody or fragment thereof of claim 1, wherein the antibody or fragment thereof comprises:
(i) a VH comprising the amino acid sequence of SEQ ID NO: 37 and a VL comprising the amino acid sequence of SEQ ID NO: 38;
(ii) a VH comprising the amino acid sequence of SEQ ID NO: 75 and a VL comprising the amino acid sequence of SEQ ID NO: 76;
(iii) a VH comprising the amino acid sequence of SEQ ID NO: 113 and a VL comprising the amino acid sequence of SEQ ID NO: 114;
(iv) a VH comprising the amino acid sequence of SEQ ID NO: 151 and a VL comprising the amino acid sequence of SEQ ID NO: 152;
(v) a VH comprising the amino acid sequence of SEQ ID NO: 189 and a VL comprising the amino acid sequence of SEQ ID NO: 190;
(vi) a VH comprising the amino acid sequence of SEQ ID NO: 227 and a VL comprising the amino acid sequence of SEQ ID NO: 228;
(vii) a VH comprising the amino acid sequence of SEQ ID NO: 265 and a VL comprising the amino acid sequence of SEQ ID NO: 266;
(viii) a VH comprising the amino acid sequence of SEQ ID NO: 303 and a VL comprising the amino acid sequence of SEQ ID NO: 304; or
(ix) a VH comprising the amino acid sequence of SEQ ID NO: 341 and a VL comprising the amino acid sequence of SEQ ID NO: 342.

21. An antibody or fragment thereof that binds to CD16a, wherein the antibody or fragment thereof binds to both CD16a-F and CD16a-V.

22. The antibody or fragment thereof of claim 21, wherein the antibody or fragment thereof does not bind to CD16b.

23. The antibody or fragment thereof of claim 21 or 22, wherein the antibody or fragment thereof does not bind to CD16b-NA1 and/or CD16b-NA2.

24. A multispecific antibody or fragment thereof, comprising a first binding domain that binds to CD16a and a second binding domain that binds to a second antigen, wherein the first binding domain comprises an antibody or fragment thereof according to any one of claims 1 to 23.

25. The multispecific antibody or fragment thereof of claim 24, wherein the second antigen is expressed on a tumor cell.

26. The multispecific antibody or fragment thereof of claim 24 or 25, wherein the second antigen is CD19.

27. The multispecific antibody or fragment thereof of any one of claims 24 to 26, wherein the first binding domain comprises any one or more of (i)-(ix):
(i) a VH CDR1, a VH CDR2, and a VH CDR3 as set forth in a VH comprising the amino acid sequence of SEQ ID NO: 37 and a VL CDR1, a VL CDR2, and a VL CDR3 as set forth in a VL comprising the amino acid sequence of SEQ ID NO: 38;
(ii) a VH CDR1, a VH CDR2, and a VH CDR3 as set forth in a VH comprising the amino acid sequence of SEQ ID NO: 75 and a VL CDR1, a VL CDR2, and a VL CDR3 as set forth in a VL comprising the amino acid sequence of SEQ ID NO: 76;
(iii) a VH CDR1, a VH CDR2, and a VH CDR3 as set forth in a VH comprising the amino acid sequence of SEQ ID NO: 113 and a VL CDR1, a VL CDR2, and a VL CDR3 as set forth in a VL comprising the amino acid sequence of SEQ ID NO: 114;
(iv) a VH CDR1, a VH CDR2, and a VH CDR3 as set forth in a VH comprising the amino acid sequence of SEQ ID NO: 151 and a VL CDR1, a VL CDR2, and a VL CDR3 as set forth in a VL comprising the amino acid sequence of SEQ ID NO: 152;
(v) a VH CDR1, a VH CDR2, and a VH CDR3 as set forth in a VH comprising the amino acid sequence of SEQ ID NO: 189 and a VL CDR1, a VL CDR2, and a VL CDR3 as set forth in a VL comprising the amino acid sequence of SEQ ID NO: 190;
(vi) a VH CDR1, a VH CDR2, and a VH CDR3 as set forth in a VH comprising the amino acid sequence of SEQ ID NO: 227 and a VL CDR1, a VL CDR2, and a VL CDR3 as set forth in a VL comprising the amino acid sequence of SEQ ID NO: 228;
(vii) a VH CDR1, a VH CDR2, and a VH CDR3 as set forth in a VH comprising the amino acid sequence of SEQ ID NO: 265 and a VL CDR1, a VL CDR2, and a VL CDR3 as set forth in a VL comprising the amino acid sequence of SEQ ID NO: 266;
(vii) a VH CDR1, a VH CDR2, and a VH CDR3 as set forth in a VH comprising the amino acid sequence of SEQ ID NO: 303 and a VL CDR1, a VL CDR2, and a VL CDR3 as set forth in a VL comprising the amino acid sequence of SEQ ID NO: 304; or
(ix) a VH CDR1, a VH CDR2, and a VH CDR3 as set forth in a VH comprising the amino acid sequence of SEQ ID NO: 341 and a VL CDR1, a VL CDR2, and a VL CDR3 as set forth in a VL comprising the amino acid sequence of SEQ ID NO: 342; and
wherein the second binding domain comprises a VH CDR1, a VH CDR2, and a VH CDR3 as set forth in a VH comprising the amino acid sequence of SEQ ID NO: 343 and a VL CDR1, a VL CDR2, and a VL CDR3 as set forth in a VL comprising the amino acid sequence of SEQ ID NO: 344.

28. The multispecific antibody or fragment thereof of claim 27,
wherein the first binding domain comprises any one or more of (i)-(ix):
(i) a VH comprising the amino acid sequence of SEQ ID NO: 37 and a VL comprising the amino acid sequence of SEQ ID NO: 38;
(ii) a VH comprising the amino acid sequence of SEQ ID NO: 75 and a VL comprising the amino acid sequence of SEQ ID NO: 76;
(iii) a VH comprising the amino acid sequence of SEQ ID NO: 113 and a VL comprising the amino acid sequence of SEQ ID NO: 114;
(iv) a VH comprising the amino acid sequence of SEQ ID NO: 151 and a VL comprising the amino acid sequence of SEQ ID NO: 152;
(v) a VH comprising the amino acid sequence of SEQ ID NO: 189 and a VL comprising the amino acid sequence of SEQ ID NO: 190;
(vi) a VH comprising the amino acid sequence of SEQ ID NO: 227 and a VL comprising the amino acid sequence of SEQ ID NO: 228;
(vii) a VH comprising the amino acid sequence of SEQ ID NO: 265 and a VL comprising the amino acid sequence of SEQ ID NO: 266;
(viii) a VH comprising the amino acid sequence of SEQ ID NO: 303 and a VL comprising the amino acid sequence of SEQ ID NO: 304; or
(ix) a VH comprising the amino acid sequence of SEQ ID NO: 341 and a VL comprising the amino acid sequence of SEQ ID NO: 342; and
wherein the second binding domain comprises a VH comprising the amino acid sequence of SEQ ID NO: 343 and a VL comprising the amino acid sequence of SEQ ID NO: 344;

29. The multispecific antibody or fragment thereof of any one of claims 24-28, wherein the first binding domain and the second binding domain can be linked through a peptide bond or a peptide linker.

30. The multispecific antibody or fragment thereof of any one of claims 26-29, wherein the multispecific antibody or fragment thereof comprises one polypeptide chain comprising the first binding domain and the second binding domain, wherein the first binding domain comprises a first scFv that binds to CD16, and the second binding domain comprises a second scFv that binds to CD19.

31. The multispecific antibody or fragment thereof of claim 30, wherein the first scFv is linked to the N terminal of the second scFv.

32. The multispecific antibody or fragment thereof of claim 30, wherein the first scFv is linked to the C terminal of the second scFv.

33. The multispecific antibody or fragment thereof of any one of claims 30-32, wherein the polypeptide chain further comprises a CH2 and a CH3 of an IgG Fc region.

34. The multispecific antibody or fragment thereof of any one of claims 30-33, wherein the multispecific antibody or fragment comprises two identical polypeptide chains of claim 33.

35. The multispecific antibody or fragment thereof of claim 34, wherein the first polypeptide and the second polypeptide form a bivalent format through dimerization of the CH2 and/or CH3 regions.

36. An isolated nucleic acid comprising a nucleic acid sequence encoding the antibody of any one of claims 1-23, or the multispecific antibody of any one of claims 24-35.

37. A vector comprising the isolated nucleic acid of claim 36.

38. A pharmaceutical composition, comprising the antibody of any one of claims 1-23, the multispecific antibody of any one of claims 24-35, or the vector of claim 37, and a pharmaceutically acceptable excipient.

39. A method of treating a disease or disorder in a subject, comprising administering to the subject an effective amount of the antibody of any one of claims 1-23, the multispecific antibody of any one of claims 24-35, or the vector of claim 37, or the pharmaceutical composition of claim 38.

40. The method of claim 39, wherein the disease or disorder is a cancer.

41. The method of claim 39, wherein the disease or disorder is leukemia or lymphoma.

42. A method of activating NK cell activity in a subject, comprising administering to the subject an effective amount of the antibody of any one of claims 1-23, the multispecific antibody of any one of claims 24-35, or the vector of claim 37, or the pharmaceutical composition of claim 38.

43. A method of activating NK cell activity without activating neutrophils in a subject, comprising administering to the subject an effective amount of the antibody of any one of claims 1-23, the multispecific antibody of any one of claims 24-35, or the vector of claim 37, or the pharmaceutical composition of claim 38.

44. The method of any one of claims 39 to 43, wherein the subject has CD16a-F/F, CD16a-F/V, or CD16a-V/V genotype.

45. The method of any one of claims 39 to 44, wherein the subject has CD16b-NA1/CD16b-NA2, CD16b-NA1/CD16b-NA1, or CD16b-NA2/CD16b-NA2 genotype.

46. An antibody or fragment thereof which specifically recognizes an epitope comprising the residue Asp 82 of human CD16a with an affinity for CD16a at least 10-fold higher than for CD16b.
